(19) United States
(12) Patent Application Publication
(10) Pub. No.: US 2024/0374761 A1
(43) Pub. Date: Nov. 14, 2024

(54) ANIMAL MODEL HAVING HOMOLOGOUS RECOMBINATION OF MOUSE PTH1 RECEPTOR

(71) Applicants: RADIUS PHARMACEUTICALS, INC., Boston, MA (US); THE GENERAL HOSPITAL CORPORATION, Boston, MA (US)

(72) Inventors: Beate Klara Maria MANNSTADT, Arlington, MA (US); Thomas James GARDELLA, Needham, MA (US)

(21) Appl. No.: 18/289,444

(22) PCT Filed: May 5, 2022
(86) PCT No.: PCT/US2022/027864

§ 371 Date: Nov. 3, 2023

Related U.S. Application Data
(60) Provisional application No. 63/184,688, filed on May 5, 2021.
(60) Provisional application No. 63/250,647, filed on Sep. 30, 2021.

(51) Int. Cl. A61K 49/00; A01K 67/0278

(52) U.S. Cl. CPC A61K 49/0008; A01K 67/0278; A01K 2217/072; A01K 2227/105; A01K 2267/03

(57) ABSTRACT

New transgenic non-human animals comprising human Parathyroid Hormone 1 Receptor (hPTH1R), and methods of producing the same; new assays and screening techniques to evaluate the hPTH1R; and methods and transgenic animals to evaluate the response of hPTH1R to one or more candidate agents.

### CROSS REFERENCE TO RELATED APPLICATIONS

This application claims the benefit of, and priority to, U.S. Provisional Application Ser. No. 63/250,647, filed on Sep. 30, 2021, and U.S. Provisional Application Ser. No. 63/184,688, filed on May 5, 2021. The entire contents of the aforementioned applications are incorporated herein.

### SEQUENCE

This application incorporates by reference in its entirety the Sequence Listing entitled “265853-507889_ST25.txt” (46 KB), which was created on Apr. 20, 2022, at 11:58 AM, and filed electronically herewith.

### TECHNICAL FIELD

The present disclosure provides transgenic non-human animals, and methods of producing the same; new assays and screening techniques to evaluate the human Parathyroid Hormone 1 Receptor (hPTH1R), and methods to screen candidate therapeutic agents for the treatment of hPTH1R-related disorders.

### BACKGROUND

Parathyroid hormone 1 receptor (PTH1R), and its ligands, e.g., parathyroid hormone (PTH) and parathyroid hormone-related peptide (PTHrP), have been implicated in a variety of important roles in both development and mineral ion homeostasis. See B. Lanske and H. M. Kronenberg, Parathyroid hormone-related peptide (PTHrP) and parathyroid hormone (PTH)/PTHrP receptor. Crit Rev Eukaryot Gene Expr. 1998; 8(3-4):297-320; M. Mannstadt, H. Jüppner, and T. J. Gardella, Receptors for PTH and PTHrP: their biological importance and functional properties. Am J Physiol. 1999 November; 277(5):F665-75; H. Jüppner, Molecular cloning and characterization of a parathyroid hormone/parathyroid hormone-related peptide receptor: a member of an ancient family of G protein-coupled receptors. Curt Opin Nephrol Hypertens. 1994 July; 3(4):371-8; and T. J. Gardella and H. Jüppner, Interaction of PTH and PTHrP with their receptors. Rev Endocr Metab Disord. 2000 November; 1(4):317-29.

Defects in the normal function of PTH1R, and/or deleterious changes to the expression of one or more of its ligands, can result in developmental disorders and/or dysregulation of mineral ion homeostasis; consequently, such defects and/or dysregulation can have severe health consequences.

Evaluating the role and function of proteins can be accomplished using animal models. Generating an animal model with which to investigate the role of PTH1R could be used to evaluate the molecular and cellular underpinnings of PTH1R function. However, in some cases, the animal selected as the model organism has an endogenous protein that does not behave in an equivalent manner to the human protein (e.g., the endogenous animal protein does not respond to drugs in a manner similar to the human protein).

Transgenic animal technology presents a unique opportunity to study the characteristics of human proteins in non-human animals. Recombinant DNA and genetic engineering techniques have made it possible to introduce and express a desired sequence or gene in a recipient animal making it possible to study the effects of a particular molecule in vivo and study agents that bind to the molecule.

Accordingly, developing novel methods, models, and materials with which to evaluate the function of PTH1R is of great importance.

### SUMMARY

The present disclosure describes a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein.

In addition, the present disclosure describes a non-human recombinant cell comprising: a heterologous polynucleotide comprising human Parathyroid Hormone 1 Receptor (hPTH1R) exons 4 to 16, wherein said heterologous polynucleotide is operable to encode a human PTH1R protein.

In addition, the present disclosure describes a vector comprising: (i) a heterologous polynucleotide comprising a first nucleotide sequence comprising a coding sequence for human Parathyroid Hormone 1 Receptor (hPTH1R) exons 4 to 16 and second nucleotide sequence comprising a polyadenylation signal; (ii) a 5′-homology arm, and a 3′-homology arm, wherein said 5′-homology arm and said 3′-homology arm are located upstream and downstream of the heterologous polynucleotide, respectively; (iii) a third nucleotide sequence operable to encode a diphtheria toxin A protein, or fragment thereof; and a fourth nucleotide sequence operable to encode an neomycin phosphotransferase II (Neo); (iv) an upstream self-deletion anchor (SDA) nucleotide sequence, and a downstream SDA nucleotide sequence; wherein said upstream SDA nucleotide sequence and downstream SDA nucleotide sequences flank the fourth nucleotide sequence; wherein said vector is operable to allow a homologous recombination-mediated integration of the heterologous polynucleotide into an endogenous non-human animal PTH1R gene locus; and wherein said homologous recombination-mediated integration results in a replacement of an endogenous non-human animal genomic DNA segment with the heterologous polynucleotide.

In addition, the present disclosure describes a method of making a transgenic non-human animal comprising: (i) introducing a heterologous polynucleotide comprising human PTH1R exons 4 to 16 into a non-human animal embryonic stem (ES) cell, such that the heterologous polynucleotide integrates into an endogenous non-human animal PTH1R locus; (ii) obtaining a non-human animal ES cell comprising a modified genome, wherein the heterologous polynucleotide has integrated into an endogenous non-human animal PTH1R locus; and (iii) generating a non-human animal using the non-human animal ES cell comprising the modified genome.

In addition, the present disclosure describes an assay to identify a candidate agent that modulates the activity or function of a human PTH1R protein (hPTH1R), comprising: (a) obtaining an experimental animal or a cell therefrom; wherein said experimental animal is a transgenic non-human animal having a heterologous polynucleotide comprising human PTH1R exons 4 to 16 that is operable to encode a hPTH1R; and wherein said experimental animal or a cell therefrom is operable to express the hPTH1R; (b) admixing the candidate agent with the hPTH1R present in the experimental animal or cell therefrom; (c) measuring whether said candidate agent modulates the activity or function of said hPTH1R, wherein a modulation in the activity or function of said hPTH1R in the presence of said candidate agent, as compared to the activity or function of said hPTH1R that is not exposed said candidate agent, is indicative that said candidate agent modulates the activity or function of said hPTH1R.

In addition, the present disclosure describes a transgenic mouse comprising a heterologous polynucleotide comprising human Parathyroid Hormone 1 Receptor (hPTH1R) exons 4 to 16; wherein said heterologous polynucleotide is operable to encode a human PTH1R protein having an amino acid sequence as set forth in SEQ ID NO: 1.

In addition, the present disclosure describes a transgenic mouse comprising a heterologous polynucleotide comprising human Parathyroid Hormone 1 Receptor (hPTH1R) exons 4 to 16; wherein said heterologous polynucleotide is operable to encode a human PTH1R protein having an amino acid sequence as set forth in SEQ ID NO: 29.

In addition, the present disclosure describes a non-human recombinant cell comprising: a heterologous polynucleotide comprising human Parathyroid Hormone 1 Receptor (hPTH1R) exons 4 to 16, wherein said heterologous polynucleotide is operable to encode a human PTH1R protein having an amino acid sequence as set forth in SEQ ID NO: 1.

In addition, the present disclosure describes a non-human recombinant cell comprising: a heterologous polynucleotide comprising human Parathyroid Hormone 1 Receptor (hPTH1R) exons 4 to 16, wherein said heterologous polynucleotide is operable to encode a human PTH1R protein having an amino acid sequence as set forth in SEQ ID NO: 29.

### BRIEF DESCRIPTION OF THE DRAWINGS

FIG. 1 shows a diagram depicting the targeting strategy. The top row shows the wild type mouse allele having 16 exons, and homology arm regions (shown as black regions). The next row shows the targeting vector. Here “DTA”=diphtheria toxin A, and “Neo′”=neomycin phosphotransferase II. The left-pointing chevrons indicate self-deletion anchor (SDA) sites. The next row shows the targeted allele after recombination with the vector, followed by constitutive knock-in allele subsequent to deletion of the positive selection marker (Neo′). The heterologous polynucleotide encoding human PTH1R exons 4 to 16 and HA tag is shown as the dotted box. “HA” refers to a human influenza hemagglutinin (HA) epitope tag.

FIG. 2 depicts a diagram showing the heterozygous genotyping strategy to assess and confirm successful integration of the polynucleotide encoding human Parathyroid Hormone 1 Receptor (hPTH1R) exons 4 to 16. “Neo′”=neomycin phosphotransferase II. Left-pointing chevrons indicate self-deletion anchor (SDA) sites. Homology arm regions are indicated as black horizontal bars along the allele. The heterologous polynucleotide encoding human PTH1R exons 4 to 16 and HA tag is shown as a dotted box. UTR=untranslated region. KO=knock-out. Primers are indicated with black arrows. “HA” refers to a human influenza hemagglutinin (HA) epitope tag.

FIG. 3 shows PCR gels confirming the successful knock-in of a nucleotide sequence comprising a coding sequence for human Parathyroid Hormone 1 Receptor (hPTH1R) exons 4 to 16. Each lane of the PCR gel indicates a mouse pup number, or a control. “M”=Marker; “ESC”=embryonic stem cell; “WT”=wild-type. For the markers, the smallest bp fragment is 100 bp, with fragments every 100 bp. Mouse pup numbers are indicated on the top of the gels. The top gel shows results from pups derived from ES clone 1A6; the bottom gel shows pups derived from ES clone 1F11. Here, pups 5 #, 8 #, 9 #, 13 # and 14 #(top gel) from clone 1A6 are positive for successful knock-in. Likewise, the bottom gel shows successful knock-in of the transgene in pups 5 #, 7 #, 11 # and 14 #, derived from clone 1F11.

FIG. 4 shows PCR gels confirming the presence of the wild type mouse Pth1r gene. Each lane of the PCR gel indicates a mouse pup number, or a control. “M”=Marker; “ESC”=embryonic stem cell; “WT”=wild-type. The DNA ladder has a smallest bp fragment of 100 bp, with fragments every 100 bp. Mouse pup numbers are indicated on the top of the gels. The top gel shows results from pups derived from ES clone 1A6; the bottom gel shows pups derived from ES clone 1F11. Here, all the pups are positive for the WT allele.

FIG. 5. shows PCR gels confirming the successful deletion of the Neo cassette in heterozygous animals. Each lane of the PCR gel indicates a mouse pup number, or a control. “M”=Marker; “ESC”=embryonic stem cell; “WT”=wild-type. The marker lane shows a DNA ladder with the smallest bp fragment of 100 bp, with fragments every 100 bp. The gel shows the results of pups derived from ES clone 1A6; the bottom gel shows pups derived from ES clone 1F11. Here, pups 5 #, 8 #, 9 #, 13 # and 14 #(top gel) from clone 1A6 are positive for successful Neo cassette deletion. Likewise, the bottom gel shows successful Neo cassette deletion in pups 5 #, 7 #, 11 # and 14 #, derived from clone 1F11.

FIG. 6 depicts a diagram showing the homozygous genotyping strategy to assess and confirm successful integration of the polynucleotide encoding human Parathyroid Hormone 1 Receptor (hPTH1R) exons 4 to 16. “Neo′”=neomycin phosphotransferase II. The left-pointing chevrons indicate self-deletion anchor (SDA) sites. Homology arm regions are indicated as black bars. The heterologous polynucleotide encoding human Parathyroid Hormone 1 Receptor (hPTH1R) exons 4 to 16 operably linked to a human influenza hemagglutinin (HA) epitope tag is indicated as a dotted box. “rBG PA” refers to rabbit β-globin polyadenylation signal—a sequence that allows transcription termination and polyadenylation of mRNA. Primers are shown as arrows and are F1, RI; F4, R2; F3, R1. “HA” refers to a human influenza hemagglutinin (HA) epitope tag.

FIG. 7 shows PCR gels confirming the successful knock-in of a nucleotide sequence comprising a coding sequence for human Parathyroid Hormone 1 Receptor (hPTH1R) exons 4 to 16 in homozygous mice. Each lane of the PCR gel indicates a mouse pup number, or a control. “M”=Marker; “ESC”=embryonic stem cell; “WT”=wild-type. The marker lane shows a DNA ladder with fragment sizes indicated to the left of the gel. Pup numbers are indicated on the top of the gels. Here, pups 43 #, 45 #, 46 #, 48 # and 50 # from clone 1A6 are positive for successful knock-in.

FIG. 8 shows PCR gels confirming the presence of the wild type mouse Pth1r gene. Each lane of the PCR gel indicates a mouse pup number, or a control. “M”=Marker; “ESC”=embryonic stem cell; “WT”=wild-type. The marker lane shows a DNA ladder with fragment sizes indicated to the left of the gel. Pup numbers are indicated on the top of the gels. Here, pups 43 #, 45 #, 46 #, 48 # and 50 # from clone 1A6 are do not show the presence of the expected 207 bp PCR product, indicating the corresponding pups are homozygous.

FIG. 9. shows PCR gels confirming the successful deletion of the Neo cassette in heterozygous animals. Each lane of the PCR gel indicates a mouse pup number, or a control. “M”=Marker; “ESC”=embryonic stem cell; “WT”=wild-type. The marker lane shows a DNA ladder with fragment sizes indicated to the left of the gel. Pup numbers are indicated on the top of the gels. Here, pups 43 #, 45 #, 46 #, 48 # and 50 # from clone 1A6 show the expected 407 bp PCR product, indicating successful Neo cassette deletion.

FIG. 10 shows the PCR results for 3′ junction region analysis. The lanes, from left to right, show the following samples; and expected band size (in parentheses): C57BL-KI-hP1R-1-15 (407 bp); C57BL-KI-hP1R-2-16 (407 bp); C57BL-WT-1 (none); C57BL-WT-1 (none); CD1-KI-hP1R-XL130 (407 bp); Ladder. The expected PCR product size (in base pairs, “bp”) corresponds with the results shown in the gel.

FIG. 11 shows a CLUSTAL alignment of the consensus F2-R1 sequence and the six original DNA sequences obtained from the three knock-in mice comprising the heterologous polynucleotide operable to encode hPTH1R exons 4-16, for the F2-R1 PCR product. Also included is mouse Intron-4 sequence, which aligns with the 3′ ends of the sequences obtained from the KI mice.

FIG. 12 depicts a schematic showing the hPTH1R knock-in genome along with the location of the F4 and R-291 primer sites. The heterologous polynucleotide encoding human Parathyroid Hormone 1 Receptor (hPTH1R) exons 4 to 16 operably linked to a human influenza hemagglutinin (HA) epitope tag is indicated as a dotted box. The left-pointing chevrons indicate self-deletion anchor (SDA) sites. Homology arm regions are indicated as black bars. “rBG PA” refers to rabbit β-globin polyadenylation signal—a sequence that allows transcription termination and polyadenylation of mRNA. Primers are shown as arrows and are F3, R2, R-291; F2, R1.

FIG. 13 shows a PCR gel providing the results of an analysis of the DNA sequence of the HA-tag hPTH1R region. Here, the lanes and expected product size (in parentheses) are as follows: Ladder; C57BL-KI-hP1R-1-15 (928 bp); C57BL-KI-hP1R-2-16 (928 bp); CD1-KI-hP1R-XL130 (928 bp); Ladder. As shown here, the PCR yielded two products: one product around 900 bp, and the other product around 400 bp.

FIG. 14 shows the a CLUSTAL O protein alignment of the translated consensus sequence (Consensus.F3_R-291 Sequence.vers 3); the human PTH1R protein consensus sequence above translated into an amino acid sequence, and compared with the amino acid sequences of the hPTHR1-HA and mouse PTHR1 proteins. The HA tag (YPYDVPDYA) is highlighted in bold, and residues unique to the WT mouse PTH1R protein are highlighted in blue. None of the residues unique to the WT mouse PTH1R protein were found in the translated consensus sequence. Asterisks (“*”) indicate matching residues in all sequences; colons (“:”) indicate conserved changes.

FIG. 15 shows a CLUSTAL O Alignment of DNA sequences obtained in three sequencing reactions (Rxns-1, -5 and -9), performed using primer F4 and F4-R291 PCR products generated from three hPTH1R-KI mice, and the consensus sequence (con.Rxns.1.5.9_F4) derived from those three sequences. The letter “N” indicates a position that is not determined. Asterisks (“*”) indicate matching residues in all sequences.

FIG. 16 shows a diagram providing a comparison of the WT and hPTH1R-KI mouse. Top: shows a schematic of the mouse PTH1R gene (NCBI Reference Sequence: NM_011199.2) located on chromosome 9 and containing 16 exons that either protein-coding (filled boxes) or non-coding (open boxes). Center: the region of the wild-type (WT) mouse PTH1R gene targeted for homologous recombination. Bottom: the corresponding region of the knock-in (KI) allele in which exon 4 and a 5′ end portion of intron 4 is replaced by a cassette containing the cDNA for hPTH1R residues Val-26 to Met-593, followed by a TGA stop codon, and a transcription termination/polyadenylation sequence from the rabbit beta globin gene (rbgPA); not shown is a short (143 bp) segment between the rbgPA and the 3′ junction site in intron 4 that is derived from the self-deleting anchor of the targeting vector used for neomycin gene excision. The 5′ junction site is at the 3′ end of mouse intron 3, such that removal of intron 3 by mRNA splicing joins exon 3 encoding the Metl-Leu25 portion of the mouse PTH1R protein to the human PTH1R cDNA sequence at the codon for Val26. The expressed PTH1R protein contains a signal sequence, Metl-Ala22, derived from mouse exon 3. Removal of the signal sequence during processing leaves three residues Tyr23-Ala22-Leu25, at the N-terminus of the protein that are derived from the mouse gene, but as these three residues are identical in mouse and human receptors, the expressed and processed PTH1R is fully identical to the native mature human PTH1R, except for residues 88-96 which are replaced by an HA tag.

FIG. 17 shows a primer map of the knock-in polynucleotide sequence and positions of primers used for PCR and Sanger sequence analysis (nucleotide position 1-900).

FIG. 18 shows a primer map of the knock-in polynucleotide sequence and positions of primers used for PCR and Sanger sequence analysis (nucleotide position 901-1740).

FIG. 19 shows a primer map of the knock-in polynucleotide sequence and positions of primers used for PCR and Sanger sequence analysis (nucleotide position 1741-2600).

FIG. 20 shows an alignment of mouse PTH1R and the human PTH1R-HA protein sequences. Asterisks below the lines indicate identical amino acids.

FIG. 21 depicts a Western Blot analysis of hPTH1R in kidneys isolated from 5-month-old hPTH1R-KI and WT mice. Kidneys isolated from two wild-type mice (WT-1, WT-2) and two mice transformed with a heterologous polynucleotide comprising human Parathyroid Hormone 1 Receptor (hPTH1R) exons 4 to 16 (hereinafter “hPTH1R-KI” mice). The two hPTH1R-KI mice (lanes 1 and 2 underneath “Ki”) and two WT mice (lanes 1 and 2 underneath “WT”) were analyzed by SDS gel electrophoresis and western blotting. Panel (A) shows the gel stained with anti-HA antibody. Panel (B) shows the gel stained with anti-Glyceraldehyde-3-phosphate dehydrogenase (GAPDH) antibody for sample loading control. Panel (C) shows a higher magnification copy of the anti-HA antibody stain of Panel (A), and a duplicate gel stained for GAPDH antibody for comparison below.

FIG. 22 shows the body weight of WT and hPTH1R-KI mice over time. Body weight was observed in WT and hPTH1R-KI mice at 8, 16, 24, and 56 weeks of age. The body weights of hPTH1R-KI mice did not substantially differ from WT controls, supporting the notion that the heterologous polynucleotide comprising human Parathyroid Hormone 1 Receptor (hPTH1R) exons 4 to 16 functions appropriately when knocked-in to a transgenic mouse. Data is shown as mean standard error (SE).

FIG. 23 shows the representative sagittal views of the distal femur in 6-month-old wild-type (WT) and hPTH1R-KI (KI) mice. Femurs were isolated from the mice at 26 weeks of age and analyzed by pCT.

FIG. 24 shows the quantification of bone parameters from pCT in 6-month-old wild-type (WT) and hPTH1R-KI mice (males and females combined). Parameters shown here are total femur length, and trabecular bone at the distal metaphysis as bone volume relative to tissue volume (BV/TV, %). ♀=female; ♂=male, and are labeled with corresponding mouse identification number. Data is shown as mean±standard error (SE).

FIG. 25 shows the quantification of trabecular thickness and trabecular spacing, as determined via pCT, in 6-month-old WT and hPTH1R-KI mice (males and females combined). ♀=female; ♂=male, and are labeled with corresponding mouse identification number. Data is shown as mean standard error (SE).

FIG. 26 shows the quantification of trabecular number and cortical area over total area, as determined via pCT, in 6-month-old WT and hPTH1R-KI mice (males and females combined). ♀=female; ♂=male, and are labeled with corresponding mouse identification number. Data is shown as mean standard error (SE).

FIG. 27 shows the quantification of cortical thickness and cortical porosity, as determined via pCT, in 6-month-old WT and hPTH1R-KI mice (males and females combined). ♀=female; ♂=male, and are labeled with corresponding mouse identification number. Here, cortical bone thickness was slightly greater in the hPTH1R-KI mice (P=0.049). Data is shown as mean standard error (SE).

FIG. 28 shows the representative sagittal views of the distal femur in 13-month-old wild-type (WT) and hPTH1R-KI (KI) mice. Femurs were isolated from the mice at 13 months of age and analyzed by pCT.

FIG. 29 shows the quantification of trabecular BV/TV and trabecular number, as determined via pCT, in 13-month-old WT and hPTH1R-KI mice (males and females combined). ♀=female; ♂=male, and are labeled with corresponding mouse identification number. Data is shown as mean standard error (SE).

FIG. 30 shows the quantification of trabecular thickness and trabecular spacing, as determined via pCT, in 13-month-old WT and hPTH1R-KI mice (males and females combined). ♀=female; ♂=male, and are labeled with corresponding mouse identification number. Data is shown as mean standard error (SE).

FIG. 31 shows the quantification of cortical area over total area, and cortical thickness, as determined via pCT, in 13-month-old WT and hPTH1R-KI mice (males and females combined). ♀=female; ♂=male, and are labeled with corresponding mouse identification number. Data is shown as mean standard error (SE).

FIG. 32 shows the quantification of cortical porosity, as determined via pCT, in 13-month-old WT and hPTH1R-KI mice (males and females combined). ♀=female; ♂=male, and are labeled with corresponding mouse identification number. Data is shown as mean±standard error (SE).

FIG. 33 shows the quantification of femur length and trabecular BV/TV, as determined via μCT, in 6-month-old female WT and hPTH1R-KI mice. Data is shown as mean±standard error of the mean (SEM).

FIG. 34 shows the quantification of trabecular number and trabecular thickness, as determined via μCT, in 6-month-old female WT and hPTH1R-KI mice. Data is shown as mean standard error of the mean (SEM).

FIG. 35 shows the quantification of trabecular spacing and cortical area over total area, as determined via μCT, in 6-month-old female WT and hPTH1R-KI mice. Data is shown as mean standard error of the mean (SEM).

FIG. 36 shows the quantification of cortical thickness and cortical porosity, as determined via μCT, in 6-month-old female WT and hPTH1R-KI mice. Data is shown as mean±standard error of the mean (SEM).

FIG. 37 shows the quantification of femur length and trabecular BV/TV, as determined via μCT, in 6-month-old male WT and hPTH1R-KI mice. Data is shown as mean standard error of the mean (SEM).

FIG. 38 shows the quantification of trabecular number and trabecular thickness, as determined via μCT, in 6-month-old male WT and hPTH1R-KI mice. Data is shown as mean standard error of the mean (SEM).

FIG. 39 shows the quantification of trabecular spacing and cortical area over total area, as determined via μCT, in 6-month-old male WT and hPTH1R-KI mice. Data is shown as mean standard error of the mean (SEM).

FIG. 40 shows the quantification of cortical thickness and cortical porosity, as determined via μCT, in 6-month-old male WT and hPTH1R-KI mice. Data is shown as mean standard error of the mean (SEM).

FIG. 41 shows the quantification of femur length and trabecular BV/TV, as determined via μCT, in 13-month-old female WT and hPTH1R-KI mice. Data is shown as mean±standard error of the mean (SEM).

FIG. 42 shows the quantification of trabecular number and trabecular thickness, as determined via μCT, in 13-month-old female WT and hPTH1R-KI mice. Data is shown as mean standard error of the mean (SEM).

FIG. 43 shows the quantification of trabecular spacing and cortical area over total area, as determined via μCT, in 13-month-old female WT and hPTH1R-KI mice. Data is shown as mean standard error of the mean (SEM).

FIG. 44 shows the quantification of cortical thickness and cortical porosity, as determined via μCT, in 13-month-old female WT and hPTH1R-KI mice. Data is shown as mean±standard error of the mean (SEM).

FIG. 45 shows the quantification of femur length and trabecular BV/TV, as determined via μCT, in 13-month-old male WT and hPTH1R-KI mice. Data is shown as mean±standard error of the mean (SEM).

FIG. 46 shows the quantification of trabecular number and trabecular thickness, as determined via μCT, in 13-month-old male WT and hPTH1R-KI mice. Data is shown as mean standard error of the mean (SEM).

FIG. 47 shows the quantification of trabecular spacing and cortical area over total area, as determined via μCT, in 13-month-old male WT and hPTH1R-KI mice. There was a slight increase in cortical area over total area in the male hPTH1R-KI mice relative to male WT mice (P=0.017). Data is shown as mean standard error of the mean (SEM).

FIG. 48 shows the quantification of cortical thickness and cortical porosity, as determined via μCT, in 13-month-old male WT and hPTH1R-KI mice. Data is shown as mean±standard error of the mean (SEM).

FIG. 49 depicts a μCT 3D reconstruction of the side and superior views of skulls from WT and hPTH1R-KI mice at age 6 months. Here, three representative mice from the WT and hPTH1R-KI groups are shown. The top row shows CT images of skulls obtained from WT mice. The images of the WT skulls were obtained from two males: 1 WTM1 and 2 WTM2; and one female: 4 WTF1. The bottom row (“hP1R-ki”) shows the transgenic mice comprising a heterologous polynucleotide comprising human Parathyroid Hormone 1 Receptor (hPTH1R) exons 4 to 16, wherein said heterologous polynucleotide is operable to encode a human PTH1R protein. The transgenic hPTH1R-KI mice skull images on the bottom row were obtained from two males: 1 hP1RM1 and 2 hP1RM1; and one female: 6 hP1RF1.

FIG. 50 depicts the results of the biomarker analysis showing the levels of serum calcium (“Ca”) the serum of 5-month-old WT and hPTH1R-KI mice. Serum samples obtained from wild-type (WT) and hPTH1R-KI (KI) mice and were analyzed for serum calcium (“Ca”). ♀=female; ♂=male, and are labeled with corresponding mouse identification number. Data is shown as mean standard error (SE). There was no significant difference (P<0.05) between WT and hPTH1R-KI mice.

FIG. 51 depicts the results of the biomarker analysis showing inorganic phosphorous (Pi) levels in the serum of 5-month-old WT and hPTH1R-KI mice. ♀=female; ♂=male, and are labeled with corresponding mouse identification number. Data is shown as mean±standard error (SE). There was no significant difference (P<0.05) between WT and hPTH1R-KI mice.

FIG. 52 depicts the results of the biomarker analysis showing the ratio of calcium to creatinine (Ca/Cre) in the urine of 5-month-old WT and hPTH1R-KI mice. ♀=female; ♂=male, and are labeled with corresponding mouse identification number. Data is shown as mean standard error (SE). There was no significant difference (P<0.05) between WT and hPTH1R-KI mice.

FIG. 53 depicts the results of the biomarker analysis showing the ratio of inorganic phosphorous to creatinine (Pi/Cre) in the urine of 5-month-old WT and hPTH1R-KI mice. ♀=female; ♂=male, and are labeled with corresponding mouse identification number. Data is shown as mean standard error (SE). There was no significant difference (P<0.05) between WT and hPTH1R-KI mice.

FIG. 54 depicts the results of the biomarker analysis showing the levels of CTX-1 (i.e., C-terminal telopeptides of type I collagen, or the degradation products therefrom) in the serum of 5-month-old WT and hPTH1R-KI mice. ♀=female; ♂=male, and are labeled with corresponding mouse identification number. Data is shown as mean±standard error (SE). There was no significant difference (P<0.05) between WT and hPTH1R-KI mice.

FIG. 55 depicts the results of the biomarker analysis showing the levels of PINP (N-terminal propeptide of type I procollagen) in the serum of 5-month-old WT and hPTH1R-KI mice. ♀=female; ♂=male, and are labeled with corresponding mouse identification number. Data is shown as mean standard error (SE). Here, serum levels of P1NP differed (P<0.05; Student's T test) between WT and hPTH1R-KI mice; however, the difference was marginal.

FIG. 56 depicts the results of the biomarker analysis showing the levels of PTH(1-84) in the serum of 5-month-old WT and hPTH1R-KI mice. ♀=female; ♂=male, and are labeled with corresponding mouse identification number. Data is shown as mean±standard error (SE). There was no significant difference (P<0.05) between WT and hPTH1R-KI mice.

FIG. 57 depicts the results of the biomarker analysis showing the levels of 1,25-Dihydroxy Vitamin D (“1.25 VitD”) in the serum of 5-month-old WT and hPTH1R-KI mice. ♀=female; ♂=male, and are labeled with corresponding mouse identification number. Data is shown as mean standard error (SE). There was no significant difference (P<0.05) between WT and hPTH1R-KI mice.

FIG. 58 depicts the results of the biomarker analysis showing the levels of calcium (Ca) in the serum of 13-month-old WT and hPTH1R-KI mice. ♀=female; ♂=male, and are labeled with corresponding mouse identification number. Data is shown as mean±standard error (SE). There was no significant difference (P<0.05) between WT and hPTH1R-KI mice.

FIG. 59 depicts the results of the biomarker analysis showing the levels of inorganic phosphorous (Pi) in the serum of 13-month-old WT and hPTH1R-KI mice. ♀=female; ♂=male, and are labeled with corresponding mouse identification number. Data is shown as mean standard error (SE). There was no significant difference (P<0.05) between WT and hPTH1R-KI mice.

FIG. 60 depicts the results of the biomarker analysis showing the ratio of calcium to creatinine (Ca/Cre) in the urine of 13-month-old WT and hPTH1R-KI mice. ♀=female; ♂=male, and are labeled with corresponding mouse identification number. Data is shown as mean standard error (SE). There was no significant difference (P<0.05) between WT and hPTH1R-KI mice.

FIG. 61 depicts the results of the biomarker analysis showing the ratio of inorganic phosphorous to creatinine (Pi/Cre) in the urine of 13-month-old WT and hPTH1R-KI mice. ♀=female; ♂=male, and are labeled with corresponding mouse identification number. Data is shown as mean standard error (SE). The double-asterisk (“**”) indicates a P-value <0.05 (Student's T test).

FIG. 62 depicts the results of the biomarker analysis showing the levels of CTX-1 (i.e., C-terminal telopeptides of type I collagen, or the degradation products therefrom) in the serum of 13-month-old WT and hPTH1R-KI mice. ♀=female; ♂=male, and are labeled with corresponding mouse identification number. Data is shown as mean±standard error (SE). There was no significant difference (P<0.05) between WT and hPTH1R-KI mice.

FIG. 63 depicts the results of the biomarker analysis showing the levels of PINP (N-terminal propeptide of type I procollagen) in the serum of 13-month-old WT and hPTH1R-KI mice. ♀=female; ♂=male, and are labeled with corresponding mouse identification number. Data is shown as mean standard error (SE). Here, serum levels of P1NP differed (P<0.05; Student's T test) between WT and hPTH1R-KI mice; however, the difference was marginal.

FIG. 64 depicts the results of the biomarker analysis showing the levels of PTH(1-84) in the serum of 13-month-old WT and hPTH1R-KI mice. ♀=female; ♂=male, and are labeled with corresponding mouse identification number. Data is shown as mean standard error (SE). The double-asterisk (“**”) indicates a P-value <0.05 (Student's T test).

FIG. 65 depicts the results of the biomarker analysis showing the levels of 1,25-Dihydroxy Vitamin D (“1.25 VitD”) in the serum of 13-month-old WT and hPTH1R-KI mice. ♀=female; ♂=male, and are labeled with corresponding mouse identification number. Data is shown as mean standard error (SE). There was no significant difference (P<0.05) between WT and hPTH1R-KI mice.

FIG. 66 shows serum blood urea nitrogen (BUN) levels in 6-month-old (left) and 13-month-old (right) WT and hPTH1R-KI mice. ♀=female; ♂=male, and are labeled with corresponding mouse identification number. Data is shown as mean standard error (SE).

FIG. 67 depicts a graph showing the responses to PTH ligand analog injection in 10-week-old wild-type (WT) C57BL/6 (left) and homozygous hPTHR1-KI mice (right). Blood ionized calcium (Ca⁺⁺) levels were measured just prior to injection (t=0) and at 1, 2, 4 and 8 hours after injection after subcutaneous injection with vehicle (5 mM citrate, 150 mM NaCl, 0.05% Tween80, pH 5.0) or vehicle containing either PTH(1-34), PTHrP(1-36), or abaloparatide (ABL), with each peptide at a dose of 40 nmol/kg of body weight. At each time point, blood was collected from the tail and measured immediately for Ca⁺⁺ and pH using a Siemens model 348 blood analyzer. The blood Ca⁺⁺ values are plotted as means±SEM of 10 values obtained from two replicate experiments, each with five mice per group, and were statistically analyzed by Student's test (P vs. vehicle: *, <0.05; **, <0.001; P vs. PTH(1-34): #, <0.05, ##<0.01).

FIG. 68 depicts a graph showing the responses to PTH ligand analog injection in 10-week-old wild-type (WT) C57BL/6 (left) and homozygous hPTHR1-KI mice (right). Serum inorganic phosphorus (Pi) levels were measured just prior to injection (t=0) and at 1, 2, 4 and 8 hours after injection after subcutaneous injection with vehicle (5 mM citrate, 150 mM NaCl, 0.05% Tween80, pH 5.0) or vehicle containing either PTH(1-34), PTHrP(1-36), or abaloparatide (ABL), with each peptide at a dose of 40 nmol/kg of body weight. At each time point, blood was collected from the tail and measured immediately. The Pi values are plotted as means±SEM of 5 values obtained from a single experiment with five mice per group, and were statistically analyzed by Student's test (P vs. vehicle: *, <0.05; **, <0.001; P vs. PTH(1-34): #, <0.05, ##<0.01).

FIG. 69 shows the response of antagonist in 3-month-old hPTH1R-KI and wild-type (WT) mice. Blood ionized calcium (Ca⁺⁺) levels were measured in 3-month-old WT mice (A) and hPTH1R-KI mice (B), just prior to injection (t=0) and at 1, 2, and 4-hours after subcutaneous (SC) injection with either (1) vehicle, (2) PTH(1-34) alone at a dose of 40 nmol/kg, or (3) PTH(1-34) at 40 nmol/kg together with an antagonist peptide, i.e., LA-PTH(7-36) or [Leu¹¹,dTrp¹²,Trp²³,Tyr³⁶]-PTHrP(7-36), each antagonist at a dose of 500 nmol/kg. Data are means±SEM, with 5 mice per group (P vs. vehicle: *, <0.05; **, <0.01).

### DETAILED DESCRIPTION

### Definitions

“5′-end” and “3′-end” refers to the directionality, i.e., the end-to-end orientation of a nucleotide polymer (e.g., DNA). The 5′-end of a polynucleotide is the end of the polynucleotide that has the fifth carbon.

“5′-homology arm and 3′-homology arm” or “5′- and 3′-homology arms” or “left and right arms” refer to the polynucleotide sequences in a vector and/or targeting vector that are operable to homologously recombine with a target genome sequence and/or endogenous gene of interest and/or endogenous locus in a host organism, in order to achieve successful genetic modification of the host organism's chromosomal locus. For example, in some embodiments, the 5′-homology arm and 3′-homology arm can flank a transgene and, optionally, one or more regulatory elements, thus allowing the homologous recombination-mediated integration of the said transgene and optional one or more regulatory elements into the endogenous genome locus.

“Admixing” refers to contacting one component with another, e.g., a candidate agent with an hPTH1R protein, in any order, any combination and/or sub-combination.

“Alignment” refers to a method of comparing two or more sequences (e.g., nucleotide, polynucleotide, amino acid, peptide, polypeptide, or protein sequences) for the purpose of determining their relationship to each other. Alignments are typically performed by computer programs that apply various algorithms, however, it is also possible to perform an alignment by hand. Alignment programs typically iterate through potential alignments of sequences and score the alignments using substitution tables, employing a variety of strategies to reach a potential optimal alignment score. Commonly-used alignment algorithms include, but are not limited to, CLUSTALW (see Thompson J. D., Higgins D. G., Gibson T. J., CLUSTAL W: improving the sensitivity of progressive multiple sequence alignment through sequence weighting, position-specific gap penalties and weight matrix choice, Nucleic Acids Research 22: 4673-4680, 1994); CLUSTALV (see Larkin M. A., et al., CLUSTALW2, ClustalW and ClustalX version 2, Bioinformatics 23(21): 2947-2948, 2007); Mafft; Kalign; ProbCons; and T-Coffee (see Notredame et al., T-Coffee: A novel method for multiple sequence alignments, Journal of Molecular Biology 302: 205-217, 2000). Exemplary programs that implement one or more of the foregoing algorithms include, but are not limited to, MegAlign from DNAStar (DNAStar, Inc. 3801 Regent St. Madison, Wis. 53705), MUSCLE, T-Coffee, CLUSTALX, CLUSTALV, JalView, Phylip, and Discovery Studio from Accelrys (Accelrys, Inc., 10188 Telesis Ct, Suite 100, San Diego, Calif. 92121). In some embodiments, an alignment will introduce “phase shifts” and/or “gaps” into one or both of the sequences being compared in order to maximize the similarity between the two sequences, and scoring refers to the process of quantitatively expressing the relatedness of the aligned sequences.

“bp” or “base pair” refers to a molecule comprising two chemical bases bonded to one another forming a. For example, a DNA molecule consists of two winding strands, wherein each strand has a backbone made of an alternating deoxyribose and phosphate groups. Attached to each deoxyribose is one of four bases, i.e., adenine (A), cytosine (C), guanine (G), or thymine (T), wherein adenine forms a base pair with thymine, and cytosine forms a base pair with guanine.

“C-terminal” or “C-terminus” refers to the free carboxyl group (i.e., —COOH) that is positioned on the terminal end of a polypeptide.

“C57BL/6 mouse” refers to a common inbred strain of laboratory mouse that is well known in the art.

“Candidate agent” refers to one or more chemical substances, molecules, nucleotides, polynucleotides, RNA, DNA, peptides, polypeptides, proteins, lipids, glycolipids, enzymes, pharmaceuticals, drugs, organic compounds, inorganic compounds, prokaryote organisms or eukaryote organisms (and the agents produced from said prokaryote or eukaryote organisms), and/or combinations thereof, that can be screened using an assay and/or other method described herein.

“cDNA” or “copy DNA” or “complementary DNA” refers to a molecule that is complementary to a molecule of RNA. In some embodiments, cDNA may be either single-stranded or double-stranded. In some embodiments, cDNA can be a double-stranded DNA synthesized from a single stranded RNA template in a reaction catalyzed by a reverse transcriptase. In yet other embodiments, “cDNA” refers to all nucleic acids that share the arrangement of sequence elements found in native mature mRNA species, where sequence elements are exons and 3′ and 5′ non-coding regions. Normally mRNA species have contiguous exons, with the intervening introns removed by nuclear RNA splicing, to create a continuous open reading frame encoding the protein. In some embodiments, “cDNA” refers to a DNA that is complementary to and derived from an mRNA template.

“Chimera” refers to an is an entity having two or more incongruous or heterogeneous parts or regions. For example, as used herein, chimera can refer to a single organism composed of genetically distinct cells, i.e., an organism composed of at least two genetically distinct cell lineages originating from different zygotes.

“Cloning” refers to the process and/or methods concerning the insertion of a DNA segment (e.g., usually a gene of interest, for example human pth1r) from one source and recombining it with a DNA segment from another source (e.g., usually a vector, for example, a plasmid) and directing the recombined DNA, or “recombinant DNA” to replicate, usually by transforming the recombined DNA into a bacteria or yeast host.

“Coding sequence” or “CDS” refers to a polynucleotide or nucleic acid sequence that can be transcribed (e.g., in the case of DNA) or translated (e.g., in the case of mRNA) into a peptide, polypeptide, or protein, when placed under the control of appropriate regulatory sequences and in the presence of the necessary transcriptional and/or translational molecular factors. The boundaries of the coding sequence are determined by a translation start codon at the 5′ (amino) terminus and a translation stop codon at the 3′ (carboxy) terminus. A transcription termination sequence will usually be located 3′ to the coding sequence. In some embodiments, a coding sequence may be flanked on the 5′ and/or 3′ ends by untranslated regions. In some embodiments, a coding sequence can be used to produce a peptide, a polypeptide, or a protein product. In some embodiments, the coding sequence may or may not be fused to another coding sequence or localization signal, such as a nuclear localization signal. In some embodiments, the coding sequence may be cloned into a vector or expression construct, may be integrated into a genome, or may be present as a DNA fragment.

“Codon optimization” refers to the production of a gene in which one or more endogenous, native, and/or wild-type codons are replaced with codons that ultimately still code for the same amino acid, but that are of preference in the corresponding host.

“Complementary” refers to the topological compatibility or matching together of interacting surfaces of two polynucleotides as understood by those of skill in the art. Thus, two sequences are “complementary” to one another if they are capable of hybridizing to one another to form a stable anti-parallel, double-stranded nucleic acid structure. A first polynucleotide is complementary to a second polynucleotide if the nucleotide sequence of the first polynucleotide is substantially identical to the nucleotide sequence of the polynucleotide binding partner of the second polynucleotide, or if the first polynucleotide can hybridize to the second polynucleotide under stringent hybridization conditions. Thus, the polynucleotide whose sequence 5′-TATAC-3′ is complementary to a polynucleotide whose sequence is 5′-GTATA-3′.

“Culture” or “cell culture” refers to the maintenance of cells in an artificial, in vitro environment.

“Culturing” refers to the propagation of organisms on or in various kinds of media. For example, the term “culturing” can mean growing a population of cells under suitable conditions in a liquid or solid medium. In some embodiments, culturing refers to fermentative recombinant production of a heterologous polypeptide of interest and/or other desired end products (typically in a vessel or reactor).

“Degeneracy” or “codon degeneracy” refers to the phenomenon that one amino acid can be encoded by different nucleotide codons. Thus, the nucleic acid sequence of a nucleic acid molecule that encodes a protein or polypeptide can vary due to degeneracies. As a result of the degeneracy of the genetic code, many nucleic acid sequences can encode a given polypeptide with a particular activity; such functionally equivalent variants are contemplated herein.

“DNA” refers to deoxyribonucleic acid, comprising a polymer of one or more deoxyribonucleotides or nucleotides (i.e., adenine [A], guanine [G], thymine [T], or cytosine [C]), which can be arranged in single-stranded or double-stranded form. For example, one or more nucleotides creates a polynucleotide.

“dNTPs” refers to the nucleoside triphosphates that compose DNA and RNA.

“Endogenous” refers to a polynucleotide, peptide, polypeptide, protein, or process that naturally occurs and/or exists in an organism, e.g., a molecule or activity that is already present in the host cell before a particular genetic manipulation.

“Exon” refers to a defined section of nucleic acid that encodes for a protein, or a nucleic acid sequence that is represented in the mature form of an RNA molecule after either portions of a pre-processed (or precursor) RNA have been removed by splicing. The mature RNA molecule can be a messenger RNA (mRNA) or a functional form of a non-coding RNA, such as rRNA or tRNA.

“Expression cassette” refers to (1) a DNA sequence of interest, e.g., a heterologous polynucleotide comprising human Parathyroid Hormone 1 Receptor (hPTH1R) exons 4 to 16, wherein said heterologous polynucleotide is operable to encode a human PTH1R protein; and one or more of the following: (2) promoters, terminators, and/or enhancer elements; (3) an appropriate mRNA stabilizing polyadenylation signal; (4) an internal ribosome entry site (IRES); (5) introns; and/or (6) post-transcriptional regulatory elements. The combination (1) with at least one of (2)-(6) is called an “expression cassette.” In some embodiments, there can be numerous expression cassettes cloned into a vector. For example, in some embodiments, there can be a first expression cassette comprising a heterologous polynucleotide comprising hPTH1R exons 4 to 16, operable to encode a human PTH1R protein. In alternative embodiments, there are two expression cassettes operable to encode a human PTH1R protein (i.e., a double expression cassette). In other embodiments, there are three expression cassettes operable to encode a human PTH1R protein (i.e., a triple expression cassette). In some embodiments, a double expression cassette can be generated by subcloning a second expression cassette into a vector containing a first expression cassette. In some embodiments, a triple expression cassette can be generated by subcloning a third expression cassette into a vector containing a first and a second expression cassette. Methods concerning expression cassettes and cloning techniques are well-known in the art and described herein.

“Heterologous” refers to generally refers to a polynucleotide or protein that is not endogenous to the host cell or host organism, and/or or is not endogenous to the location in the native genome in which it is present and has been added to the cell or organism by recombinant techniques (e.g., infection, transfection, microinjection, electroporation, microprojection, or the like).

“Heterozygote” or “heterozygous individual” or heterozygous animal” refers to a diploid or polyploid individual cell or organism having different alleles (forms of a given gene) at least at one locus.

“Heterozygous” refers to the presence of different alleles (forms of a given gene) at a particular gene locus.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. Thus, in some embodiments, the term “homologous” refers to the sequence similarity between two polypeptide molecules, or between two nucleic acid molecules. When a position in both of the two compared sequences is occupied by the same base or amino acid monomeric subunit, e.g., if a position in each of two DNA molecules is occupied by adenine, then the molecules are homologous at that position. The homology between two sequences is a function of the number of matching or homologous positions shared by the two sequences. For example, if 6 of 10 of the positions in two sequences are matched or homologous then the two sequences are 60% homologous. By way of example, the DNA sequences ATTGCC and TATGGC share 50% homology.

In some embodiments, there may be partial homology, or complete homology and thus identical sequences. “Sequence identity” refers to a measure of relatedness between two or more nucleic acids, and is given as a percentage with reference to the total comparison length. The identity calculation takes into account those nucleotide residues that are identical and in the same relative positions in their respective larger sequences.

“Homologous recombination” refers to the event of substitution of a segment of DNA by another one that possesses identical regions (homologous) or nearly so. For example, in some embodiments, “homologous recombination” refers to a type of genetic recombination in which nucleotide sequences are exchanged between two similar or identical molecules of DNA. Briefly, homologous recombination is most widely used by cells to accurately repair harmful breaks that occur on both strands of DNA, known as double-strand breaks. Although homologous recombination varies widely among different organisms and cell types, most forms involve the same basic steps: after a double-strand break occurs, sections of DNA around the 5′ ends of the break are cut away in a process called resection. In the strand invasion step that follows, an overhanging 3′ end of the broken DNA molecule then “invades” a similar or identical DNA molecule that is not broken. After strand invasion, the further sequence of events may follow either of two main pathways, i.e., the double-strand break repair pathway, or the synthesis-dependent strand annealing pathway. Homologous recombination is conserved across all three domains of life as well as viruses, suggesting that it is a nearly universal biological mechanism. For example, in some embodiments, homologous recombination can occur using a site-specific integration (SSI) sequence, whereby there is a strand exchange crossover event between nucleic acid sequences substantially similar in nucleotide composition. These crossover events can take place between sequences contained in the targeting construct of the invention (i.e., the SSI sequence) and endogenous genomic nucleic acid sequences (e.g., the polynucleotide encoding the peptide subunit). In addition, in some embodiments, it is possible that more than one site-specific homologous recombination event can occur, which would result in a replacement event in which nucleic acid sequences contained within the targeting construct have replaced specific sequences present within the endogenous genomic sequences.

“Homozygote” or “homozygous individual” or homozygous animal” refers to an individual cell or organism having the same alleles at one or more loci.

“Homozygous” refers to the presence of identical alleles at one or more loci in homologous chromosomal segments.

“hPTH1R” refers to human PTH1R.

“hPTH1R-KI” is context dependent, and can refer to a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein, or the polynucleotide itself. For example, “hPTH1R-KI mice” refers to transgenic mice comprising a heterologous polynucleotide comprising human Parathyroid Hormone 1 Receptor (hPTH1R) exons 4 to 16; wherein said heterologous polynucleotide is operable to encode a human PTH1R protein.

“Identity” refers to a relationship between two or more polypeptide sequences or two or more polynucleotide sequences, as determined by comparing said sequences. The term “identity” also means the degree of sequence relatedness between polypeptide or polynucleotide sequences, as the case may be, as determined by the match between strings of such sequences. “Identity” and “similarity” can be readily calculated by any one of the myriad methods known to those having ordinary skill in the art, including but not limited to those described in: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994: Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988), the disclosures of which are incorporated herein by reference in their entireties. Furthermore, methods to determine identity and similarity are codified in publicly available computer programs. For example in some embodiments, methods to determine identity and similarity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990), the disclosures of which are incorporated herein by reference in their entireties.

“in vivo” refers to the natural environment (e.g., an animal or a cell) and to processes or reactions that occur within a natural environment.

“Inoperable” refers to the condition of a thing not functioning, malfunctioning, or no longer able to function. For example, when used in the context of a gene or when referring to a gene, the term inoperable means said gene is no longer able to operate as it normally would, either permanently or transiently. For example, “inoperable,” in some embodiments, means that a gene is no longer able to synthesize a gene product, having said gene product translated into a protein, or is otherwise unable to gene perform its normal function. For example, in some embodiments, the term inoperable can refer the failure of a gene to transcribe RNA, a failure of RNA processing (e.g., pre-mRNA processing; RNA splicing; or other post-transcriptional modifications); interference with non-coding RNA maturation; interference with RNA export (e.g., from the nucleus to the cytoplasm); interference with translation; protein folding; translocation; protein transport; and/or inhibition and/or interference with any of the molecules polynucleotides, peptides, polypeptides, proteins, transcription factors, regulators, inhibitors, or other factors that take part in any of the aforementioned processes.

“kb” refers to kilobase, i.e., 1000 bases. As used herein, the term “kb” means a length of nucleic acid molecules. For example, 1 kb refers to a nucleic acid molecule that is 1000 nucleotides long. A length of double-stranded DNA that is 1 kb long, contains two thousand nucleotides (i.e., one thousand on each strand). Alternatively, a length of single-stranded RNA that is 1 kb long, contains one thousand nucleotides.

“kDa” refers to kilodalton, a unit equaling 1,000 daltons; a “dalton” or “Da” is a unit of molecular weight (MW).

“Knock in” or “knock-in” or “knocks-in” or “knocking-in” refers to the replacement of an endogenous gene with an exogenous or heterologous gene, or part thereof. For example, in some embodiments, the term “knock-in” refers to the introduction of a nucleic acid sequence encoding a desired protein to a target gene locus by homologous recombination, thereby causing the expression of the desired protein. In some embodiments, a “knock-in” mutation can modify a gene sequence to create a loss-of-function or gain-of-function mutation. The term “knock-in” can refer to the procedure by which a exogenous or heterologous polynucleotide sequence or fragment thereof is introduced into the genome, (e.g., “they performed a knock-in” or “they knocked-in the heterologous gene”), or the resulting cell and/or organism (e.g., “the cell is a “knock-in” or “the animal is a “knock-in”).

“Knock out” or “knockout” or “knock-out” or “knocks-out” or “knocking-out” refers to a partial or complete suppression of the expression gene product (e.g., mRNA) of a protein encoded by an endogenous DNA sequence in a cell. In some embodiments, the “knock-out” can be effectuated by targeted deletion of a whole gene, or part of a gene encoding a peptide, polypeptide, or protein. As a result, the deletion may render a gene inactive, partially inactive, inoperable, partly inoperable, or otherwise reduce the expression of the gene or its products in any cell in the whole organism and/or cell in which it is normally expressed. The term “knock-out” can refer to the procedure by which an endogenous gene is made completely or partially inactive or inoperable (e.g., “they performed a knock-out” or “they knocked-out the endogenous gene”), or the resulting cell and/or organism (e.g., “the cell is a “knock-out” or “the animal is a “knock-out”).

“Locus” (plural: “loci”) refers to any site that has been defined genetically. A locus may be a gene, or part of a gene, or a DNA sequence that has some regulatory role, and may be occupied by different sequences.

“Molecular weight (MW)” refers to the mass or weight of a molecule, and is typically measured in “daltons (Da)” or kilodaltons (kDa). In some embodiments, MW can be calculated using sodium dodecyl sulfate polyacrylamide gel electrophoresis (SDS-PAGE), analytical ultracentrifugation, or light scattering. In some embodiments, the SDS-PAGE method is as follows: the sample of interest is separated on a gel with a set of molecular weight standards. The sample is run, and the gel is then processed with a desired stain, followed by destaining for about 2 to 14 hours. The next step is to determine the relative migration distance (Rf) of the standards and protein of interest. The migration distance can be determined using the following equation:

$$Rf = \frac{Migration distance of the protein}{Migration distance of the dye front} \qquad Formula ( I )$$

Next, the logarithm of the MW can be determined based on the values obtained for the bands in the standard; e.g., in some embodiments, the logarithm of the molecular weight of an SDS-denatured polypeptide and its relative migration distance (Rf) is plotted into a graph. After plotting the graph, interpolating the value derived will provide the molecular weight of the unknown protein band.

“Mutant” refers to an organism, DNA sequence, peptide sequence, or polypeptide sequence, that has an alteration (for example, in the DNA sequence), which causes said organism and/or sequence to be different from the naturally occurring or wild-type organism and/or sequence.

“N-terminal” or “N-terminus” refers to the free amine group (i.e., —NH₂) that is positioned on beginning or start of a polypeptide.

“NCBI” refers to the National Center for Biotechnology Information.

“nm” refers to nanometers.

“One letter code” means the peptide sequence which is listed in its one letter code to distinguish the various amino acids in the primary structure of a protein: alanine=A, arginine=R, asparagine=N, aspartic acid=D, asparagine or aspartic acid=B, cysteine=C, glutamic acid=E, glutamine=Q, glutamine or glutamic acid=Z, glycine=G, histidine=H, isoleucine=I, leucine=L, lysine=K, methionine=M, phenylalanine=F, proline=P, serine=S, threonine=T, tryptophan=W, tyrosine=Y, and valine=V.

“Open reading frame” or “ORF” refers to a length of RNA or DNA sequence, between a translation start signal (e.g., AUG or ATG, respectively) and any one or more of the known termination codons, which encodes one or more polypeptide sequences. Put another way, the ORF describes the frame of reference as seen from the point of view of a ribosome translating the RNA code, insofar that the ribosome is able to keep reading (i.e., adding amino acids to the nascent protein) because it has not encountered a stop codon. Thus, “open reading frame” or “ORF” refers to the amino acid sequence encoded between translation initiation and termination codons of a coding sequence. Here, the terms “initiation codon” and “termination codon” refer to a unit of three adjacent nucleotides (i.e., a codon) in a coding sequence that specifies initiation and chain termination, respectively, of protein synthesis (mRNA translation).

In some embodiments, an ORF is a continuous stretch of codons that begins with a start codon (usually ATG for DNA, and AUG for RNA) and ends at a stop codon (usually UAA, UAG or UGA). In other embodiments, an ORF can be length of RNA or DNA sequence, between a translation start signal (e.g., AUG or ATG) and any one or more of the known termination codons, wherein said length of RNA or DNA sequence encodes one or more polypeptide sequences. In some other embodiments, an ORF can be a DNA sequence encoding a protein which begins with an ATG start codon and ends with a TGA, TAA or TAG stop codon. ORF can also mean the translated protein that the DNA encodes. Generally, those having ordinary skill in the art distinguish the terms “open reading frame” and “ORF,” from the term “coding sequence,” based upon the fact that the broadest definition of “open reading frame” simply contemplates a series of codons that does not contain a stop codon. Accordingly, while an ORF may contain introns, the coding sequence is distinguished by referring to those nucleotides (e.g., concatenated exons) that can be divided into codons that are actually translated into amino acids by the ribosomal translation machinery (i.e., a coding sequence does not contain introns); however, as used herein, the terms “coding sequence”; “CDS”; “open reading frame”; and “ORF,’ are used interchangeably.

“Operable” refers to the ability to be used, the ability to do something, and/or the ability to accomplish some function or result. For example, in some embodiments, “operable” refers to the ability of a polynucleotide, DNA sequence, RNA sequence, or other nucleotide sequence or gene to encode a peptide, polypeptide, and/or protein. For example, in some embodiments, a polynucleotide may be operable to encode a protein, which means that the polynucleotide contains information that imbues it with the ability to create a protein (e.g., by transcribing mRNA, which is in turn translated to protein).

“Operably linked” refers to a juxtaposition wherein the components so described are in a relationship permitting them to function in their intended manner. For example, in some embodiments, operably linked can refer to two or more DNA, peptide, or polypeptide sequences. In other embodiments, operably linked can mean that the two adjacent DNA sequences are placed together such that the transcriptional activation of one DNA sequence can act on the other DNA sequence. In yet other embodiments, the term “operably linked” can refer to two or more peptides and/or polypeptides, wherein said two or more peptides and/or polypeptides are connected in such a way as to yield a single polypeptide chain; alternatively, the term operably linked can refer to two or more peptides that are connected in such a way that one peptide exerts some effect on the other. In yet other embodiments, operably linked can refer to two adjacent DNA sequences are placed together such that the transcriptional activation of one can act on the other.

“Plasmid” refers to a DNA segment that acts as a carrier for a gene of interest, and, when transformed or transfected into an organism, can replicate and express the DNA sequence contained within the plasmid independently of the host organism. Plasmids are a type of vector, and can be “cloning vectors” (i.e., simple plasmids used to clone a DNA fragment and/or select a host population carrying the plasmid via some selection indicator) or “expression plasmids” (i.e., plasmids used to produce large amounts of polynucleotides and/or polypeptides).

“Polynucleotide” refers to a polymeric-form of nucleotides (e.g., ribonucleotides, deoxyribonucleotides, or analogs thereof) of any length; e.g., a sequence of two or more ribonucleotides or deoxyribonucleotides. As used herein, the term “polynucleotide” includes double- and single-stranded DNA, as well as double- and single-stranded RNA; it also includes modified and unmodified forms of a polynucleotide (modifications to and of a polynucleotide, for example, can include methylation, phosphorylation, and/or capping). In some embodiments, a polynucleotide can be one of the following: a gene or gene fragment (for example, a probe, primer, EST, or SAGE tag); genomic DNA; genomic DNA fragment; exon; intron; messenger RNA (mRNA); transfer RNA; ribosomal RNA; ribozyme; cDNA; recombinant polynucleotide; branched polynucleotide; plasmid; vector; isolated DNA of any sequence; isolated RNA of any sequence; nucleic acid probe; primer or amplified copy of any of the foregoing.

In yet other embodiments, a polynucleotide can refer to a polymeric-form of nucleotides operable to encode the open reading frame of a gene.

In some embodiments, a polynucleotide can refer to cDNA.

In some embodiments, polynucleotides can have any three-dimensional structure and may perform any function, known or unknown. The structure of a polynucleotide can also be referenced to by its 5′- or 3′—end or terminus, which indicates the directionality of the polynucleotide. Adjacent nucleotides in a single-strand of polynucleotides are typically joined by a phosphodiester bond between their 3′ and 5′ carbons. However, different internucleotide linkages could also be used, such as linkages that include a methylene, phosphoramidate linkages, etc. This means that the respective 5′ and 3′ carbons can be exposed at either end of the polynucleotide, which may be called the 5′ and 3′ ends or termini. The 5′ and 3′ ends can also be called the phosphoryl (PO₄) and hydroxyl (OH) ends, respectively, because of the chemical groups attached to those ends. The term polynucleotide also refers to both double- and single-stranded molecules. Unless otherwise specified or required, any embodiment that makes or uses a polynucleotide encompasses both the double-stranded form and each of two complementary single-stranded forms known or predicted to make up the double-stranded form.

In some embodiments, a polynucleotide can include modified nucleotides, such as methylated nucleotides and nucleotide analogs (including nucleotides with non-natural bases, nucleotides with modified natural bases such as aza- or deaza-purines, etc.). If present, modifications to the nucleotide structure can be imparted before or after assembly of the polynucleotide.

In some embodiments, a polynucleotide can also be further modified after polymerization, such as by conjugation with a labeling component. Additionally, the sequence of nucleotides in a polynucleotide can be interrupted by non-nucleotide components. One or more ends of the polynucleotide can be protected or otherwise modified to prevent that end from interacting in a particular way (e.g. forming a covalent bond) with other polynucleotides.

In some embodiments, a polynucleotide can be composed of a specific sequence of four nucleotide bases: adenine (A); cytosine (C); guanine (G); and thymine (T). Uracil (U) can also be present, for example, as a natural replacement for thymine when the polynucleotide is RNA. Uracil can also be used in DNA. Thus, the term “sequence” refers to the alphabetical representation of a polynucleotide or any nucleic acid molecule, including natural and non-natural bases.

The term “RNA molecule” or ribonucleic acid molecule refers to a polynucleotide having a ribose sugar rather than deoxyribose sugar and typically uracil rather than thymine as one of the pyrimidine bases. An RNA molecule of the invention is generally single-stranded, but can also be double-stranded. In the context of an RNA molecule from an RNA sample, the RNA molecule can include the single-stranded molecules transcribed from DNA in the cell nucleus, mitochondrion or chloroplast, which have a linear sequence of nucleotide bases that is complementary to the DNA strand from which it is transcribed.

In some embodiments, a polynucleotide can further comprise one or more heterologous regulatory elements. For example, in some embodiments, the regulatory element is one or more promoters; enhancers; silencers; operators; splicing signals; polyadenylation signals; termination signals; RNA export elements, internal ribosomal entry sites (IRES); poly-U sequences; or combinations thereof.

“Post-transcriptional regulatory elements” are DNA segments and/or mechanisms that affect mRNA after it has been transcribed. Mechanisms of post-transcriptional mechanisms include splicing events; capping, splicing, and addition of a Poly (A) tail, and other mechanisms known to those having ordinary skill in the art.

“Promoter” refers to a region of DNA to which RNA polymerase binds and initiates the transcription of a gene.

“PTH” refers to parathyroid hormone.

“PTH1R” refers to parathyroid hormone 1 receptor.

“PTHrP” refers to parathyroid hormone-related protein.

“Ratio” refers to the quantitative relation between two amounts showing the number of times one value contains or is contained within the other.

“Reading frame” refers to one of the six possible reading frames, three in each direction, of the double stranded DNA molecule. The reading frame that is used determines which codons are used to encode amino acids within the coding sequence of a DNA molecule. In some embodiments, a reading frame is a way of dividing the sequence of nucleotides in a polynucleotide and/or nucleic acid (e.g., DNA or RNA) into a set of consecutive, non-overlapping triplets.

“Regulatory elements” refers to a genetic element that controls some aspect of the expression and/or processing of nucleic acid sequences. For example, in some embodiments, a regulatory element can be found at the transcriptional and post-transcriptional level. Regulatory elements can be cis-regulatory elements (CREs), or trans-regulatory elements (TREs). In some embodiments, a regulatory element can be one or more promoters; enhancers; silencers; operators; splicing signals; polyadenylation signals; termination signals; RNA export elements, internal ribosomal entry sites (IRES); poly-U sequences; and/or other elements that influence gene expression, for example, in a tissue-specific manner; temporal-dependent manner; to increase or decrease expression; and/or to cause constitutive expression.

“SSI” or “site-specific integration,” refers to a sequence that will permit in vivo homologous recombination to occur at a specific site within a host organism's genome. Thus, in some embodiments, the term “site-specific integration” refers to the process directing a transgene to a target site in a host-organism's genome, allowing the integration of genes of interest into pre-selected genome locations of a host-organism.

“Transfection” and “transformation” both refer to the process of introducing exogenous and/or heterologous DNA or RNA (e.g., a vector containing a polynucleotide that encodes an hPTH1R) into a host organism (e.g., a prokaryote or a eukaryote). Generally, those having ordinary skill in the art sometimes reserve the term “transformation” to describe processes where exogenous and/or heterologous DNA or RNA are introduced into a bacterial cell; and reserve the term “transfection” for processes that describe the introduction of exogenous and/or heterologous DNA or RNA into eukaryotic cells. However, as used herein, the term “transformation” and “transfection” are used synonymously, regardless of whether a process describes the introduction exogenous and/or heterologous DNA or RNA into a prokaryote (e.g., bacteria) or a eukaryote (e.g., yeast, plants, or animals).

“Transgene” means a heterologous and/or exogenous polynucleotide (e.g., DNA sequence) encoding a protein which is transformed into a host.

“Transgenic non-human animal” or “transgenic animal” refers to a non-human animal, e.g., mammals, amphibians, birds, and the like, whose somatic or germ line cells bear genetic information received, directly or indirectly, by deliberate genetic manipulation at the subcellular level, e.g., by microinjection or infection with recombinant virus. The term “transgenic” further includes cells or tissues (e.g., “transgenic cell,” and “transgenic tissue”) obtained from a transgenic animal genetically manipulated as described herein. In the present context, a “transgenic non-human animal” does not encompass animals produced by classical crossbreeding or in vitro fertilization, but rather denotes animals in which one or more cells receive a heterologous polynucleotide, e.g., via methods such as a recombinant nucleic acid molecule (e.g., a vector).

In some embodiments, the recombinant nucleic acid molecule may be specifically targeted to a defined genetic locus, be randomly integrated within a chromosome, or it may be extra-chromosomally replicating DNA. In some embodiments, a transgenic animal may comprise a genetic alteration to its germ line cells, or genetic information may be introduced into a germ line cell, thereby conferring onto the transgenic animal the ability to transfer the genetic information to its offspring; if such offspring, in fact, possess some or all of the alteration to the germline as the parent and/or possess all or some of the genetic information introduced to the parent, then the offspring are likewise, transgenic animals.

In some embodiments, transgenic non-human animals provided herein can be either heterozygous or homozygous with respect to the transgene. Also provided are transgenic animals that include a heterologous polynucleotide operable to encode a human PTH1R protein. In some embodiments, the transgenic animal can be sheep, feline, bovines, ovines, pigs, horses, rabbits, guinea pigs, mice, hamsters, rats, non-human primates, and the like.

Methods for producing transgenic animals, including mice, sheep, pigs and frogs, are well known in the art. Exemplary methods of producing transgenic animals are provided in U.S. Pat. Nos. 5,721,367; 5,695,977; 5,650,298; 5,614,396; 6,133,502; 6,175,057; 6,180,849; Wagner et al. (1981, PNAS USA, 78:5016-5020); Stewart et al. (1982, Science, 217:1046-1048); Constantini et al. (1981, Nature, 294:92-94); Lacy et al. (1983, Cell, 34:343-358); McKnight et al. (1983, Cell, 34:335-341); Brinstar et al. (1983, Nature, 306:332-336); Palmiter et al. (1982, Nature, 300:611-615); Palmiter et al. (1982, Cell, 29:701-710); and Palmiter et al. (1983, Science, 222:809-814); the disclosures of which are incorporated herein by reference in their entireties.

“Variant” or “variant sequence” or “variant protein” or “variant thereof” refer to an amino acid sequence that possesses one or more amino acid substitutions or modifications (e.g., deletion or addition). In some embodiments, the one or more amino acid substitutions or modifications can be conservative; here, such a conservative amino acid substitution and/or modification in a “variant” does not substantially diminish the activity of the variant in relation to its non-varied form. For example, in some embodiments, a “variant” possesses one or more conservative amino acid substitutions when compared to a peptide with a disclosed and/or claimed sequence, as indicated by a SEQ ID NO.

“Vector” refers to a DNA segment that accepts a foreign polynucleotide (e.g., a DNA sequence or gene of interest). The foreign polynucleotide of interest is known as an “insert” or “transgene.”

“Wild type” or “WT” refer to the phenotype and/or genotype (i.e., the appearance or sequence) of an organism, polynucleotide sequence, and/or polypeptide sequence, as it is found and/or observed in its naturally occurring state or condition.

Throughout this specification, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or group of compositions of matter.

The present disclosure is performed without undue experimentation using, unless otherwise indicated, conventional techniques of molecular biology, microbiology, virology, recombinant DNA technology, solid phase and liquid nucleic acid synthesis, peptide synthesis in solution, solid phase peptide synthesis, immunology, cell culture, and formulation. Such procedures are described, for example, in Sambrook, Fritsch & Maniatis, Molecular Cloning: A Laboratory Manual, Cold Spring Harbor Laboratories, New York, Second Edition (1989), whole of Vols I, II, and III; DNA Cloning: A Practical Approach, Vols. I and II (D. N. Glover, ed., 1985), IRL Press, Oxford, whole of text; Oligonucleotide Synthesis: A Practical Approach (M. J. Gait, ed, 1984) IRL Press, Oxford, whole of text, and particularly the papers therein by Gait, ppl-22; Atkinson et al, pp 35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984); Methods In Enzymology (S. Colowick and N. Kaplan, eds., Academic Press, Inc.), whole of series; J. F. Ramalho Ortigao, “The Chemistry of Peptide Synthesis” In: Knowledge database of Access to Virtual Laboratory website (Interactiva, Germany); Sakakibara, D., Teichman, J., Lien, E. Land Fenichel, R. L. (1976). Biochem. Biophys. Res. Commun. 73 336-342; Merrifield, R. B. (1963). J. Am. Chem. Soc. 85, 2149-2154; Barany, G. and Merrifield, R. B. (1979) in The Peptides (Gross, E. and Meienhofer, 3. eds.), vol. 2, pp. 1-284, Academic Press, New York. 12. Wiinsch, E., ed. (1974) Synthese von Peptiden in Houben-Weyls Metoden der Organischen Chemie (Muler, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474; Handbook of Experimental Immunology, Vols. I-IV (D. M. Weir and C. C. Blackwell, eds., 1986, Blackwell Scientific Publications); and Animal Cell Culture: Practical Approach, Third Edition (John R. W. Masters, ed., 2000); each of these references are incorporated herein by reference in their entireties.

Although the disclosure of the invention has been described in detail for purposes of clarity and understanding, it will be obvious to those with skill in the art that certain modifications can be practiced within the scope of the appended claims. All publications and patent documents cited herein are hereby incorporated by reference in their entirety for all purposes to the same extent as if each were so individually denoted.

Throughout this specification, unless the context requires otherwise, the word “comprise,” or variations such as “comprises” or “comprising,” will be understood to imply the inclusion of a stated step or element or integer or group of steps or elements or integers but not the exclusion of any other step or element or integer or group of elements or integers.

All patent applications, patents, and printed publications referred to herein are incorporated by reference in their entirety to the same extent as if each individual publication, patent, or patent application was specifically and individually indicated to be incorporated by reference in its entirety. And, all patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

### Proteins of the Present Disclosure

### Overview: Parathyroid Hormone 1 Receptor (PTH1R)

The parathyroid hormone 1 receptor (PTH1R) is a seven transmembrane, class B, G-protein coupled receptor that is linked to heterotrimeric G-proteins, e.g., Gs and Gq, PTH1R is the receptor for two ligands: parathyroid hormone (PTH), and parathyroid hormone-related protein (PTHrP). These ligands—each of which is responsible for distinct biological functions-both act through PTH1R. See Pioszak et al., Structural Basis for Parathyroid Hormone-related Protein Binding to the Parathyroid Hormone Receptor and Design of Conformation-selective Peptide. J Biol Chem. 2009 Oct. 9; 284(41): 28382-28391.

PTH is involved, inter alia, in calcium and/or phosphate homeostasis, and stimulates kidney and bone cells. See Murray et al., Parathyroid hormone secretion and action: evidence for discrete receptors for the carboxyl-terminal region and related biological actions of carboxyl- terminal ligands. Endocr Rev. 2005 February; 26(1):78-113. The protein, PTHrP, is involved in endochondral bone formation and tissue development. See Kronenberg, PTHrP and skeletal development. Ann N Y Acad Sci. 2006 April; 1068( ):1-13.

PTH1R can exist in two different conformations: (1) the “RG” conformation; and (2) the “R⁰” conformation. The RG conformation is sensitive to GTPγS (guanosine 5′-O-[gamma-thio]triphosphate), which is a non-hydrolyzable or slowly hydrolyzable G-protein-activating analog of guanosine triphosphate (GTP); alternatively, the R⁰ conformation is insensitive to GTPγS.

An exemplary description of PTH1R, its ligands, and its signaling, is provided in: Pioszak et al., Structural Basis for Parathyroid Hormone-related Protein Binding to the Parathyroid Hormone Receptor and Design of Conformation-selective Peptide. J Biol Chem. 2009 Oct. 9; 284(41): 28382-28391; Dean et al., Altered Selectivity of Parathyroid Hormone (PTH) and PTH-Related Protein (PTHrP) for Distinct Conformations of the PTH/PTHrP Receptor. Mol Endocrinol. 2008 January; 22(1): 156-166; Dean et al., Mechanisms of Ligand Binding to the Parathyroid Hormone (PTH)/PTH-Related Protein Receptor: Selectivity of a Modified PTH(1-15) Radioligand for Gas-Coupled Receptor Conformations. Mol Endocrinol. 2006 April; 20(4): 931-943; Okazaki et al., Prolonged signaling at the parathyroid hormone receptor by peptide ligands targeted to a specific receptor conformation. Proc Natl Acad Sci USA. 2008 Oct. 28; 105(43): 16525-16530; and Brent et al., The Efficacy of PTH and Abaloparatide to Counteract Immobilization-Induced Osteopenia Is in General Similar. Front Endocrinol (Lausanne). 2020 Oct. 9; 11:588773; the disclosures of which are incorporated herein by reference in their entireties.

The Parathyroid Hormone 1 Receptor (PTH1R) is a receptor for parathyroid hormone (PTH) and parathyroid hormone-like hormone (PTHLH). PTH1R is a member of the G-protein coupled receptor family 2. Briefly, the activity of PTH1R is mediated via G proteins, which activate adenylyl cyclase, and also a phosphatidylinositol-calcium second messenger system. See Bastepe et al., G Proteins in The Control of Parathyroid Hormone Actions. J Mol Endocrinol. 2017 May; 58(4): R203-R224.

The mouse Pth1r gene is located on mouse chromosome 9. Sixteen exons of the mouse Pth1r gene have been identified, with an ATG start codon in exon 3, and a TGA stop codon in exon 16. An exemplary mouse Pth1r nucleotide sequence is provide in SEQ ID NO: 5 (NCBI Reference Sequence: NM_011199.2; NCBI Gene ID NO: 19228). See Nishimori et al., Salt-inducible kinases dictate parathyroid hormone 1 receptor action in bone development and remodeling. J Clin Invest 129 (12), 5187-5203 (2019).

The human PTH1R gene is located on human chromosome 3. Sixteen exons have been identified for the human PTH1R gene, with the ATG start codon located in exon 3 and TGA stop codon in exon 16. Two transcript variants encoding the same protein have been found for human PTH1R gene; transcript variant 1 is longer than variant 2, however, both transcripts encode the same protein. An exemplary human PTH1R nucleotide sequence is provided in SEQ ID NO: 6 (NCBI Reference Sequence: NM_000316.2). See Luck et al., A reference map of the human binary protein interactome. Nature. 2020 April; 580 (7803):402-408.

hPTH1R Exons 4-16

In some embodiments, the present disclosure comprises, consists essentially of, or consists of, a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein.

An exemplary WT human PTH1R protein is provided below:

(SEQ ID NO: 28)
MGTARIAPGLALLLCCPVLSSAYALVDADDVMTKEEQIFLLHRAQAQCEK
RLKEVLQRPASIMESDKGWTSASTSGKPRKDKASGKLYPESEEDKEAPTG
SRYRGRPCLPEWDHILCWPLGAPGEVVAVPCPDYIYDENHKGHAYRRCDR
NGSWELVPGHNRTWANYSECVKELTNETREREVEDRLGMIYTVGYSVSLA
SLTVAVLILAYFRRLHCTRNYIHMHLFLSFMLRAVSIFVKDAVLYSGATL
DEAERLTEEELRAIAQAPPPPATAAAGYAGCRVAVTFFLYFLATNYYWIL
VEGLYLHSLIFMAFFSEKKYLWGFTVFGWGLPAVEVAVWVSVRATLANTG
CWDLSSGNKKWIIQVPILASIVLNFILFINIVRVLATKLRETNAGRCDTR
QQYRKLLKSTLVLMPLFGVHYIVFMATPYTEVSGTLWQVQMHYEMLENSF
QGFFVAIIYCFCNGEVQAEIKKSWSRWTLALDEKRKARSGSSSYSYGPMV
SHTSVTNVGPRVGLGLPLSPRLLPTATTNGHPQLPGHAKPGTPALETLET
TPPAMAAPKDDGFLNGSCSGLDEEASGPERPPALLQEEWETVM

In the foregoing sequence, residues M1-A22, i.e., “MGTARIAPGLALLLCCPVLSSA” (SEQ ID NO: 26) correspond to a signal sequence. The short segment following the signal sequence, i.e., Y23-A24-L25, corresponds to a portion of the mature hPTH1R protein that is encoded by exon 3.

Human PTH1R exons 4-16 encode a protein starting at position V26 of the SEQ ID NO: 28, and have the following sequence:

(SEQ ID NO: 1)
VDADDVMTKEEQIFLLHRAQAQCEKRLKEVLORPASIMESDKGWTSASTS
GKPRKDKASGKLYPESEEDKEAPTGSRYRGRPCLPEWDHILCWPLGAPGE
VVAVPCPDYIYDENHKGHAYRRCDRNGSWELVPGHNRTWANYSECVKELT
NETREREVEDRLGMIYTVGYSVSLASLTVAVLILAYFRRLHCTRNYIHMH
LFLSFMLRAVSIFVKDAVLYSGATLDEAERLTEEELRAIAQAPPPPATAA
AGYAGCRVAVTFFLYFLATNYYWILVEGLYLHSLIFMAFFSEKKYLWGFT
VFGWGLPAVEVAVWVSVRATLANTGCWDLSSGNKKWIIQVPILASIVLNF
ILFINIVRVLATKLRETNAGRCDTRQQYRKLLKSTLVLMPLFGVHYIVFM
ATPYTEVSGTLWQVQMHYEMLENSFQGFFVAIIYCFCNGEVQAEIKKSWS
RWTLALDFKRKARSGSSSYSYGPMVSHTSVTNVGPRVGLGLPLSPRLLPT
ATTNGHPQLPGHAKPGTPALETLETTPPAMAAPKDDGFLNGSCSGLDEEA
SGPERPPALLQEEWETVM

In some embodiments, the present disclosure comprises, consists essentially of, or consists of, a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.1% identical, at least 99.2% identical, at least 99.3% identical, at least 99.4% identical, at least 99.5% identical, at least 99.6% identical, at least 99.7% identical, at least 99.8% identical, at least 99.9% identical, or 100% identical to an amino acid sequence set forth in SEQ ID NO: 1.

In some embodiments, the present disclosure comprises, consists essentially of, or consists of, a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein having an amino acid sequence set forth in SEQ ID NO: 1.

### Protein Tags

In some embodiments, the present disclosure comprises, consists essentially of, or consists of, a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein, and wherein said human PTH1R protein further comprises, consists essentially of, or consists of, a tag.

In some embodiments, the tag can allow detection of the recombinant protein (e.g., through the use of standard immunohistochemistry techniques). For example, in some embodiments, the tag can be an epitope tag.

In other embodiments, the tag can allow isolation of the recombinant protein. For example, in some embodiments, the tag can be a capture tag.

In some embodiments the tag is an epitope tag, which can be detected with an antibody. For example, in some embodiments, the epitope tag can be detected with an antibody specifically immunoreactive with the epitope tag is used to isolate the protein.

In some embodiments, the tag can be, without limitation, one or more of the following tags peptides, polypeptides, proteins, and/or fragments thereof: human influenza hemagglutinin (HA); Myc (a polypeptide protein tag derived from the c-myc gene or a fragment thereof); FLAG; IRS; HIS; AU1 and/or Au5 (peptide sequences derived from the major capsid protein of bovine papillomavirus-1 (BPV-1)); glu-glu (a 9 amino acid epitope from polyoma virus medium T antigen); KT3 (an 11 amino acid epitope from the SV40 large T antigen); T7 (an 11 amino acid leader peptide from T7 major capsid protein); HSV (an 11 amino acid peptide from herpes simplex virus glycoprotein D); VSV-G (an 11 amino acid epitope from the carboxy terminus of vesicular stomatitis virus glycoprotein); V5 (14 amino acid epitope from paramyxovirus); S-TAG (an oligopeptide derived from pancreatic ribonuclease A); Streptavidin, or fragments thereof (a tetrameric protein expressed in Streptomyces avidinii); Maltose-binding protein (MBP); NE-tag (See U.S. Pat. No. 8,927,225); Streptavidin-Binding Peptide (SBP)-Tag; Spot-tag; Isopeptag; Glutathione S-transferase (GST); fluorescent proteins (e.g., green fluorescent protein or GFP); HaloTag (a 297 residue peptide (33 kDa) derived from a bacterial haloalkane dehalogenase); commercially available tags, e.g., Xpress synthetic peptide (available from Invitrogen®, Catalog No. R910-25); SNAP-tag, CLIP-tag, ACP-tag, or MCP-tag (available from New England Biolabs®); or any combination thereof.

The use of tags in the production or recombinant proteins are well known in the art. Exemplary descriptions regarding the use of tags are provided in: Wilson et al., “The Structure of an Antigenic Determinant in a Protein” Cell, vol. 37, Jul. 1984, pp. 767-778; Roth et al., “A Conserved Family of Nuclear Phosphoproteins Localized to Sites of Polymerase II Transcription” The Journal Of Cell Biology, vol. 115, No. 3, Nov. 1991, pp. 587-596; Los et al. (June 2008). “HaloTag: a novel protein labeling technology for cell imaging and protein analysis.” ACS Chemical Biology. 3 (6): 373-82; and U.S. Pat. Nos. 4,793,004; 4,851,341; 5,283,179; 6,462,254; 8,927,225; and 9,580,479; the disclosures of which are incorporated herein by reference in their entireties.

In some embodiments, the present disclosure comprises, consists essentially of, or consists of, a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein, and wherein said human PTH1R protein further comprises, consists essentially of, or consists of, a tag. In some embodiments, the tag can be operably linked to the hPTH1R protein at the 5′ end (upstream). In some embodiments, the tag can be operably linked to the hPTH1R protein at the 3′ end (downstream). In some embodiments, the tag can be a peptide sequence that replaces a peptide sequence of the hTPH1R protein.

In some embodiments, the tag can be a human influenza hemagglutinin (HA) epitope tag.

In some embodiments, the HA epitope tag can have an amino acid sequence of “YPYDVPDYA” (SEQ ID NO: 2).

In some embodiments, the HA epitope tag can have an amino acid sequence of “YPYDVPDYA” (SEQ ID NO: 2), wherein said HA epitope tag can replace residues 88-96, “YPESEEDKE” (SEQ ID NO: 3), of the hPTH1R protein.

In some embodiments, the present disclosure comprises, consists essentially of, or consists of, a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.1% identical, at least 99.2% identical, at least 99.3% identical, at least 99.4% identical, at least 99.5% identical, at least 99.6% identical, at least 99.7% identical, at least 99.8% identical, at least 99.9% identical, or 100% identical to an amino acid sequence set forth below, and in SEQ ID NO: 29:

(SEQ ID NO: 29)
VDADDVMTKEEQIFLLHRAQAQCEKRLKEVLORPASIMESDKGWTSASTS
GKPRKDKASGKLYPYDVPDYAAPTGSRYRGRPCLPEWDHILCWPLGAPGE
VVAVPCPDYIYDENHKGHAYRRCDRNGSWELVPGHNRTWANYSECVKELT
NETREREVEDRLGMIYTVGYSVSLASLTVAVLILAYFRRLHCTRNYIHMH
LFLSFMLRAVSIFVKDAVLYSGATLDEAERLTEEELRAIAQAPPPPATAA
AGYAGCRVAVTFFLYFLATNYYWILVEGLYLHSLIFMAFFSEKKYLWGFT
VFGWGLPAVEVAVWVSVRATLANTGCWDLSSGNKKWIIQVPILASIVLNF
ILFINIVRVLATKLRETNAGRCDTRQQYRKLLKSTLVLMPLFGVHYIVFM
ATPYTEVSGTLWQVQMHYEMLENSFQGFFVAIIYCFCNGEVQAEIKKSWS
RWTLALDEKRKARSGSSSYSYGPMVSHTSVTNVGPRVGLGLPLSPRLLPT
ATTNGHPQLPGHAKPGTPALETLETTPPAMAAPKDDGFLNGSCSGLDEEA
SGPERPPALLQEEWETVM

As shown in the foregoing sequence (SEQ ID NO: 29), the residues “YPYDVPDYA” (SEQ ID NO: 2) (underlined) represents the a human influenza hemagglutinin (HA) epitope tag, which has replaced the residues 88-96, “YPESEEDKE” (SEQ ID NO: 3), of the hPTH1R protein.

Polynucleotides Encoding hPTH1R

In some embodiments, an exemplary heterologous polynucleotide comprising human Parathyroid Hormone 1 Receptor (hPTH1R) exons 4 to 16, or a complementary nucleotide sequence thereof; wherein said heterologous polynucleotide is operable to encode a human PTH1R protein.

In some embodiments, a heterologous polynucleotide of the present disclosure comprises, consists essentially of, or consists of, a polynucleotide operable to encode a human PTH1R protein having an amino acid sequence that is at least is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence set forth in any one of SEQ ID NOs: 1, 29, or 30, or a complementary nucleotide sequence thereof.

In some embodiments, a heterologous polynucleotide of the present disclosure comprises, consists essentially of, or consists of, a polynucleotide operable to encode a human PTH1R protein having an amino acid sequence that is at least is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence set forth in SEQ ID NO: 1, or a complementary nucleotide sequence thereof.

In some embodiments, a heterologous polynucleotide of the present disclosure comprises, consists essentially of, or consists of, a polynucleotide operable to encode a human PTH1R protein having an amino acid sequence that is at least is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence set forth in SEQ ID NO: 29, or a complementary nucleotide sequence thereof.

In some embodiments, a heterologous polynucleotide of the present disclosure comprises, consists essentially of, or consists of, a polynucleotide operable to encode a human PTH1R protein having an amino acid sequence that is at least is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the amino acid sequence set forth in SEQ ID NO: 30, or a complementary nucleotide sequence thereof.

In some embodiments, a heterologous polynucleotide of the present disclosure comprises, consists essentially of, or consists of, a polynucleotide human Parathyroid Hormone 1 Receptor (hPTH1R) exons 4 to 16, or a complementary nucleotide sequence thereof.

In some embodiments, a heterologous polynucleotide of the present disclosure comprises, consists essentially of, or consists of, a polynucleotide human Parathyroid Hormone 1 Receptor (hPTH1R) exons 4 to 16 having a nucleotide sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to the nucleotide sequence set forth in SEQ ID NO: 4, or a complementary nucleotide sequence thereof.

In some embodiments, an exemplary heterologous polynucleotide comprising human Parathyroid Hormone 1 Receptor (hPTH1R) exons 4 to 16; wherein said heterologous polynucleotide is operable to encode a human PTH1R protein, and wherein said heterologous polynucleotide has an nucleotide sequence as set forth in SEQ ID NO: 4.

### Non-Human Animals

In some embodiments, the present disclosure comprises, consists essentially of, or consists of, a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein, and wherein the non-human animal is can be any non-human animal. For example, in some embodiments, the non-human animal can be a fungus (e.g., a yeast cell); an invertebrate animal (e.g. fruit fly, cnidarian, echinoderm, nematode, etc.); or vertebrate animal (e.g., fish, amphibian, reptile, bird, mammal, or non-human primate).

In some embodiments, the present disclosure comprises, consists essentially of, or consists of, a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein, and wherein the non-human animal is a vertebrate. For example, in some embodiments, the vertebrate can be, without limitation: a fish (e.g., zebra fish, gold fish, puffer fish, cave fish, etc.); an amphibian (frog, salamander, etc.); a bird (e.g., chicken, turkey, etc.); a reptile (e.g., snake, lizard, etc.); a mammal (e.g., an ungulate, e.g., a pig, a cow, a goat, a sheep, etc.); a lagomorph (e.g., a rabbit); a rodent (e.g., a rat, a mouse); or a non-human primate.

In some embodiments, the transgenic animal can be a mammal.

In some embodiments, a transgenic non-human animals of the present disclosure can be a member selected from the order, Rodentia.

In some embodiments, a transgenic non-human animal of the present disclosure can be a member selected from the following suborders: Anomaluromorpha; Castorimorpha; Hystricomorpha; Myomorpha; or Sciuromorpha.

In some embodiments, the transgenic non-human animal is selected from the suborder Myomorpha. For example, in some embodiments, the transgenic non-human animal is selected from the superfamilies: Dipodoidea or Muroidea.

In some embodiments, the transgenic non-human animal can be a member selected from the Muroidea superfamily. For example, in some embodiments, the transgenic non-human animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, white-tailed rats, Malagasy rats and mice), Platacanthomyidae (e.g., spiny dormice), and Spalacidae (e.g., mole rats, bamboo rats, and zokors).

In some embodiments, transgenic non-human animals of the present disclosure can be a mouse; a rat; a guinea pig; a hamster; or a gerbil.

In some embodiments, a transgenic non-human animals of the present disclosure can be a member selected from the genera, Mus.

In some embodiments, a transgenic non-human animals of the present disclosure can be a Mus musculus (house mouse).

In some embodiments, the transgenic non-human animal can be subspecies selected from following group: Mus musculus albula; Mus musculus bactrianus (southwestern Asian house mouse); Mus musculus brevirostris; Mus musculus castaneus (southeastern Asian house mouse); Mus musculus domesticus (western European house mouse); Mus musculus domesticus x M. m. molossinus; Mus musculus gansuensis; Mus musculus gentilulus; Mus musculus helgolandicus; Mus musculus homourus; Mus musculus isatissus; Mus musculus molossinus (Japanese wild mouse); Mus musculus musculus (eastern European house mouse); Mus musculus musculus x M. m. castaneus; Mus musculus musculus x M. m. domesticus; and/or Mus musculus wagneri.

In some embodiments, a transgenic non-human animal can be a mouse, wherein the mouse is: a 129 mouse; an A mouse; a BALB/c mouse; a C3H mouse; a C57BL mouse; a C57BR mouse; a C57L mouse; a CB17 mouse; a CD-1 mouse; a DBA mouse; an FVB mouse; an SJL mouse; an SWR mouse; any substrain thereof; any hybrid strain thereof; any congenic strain thereof; or any mutant strain thereof.

In some embodiments, a transgenic non-human animal can be a C57BL/6 mouse, or a C57BL/10 mouse. For example, in some embodiments, the transgenic non-human animal can be selected from the group consisting of: C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, or C57BL/10a.

In some embodiments, a transgenic non-human animal can be a C57BL/6 mouse.

In some embodiments, the present disclosure comprises, consists essentially of, or consists of, a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein, and wherein the heterologous polynucleotide comprising human PTH1R exons 4 to 16 is operable to encode a polypeptide having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.1% identical, at least 99.2% identical, at least 99.3% identical, at least 99.4% identical, at least 99.5% identical, at least 99.6% identical, at least 99.7% identical, at least 99.8% identical, at least 99.9% identical, or 100% identical to an amino acid sequence set forth in SEQ ID NOs: 1 or 29.

### Genome Integration

In some embodiments, the present disclosure comprises, consists essentially of, or consists of, a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein, and wherein the heterologous polynucleotide comprising human PTH1R exons 4 to 16 is stably integrated in the genome of the non-human animal.

As used herein, “stably integrated” means that the exogenous heterologous polynucleotide comprising human Parathyroid Hormone 1 Receptor (hPTH1R) exons 4 to 16, incorporates into the genome DNA of the host animal, and can be passed into daughter cells for at least multiple generations, preferably for unlimited generations. Accordingly, the heterologous polynucleotide comprising human Parathyroid Hormone 1 Receptor (hPTH1R) exons 4 to 16, or portion thereof, is expressed in the transgenic non-human animal, and, as a result of the expression, the transgenic non-human animal has an increased level of human PTH1R protein relative to the human PTH1R protein level in a mouse that does not express the same transgenic heterologous polynucleotide comprising human Parathyroid Hormone 1 Receptor (hPTH1R) exons 4 to 16, or portion thereof.

In some embodiments, the present disclosure comprises, consists essentially of, or consists of, a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein, and wherein the heterologous polynucleotide comprising human PTH1R exons 4 to 16 is stably integrated in an endogenous non-human animal PTH1R gene locus.

In some embodiments, the present disclosure comprises, consists essentially of, or consists of, a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein, wherein the heterologous polynucleotide comprising human PTH1R exons 4 to 16 is stably integrated in an endogenous non-human animal PTH1R gene locus that causes a replacement of a genomic DNA segment comprising non-human animal PTH1R exon 4, with the heterologous polynucleotide comprising human PTH1R exons 4 to 16.

In some embodiments, the present disclosure comprises, consists essentially of, or consists of, a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein, wherein the heterologous polynucleotide comprising human PTH1R exons 4 to 16 is stably integrated in an endogenous non-human animal PTH1R gene locus that causes a replacement of a genomic DNA segment comprising non-human animal PTH1R exon 4, with the heterologous polynucleotide comprising human PTH1R exons 4 to 16, and wherein the replacement results in a heterozygous transgenic non-human animal, or a homozygous transgenic non-human animal.

### Recombinant Cells

Another aspect of the present disclosure contemplates the use of non-human recombinant cells to evaluate hPTH1R. For the purposes of this section of the disclosure, the terms “non-human recombinant cells” and “recombinant cells” and “non-human animal recombinant cells” are used interchangeably.

Methods for generating recombinant cells, and recombinant techniques in general, are well-known to those having ordinary skill in the art. Recombinant methods and methods for generating recombinant cells are described herein. See, e.g., Craig, Ann. Rev. Genet. 1988, 22:77; Cox. In Genetic Recombination (R. Kucherlapati and G. R. Smith, eds.) 1988, American Society for Microbiology, Washington, D.C., pages 429-493; and Hoess. In Nucleic Acid and Molecular Biology (F. Eckstein and D. M. J. Lilley eds.) Vol. 4, 1990, Springer-Verlag, Berlin, pages 99-109, the disclosures of which are incorporated herein by reference in their entireties.

Recombinant cells of the invention can created in a variety of ways. For example, in one embodiment, recombinant cells can be generated using any of the recombinant techniques described herein, e.g., transformation of primary cell cultures.

In some embodiments, primary cells can be isolated from a wild-type organism, and subsequently transformed. For example, in some embodiments, the wild-type organism can be transformed with a vector comprising, inter alia, a heterologous polynucleotide comprising human Parathyroid Hormone 1 Receptor (PTH1R) exons 4 to 16, wherein said heterologous polynucleotide is operable to encode a human PTH1R protein.

As used herein, the term “isolated” refers to separating a thing and/or a component from its natural environment, e.g., a cell isolated from an organism means that said cell is separated from its natural environment, i.e., taken out of the organism. The term “derived” can be context dependent. For example, the term “derived” can have the same meaning as “isolated” (as defined above), or (in some contexts) it can describe a characteristic of a present condition or object in relationship to and not present in the ancestral and/or original form, e.g., when describing a non-naturally occurring mutation induced to a gene, one can describe the mutated gene as being derived from a gene that does not possess the mutation. However, for the purposes of this disclosure, the terms “isolated” and “derived” are used interchangeably, and mean separating a thing and/or a component from its natural environment.

In another embodiment, recombinant cells can generated by creating a transgenic non-human animal of the invention, and isolating a cell therefrom.

In some embodiments, a recombinant cell can be isolated from the transgenic non-human animal at any stage of its development, following the initial transformation of said transgenic non-human animal with a heterologous polynucleotide comprising human Parathyroid Hormone 1 Receptor (PTH1R) exons 4 to 16, wherein said heterologous polynucleotide is operable to encode a human PTH1R protein. For example, in some embodiments, a recombinant cell can be obtained by taking an embryonic stem cell (ESC), and transforming it with a heterologous polynucleotide comprising human Parathyroid Hormone 1 Receptor (PTH1R) exons 4 to 16, wherein said heterologous polynucleotide is operable to encode a human PTH1R protein.

In some embodiments, the present disclosure comprises, consists essentially of, or consists of, a non-human recombinant cell comprising: a heterologous polynucleotide comprising human Parathyroid Hormone 1 Receptor (PTH1R) exons 4 to 16, wherein said heterologous polynucleotide is operable to encode a human PTH1R protein.

In some embodiments, the present disclosure comprises, consists essentially of, or consists of, a non-human recombinant cell, wherein the non-human recombinant cell is derived from any non-human animal. For example, in some embodiments, the non-human animal can be a fungus (e.g., a yeast cell); an invertebrate animal (e.g. fruit fly, cnidarian, echinoderm, nematode, etc.); or vertebrate animal (e.g., fish, amphibian, reptile, bird, mammal, or non-human primate). In some embodiments, the non-human animal is a mouse.

In some embodiments, the present disclosure comprises, consists essentially of, or consists of, a non-human animal recombinant cell comprising a heterologous polynucleotide comprising human Parathyroid Hormone 1 Receptor (hPTH1R) exons 4 to 16; wherein said heterologous polynucleotide is operable to encode a human PTH1R protein, and wherein the non-human animal is a vertebrate. For example, in some embodiments, the vertebrate can be, without limitation: a fish (e.g., zebra fish, gold fish, puffer fish, cave fish, etc.); an amphibian (frog, salamander, etc.); a bird (e.g., chicken, turkey, etc.); a reptile (e.g., snake, lizard, etc.); a mammal (e.g., an ungulate, e.g., a pig, a cow, a goat, a sheep, etc.); a lagomorph (e.g., a rabbit); a rodent (e.g., a rat, a mouse); or a non-human primate.

In some embodiments, the non-human animal recombinant cell can be isolated from a mammal.

In some embodiments, a non-human animal recombinant cell of the present disclosure can be a cell isolated from a member selected from the order, Rodentia.

In some embodiments, a non-human animal recombinant cell of the present disclosure can be a cell isolated from a member selected from the following suborders: Anomaluromorpha; Castorimorpha; Hystricomorpha; Myomorpha; or Sciuromorpha.

In some embodiments, the non-human animal recombinant cell can be isolated from an animal that is selected from the suborder Myomorpha. For example, in some embodiments, the non-human animal recombinant cell is isolated from a member selected from the superfamilies: Dipodoidea or Muroidea.

In some embodiments, the non-human animal recombinant cell can be isolated from an animal that is a member selected from the Muroidea superfamily. For example, in some embodiments, the non-human animal recombinant cell is isolate from an animal in the family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, white-tailed rats, Malagasy rats and mice), Platacanthomyidae (e.g., spiny dormice), and Spalacidae (e.g., mole rats, bamboo rats, and zokors).

In some embodiments, a non-human animal recombinant cell of the present disclosure can be isolated from a mouse; a rat; a guinea pig; a hamster; or a gerbil.

In some embodiments, a non-human animal recombinant cell of the present disclosure can be isolated from a member selected from the genera, Mus.

In some embodiments, a non-human animal recombinant cell of the present disclosure can be isolated from a Mus musculus (house mouse).

In some embodiments, the non-human animal recombinant cell can be isolated from a subspecies selected from following group: Mus musculus albula; Mus musculus bactrianus (southwestern Asian house mouse); Mus musculus brevirostris; Mus musculus castaneus (southeastern Asian house mouse); Mus musculus domesticus (western European house mouse); Mus musculus domesticus x M. m. molossinus; Mus musculus gansuensis; Mus musculus gentilulus; Mus musculus helgolandicus; Mus musculus homourus; Mus musculus isatissus; Mus musculus molossinus (Japanese wild mouse); Mus musculus musculus (eastern European house mouse); Mus musculus musculus x M. m. castaneus; Mus musculus musculus x M. m. domesticus; and/or Mus musculus wagneri.

In some embodiments, the present disclosure comprises, consists essentially of, or consists of, a non-human recombinant cell, wherein the non-human recombinant cell is a mammalian recombinant cell.

In some embodiments, the present disclosure comprises, consists essentially of, or consists of, a non-human recombinant cell, wherein the recombinant cell is selected from the group consisting of: a mouse; a rat; a guinea pig; a hamster; and a gerbil.

In some embodiments, the present disclosure comprises, consists essentially of, or consists of, a non-human recombinant cell, wherein the recombinant cell is a mouse recombinant cell.

In some embodiments, the present disclosure comprises, consists essentially of, or consists of, a non-human recombinant cell, wherein the non-human recombinant cell is: a 129 recombinant cell; an A recombinant cell; a BALB/c recombinant cell; a C3H recombinant cell; a C57BL recombinant cell; a C57BR recombinant cell; a C57L recombinant cell; a CB17 recombinant cell; a CD-1 recombinant cell; a DBA recombinant cell; an FVB recombinant cell; an SJL recombinant cell; an SWR recombinant cell; a cell from any substrain thereof; a cell from any hybrid strain thereof; a cell from any congenic strain thereof; or a cell from any mutant strain thereof.

In some embodiments, the present disclosure comprises, consists essentially of, or consists of, a non-human recombinant cell, wherein the non-human recombinant cell is a C57BL/6 mouse recombinant cell, or a C57BL/10 mouse recombinant cell.

In some embodiments, a non-human animal recombinant cell can be isolated from a C57BL/6 mouse, or a C57BL/10 mouse. For example, in some embodiments, the transgenic non-human animal can be selected from the group consisting of: C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, or C57BL/10a.

In some embodiments, the present disclosure comprises, consists essentially of, or consists of, a non-human recombinant cell, wherein the non-human recombinant cell is a C57BL/6 mouse recombinant cell comprising a heterologous polynucleotide comprising human Parathyroid Hormone 1 Receptor (hPTH1R) exons 4 to 16; wherein said heterologous polynucleotide is operable to encode a human PTH1R protein.

In some embodiments, a non-human animal recombinant cell a C57BL/6 cell.

In some embodiments, the present disclosure comprises, consists essentially of, or consists of, a non-human animal recombinant cell comprising a heterologous polynucleotide comprising human Parathyroid Hormone 1 Receptor (hPTH1R) exons 4 to 16; wherein said heterologous polynucleotide is operable to encode a human PTH1R protein, and wherein the heterologous polynucleotide comprising human PTH1R exons 4 to 16 is operable to encode a polypeptide having an amino acid sequence with at least having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.1% identical, at least 99.2% identical, at least 99.3% identical, at least 99.4% identical, at least 99.5% identical, at least 99.6% identical, at least 99.7% identical, at least 99.8% identical, at least 99.9% identical, or 100% identical to an amino acid sequence set forth in SEQ ID NOs: 1 or 29.

In some embodiments, the present disclosure comprises, consists essentially of, or consists of, a non-human animal recombinant cell comprising a heterologous polynucleotide comprising human Parathyroid Hormone 1 Receptor (hPTH1R) exons 4 to 16; wherein said heterologous polynucleotide is operable to encode a human PTH1R protein, wherein the heterologous polynucleotide comprising human PTH1R exons 4 to 16 is stably integrated in the genome of the non-human recombinant cell.

In some embodiments, the present disclosure comprises, consists essentially of, or consists of, a non-human animal recombinant cell comprising a heterologous polynucleotide comprising human Parathyroid Hormone 1 Receptor (hPTH1R) exons 4 to 16; wherein said heterologous polynucleotide is operable to encode a human PTH1R protein, wherein the heterologous polynucleotide comprising human PTH1R exons 4 to 16 is stably integrated in an endogenous non-human animal PTH1R gene locus.

In some embodiments, the present disclosure comprises, consists essentially of, or consists of, a non-human animal recombinant cell comprising a heterologous polynucleotide comprising human Parathyroid Hormone 1 Receptor (hPTH1R) exons 4 to 16; wherein said heterologous polynucleotide is operable to encode a human PTH1R protein, wherein the heterologous polynucleotide comprising human PTH1R exons 4 to 16 is stably integrated in an endogenous non-human animal PTH1R gene locus that causes a replacement of a genomic DNA segment comprising non-human animal PTH1R exon 4, with the heterologous polynucleotide comprising human PTH1R exons 4 to 16.

In some embodiments, the present disclosure comprises, consists essentially of, or consists of, a non-human animal recombinant cell comprising a heterologous polynucleotide comprising human Parathyroid Hormone 1 Receptor (hPTH1R) exons 4 to 16; wherein said heterologous polynucleotide is operable to encode a human PTH1R protein, wherein the replacement results in a heterozygous recombinant cell, or a homozygous recombinant cell.

### Generating Polynucleotides

Methods of generating polynucleotides operable to encode an hPTH1R protein are well known in the art. Any method described herein or known in the art may be used to generate polynucleotides of the present disclosure.

In some embodiments, a polynucleotide operable to encode an hPTH1R protein (e.g., a heterologous polynucleotide comprising human Parathyroid Hormone 1 Receptor (hPTH1R) exons 4 to 16, wherein said heterologous polynucleotide is operable to encode a human PTH1R protein) can be chemically synthesized. For example, in some embodiments, a polynucleotide operable to encode an hPTH1R protein can be chemically synthesized using commercially available polynucleotide synthesis services such as those offered by Genewiz® (e.g., TurboGENE™; PriorityGENE; and FragmentGENE), or Sigma-Aldrich® (e.g., Custom DNA and RNA Oligos Design and Order Custom DNA Oligos). Exemplary method for generating DNA and or custom chemically synthesized polynucleotides are well known in the art, and are illustratively provided in U.S. Pat. No. 5,736,135, Ser. No. 8/389,615, filed on Feb. 13, 1995, the disclosure of which is incorporated herein by reference in its entirety. See also Agarwal, et al., Chemical synthesis of polynucleotides. Angew Chem Int Ed Engl. 1972 June; 11(6):451-9; Ohtsuka et al., Recent developments in the chemical synthesis of polynucleotides. Nucleic Acids Res. 1982 Nov. 11; 10(21): 6553-6570; Sondek & Shortle. A general strategy for random insertion and substitution mutagenesis: substoichiometric coupling of trinucleotide phosphoramidites. Proc Natl Acad Sci USA. 1992 Apr. 15; 89(8): 3581-3585; Beaucage S. L., et al., Advances in the Synthesis of Oligonucleotides by the Phosphoramidite Approach. Tetrahedron, Elsevier Science Publishers, Amsterdam, NL, vol. 48, No. 12, 1992, pp. 2223-2311; Agrawal (1993) Protocols for Oligonucleotides and Analogs: Synthesis and Properties; Methods in Molecular Biology Vol. 20, the disclosures of which are incorporated herein by reference in their entireties.

In some embodiments, a polynucleotide sequence can be generated using the oligonucleotide synthesis methods, such as the phosphoramidite; triester, phosphite, or H-Phosphonate methods. See Engels, J. W. and Uhlmann, E. (1989), Gene Synthesis [New Synthetic Methods (77)]; and Angew. Chem. Int. Ed. Engl., 28: 716-734, the disclosures of which are incorporated herein by reference in their entireties.

Chemically synthesizing polynucleotides allows for a DNA sequence to be generated that is tailored to produce a desired polypeptide based on the arrangement of nucleotides within said sequence (i.e., the arrangement of cytosine [C], guanine [G], adenine [A] or thymine [T] molecules); the mRNA sequence that is transcribed from the chemically synthesized DNA polynucleotide can be translated to a sequence of amino acids, each amino acid corresponding to a codon in the mRNA sequence. Accordingly, the amino acid composition of a polypeptide chain that is translated from an mRNA sequence can be altered by changing the underlying codon that determines which of the 20 amino acids will be added to the growing polypeptide; thus, mutations in the DNA such as insertions, substitutions, deletions, and frameshifts may cause amino acid insertions, substitutions, or deletions, depending on the underlying codon.

Polynucleotide sequences (e.g., a DNA sequence) can be obtained by cloning the DNA sequence into an appropriate vector. There are a variety of expression vectors available, host organisms, and cloning strategies known to those having ordinary skill in the art, and described herein. For example, the vector can be a plasmid, which can introduce a heterologous gene and/or expression cassette into host cells to be transcribed and translated. The term “vector” is used to refer to a carrier nucleic acid molecule into which a nucleic acid sequence can be inserted for introduction into a cell where it can be replicated. A vector may contain “vector elements” such as an origin of replication (ORI); a gene that confers antibiotic resistance to allow for selection; multiple cloning sites; a promoter region; a selection marker for non-bacterial transfection; and a primer binding site. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al., 1989 and Ausubel et al., 1996, both incorporated herein by reference in their entireties. Vectors suitable to practice the invention are described in detail below.

The host organisms used to clone the polynucleotides of the present disclosure can be any cell type, e.g., a eukaryotic or prokaryotic cell. In some embodiments, the host cells can be a bacteria. In other embodiments, the cells can be yeast cells.

### Transformation Techniques

The terms “transformation” and “transfection” both describe the process of introducing exogenous and/or heterologous DNA or RNA to a host organism. Generally, those having ordinary skill in the art sometimes reserve the term “transformation” to describe processes where exogenous and/or heterologous DNA or RNA are introduced into a bacterial cell; and reserve the term “transfection” for processes that describe the introduction of exogenous and/or heterologous DNA or RNA into eukaryotic cells. However, as used herein, the term “transformation” and “transfection” are used synonymously, regardless of whether a process describes the introduction exogenous and/or heterologous DNA or RNA into a prokaryote (e.g., bacteria) or a eukaryote (e.g., yeast, plants, or animals).

Transformation can be carried out by a variety of known techniques, depending on the organism, characteristics of the organism's cells, and of its biology. Stable transformation involves DNA entry into cells and into the cell nucleus. For organisms that are regenerated from single cells (which includes some mammals), transformation is carried out in in vitro culture, followed by selection for transformants and regeneration of the transformants. Methods often used for transferring DNA or RNA into cells include micro-injection, particle gun bombardment, forming DNA or RNA complexes with cationic lipids, liposomes or other carrier materials, electroporation, and incorporating transforming DNA or RNA into virus vectors. Other techniques are known in the art. DNA transfer into the cell nucleus occurs by cellular processes, and can sometimes be aided by choice of an appropriate vector, by including integration site sequences which are acted upon by an intracellular transposase or recombinase.

In some embodiments, a polynucleotide operable to encode a hPTH1R protein can be transformed into a host cell using micro-injection, particle gun bombardment, forming DNA or RNA complexes with cationic lipids, liposomes, electroporation, and/or incorporating transforming DNA or RNA into virus vectors.

The gene encoding hPTH1R is provided herein, having an NCBI Gene ID No. 5745. The WT mRNA operable to encode hPTH1R is provided herein, having the NCBI Reference Sequence: NM_000316.3 (SEQ ID NO: 27).

In some embodiments, a polynucleotide operable to encode a hPTH1R protein can be cloned into a vector, and transformed into a host cell using electroporation. For example, in some embodiments, a heterologous polynucleotide comprising human Parathyroid Hormone 1 Receptor (hPTH1R) exons 4 to 16, wherein said heterologous polynucleotide is operable to encode a human PTH1R protein, can be cloned into a vector and electroporated into a cell.

Exemplary descriptions of vectors are provided below. Exemplary methods for transformation are provided in Craig, Ann. Rev. Genet. 1988, 22:77; Cox. In Genetic Recombination (R. Kucherlapati and G. R. Smith, eds.) 1988, American Society for Microbiology, Washington, D.C., pages 429-493; and Hoess. In Nucleic Acid and Molecular Biology (F. Eckstein and D. M. J. Lilley eds.) Vol. 4, 1990, Springer-Verlag, Berlin, pages 99-109, the disclosures of which are incorporated herein by reference in their entireties.

### Homologous Recombination

Typically, the transformation of cells utilizes the power of homologous recombination. Homologous recombination generally describes a process in which nucleotide sequences are exchanged between similar or homologous DNA sequences. Homologous recombination is an intrinsic property of many cells, and is used by cells in certain circumstances to repair DNA damage; homologous recombination also occurs during meiosis, resulting in new combinations of DNA sequences. The molecular machinery underpinning the process of homologous recombination can be harnessed to practice the present disclosure in order to modify an organism's genome and/or DNA sequences.

For example, by harnessing the process of homologous recombination, one or more polynucleotides, e.g., a gene (or part of a gene) contained within an organism's genome, can be removed or replaced with a heterologous polynucleotide (also referred to as a “transgene”) or allele created in vitro. Indeed, the process is so precise, and can be reproduced with such fidelity, that the only genetic difference between the initial organism and the organism post-modification, is the modification itself.

Homologous recombination can also be used to modify genes via the attachment of an epitope tag (e.g., FLAG, myc, or HA); alternatively, a gene of interest can be operably linked to the coding sequence of a fluorescent protein, e.g., green fluorescent protein (GFP). And, because a given epitope tag or fusion is created within the context of the organism and/or its genome, said gene of interest is subjected to the inherent regulatory elements and regulatory events that normally would occur in host organism-both spatially and temporally. Accordingly, tagged transgenes (e.g., a heterologous polynucleotide of interest tagged with an epitope tag or operably linked to GFP) can be compared to an isogenic wild-type organism in order to examine gene function, peptide localization, and/or regulation.

In some embodiments, a polynucleotide of interest can be integrated into a host animal's genome through homologous recombination. For example, in some embodiments, a polynucleotide operable to encode an hPTH1R protein (e.g., a heterologous polynucleotide comprising human Parathyroid Hormone 1 Receptor (hPTH1R) exons 4 to 16, wherein said heterologous polynucleotide is operable to encode a human PTH1R protein) can be incorporated into an animal's genome via homologous recombination.

In some embodiments, homologous recombination can be harnessed to add or remove polynucleotides to or from a non-human animal. For example, in some embodiments, the present disclosure provides for a transgenic non-human animal comprising a heterologous polynucleotide comprising human Parathyroid Hormone 1 Receptor (hPTH1R) exons 4 to 16, wherein said heterologous polynucleotide is operable to encode a human PTH1R protein, and wherein the heterologous polynucleotide comprising human PTH1R exons 4 to 16 is stably integrated in the genome of the non-human animal. In some embodiments, the stable integration of the heterologous polynucleotide comprising hPTH1R exons 4 to 16 is achieved via homologous recombination.

In some embodiments, homologous recombination can be utilized to insert a heterologous polynucleotide comprising human Parathyroid Hormone 1 Receptor (PTH1R) exons 4 to 16, into the genome of a non-human animal.

In some embodiments, homologous recombination allows the heterologous polynucleotide comprising human PTH1R exons 4 to 16 is stably integrated in an endogenous non-human animal PTH1R gene locus.

In some embodiments, homologous recombination allows the heterologous polynucleotide comprising hPTH1R exons 4 to 16 to be stably integrated in an endogenous non-human animal PTH1R gene locus, which causes a replacement of a genomic DNA segment comprising non-human animal PTH1R exon 4, with the heterologous polynucleotide comprising human PTH1R exons 4 to 16.

### Vectors

A vector of the present disclosure refers to a means for introducing one or more polynucleotides into a host cell. There are a variety of vectors available, host organisms, and cloning strategies known to those having ordinary skill in the art.

As used herein, the term “vector” refers to a carrier nucleic acid molecule into which a polynucleotide can be inserted for introduction into a cell, and where it can be replicated. A vector may contain “vector elements” such as an origin of replication (ORI); a gene that confers antibiotic resistance to allow for selection; multiple cloning sites; a promoter region; a selection marker; a primer binding site; and/or a combination thereof. The polynucleotide inserted into the vector can be “heterologous” or “exogenous,” which means that it is foreign to the cell into which the vector is being introduced, or that the sequence is homologous to a sequence in the cell but in a position within the host cell nucleic acid in which the sequence is ordinarily not found. Vectors can be used to prepare polynucleotides of the present disclosure, or to ultimately transform the cells used to generate a transgenic animal (e.g., an ESC).

In some embodiments, vectors include plasmids, cosmids, viruses (bacteriophage, animal viruses, and plant viruses), and artificial chromosomes (e.g., YACs). For example, in some embodiments, a vector can be a plasmid, which can introduce a heterologous gene and/or expression cassette into host cells to be transcribed and translated.

One having ordinary skill in the art would be well equipped to construct a vector through standard recombinant techniques, which are described in Sambrook et al., 1989 and Ausubel et al., 1996, both incorporated herein by reference in their entireties. In addition to encoding polynucleotide (e.g., a polynucleotide operable to encode a hPTH1R protein), a vector may also encode a targeting molecule. A targeting molecule is one that directs the desired nucleic acid to a particular tissue, cell, or other location.

In some embodiments, a polynucleotide operable to encode a human PTH1R protein can be inserted into any suitable vector, e.g., a plasmid, bacteriophage, or viral vector for amplification, and may thereby be propagated using methods known in the art, such as those described in Molecular Cloning A Laboratory Manual, 2nd Ed., ed. by Sambrook, Fritsch and Maniatis (Cold Spring Harbor Laboratory Press: 1989), the disclosure of which is incorporated herein by reference in its entirety.

In addition to a polynucleotide sequence operable to encode a hPTH1R protein, additional DNA segments known as regulatory elements can be cloned into a vector that allow for enhanced expression of a foreign DNA, heterologous polynucleotide, or transgene; examples of such regulatory elements include (1) promoters, terminators, and/or enhancer elements; (2) an appropriate mRNA stabilizing polyadenylation signal; (3) an internal ribosome entry site (IRES); (4) introns; and (5) post-transcriptional regulatory elements. The combination of a DNA segment of interest (e.g., a polynucleotide operable to encode a hPTH1R protein) with any one of the foregoing cis-acting elements is called an “expression cassette.”

In some embodiments, an expression cassette can contain one or more polynucleotides operable to encode an hPTH1R protein.

In some embodiments, an expression cassette can contain one or more polynucleotides operable to encode an hPTH1R protein, and one or more additional regulatory elements such as: (1) promoters, terminators, and/or enhancer elements; (2) an appropriate mRNA stabilizing polyadenylation signal; (3) an internal ribosome entry site (IRES); (4) introns; and (5) post-transcriptional regulatory elements.

Insertion of the appropriate polynucleotide (e.g., a DNA sequence) into a vector can be performed by a variety of procedures. In general, the DNA sequence is ligated to the desired position in the vector following digestion of the insert and the vector with appropriate restriction endonucleases. Alternatively, blunt ends in both the insert and the vector may be ligated. A variety of cloning techniques are disclosed in Ausubel et al. Current Protocols in Molecular Biology, John Wiley & Sons, Inc. 1997 and Sambrook et al., Molecular Cloning: A Laboratory Manual 2nd Ed., Cold Spring Harbor Laboratory Press (1989); the disclosures of which are incorporated herein by reference in their entireties. Such procedures and others are deemed to be within the scope of those skilled in the art.

In some embodiments, a polynucleotide encoding an hPTH1R protein can be inserted into other commercially available plasmids and/or vectors that are readily available to those having skill in the art, e.g., plasmids are available from Addgene (a non-profit plasmid repository); GenScript®; Takara®; Qiagen®; and Promega™

In some embodiments, a vector can be, for example, in the form of a plasmid, a viral particle, or a phage. In other embodiments, a vector can include chromosomal, non-chromosomal and synthetic DNA sequences, derivatives of SV40; bacterial plasmids, phage DNA, baculovirus, yeast plasmids, vectors derived from combinations of plasmids and phage DNA, viral DNA such as vaccinia, adenovirus, fowl pox virus and pseudorabies.

In some embodiments, vectors compatible with eukaryotic cells, such as vertebrate cells, can be used. Eukaryotic cell vectors are well known in the art and are available from commercial sources. Contemplated vectors may contain both prokaryotic sequences (to facilitate the propagation of the vector in bacteria), and one or more eukaryotic transcription units that are functional in non-bacterial cells. Typically, such vectors provide convenient restriction sites for insertion of the desired recombinant DNA molecule. The pcDNAI, pSV2, pSVK, pMSG, pSVL, pPVV-1/PML2d and pTDTI (ATCC No. 31255) derived vectors are examples of mammalian vectors suitable for transfection of non-human cells. In some embodiments, some of the foregoing vectors may be modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) may be used for expression of proteins in swine cells. The various methods employed in the preparation of the plasmids and the transformation of host cells are well known in the art.

In some embodiments, and in addition to a polynucleotide of interest, a vector may include a signal sequence or a leader sequence for targeting membranes or secretion as well as expression regulatory elements, such as a promoter, an operator, an initiation codon, a stop codon, a polyadenylation signal, and/or an enhancer; and can be constructed in various forms depending on the purpose thereof. The initiation codon and stop codons are generally considered to be a portion of a nucleotide sequence coding for a target protein, are necessary to be functional in a subject to which a genetic construct has been administered, and must be in frame with the coding sequence.

In some embodiments, the promoter of the vector may be constitutive or inducible. In addition, expression vectors may include a selectable marker that allows the selection of host cells containing the vector, and replicable expression vectors include a replication origin. The vector may be self-replicable, or may be integrated into the host DNA.

Use of promoters may not be required in cases in which transcriptionally active genes are targeted, if the design of the construct results in the marker being transcribed as directed by an endogenous promoter. Exemplary constructs and vectors for carrying out such targeted modification are described herein. However, other vectors that can be used in such approaches are known in the art and can readily be adapted for use in the invention.

In some embodiments, a targeting vector can be used. A basic targeting vector comprises a site-specific integration (SSI) sequence, e.g., 5′- and 3′—homology arms of sequence that is homologous to an endogenous DNA segment that is being targeted.

In some embodiments, a targeting vector can also optionally include one or more positive and/or negative selection markers. In some embodiments, the selection markers can be used to disrupt gene function and/or to identify ESC clones that integrated targeting vector DNA following transformation.

In some embodiments, a vector may comprise vector elements allowing for the deletion of incorporated sequences (e.g., at later stages of development and/or in specific tissues) can be included.

In some embodiments, the use of a targeting vector may utilize a heterologous polynucleotide comprising one or more mutations, in order to create restriction patterns that are distinguishable from the endogenous gene (if the transgene and endogenous gene are similar).

In some embodiments, during the introduction of the transgene into the animal to be modified, the transgene can be inserted into the locus of a similar endogenous gene, thereby knocking-out function of the similar endogenous gene.

In other embodiments, the exogenous gene is inserted into the animal genome in a location such that the expression of the endogenous gene is preserved. Thus, in some embodiments, the transgenic animal may express all or part of the endogenous polynucleotide that corresponds to the human transgene polynucleotide inserted into the animal.

In some embodiments, the present disclosure comprises, consists essentially of, or consists of, a vector comprising: (i) a heterologous polynucleotide comprising a first nucleotide sequence comprising a coding sequence for human Parathyroid Hormone 1 Receptor (hPTH1R) exons 4 to 16 and second nucleotide sequence comprising a polyadenylation signal; (ii) a 5′-homology arm, and a 3′-homology arm, wherein said 5′-homology arm and said 3′-homology arm are located upstream and downstream of the heterologous polynucleotide, respectively; (iii) a third nucleotide sequence operable to encode a diphtheria toxin A protein, or fragment thereof; and a fourth nucleotide sequence operable to encode an neomycin phosphotransferase II (Neo); (iv) an upstream self-deletion anchor (SDA) nucleotide sequence, and a downstream SDA nucleotide sequence; wherein said upstream SDA nucleotide sequence and downstream SDA nucleotide sequences flank the fourth nucleotide sequence; wherein said vector is operable to allow a homologous recombination-mediated integration of the heterologous polynucleotide into an endogenous non-human animal PTH1R gene locus; and wherein said homologous recombination-mediated integration results in a replacement of an endogenous non-human animal genomic DNA segment with the heterologous polynucleotide.

### Homology Arms

Those having ordinary skill in the art will recognize that targeted gene modification requires the use of nucleic acid molecule vectors comprising regions of homology with a targeted gene (or flanking regions thereof), such that integration of the vector into the genome can be facilitated. Thus, a targeting vector is generally designed to contain three main regions: (1) a first region that is homologous to the locus to be targeted (e.g., a non-human animal Pth1r genes or a flanking sequence); (2) a second region that is a heterologous polynucleotide sequence (e.g., encoding a selectable marker, such as an antibiotic resistance protein) that is to specifically replace a portion of the targeted locus or is inserted into the targeted locus; and (3) a third region that, like the first region, is homologous to the targeted locus, but typically is not contiguous with the first region of the genome.

Homologous recombination between the targeting vector and the targeted wild-type locus results in deletion of any locus sequences between the two regions of homology represented in the targeting vector and replacement of that sequence with, or insertion into that sequence of, a heterologous sequence that, for example, encodes the polynucleotide of interest and optionally a selectable marker.

In order to facilitate homologous recombination, the first and third regions of the targeting vectors (see above) include sequences that exhibit substantial identity to the genes to be targeted (or flanking regions). By “substantially identical” is meant having a sequence that is at least 80%, preferably at least 85%, preferably at least 90%, more preferably at least 95%, even more preferably at least 98%, and even more preferably 100% identical to that of another sequence. Sequence identity is typically measured using BLAST® (Basic Local Alignment Search Tool) or BLAST® 2 with the default parameters specified therein (see, Altschul et al., J. Mol. Biol. 215: 403-410, 1990; Tatiana et al., FEMS Microbiol. Lett. 174: 247-250, 1999). These software programs match similar sequences by assigning degrees of homology to various substitutions, deletions, and other modifications. Thus, sequences having at least 80%, preferably at least 85%, preferably at least 90%, more preferably at least 95%, even more preferably at least 98%, and even more preferably 100% sequence identity with the targeted gene loci can be used in the invention to facilitate homologous recombination.

The total size of the two regions of homology (i.e., the first and third regions noted above) can be, for example, approximately between 1-25 kilobases (kb) (for example, approximately between 2-20 kb, approximately between 5-15 kb, or approximately between 6-10 kb), and the size of the second region that replaces a portion of the targeted locus can be, for example, approximately between 0.5-5 kb (for example, approximately between 1-4 kb, approximately between 1-3 kb, approximately between 1-2 kb, or approximately between 3-4 kb).

In some embodiments, a targeting vector generally can comprise a selection marker and a site-specific integration (SSI) sequence. The SSI sequence can comprise a transgene of interest (e.g., a transgene encoding hPTH1R), which is flanked with two genomic DNA fragments called “5′- and 3′-homology arms” or “5′ and 3′ arms” or “left and right arms” or “homology arms.” These homology arms recombine with the target genome sequence and/or endogenous gene of interest in the host organism in order to achieve successful genetic modification of the host organism's chromosomal locus.

When designing the homology arms for a targeting vector, both the 5′- and 3′-arms should possess sufficient sequence homology with the endogenous sequence to be targeted in order to engender efficient in vivo pairing of the sequences, and cross-over formation. And, while homology arm length is variable, a homology covering at least 5-8 kb in total for both arms (with the shorter arm having no less than 1 kb in length), is a general guideline that can be followed to help ensure successful recombination.

In some embodiments, the 5′- and/or 3′-homology arms may vary. For example, in some embodiments, different loci could be targeted by the 5′- and/or 3′-homology arms, e.g., either upstream and/or downstream from a homology arm described herein to exchange the sequence of interest at a different location. For example, in some embodiments, the 5′- and/or 3′-homology arms can be modified in order integrate a heterologous polynucleotide comprising human Parathyroid Hormone 1 Receptor (hPTH1R) exons 4 to 16, wherein said heterologous polynucleotide is operable to encode a human PTH1R protein, into the non-human animal genome, and cause a replacement of an endogenous DNA segment (e.g., the entire non-human animal PTH1R gene).

Additional exemplary methods of vector design and in vivo homologous recombination can be found in U.S. Pat. No. 5,464,764, entitled “Positive-negative selection methods and vectors” (filed Feb. 4, 1993; assignee University of Utah Research Foundation, Salt Lake City, UT); U.S. Pat. No. 5,733,761, entitled “Protein production and protein delivery” (filed May 26, 1995; assignee Transkaryotic Therapies, Inc., Cambridge, MA); U.S. Pat. No. 5,789,215, entitled “Gene targeting in animal cells using isogenic DNA constructs” (filed Aug. 7, 1997; assignee GenPharm International, San Jose, CA); U.S. Pat. No. 6,090,554, entitled “Efficient construction of gene targeting vectors” (filed Oct. 31, 1997; assignee Amgen, Inc., Thousand Oaks, CA); U.S. Pat. No. 6,528,314, entitled “Procedure for specific replacement of a copy of a gene present in the recipient genome by the integration of a gene different from that where the integration is made” (filed Jun. 6, 1995; assignee Institut, Pasteur); U.S. Pat. No. 6,537,542, entitled “Targeted introduction of DNA into primary or secondary cells and their use for gene therapy and protein production (filed Apr. 14, 2000; assignee Transkaryotic Therapies, Inc., Cambridge, MA); U.S. Pat. No. 8,048,645, entitled “Method of producing functional protein domains (filed Aug. 1, 2001; assignee Merck Serono SA); and U.S. Pat. No. 8,173,394, entitled “Systems and methods for protein production” (filed Apr. 6, 2009; assignee Wyeth LLC, Madison, NJ); the disclosures of which are incorporated herein by reference in their entirety.

### Selection Markers

Genetically targeted cells are typically identified using a selectable marker, e.g., a marker that allows selection of successfully transformed cells by conferring some property (e.g., color change or trait, e.g., survival in the presence of one or more chemicals and/or drugs). If a cell already contains a selectable marker, however, a new targeting construct containing a different selectable marker can be used. Alternatively, if the same selectable marker is employed, cells can be selected in the second targeting round by raising the drug concentration (for example, by doubling the drug concentration), as is known in the art. As is noted above, targeting vectors can include selectable markers flanked by sites facilitating excision of the marker sequences. In one example, constructs can include loxP sites to facilitate the efficient deletion of the marker using the cre/lox system. In yet another example, a self-deletion site can be used to allow for self-excision the marker. Use of such systems is well known in the art, and a specific example of use of this system is provided below, in the experimental examples. An exemplary description of self-excision DNA sequences is provided in Bunting et al., Targeting genes for self-excision in the germ line. Genes Dev. 1999 Jun. 15; 13(12): 1524-1528, the disclosure of which is incorporated herein by reference in its entirety.

In some embodiments, a selection marker is a molecule (e.g., a polynucleotide, peptide, polypeptide, or protein), the expression of which in a cell confers a detectable trait to said cell.

In some embodiments, selection markers can be polynucleotides and/or the proteins translated therefrom, that confer resistance to compounds such as antibiotics; confer the ability to grow on selected substrates; or that produce detectable signals such as luminescence, catalytic RNAs and antisense RNAs.

In some preferred embodiments, the selection marker is a polynucleotide, e.g., a nucleotide sequence introduced into a recombinant vector that encodes a polypeptide that confers a trait suitable for artificial selection or identification (e.g., a reporter gene).

In some embodiments, the selection marker can be a polynucleotide that encodes an enzymatic activity that confers the ability to grow in medium lacking what would otherwise be an essential nutrient; in addition, a selection marker may confer resistance to an antibiotic or drug upon the cell in which the selection gene is expressed. In some embodiments, a selection marker may be used to confer a particular phenotype upon a host cell. For example, in some embodiments, when a host cell must express a selection gene to grow in selective medium, the gene is said to be a positive selection gene. A selection gene can also be used to select against host cells containing a particular gene; a selection gene used in this manner is referred to as a negative selection gene.

In some embodiments, the selection markers can be a tag. For example, in some embodiments, tags include, but are not limited to: affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with F1AsH-EDT2 for fluorescence imaging), DNA and/or RNA segments that contain restriction enzyme or other enzyme cleavage sites; epitope tags (e.g. GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

In some embodiments, a selection marker can be one or more tags, e.g., to facilitate identification and/or purification of a target protein. Tags for use in the methods of the present disclosure include, but are not limited to: AviTag; Calmodulin; chitin binding protein (CBP); maltose binding protein (MBP); glutathione-S-transferase (GST); poly(His); biotin/streptavidin; Myc-tag; HA-tag; NE-tag; His-tag; Isopeptag; Flag tag; Halo-tag; Snap-tag; Fc-tag; Nus-tag; BCCP; Thioredoxin; SnooprTag; SpyTag; SBP-tag; S-tag; V5-tag; or any combination of sequences appropriate for use in a method of tagging a protein. The protein of interest and associated tag can be purified from target cells, or target cell culture medium by any method known in the art for purifying polypeptides; e.g., affinity tag column chromatography, antibody column chromatography, acrylamide gel electrophoresis, high pressure liquid chromatography, and salt fractionation. Such methods are well known to those skilled in the art.

In some embodiments, the selection marker can be a polynucleotide (e.g., DNA and/or RNA segments) that encode products that are otherwise lacking in the recipient cell (e.g., tRNA genes, auxotrophic markers).

For example, in some embodiments, there can be one or more selection markers that can complement to the inability of an expression organism to synthesize a particular compound required for its growth (auxotrophic) and one able to convert a compound to another that is toxic for growth. For example, URA3, an orotidine-5′ phosphate decarboxylase, is necessary for uracil biosynthesis and can complement ura3 mutants that are auxotrophic for uracil. URA3 also converts 5-fluoroorotic acid into the toxic compound 5-fluorouracil.

In some embodiments, the selection marker can be one or more polynucleotides that encode products providing resistance against otherwise toxic compounds, including antibiotics. For example, in some embodiments, the selection marker can be neomycin phosphotransferase II, hygromycin phosphotransferase (HPT)), and the like.

In some embodiments, the selection marker can include any genes that impart antibacterial resistance or express a fluorescent protein. For example in some embodiments, selection markers include, but are not limited to, the following genes: amp^{r}, camr, tet^{r}, blasticidin^{r}, neo^{r}, hyg^{r}, abx^{r}, neomycin phosphotransferase type II gene (nptII), p-glucuronidase (gus), green fluorescent protein (GFP), EGFP, YFP, mCherry, p-galactosidase (lacZ), lacZa, lacZAM15, chloramphenicol acetyltransferase (cat), alkaline phosphatase (phoA), bacterial luciferase (luxAB), bialaphos resistance gene (bar), phosphomannose isomerase (pmi), xylose isomerase (xylA), arabitol dehydrogenase (atlD), UDP-glucose:galactose-1-phosphate uridyltransferase I (galT), feedback-insensitive a subunit of anthranilate synthase (OASA1D), 2-deoxyglucose (2-DOGR), benzyladenine-N-3-glucuronide, E. coli threonine deaminase, glutamate 1-semialdehyde aminotransferase (GSA-AT), D-amino acid oxidase (DAAO), salt-tolerance gene (rstB), ferredoxin-like protein (pflp), trehalose-6-P synthase gene (AtTPS1), lysine racemase (lyr), dihydrodipicolinate synthase (dapA), tryptophan synthase beta 1 (AtTSB 1), dehalogenase (dhlA), mannose-6-phosphate reductase gene (M6PR), hygromycin phosphotransferase (HPT), and D-serine ammonia-lyase (dsdA).

In some embodiments, the antibiotic used for selection can be, but is not limited to, spectinomycin, ampicillin, kanamycin, tetracycline, and Basta (e.g., herbicides containing phosphinothricin).

In some embodiments, expression of a fluorescent protein can be detected using a fluorescent activated cell sorter (FACS). Expression of 3-galactosyltransferase also can be sorted by FACS, coupled with staining of living cells with a suitable substrate for p-galactosidase. A selection marker also may be a cell-substrate adhesion molecule, such as integrins which normally are not expressed by the mouse embryonic stem cells, miniature swine embryonic stem cells, and mouse, porcine and human hematopoietic stem cells. Target cell selection marker can be of mammalian origin and can be thymidine kinase, aminoglycoside phosphotransferase, asparagine synthetase, adenosine deaminase or metallothionien. The cell selection marker can also be neomycin phosphotransferase, hygromycin phosphotransferase or puromycin phosphotransferase, which confer resistance to G418, hygromycin and puromycin, respectively.

In some embodiments, the selection marker can allow selection based on the ability to distinguish between wanted and unwanted cells via the presence or absence of an expected color. For example, the lac-z-gene produces a beta-galactosidase enzyme which confers a blue color in the presence of X-gal (5-bromo-4-chloro-3-indolyl-β-D-galactoside). If recombinant insertion inactivates the lac-z-gene, then the resulting colonies are colorless.

In yet other embodiments, the selection marker can be a polynucleotide (e.g., DNA and/or RNA segments) that encode products which can be readily identified by a color-change reaction, or encodes a fluorescent protein (e.g., phenotypic markers such as 3-galactosidase, GUS; fluorescent proteins such as green fluorescent protein (GFP), cyan (CFP), yellow (YFP), red (RFP), luciferase, and cell surface proteins).

For example, in various embodiments, selection markers include, but are not limited to, alkaline phosphatase, β-galactosyltransferase, chloramphenicol-acetyl transferase (CAT), horseradish peroxidase, luciferase, and NanoLuc®.

In some embodiments, the selection marker can be one or more fluorescent proteins including, but not limited to: green fluorescent proteins (e.g. GFP, TagGFP, T-Sapphire, Azami Green, Emerald, mWasabi, and mClover3); red fluorescent proteins (e.g. mRFP1, JRed, HcRed1, AsRed2, AQ143, mCherry, mRuby3, and mPlum); yellow fluorescent proteins (e.g. EYFP, mBanana, mCitrine, PhiYFP, TagYFP, Topaz, and Venus); orange fluorescent proteins (e.g. DsRed, Tomato, Kusabria Orange, mOrange, mTangerine, and TagRFP); cyan fluorescent proteins (e.g. CFP, mTFP1, Cerulean, CyPet, and AmCyan1); blue fluorescent proteins (e.g. Azurite, mtagBFP2, EBFP, EBFP2, and Y66H); near-infrared fluorescent proteins (e.g. iRFP670, iRFP682, iRFP702, iRFP713 and iRFP720); infrared fluorescent proteins (e.g. IFP1.4); and photoactivatable fluorescent proteins (e.g. Kaede, Eos, IrisFP, PS-CFP).

In other embodiments, the selection marker can be one or more polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.

In some preferred embodiments, a selection marker or polynucleotide encoding the same, can be used to eliminate target cells in which an expression cassette has not been properly inserted, or to eliminate host cells in which the vector has not been properly transformed.

In some embodiments, a selection marker can be a positive selection marker, or negative selection marker. Positive selection markers permit the selection for cells in which the gene product of the marker is expressed. This generally comprises contacting cells with an appropriate agent that, but for the expression of the positive selection marker, kills or otherwise selects against the cells. An exemplary method of using selection markers is disclosed in U.S. Pat. No. 5,464,764, the disclosure of which is incorporated herein by reference in its entirety.

In some embodiments, suitable positive selection markers, and their corresponding selection agent, include, but are not limited to the following: Neo with G418; Neo with Kanamycin; Hyg with Hygromycin; hisD with Histidinol; Gpt with Xanthine; Ble with Bleomycin; and Hprt with Hypoxanthine.

A wide variety of such markers are known and available, including, for example, a Zeocin™ resistance marker, a blasticidin resistance marker, a neomycin resistance (neo) marker (Southern & Berg, J. Mol. Appl. Genet. 1: 327-41 (1982)), a puromycin (puro) resistance marker; a hygromycin resistance (hyg) marker (Te Riele et al., Nature 348:649-651 (1990)), thymidine kinase (tk), hypoxanthine phosphoribosyltransferase (hprt), and the bacterial guanine/xanthine phosphoribosyltransferase (gpt), which permits growth on MAX (mycophenolic acid, adenine, and xanthine) medium. See Song et al., Proc. Nat'l Acad. Sci. U.S.A. 84:6820-6824 (1987). Other selection markers include histidinol-dehydrogenase, chloramphenicol-acetyl transferase (CAT), dihydrofolate reductase (DHFR), 3-galactosyltransferase and fluorescent proteins such as GFP.

In some embodiments, the present disclosure provides for the use of a negative selection marker. For example, in some embodiments, a negative selection marker can include a polypeptide or a polynucleotide that, upon expression in a cell, allows for negative selection of the cell.

In one embodiment, a negative selection markers can be herpes simplex virusthymidine kinase (HSV-TK) marker, for negative selection in the presence of any of the nucleoside analogs acyclovir, gancyclovir, and 5-fluoroiodoamino-Uracil (FIAU). In yet other embodiments, the negative selection marker can be a toxin, such as the diphtheria toxin, the tetanus toxin, the cholera toxin and the pertussis toxin.

In still other embodiments, a negative selection marker can be hypoxanthine-guanine phosphoribosyl transferase (HPRT), for negative selection in the presence of 6-thioguanine.

In still other embodiments, the negative selection marker can be activators of apoptosis, or programmed cell death, such as the bcl2-binding protein (BAX). In some embodiments, the negative selection marker can be a cytidine deaminase (codA) gene of E. coli. or phosphotidyl choline phospholipase D. In one embodiment, the negative selection marker requires host genotype modification (e.g. ccdB, tolC, thyA, rpsl and thymidine kinases.)

In some embodiments, suitable negative selection markers, and their corresponding selection agent, include, but are not limited to the following: HSV-tk with Acyclovir; HSV-tk with Gancyclovir; herpes simplex virus-thymidine kinase (HSV-tk) with FIAU; Hprt with 6-thioguanine; Gpt with 6-thioxanthine; diphtheria toxin (DPT) (alone); diphtheria toxin fragment A (DPT-A) (alone); ricin toxin (alone); and Cytosine deaminase with 5-fluoro-cystosine.

In some embodiments the selection marker usually is chosen based on the type of the cell undergoing selection. For example, the cell can be eukaryotic (e.g., yeast), prokaryotic (e.g., bacterial), or viral. In some embodiments, the selection marker sequence can be operably linked to a promoter that is suited for that type of cell.

In another embodiment, more than one selection marker can be used. In such an embodiment, selection markers can be introduced wherein at least one selection marker is suited for one or more of target or host cells. In one embodiment, the host cell selection marker sequence and the target cell selection marker sequence are within the same open-reading frame and are expressed as a single protein. For example, the host cell and target cell selection marker sequence may encode the same protein, such as blasticidin S deaminase, which confers resistance to Blasticidin for both prokaryotic and eukaryotic cells. The host cell and the target cell marker sequence also may be expressed as a fusion protein. In another embodiment, the host cell and the target cell selection marker sequence are expressed as separate proteins.

In some embodiments, selection methods such as acetamide prototrophy selection; zeocin-resistance selection; geneticin-resistance selection; nourseothricin-resistance selection; uracil deficiency selection; and/or other selection methods may be used. For example, in some embodiments, the Aspergillus nidulans amdS gene can be used as selectable marker.

In some embodiments, vectors containing the targeted DNA constructs can be h altered to contain the neomycin phosphotransferase (neo^{r}) gene inside of them instead of the naturally occurring gene. In some embodiments, the neomycin phosphotransferase which is labeled neo^{r} is a gene that codes for a protein that makes the cell resistant to neomycin, a common antibiotic.

Exemplary descriptions and methods concerning selection markers are provided in Wigler et al., Cell 11:223 (1977); Szybalska & Szybalski, Proc. Natl. Acad. Sci. USA 48:202 (1992); Lowy et al., Cell 22:817 (1980); Wigler et al., Natl. Acad. Sci. USA 77:357 (1980); O'Hare et al., Proc. Natl. Acad. Sci. USA 78:1527 (1981); Mulligan & Berg, Proc. Natl. Acad. Sci. USA 78:2072 (1981); Wu and Wu, Biotherapy 3:87-95 (1991); Tolstoshev, Ann. Rev. Pharmacol. Toxicol. 32:573-596 (1993); Mulligan, Science 260:926-932 (1993); Morgan and Anderson, Ann. Rev. Biochem. 62:191-217 (1993); Santerre et al., Gene 30:147 (1984); Ausubel et al. (eds.), Current Protocols in Molecular Biology, John Wiley & Sons, N Y (1993); Kriegler, Gene Transfer and Expression, A Laboratory Manual, Stockton Press, N Y (1990); in Chapters 12 and 13, Dracopoli et al. (eds), Current Protocols in Human Genetics, John Wiley & Sons, N Y (1994); Colberre-Garapin et al., J. Mol. Biol. 150:1 (1981); U.S. Pat. No. 6,548,285 (filed Apr. 3, 1997); U.S. Pat. No. 6,165,715 (filed Jun. 22, 1998); and 6,110,707 (filed Jan. 17, 1997), the disclosures of which are incorporated by reference herein in their entireties.

### Viral Vectors

In some embodiments, delivery of a heterologous polynucleotide or transgene may be accomplished by a retroviral delivery system. See e.g., Eglitis et al., Adv. Exp. Med. Biol. 241:19, 1988. For example, in some embodiments, a retroviral construct can comprise a construct wherein the structural genes of the virus are replaced by a single gene which is then transcribed under the control of regulatory elements contained in the viral long terminal repeat (LTR). A variety of single-gene-vector backbones have been used, including the Moloney murine leukemia virus (MoMuLV). In one embodiment, retroviral vectors which permit multiple insertions of different genes such as a gene for a selectable marker and a second gene of interest, under the control of an internal promoter are derived from this type of backbone. See e.g., Gilboa, Adv. Exp. Med Biol. 241:29, 1988.

The use of packaging cell lines can increase the efficiency and the infectivity of the produced recombinant virions. See Miller, 1990, Human Gene Therapy 1:5. Murine retroviral vectors have been useful for transferring genes efficiently into murine embryonic. See e.g., Wagner et al., 1985, EMBO J. 4:663; Griedley et al., Trends Genet. 3:162, 1987, and hematopoietic stem cells, see e.g., Lemischka et al., Cell 45:917-927, 1986; Dick et al., Trends in Genetics 2:165-170, 1986.

An additional retroviral technology that permits attainment of much higher viral titers than were previously possible involves amplification by consecutive transfer between ecotropic and amphotropic packaging cell lines, the so-called “ping-pong” method. See, e.g., Kozak et al., J. Virol. 64:3500-3508, 1990; Bodine et al., Prog. Clin. Biol. Res. 319: 589-600, 1989. In addition, a techniques for increasing viral titers permit the use of virus-containing supernatants rather than direct incubation with virus-producing cell lines to attain efficient transduction. See e.g., Bodine et al., Prog. Clin. Biol. Res. 319:589-600, 1989. Because replication of cellular DNA is required for integration of retroviral vectors into the host genome, it may be desirable to increase the frequency at which target stem cells which are actively cycling e.g., by inducing target cells to divide by treatment in vitro with growth factors. See e.g., Lemischka et al., Cell 45:917-927, 1986; Bodine et al., Proc. Natl. Acad. Sci. 86:8897-8901, 1989. Alternatively, one may expose the recipient to 5-fluorouracil. See Mori et al., Jpn. J. Clin. Oncol. 14 Suppl. 1:457-463, 1984.

In some embodiments, lentiviral vectors/particles may be used as vehicles and delivery modalities. Lentiviruses are subgroup of the Retroviridae family of viruses, named because reverse transcription of viral RNA genomes to DNA is required before integration into the host genome. As such, the most important features of lentiviral vehicles/particles are the integration of their genetic material into the genome of a target/host cell. Some examples of lentivirus include the Human Immunodeficiency Viruses: HIV-1 and HIV-2, the Simian Immunodeficiency Virus (SIV), feline immunodeficiency virus (FIV), bovine immunodeficiency virus (BIV), Jembrana Disease Virus (JDV), equine infectious anemia virus (EIAV), equine infectious anemia virus, visna-maedi and caprine arthritis encephalitis virus (CAEV).

Typically, lentiviral particles making up the gene delivery vehicle are replication defective on their own (also referred to as “self-inactivating”). Lentiviruses can infect both dividing and non-dividing cells by virtue of the entry mechanism through the intact host nuclear envelope (Naldini L et al., Curr. Opin. Biotechnol, 1998, 9: 457-463). Recombinant lentiviral vehicles/particles have been generated by multiply attenuating the HIV virulence genes, for example, the genes Env, Vif, Vpr, Vpu, Nef and Tat are deleted making the vector biologically safe. Correspondingly, lentiviral vehicles, for example, derived from HIV-1/HIV-2 can mediate the efficient delivery, integration and long-term expression of transgenes into non-dividing cells. As used herein, the term “recombinant” refers to a vector or other nucleic acid containing both lentiviral sequences and non-lentiviral retroviral sequences.

Lentiviral particles may be generated by co-expressing the virus packaging elements and the vector genome itself in a producer cell such as human HEK293T cells. These elements are usually provided in three (in second generation lentiviral systems) or four separate plasmids (in third generation lentiviral systems). The producer cells are co-transfected with plasmids that encode lentiviral components including the core (i.e. structural proteins) and enzymatic components of the virus, and the envelope protein(s) (referred to as the packaging systems), and a plasmid that encodes the genome including a foreign transgene, to be transferred to the target cell, the vehicle itself (also referred to as the transfer vector). In general, the plasmids or vectors are included in a producer cell line. The plasmids/vectors are introduced via transfection, transduction or infection into the producer cell line. Methods for transfection, transduction or infection are well known by those of skill in the art. As non-limiting example, the packaging and transfer constructs can be introduced into producer cell lines by calcium phosphate transfection, lipofection or electroporation, generally together with a dominant selectable marker, such as neo, DHFR, Gln synthetase or ADA, followed by selection in the presence of the appropriate drug and isolation of clones.

The producer cell produces recombinant viral particles that contain the foreign gene, for example, the effector module of the present disclosure. The recombinant viral particles are recovered from the culture media and titrated by standard methods used by those of skill in the art. The recombinant lentiviral vehicles can be used to infect target cells.

Cells that can be used to produce high-titer lentiviral particles may include, but are not limited to, HEK293T cells, 293G cells, STAR cells (Relander et al., Mol. Ther., 2005, 11: 452-459), FreeStyle™ 293 Expression System (ThermoFisher, Waltham, MA), and other HEK293T-based producer cell lines (e.g., Stewart et al., Hum Gene Ther. 2011, 22(3):357-369; Lee et al., Biotechnol Bioeng, 2012, 10996): 1551-1560; Throm et al., Blood. 2009, 113(21): 5104-5110); the disclosures of which are incorporated herein by reference in their entireties.

In some embodiments, the envelope proteins may be heterologous envelop proteins from other viruses, such as the G protein of vesicular stomatitis virus (VSV G) or baculoviral gp64 envelop proteins. The VSV-G glycoprotein may especially be chosen among species classified in the vesiculovirus genus: Carajas virus (CJSV), Chandipura virus (CHPV), Cocal virus (COCV), Isfahan virus (ISFV), Maraba virus (MARAV), Piry virus (PIRYV), Vesicular stomatitis Alagoas virus (VSAV), Vesicular stomatitis Indiana virus (VSIV) and Vesicular stomatitis New Jersey virus (VSNJV), and/or stains provisionally classified in the vesiculovirus genus as Grass carp rhabdovirus, BeAn 157575 virus (BeAn 157575), Boteke virus (BTKV), Calchaqui virus (CQIV), Eel virus American (EVA), Gray Lodge virus (GLOV), Jurona virus (JURY), Klamath virus (KLAV), Kwatta virus (KWAV), La Joya virus (LJV), Malpais Spring virus (MSPV), Mount Elgon bat virus (MEBV), Perinet virus (PERV), Pikefry rhabdovirus (PFRV), Porton virus (PORV), Radi virus (RADIV), Spring viremia of carp virus (SVCV), Tupaia virus (TUPV), Ulcerative disease rhabdovirus (UDRV) and Yug Bogdanovac virus (YBV). The gp64 or other baculoviral env protein can be derived from Autographa californica nucleopolyhedrovirus (AcMNPV), Anagraphafalcifera nuclear polyhedrosis virus, Bombyx mori nuclear polyhedrosis virus, Choristoneura fumiferana nucleopolyhedrovirus, Orgyia pseudotsugata single capsid nuclear polyhedrosis virus, Epiphyas postvittana nucleopolyhedrovirus, Hyphantria cunea nucleopolyhedrovirus, Galleria mellonella nuclear polyhedrosis virus, Dhori virus, Thogoto virus, Antheraea pemyi nucleopolyhedrovirus or Batken virus.

Additional elements provided in lentiviral particles may comprise retroviral LTR (long-terminal repeat) at either 5′ or 3′ terminus, a retroviral export element, optionally a lentiviral reverse response element (RRE), a promoter or active portion thereof, and a locus control region (LCR) or active portion thereof. Other elements include central polypurine tract (cPPT) sequence to improve transduction efficiency in non-dividing cells, Woodchuck Hepatitis Virus (WHP) Posttranscriptional Regulatory Element (WPRE) which enhances the expression of the transgene, and increases titer. The effector module is linked to the vector.

Methods for generating recombinant lentiviral particles are discussed in the art, for example, U.S. Pat. Nos. 6,808,905; 7,179,903; 7,575,924; 7,629,153; 7,745,179; and 8,846,385; the contents of each of which are incorporated herein by reference in their entirety.

In some embodiments, lentivirus vectors may be selected from, but are not limited to pLVX, pLenti, pLenti6, pLJM1, FUGW, pWPXL, pWPI, pLenti CMV puro DEST, pLJM1-EGFP, pULTRA, pInducer20, pHIV-EGFP, pCW57.1, pTRPE, pELPS, pRRL, and pLionlI.

Lentiviral vehicles known in the art may also be used. Exemplary descriptions are provided in: U.S. Pat. Nos. 5,994,136; 6,013,516; 8,076,106; 8,329,462; 8,420,104; 8,709,799; 8,748,169; 8,900,858; 9,023,646; 9,068,199; and 9,260,725; the contents of each of which are incorporated herein by reference in their entirety.

### Methods of Making Transgenic Animals

Transgenic animal technology presents a unique opportunity to study the characteristics of human proteins in non-human animals. Recombinant DNA and genetic engineering techniques have made it possible to introduce and express a desired sequence or gene in a recipient animal making it possible to study the effects of a particular molecule in vivo and study agents that bind to the molecule. Transgenic animals are produced by introducing one or more heterologous polynucleotides (also referred to as transgenes) into the germline of the transgenic animal. The methods enabling the introduction of DNA into cells are generally available and well-known in the art and different methods of introducing transgenes could be used. See, e.g., Hogan et al. Manipulating the Mouse Embryo: A Laboratory Manual Cold Spring Harbor Laboratory, 2nd edition, Cold Spring Harbor Laboratory (1994) and U.S. Pat. Nos. 5,602,299; 5,175,384; 6,066,778 and 6,037,521, the disclosures of which are incorporated herein by reference in their entireties.

In some embodiments, the present disclosure provides a method of making a transgenic non-human animal comprising: (i) introducing a heterologous polynucleotide comprising human PTH1R exons 4 to 16 into a non-human animal embryonic stem (ES) cell, such that the heterologous polynucleotide integrates into an endogenous non-human animal PTH1R locus; (ii) obtaining a non-human animal ES cell comprising a modified genome, wherein the heterologous polynucleotide has integrated into an endogenous non-human animal PTH1R locus; and (iii) generating a non-human animal using the non-human animal ES cell comprising the modified genome.

In some embodiments, the present disclosure provides a method of making a transgenic non-human animal, wherein the non-human animal is a mammal.

In some embodiments, the present disclosure provides a method of making a transgenic non-human animal is a mammal selected from the group consisting of: a mouse; a rat; a guinea pig; a hamster; and a gerbil.

In some embodiments, the present disclosure provides a method of making a transgenic non-human animal, wherein the transgenic non-human animal is a mouse.

In some embodiments, the present disclosure provides a method of making a transgenic non-human animal, wherein the transgenic non-human animal is: a 129 mouse; an A mouse; a BALB/c mouse; a C3H mouse; a C57BL mouse; a C57BR mouse; a C57L mouse; a CB17 mouse; a CD-1 mouse; a DBA mouse; an FVB mouse; an SJL mouse; an SWR mouse; any substrain thereof; any hybrid strain thereof; any congenic strain thereof; or any mutant strain thereof.

In some embodiments, the present disclosure provides a method of making a transgenic non-human animal, wherein the transgenic non-human animal is: a C57BL/6 mouse, or a C57BL/10 mouse.

In some embodiments, the present disclosure provides a method of making a transgenic non-human animal, wherein the transgenic non-human animal is a C57BL/6 mouse.

In some embodiments, the present disclosure provides a method of making a transgenic non-human animal comprising: (i) introducing a heterologous polynucleotide comprising human PTH1R exons 4 to 16 into a non-human animal embryonic stem (ES) cell, such that the heterologous polynucleotide integrates into an endogenous non-human animal PTH1R locus; (ii) obtaining a non-human animal ES cell comprising a modified genome, wherein the heterologous polynucleotide has integrated into an endogenous non-human animal PTH1R locus; and (iii) generating a non-human animal using the non-human animal ES cell comprising the modified genome, wherein the heterologous polynucleotide comprising human PTH1R exons 4 to 16 is operable to encode a human PTH1R protein having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.1% identical, at least 99.2% identical, at least 99.3% identical, at least 99.4% identical, at least 99.5% identical, at least 99.6% identical, at least 99.7% identical, at least 99.8% identical, at least 99.9% identical, or 100% identical to an amino acid sequence set forth in SEQ ID NOs: 1 or 29

In some embodiments, the present disclosure provides a method of making a transgenic non-human animal comprising a heterologous polynucleotide comprising human PTH1R exons 4 to 16, wherein the heterologous polynucleotide comprising human PTH1R exons 4 to 16 is stably integrated in the genome of the transgenic non-human animal.

In some embodiments, the present disclosure provides a method of making a transgenic non-human animal, wherein the heterologous polynucleotide comprising human PTH1R exons 4 to 16 is stably integrated in an endogenous non-human animal PTH1R gene locus.

In some embodiments, the present disclosure provides a method of making a transgenic non-human animal, wherein the heterologous polynucleotide comprising human PTH1R exons 4 to 16 is stably integrated in an endogenous non-human animal PTH1R gene locus that causes a replacement of a genomic DNA segment comprising non-human animal PTH1R exon 4, with the heterologous polynucleotide comprising human PTH1R exons 4 to 16.

In some embodiments, the present disclosure provides a method of making a transgenic non-human animal, wherein the replacement results in a heterozygous transgenic non-human animal, or a homozygous transgenic non-human animal.

In some embodiments, the present disclosure provides a method of making a transgenic non-human animal comprising: (i) introducing a heterologous polynucleotide comprising human PTH1R exons 4 to 16 into a non-human animal embryonic stem (ES) cell, such that the heterologous polynucleotide integrates into an endogenous non-human animal PTH1R locus; (ii) obtaining a non-human animal ES cell comprising a modified genome, wherein the heterologous polynucleotide has integrated into an endogenous non-human animal PTH1R locus; and (iii) generating a non-human animal using the non-human animal ES cell comprising the modified genome.

Techniques for creating a transgenic animal, particularly a mouse or rat are well known (see, e.g., Gordon, International Review of Cytology 115:171-229, 1989). Various approaches to introducing transgenes are available, including microinjection of nucleic acids into cells, retrovirus vector methods, and gene transfer into embryonic stem cells (ESCs) by harnessing homologous recombination. These methods are described in detail below.

### Microinjection

Microinjection can be used to create transgenic animals of the present disclosure. Generally, the zygote is the best target for microinjection. In mice, for example, the male pronucleus reaches the size of approximately 20 μm in diameter, which allows reproducible injection of 1-2 pL of DNA solution. The use of zygotes as a target for gene transfer has a major advantage. In most cases, the injected DNA will be incorporated into the host gene before the first cleavage. Consequently, nearly all cells of the transgenic non-human animal will carry the incorporated transgene. Generally, this will also result in the efficient transmission of the transgene to offspring of the founder since 50% of the germ cells will harbor the transgene. Microinjection of zygotes is one method for incorporating transgenes in practicing the invention. The pronuclear microinjection method of producing a transgenic animal results in the introduction of linear DNA sequences into the chromosomes of the fertilized eggs. Bacterial Artificial Chromosome (BAC) containing the gene of interest or an alternative plasmid construct containing the gene of interest is injected into pronuclei (i.e., fertilized eggs at a pronuclear state). The manipulated pronuclei are subsequently injected into the uterus of a pseudopregnant female. Mice generated can have one or multiple copies of the transgene, which can be assayed by southern blot technology.

In some embodiments, transgenic animals can be generated via pronuclear microinjection. An exemplary description of pronuclear microinjection is provided in Gordon, J. W., PNAS 77, 7380-7384 (1980), and U.S. Pat. No. 4,873,191, the disclosures of which are incorporated herein by reference in their entireties.

In some embodiments, a transgenic non-human animal of the present disclosure can be generated by microinjection of DNA. In other embodiments, infection with a viral vector containing the gene construct can be used to insert the gene of interest into a zygote or into embryonic stem cells.

In some embodiments, the transgenic non-human animals of the present disclosure can be generated by recovering fertilized eggs from newly mated female mice, followed by microinjection of the DNA of the gene of interest into the male pronucleus of the egg. The microinjected eggs are then implanted in the oviducts of one-day pseudopregnant foster mothers and allowed to proceed to term. See, Wagner et al., Microinjection of a rabbit beta-globin gene into zygotes and its subsequent expression in adult mice and their offspring. Proc Natl Acad Sci USA. 1981 October; 78(10):6376-80; U.S. Pat. Nos. 4,873,191; and 7,294,755; the disclosures of which are incorporated herein by reference in their entireties.

Microinjection of DNA is routinely used to generate transgenic mice. Exemplary methods of creating transgenic mice via microinjection are provided in: U.S. Pat. No. 4,736,866; Hogan et al. entitled “Manipulating the Mouse Embryo: A Laboratory Manual”, Cold Spring Harbor Laboratory, Cold Spring Harbor, N.Y., U.S.A. (1986); Haren et al., “Integrating DNA: transposases and retroviral integrases,” Annu. Rev. Microbiol., 53:245-81 (1999); Ivics et al., “Genetic applications of transposons and other repetitive elements in zebrafish”, Methods Cell Biol., 60:99-131 (1999); and Han et al., FEMS Microbiol. Rev. 21:157-178 1997, the disclosures of which are incorporated herein by reference in their entireties.

### Virus-Mediated Gene Transfer.

Viral vectors may be used to produce a transgenic animal. Preferably, the viral vectors are replication defective, i.e., they are unable to replicate autonomously in the target cell.

In general, the genome of the replication defective viral vectors which are used lack at least one region which is necessary for the replication of the virus in the infected cell. These regions can either be eliminated (in whole or in part), be rendered non-functional by any technique known to a person skilled in the art. These techniques include the total removal, substitution (by other sequences, in particular by the inserted nucleic acid), partial deletion or addition of one or more bases to an essential (for replication) region. Such techniques may be performed in vitro (on the isolated DNA) or in situ, using the techniques of genetic manipulation or by treatment with mutagenic agents. Preferably, the replication defective virus retains the sequences of its genome which are necessary for encapsidating the viral particles.

The retroviruses are integrating viruses which infect dividing cells. The retrovirus genome includes two LTRs, an encapsidation sequence and three coding regions (gag, pol and env). The construction of recombinant retroviral vectors has been described: see, in particular, EP 453242, EP178220, Bernstein et al. Genet. Eng. 7 (1985) 235; McCormick, BioTechnology 3 (1985) 689, etc.

In recombinant retroviral vectors, the gag, pol and env genes are generally deleted, in whole or in part, and replaced with a heterologous nucleic acid sequence of interest. These vectors can be constructed from different types of retrovirus, such as, HIV, MoMuLV (“murine Moloney leukemia virus”), MSV (“murine Moloney sarcoma virus”), HaSV (“Harvey sarcoma virus”); SNV (“spleen necrosis virus”); RSV (“Rous sarcoma virus”) and Friend virus. Defective retroviral vectors are disclosed in WO95/02697.

In general, in order to construct recombinant retroviruses containing a nucleic acid sequence, a plasmid is constructed which contains the LTRs, the encapsidation sequence and the coding sequence. This construct is used to transfect a packaging cell line, which cell line is able to supply in trans the retroviral functions which are deficient in the plasmid. In general, the packaging cell lines are thus able to express the gag, pol and env genes. Such packaging cell lines have been described in the prior art, in particular the cell line 17 (U.S. Pat. No. 4,861,719); the PsiCrip cell line (WO90/02806) and the GP+envAm-12 cell line (WO89/07150). In addition, the recombinant retroviral vectors can contain modifications within the LTRs for suppressing transcriptional activity as well as extensive encapsidation sequences which may include a part of the gag gene. Recombinant retroviral vectors are purified by standard techniques known to those having ordinary skill in the art. Additional means of using retroviruses or retroviral vectors to create transgenic animals known to the art involve the micro-injection of retroviral particles or mitomycin C-treated cells producing retrovirus into the perivitelline space of fertilized eggs or early embryos. See WO 90/08832 (1990); Haskell and Bowen, Mol. Reprod. Dev. 40:386 (1995).

### Site-Specific Nucleases and Other Gene Editing Methods

“Site-specific nucleases” refers to nucleases that create double-stranded breaks at desired locations. In some embodiments, a site-specific nuclease can be a zinc finger nuclease (ZFN); transcription activation-like effector nuclease (TALEN); or CRISPR/Cas system. ZFNs are artificial restriction enzymes generated by fusing a zinc finger DNA-binding domain to a DNA-cleavage domain. TALENs are artificial restriction enzymes generated by fusing a TAL effector DNA binding domain to a DNA cleavage domain. ZFNs and TALENs can be quickly engineered to bind practically any desired DNA sequence because their DNA binding domains can be designed to target desired DNA sequences and this enables nucleases to target unique sequences even within complex genomes. Specificity of methods using ZFNs and TALENs is due to DNA binding domains, which direct DNA cleavages to the neighboring sequences. ZFN and TALEN techniques are described in various practical manuals describing laboratory molecular techniques, e.g., Hockemeyer et al. 2012, Nat Biotechnol 29(8): 731-734; Hockemeyer et al. 2009, Nat Biotechnol 27(9): 851-857), the disclosures of which are incorporated by reference herein in their entirety. The CRISPR/Cas system has been described by Sander and Joung (2014), Nature Biotechnology 32, 347-355, the disclosure of which is incorporated herein by reference in its entirety.

In some embodiments, a transgenic non-human animal of the present disclosure can be created using site-specific nucleases and/or gene editing methods. For example, in some embodiments, a transgenic non-human animal can be created using gene editing systems including, but not limited to: a CRISPR (Clustered Regularly Interspaced Short Palindromic Repeats), CRISPR enzyme (Cas9), CRISPR-Cas9 or CRISPR system and CRISPR-CAS9 complex. In other embodiments, Zinc finger nucleases, TALEN (Transcription activator-like effector-based nucleases) and/or meganucleases may be used.

In some embodiments, site-specific nucleases can be used to create a transgenic non-human animal of the present disclosure.

In some embodiments, nucleases can create double-strand breaks at desired locations. For example, in some embodiments, nucleases can create double-strand breaks at the or around one or more polynucleotides encoding one or more endogenous genes with shared homology to a transgene of interest, creating a repair point for recombination.

In some embodiments, a site-specific nuclease can be a zinc finger nuclease (ZFN). For example, in some embodiments, a zinc finger nuclease (ZFN) can be used can be used to create a transgenic non-human animal of the present disclosure.

In some embodiments, a site-specific nuclease can be a transcription activation-like effector nuclease (TALEN). For example, in some embodiments, a transcription activation-like effector nuclease (TALEN) can be used to create transgenic non-human animal of the present disclosure.

In some embodiments, a site-specific nuclease can be a CRISPR/Cas system. For example, in some embodiments, a CRISPR/Cas system can be used to create a transgenic non-human animal of the present disclosure.

Exemplary methods for ZFN and TALEN techniques are described in Hockemeyer et al. 2012, Nat Biotechnol 29(8): 731-734; Hockemeyer et al. 2009, Nat Biotechnol 27(9): 851-857), the disclosures of which are incorporated by reference herein in their entirety. Exemplary methods for the CRISPR/Cas system is described by Sander and Joung (2014), Nature Biotechnology 32, 347-355, the disclosure of which is incorporated herein by reference in its entirety.

In some preferred embodiments, a CRISPR-Cas9 system can be used to create transgenic non-human animals of the present disclosure. The CRISPR-Cas9 system is a novel genome editing system which has been rapidly developed and implemented in a multitude of model organisms and cell types, and supplants other genome editing technologies, such as TALENs and ZFNs. CRISPRs are sequence motifs are present in bacterial and archaeal genomes, and are composed of short (about 24-48 nucleotide) direct repeats separated by similarly sized, unique spacers See Grissa et al. BMC Bioinformatics 8, 172 (2007). They are generally flanked by a set of CRISPR-associated (Cas) protein-coding genes that are required for CRISPR maintenance and function. See Barrangou et al., Science 315, 1709 (2007); Brouns et al., Science 321, 960 (2008); and Haft et al. PLoS Comput Biol 1, e60 (2005).

CRISPR-Cas systems provide adaptive immunity against invasive genetic elements (e.g., viruses, phages and plasmids). See Horvath and Barrangou, Science, 2010, 327: 167-170; Bhaya et al., Annu. Rev. Genet., 2011, 45: 273-297; and Barrangou R, RNA, 2013, 4: 267-278. Three different types of CRISPR-Cas systems have been classified in bacteria and the type II CRISPR-Cas system is most studied. In the bacterial Type II CRISPR-Cas system, small CRISPR RNAs (crRNAs) processed from the pre-repeat-spacer transcript (pre-crRNA) in the presence of a trans-activating RNA (tracrRNA)/Cas9 can form a duplex with the tracrRNA/Cas9 complex. The mature complex is recruited to a target double strand DNA sequence that is complementary to the spacer sequence in the tracrRNA: crRNA duplex to cleave the target DNA by Cas9 endonuclease. See Garneau et al., Nature, 2010, 468: 67-71; Jinek et al., Science, 2012, 337: 816-821; Gasiunas et al., Proc. Natl Acad. Sci. USA., 109: E2579-2586; and Haurwitz et al., Science, 2010, 329: 1355-1358. Target recognition and cleavage by the crRNA: tracrRNA/Cas9 complex in the type II CRISPR-CAS system not only requires a sequence in the tracrRNA: crRNA duplex that is a complementary to the target sequence (also called “protospacer” sequence), but also requires a protospacer adjacent motif (PAM) sequence located 3′end of the protospacer sequence of a target polynucleotide. The PAM motif can vary between different CRISPR-Cas systems.

CRISPR-Cas9 systems have been developed and modified for use in genetic editing and prove to be a high effective and specific technology for editing a nucleic acid sequence even in eukaryotic cells. Many researchers disclosed various modifications to the bacterial CRISPR-Cas systems and demonstrated that CRISPR-Cas systems can be used to manipulate a nucleic acid in a cell, such as in a mammalian cell (e.g., a mouse cell).

In some embodiments, transgenic animals of the present disclosure can be created using a CRISPR/Cas9 system that includes alternative isoforms or orthologs of the Cas9 enzyme.

The most commonly used Cas9 is derived from Streptococcus pyogenes and the RuvC domain can be inactivated by a D10A mutation and the HNH domain can be inactivated by an H840A mutation.

In addition to Cas9 derived from S. pyogenes, other RNA guided endonucleases (RGEN) may also be used for programmable genome editing. Cas9 sequences have been identified in more than 600 bacterial strains. Though Cas9 family shows high diversity of amino acid sequences and protein sizes, All Cas9 proteins share a common architecture with a central HNH nuclease domain and a split RuvC/RHase H domain. Examples of Cas9 orthologs from other bacterial strains including but not limited to, Cas proteins identified in Acaryochloris marina MBIC11017; Acetohalobium arabaticum DSM 5501; Acidithiobacillus caldus; Acidithiobacillus ferrooxidans ATCC 23270; Alicyclobacillus acidocaldarius LAA1; Alicyclobacillus acidocaldarius subsp. acidocaldarius DSM 446; Allochromatium vinosum DSM 180; Ammonifex degensii KC4; Anabaena variabilis ATCC 29413; Arthrospira maxima CS-328; Arthrospira platensis str. Paraca; Arthrospira sp. PCC 8005; Bacillus pseudomycoides DSM 12442; Bacillus selenitireducens MLS10; Burkholderiales bacterium 1_1_47; Caldicelulosiruptor becscii DSM 6725; Candidatus Desulforudis audaxviator MP104C; Caldicellulosiruptor hydrothermalis_108; Clostridium phage c-st; Clostridium botulinum A3 str. Loch Maree; Clostridium botulinum Ba4 str. 657; Clostridium difficile QCD-63q42; Crocosphaera watsonii WH 8501; Cyanothece sp. ATCC 51142; Cyanothece sp. CCY0110; Cyanothece sp. PCC 7424; Cyanothece sp. PCC 7822; Exiguobacterium sibiricum 255-15; Finegoldia magna ATCC 29328; Ktedonobacter racemifer DSM 44963; Lactobacillus delbrueckii subsp. bulgaricus PB2003/044-T3-4; Lactobacillus salivarius ATCC 11741; Listeria innocua; Lyngbya sp. PCC 8106; Marinobacter sp. ELB17; Methanohalobium evestigatum Z-7303; Microcystis phage Ma-LMMO1; Microcystis aeruginosa NIES-843; Microscilla marina ATCC 23134; Microcoleus chthonoplastes PCC 7420; Neisseria meningitidis; Nitrosococcus halophilus Nc4; Nocardiopsis dassonvillei subsp. dassonvillei DSM 43111; Nodularia spumigena CCY9414; Nostoc sp. PCC 7120; Oscillatoria sp. PCC 6506; Pelotomaculum thermopropionicum SI; Petrotoga mobilis SJ95; Polaromonas naphthalenivorans CJ2; Polaromonas sp. JS666; Pseudoalteromonas haloplanktis TAC125; Streptomyces pristinaespiralis ATCC 25486; Streptomyces pristinaespiralis ATCC 25486; Streptococcus thermophilus; Streptomyces viridochromogenes DSM 40736; Streptosporangium roseum DSM 43021; Synechococcus sp. PCC 7335; and Thermosipho aficanus TCF52B (Chylinski et al., RNA Biol., 2013; 10(5): 726-737).

In addition to Cas9 orthologs, other Cas9 variants such as fusion proteins of inactive dCas9 and effector domains with different functions may be served as a platform for genetic modulation. Any of the foregoing enzymes may be useful in the present disclosure.

Exemplary descriptions of methods concerning CRISPR/Cas systems are provided in U.S. Pat. Nos. 8,697,359; 8,771,945; 8,865,406; 8,871,445; 8,889,356; 8,889,418; 8,895,308; 8,906,616; 8,932,814; 8,945,839; 8,999,641; 8,993,233; and U.S. patent publication Nos.: 20150031134; 20150203872; 20150218253; 20150176013; 20150191744; 20150071889; 20150067922; and 20150167000; the disclosures of which are incorporated herein by reference in their entireties.

### Embryonic Stem Cell-Mediated Gene Transfer

Embryonic stem cell-mediated gene transfer can be used to create transgenic animals of the present disclosure. For example, in some embodiments, transgenic animals can be generated by introduction of the targeting vectors into embryonal stem cells (ESCs). ESCs are obtained by culturing pre-implantation embryos in vitro under appropriate conditions. See Evans et al., Nature 292:154-156 (1981); Bradley et al., Nature 309:255-258 (1984); Gossler et al., PNAS 83:9065-9069 (1986); and Robertson et al., Nature 322:445-448 (1986).

Transgenes can be efficiently introduced into the ESCs by DNA transfection using a variety of methods known to the art including electroporation, calcium phosphate co-precipitation, protoplast or spheroplast fusion, lipofection and DEAE-dextran-mediated transfection. Alternatively, transgenes can also be introduced into ES cells by retrovirus-mediated transduction. Such transfected ESCs can thereafter colonize an embryo following their introduction into the blastocoel of a blastocyst-stage embryo and contribute to the germ line of the resulting chimeric animal. See Jaenisch, Science 240:1468-1474 (1988).

Prior to the introduction of transformed ESCs into the blastocoel, the transformed ESCs can be subjected to various selection protocols to enrich for ESCs that have integrated the transgene-if the transgene provides a means for such selection. Alternatively, PCR can be used to screen for ESCs that have integrated the transgene. This technique obviates the need for growth of the transformed ESCs under appropriate selective conditions prior to transfer into the blastocoel.

Accordingly, transforming ESCs with a polynucleotide of interest through the use of vectors, offers the possibility of altering ESCs in a controlled manner and therefore, of generating transgenic non-human animals with a predetermined genome. Exemplary descriptions of ESC transformation methods in the generation of transgenic animals are provided in Baribault and Kemler. Embryonic stem cell culture and gene targeting in transgenic mice. Mol Biol Med. 6:481-92, 1989; Ledermann B. Embryonic stem cells and gene targeting. Exp Physiol. 85:603-13, 2000; and Moreadith and Radford. Gene targeting in embryonic stem cells: the new physiology and metabolism. J Mol Med. 75:208-16, 1997, the disclosures of which are incorporated herein by reference in their entireties.

In some embodiments, transgenic animals can be generated using in vivo homologous recombination, e.g., transformation of ES cells, followed by transferring said ES cells into blastocysts.

In some embodiments, transgenes can be incorporated into embryonic, fetal or adult pluripotent stem cells. See Capecchi et al. Science 244:1288-1292, 1991, the disclosure of which is incorporated herein by reference in its entirety. For example, in some embodiments, embryonic stem cells can be isolated from blastocysts cultivated in vitro. These embryonic stem cells are kept stable in culture over many cell generations without differentiation. In some embodiments, the transgene is then incorporated into the embryonic stem cells by electroporation or other methods of transformation. Stem cells carrying the transgene are selected for and injected into the inner cell mass of blastocysts. The blastocysts are then implanted into pseudopregnant females. Because not all the cells of the inner cell mass of the blastocysts carry the transgenes, the animals are chimeric with respect to the transgenes. Crossbreeding of the chimeric animals allows for the production of animals which carry the transgene. An overview of the process is provided by Capecchi, Trends in Genetics 1989, 5:70-76, the disclosure of which is incorporated herein by reference in its entirety.

In some preferred embodiments, transgenic non-human animals of the present disclosure can be created by a procedure using embryonic stem cells, which are transformed with a polynucleotide of interest. For example, in some embodiments, embryonic stem cells (ESCs) are transformed with a heterologous polynucleotide comprising human Parathyroid Hormone 1 Receptor (PTH1R) exons 4 to 16; then, the transfected ESCs are injected into mouse blastocysts where they take part in the formation of all tissues, including the germ line, thus generating transgenic offspring.

In some embodiments, transgenic non-human animals (e.g., a mouse) can be created by transforming ESCs with a polynucleotide of interest, and injecting the transformed cells into a blastocyst. In some embodiments, by interbreeding heterozygous siblings, homozygous animals carrying the desired polynucleotide are obtained. An exemplary description of creating transgenic mice via transforming ESCs is provided in U.S. Pat. No. 6,492,575, the disclosure of which is incorporated herein by reference in its entirety.

In some embodiments, ESCs can be derived from the pluripotent inner cell mass (ICM) of blastocysts, e.g., a 3.5 days old pre-implantation mouse embryo; accordingly, ESCs obtained at this stage are operable to contribute to all embryonic tissues, including the germ cells, in developing mice.

In some embodiments, a 3.5-day-old mouse embryo (blastocysts) can be collected from the uterine horn of superovulated (hormone treated) mated female mice. In some embodiments, the selection of mice for this aspect of the procedure can be based on coat color, e.g., an agouti coat (129/Sv) or a C57BL/6 with a black coat or albino.

In some embodiments, ESCs can be derived from the inner cell mass of blastocysts and cultured on a feeder layer of mitotically inactivated mouse embryonic fibroblasts (MEFs), in ESC medium (supplemented with leukemia inhibitory factor (LIF).

In some embodiments, ESCs can be electroporation with a targeting vector comprising a polynucleotide of interest, e.g., a heterologous polynucleotide comprising human Parathyroid Hormone 1 Receptor (PTH1R) exons 4 to 16.

In some embodiments, the successfully transfected ESCs can be selected by adding appropriate selection agent to the ESC medium, and positive ESC clones can subsequently be chosen.

In some embodiments, homologous recombinant ESC clones can be identified by Southern blot. For example, in some embodiments, the genomic DNA isolated from ESC clones may be digested with an appropriate restriction enzyme, resulting in a single cut inside the targeting vector, and a single cut outside (i.e., upstream or downstream, respectively) the targeting vector, in the targeted chromosomal region. In some embodiments, the use of an external probe outside of the targeting construct will produce a band with a size corresponding to unmodified wild-type allele(s); and, if homologous recombination occurred, a second band of bigger or smaller size corresponding to the targeted allele, can be identified.

In some embodiments, transgenic non-human animals can be created as follows: (1) modifying the genome of a pluripotent cell (e.g., transformation with a vector); (2) selecting the modified pluripotent cell; (3) introducing the modified pluripotent cell into a host embryo; and (4) implanting the host embryo comprising the modified pluripotent cell into a surrogate mother. Subsequent to the foregoing steps, one or more progeny from the modified pluripotent cell will be generated.

In some embodiments, the donor cell can be introduced into a host embryo at any stage, e.g., the blastocyst stage or the pre-morula stage (i.e., the 4 cell stage or the 8 cell stage). Here, the goal is to develop progeny capable of transmitting the modification (e.g., a gene or trait of interest) though the germline. In some embodiments, the pluripotent cell to be modified can be an embryonic stem cell (ESC), e.g., a mouse ESC or a rat ESC.

In some embodiments, methods of generating a transgenic non-human animal can comprise the following: (1) modifying the genome of a non-human ESC (e.g., transformation with a vector comprising a gene of interest); (2) identifying a non-human ES cell comprising the targeted modification; (3) introducing the non-human ES cell comprising the targeted modification into a non-human host embryo; and (4) gestating the non-human host embryo in a surrogate mother. Here, the surrogate mother then produces the F0 generation non-human animal comprising the targeted modification. The host embryo comprising the modified non-human ESC can be incubated until the blastocyst stage and then implanted into a surrogate mother to produce an F0 animal.

In some embodiments, transgenic non-human animals may be generated to express or overexpress a protein of interest (knock-in mice) or may be generated to delete a gene of interest (knock-out mice). For example, in some embodiments, transgenic non-human animals that express a human protein molecule allow for study of said human molecules in vivo.

In some embodiments, a cre/loxP recombinase system is utilized for generation of the transgenic animals. For example, the Cre/loxP recombinase systems described in Hickman-Davis et al. (Pediatric Respiratory Reviews 2006 7: 49) can be used. For this system, the generation of two independent mouse lines requires: (1) mice that contain the target gene or gene segment flanked by two 34 bp, asymmetric nucleotide sequences (loxP) sites in the same orientation (‘floxed’ sequence) and (2) mice that contain a fusion transgene expressing the Cre recombinase of the P1 bacteriophage. The Cre recombinase promotes recombination by recognition of the loxP sites, and when these two mouse strains are crossed, the floxed gene is deleted and a null mutation is created. Cre/loxP recombinase system is also useful in the targeted mutagenesis of embryonic stem cells in vitro to create (clean) mutations that lack a selection cassette that might interfere with gene regulation, in which pluripotent stem cells containing the gene of interest and only one loxP site with foreign sequence are generated for use in the creation of a transgenic mouse. Several methods have been demonstrated for controlling Cre expression including the creation of fusion proteins containing Cre and having specific ligand-binding domains (i.e., Cre is expressed only in the presence of a specific ligand), as well as a tetracycline-inducible Cre system.

### Non-Human Animals for Embryonic Stem-Cell Transfer

In some embodiments, a transgenic non-human animal of the present disclosure can be any non-human animal. Examples of non-human animals suitable to practice the present disclosure are described above and throughout the specification.

In some embodiments, a transgenic non-human animal of the present disclosure can be a fungus (e.g., a yeast cell); an invertebrate animal (e.g. fruit fly, cnidarian, echinoderm, nematode, etc.); or vertebrate animal (e.g., fish, amphibian, reptile, bird, mammal, or non-human primate).

In some embodiments, a transgenic non-human animal of the present disclosure is a vertebrate. For example, in some embodiments, the transgenic non-human animal can be, without limitation: a fish (e.g., zebra fish, gold fish, puffer fish, cave fish, etc.); an amphibian (frog, salamander, etc.); a bird (e.g., chicken, turkey, etc.); a reptile (e.g., snake, lizard, etc.); a mammal (e.g., an ungulate, e.g., a pig, a cow, a goat, a sheep, etc.); a lagomorph (e.g., a rabbit); a rodent (e.g., a rat, a mouse); or a non-human primate.

In some preferred embodiments, a transgenic non-human animals of the present disclosure can be a member selected from the order, Rodentia.

In some embodiments, transgenic non-human animals of the present disclosure can be a mouse; a rat; a guinea pig; a hamster; or a gerbil.

In some embodiments, a transgenic non-human animals of the present disclosure can be a member selected from the genera, Mus.

In some embodiments, a transgenic non-human animals of the present disclosure can be a member selected from following group: Mus musculus (house mouse); Mus musculus albula; Mus musculus bactrianus (southwestern Asian house mouse); Mus musculus brevirostris; Mus musculus castaneus (southeastern Asian house mouse); Mus musculus domesticus (western European house mouse); Mus musculus domesticus x M. m. molossinus; Mus musculus gansuensis; Mus musculus gentilulus; Mus musculus helgolandicus; Mus musculus homourus; Mus musculus isatissus; Mus musculus molossinus (Japanese wild mouse); Mus musculus musculus (eastern European house mouse); Mus musculus musculus x M. m. castaneus; Mus musculus musculus x M. m. domesticus; and/or Mus musculus wagneri.

In some embodiments, a transgenic non-human animals of the present disclosure can be: a 129 mouse; an A mouse; a BALB/c mouse; a C3H mouse; a C57BL mouse; a C57BR mouse; a C57L mouse; a CB17 mouse; a CD-1 mouse; a DBA mouse; an FVB mouse; an SJL mouse; an SWR mouse; any substrain thereof; any hybrid strain thereof; any congenic strain thereof; or any mutant strain thereof.

### Founder Generation

The various methods provided herein allow for the generation of a genetically modified non-human F0 animal wherein the cells of the genetically modified F0 animal that comprise the targeted modification. It is recognized that depending on the method used to generate the F0 animal, the number of cells within the F0 animal that have the nucleotide sequence of interest and lack the recombinase cassette and/or the selection cassette (if included) will vary.

The introduction of the donor ESCs into a pre-morula stage embryo from a corresponding organism (e.g., an 8-cell stage mouse embryo) via for example, the VELOCIMOUSE® method allows for a greater percentage of the cell population of the F0 animal to comprise cells having the nucleotide sequence of interest comprising the targeted genetic modification. In specific instances, at least 50%, 60%, 65%, 70%, 75%, 85%, 86%, 87%, 87%, 88%, 89%, 90%, 91%, 92%, 93%, 94%, 95%, 96%, 97%, 98%, 99% or 100% of the cellular contribution of the non-human F0 animal comprises a cell population having the targeted modification. In other instances, at least one or more of the germ cells of the F0 animal have the targeted modification.

Once the founder animals are produced, they can be bred, inbred, outbred, or crossbred to produce colonies of the particular animal. Examples of such breeding strategies include but are not limited to: outbreeding of founder animals with more than one integration site in order to establish separate lines; inbreeding of separate lines in order to produce compound transgenics that express the transgene at higher levels because of the effects of additive expression of each transgene; crossing of heterozygous transgenic mice to produce mice homozygous for a given integration site in order to both augment expression and eliminate the need for screening of animals by DNA analysis; crossing of separate homozygous lines to produce compound heterozygous or homozygous lines; breeding animals to different inbred genetic backgrounds so as to examine effects of modifying alleles on expression of the transgene and the physiological effects of expression.

To assess whether the ESCs have contributed to the germ layer of the chimeras, mouse coat color markers can be used. For example, the coat color of the 129/Sv ESC is dominant over the black coat color of a C57BL/6J mice: thus, mating the chimeras with C57BL/6J mice should yield either black pups, when the germ cells of the chimera are derived from the C57BL/6J cells, or agouti-colored pups, when the ES cells have contributed to the germ cells.

The presence of agouti pups in the F1 generation when using C57BL/6J mice for breeding is thus evidence for the germline transmission of the ES cells. In ES cells, only one copy of the autosomal target gene is targeted and consequently, assuming germ line transmission occurs, 50% of the resulting agouti offspring should receive the mutated chromosome from the ES cells and 50% should receive the wild type chromosome.

### Selection and Characterization of Transgenic Non-Human Animals

The transgenic non-human animals that are produced in accordance with the procedures detailed herein should be screened and evaluated to select those animals that may be used as suitable animal models for investigating the molecular underpinnings of the PTH1R and disorders thereof.

In some embodiment, initial screening may be accomplished by Southern blot analysis or PCR techniques to analyze animal tissues to verify that integration of the transgene has taken place. The level of mRNA expression of the transgene in the tissues of the transgenic animals may also be assessed using techniques which include but are not limited to Northern blot analysis of tissue samples obtained from the animal, in situ hybridization analysis, and reverse transcriptase-PCR (RT-PCR).

Any screening method described herein, and/or known in the art, may be used to select and characterize transgenic non-human animals of the present disclosure.

### Assays and Methods of Using the Invention

The transgenic non-human animals of the present disclosure, and/or one or more cells derived therefrom, may be used as a model organism and/or a model system for investigating the function of human PTH1R (hPTH1R), and/or to generate cell lines that can be used as cell culture models for the same.

In some embodiments, the transgenic non-human animal of the present disclosure, or a cell derived therefrom, may be used to evaluate hPTH1R and its response to different chemicals, drugs, compounds, pharmaceuticals, therapies, treatments, and the like.

In some embodiments, the transgenic non-human animal of the present disclosure, or a cell derived therefrom, may be used to identify one or more candidate agents, e.g., drugs, pharmaceuticals, therapies and interventions, which may affect the normal function of hPTH1R.

In some embodiments, a transgenic non-human animal of the present disclosure, or a cell derived therefrom, may be used to evaluate the effect of said candidate agent on hPTH1R, and/or the cellular and/or molecular functions of the transgenic non-human animal.

In some embodiments, the transgenic non-human animal of the present disclosure, or a cell therefrom, may be used to test one or more candidate agents to identify drugs, pharmaceuticals, therapies and interventions, which may influence hPTH1R function or its pathway.

In some embodiments, candidate and/or therapeutic agents may be administered systemically or locally. For example, suitable routes of administration may include oral, rectal, or intestinal administration; parenteral delivery, including intramuscular, subcutaneous, intramedullary injections, as well as intrathecal, direct intraventricular, intravenous, intraperitoneal, intranasal, or intraocular injections, just to name a few.

In some embodiments, the transgenic non-human animal model systems for PTH-related disorders and/or PTH1R-related disorders may also be used as test substrates in identifying environmental factors, drugs, pharmaceuticals, and chemicals which may affect the function of hPTH1R and/or exacerbate the progression of one or more pathologies and/or disorders that the transgenic animals exhibit.

In an alternate embodiment, the transgenic non-human animals of the invention may be used to derive a cell line which may be used as a test substrate in culture, to identify both candidate agents that affect the function of hPTH1R. While primary cultures derived from the transgenic animals of the invention may be utilized, the generation of continuous cell lines is preferred. For examples of techniques which may be used to derive a continuous cell line from the transgenic animals, see Small et al., 1985, Mol. Cell Biol. 5:642-648.

The transgenic non-human animals of the present disclosure may be used as a model system for human PTH1R (hPTH1R) function, and/or to generate cell lines that can be used as cell culture models for the same.

In some embodiments, the transgenic non-human animal of the present disclosure, or a cell derived therefrom, may be used to evaluate hPTH1R and its response to different chemicals, drugs, compounds, pharmaceuticals, therapies, treatments, and the like.

In some embodiments, the transgenic non-human animal of the present disclosure, or a cell derived therefrom, may be used as a test one or more substrates to identify one or more drugs, pharmaceuticals, therapies and interventions, which may affect the normal function of hPTH1R.

In some embodiments, a transgenic non-human animal of the present disclosure, or a cell derived therefrom, may be used to evaluate the effect of one or more candidate agents on the phenotype of the transgenic non-human animal.

In some embodiments, a transgenic non-human animal of the present disclosure, or a cell derived therefrom, may be used to evaluate the effect of a candidate agent on cellular and/or molecular function of the transgenic non-human animal.

In some embodiments, the transgenic non-human animal of the present disclosure, or a cell therefrom, may be used to test one or more candidate agents to identify drugs, pharmaceuticals, therapies and interventions, with the potential to affect the function of hPTH1R. In some embodiments, candidate and/or therapeutic agents may be administered systemically or locally. For example, suitable routes of administration may include oral, rectal, or intestinal administration; parenteral delivery, including intramuscular, subcutaneous, intramedullary injections, as well as intrathecal, direct intraventricular, intravenous, intraperitoneal, intranasal, or intraocular injections, or any other route described herein.

In some embodiments, the response of the animals to a given treatment and/or one or more candidate agents may be monitored by assessing the function of hPTH1R. With regard to intervention, any treatments and/or candidate agents that affect any aspect of a disease state or disorder should be considered as candidates for therapeutic intervention. However, treatments or regimens that reverse the constellation of pathologies associated with any of these disorders may be preferred. Dosages of candidate agents may be determined by deriving dose-response curves.

In some embodiments, the transgenic non-human animal model systems for PTH-related disorders and/or PTH1R-related disorders may also be used as test substrates in identifying environmental factors, drugs, pharmaceuticals, and chemicals which may affect the function of hPTH1R and/or exacerbate the progression of one or more pathologies and/or disorders that the transgenic animals exhibit.

In an alternate embodiment, the transgenic non-human animals of the invention may be used to derive a cell line which may be used as a test substrate in culture, to identify both candidate agents that affect the function of hPTH1R. In other embodiments, candidate agents can be identified that reduce and or enhance the one or more pathologies associated with hTPH1R. While primary cultures derived from the transgenic animals of the invention may be utilized, the generation of continuous cell lines is preferred. For examples of techniques which may be used to derive a continuous cell line from the transgenic animals, see Small et al., 1985, Mol. Cell Biol. 5:642-648.

In some embodiments, the present disclosure provides a transgenic non-human animal, or a cell therefrom, with which to evaluate the function and/or activity of a human PTH1R protein (hPTH1R).

In some embodiments, the present disclosure provides a transgenic non-human animal, or a cell therefrom, to evaluate the function and/or activity of a ligand to a human PTH1R protein (hPTH1R), e.g., PTH, PTHrP, and the like.

The parathyroid hormone 1-84 (PTH [1-84]) is one of the biologically active hormones produced by the parathyroid glands. PTH is produced as a 118 residue protein that subsequently undergoes two successive cleavages resulting in an 84 residue peptide. PTH (1-84) can be produced in response to, e.g., hypocalcemia and other stimuli, which results in the systemic circulation of the protein. The effect of PTH (1-84) are exerted via interaction between the first 34 residues and PTH1R. See Brown E M. Four-parameter model of the sigmoidal relationship between parathyroid hormone release and extracellular calcium concentration in normal and abnormal parathyroid tissue. J Clin Endocrinol Metab 1983; 56:572; and Diaz R, El-Hajj Fuleihan G, Brown E M. Regulation of parathyroid function. In: Handbook of Physiology, Section 7: The Endocrine System, Fray GGS (Ed), Oxford University Press, New York 1999. In addition, PTH fragments, e.g., those containing N- or C-terminal portions of the protein that arise from either intraglandular and/or peripheral degradation may also present in the circulation.

In some embodiments, the present disclosure provides a transgenic non-human animal, or a cell therefrom, to evaluate the function and/or activity of a PTH1R ligand.

In some embodiments, the present disclosure provides a transgenic non-human animal, or a cell therefrom, to evaluate the function and/or activity of a circulating form of PTH.

In some embodiments, the present disclosure provides a transgenic non-human animal, or a cell therefrom, to evaluate the function and/or activity of PTH (1-84); PTH (1-34); PTHrP; teriparatide; abaloparatide; analogs thereof, variants thereof, and/or combinations thereof.

In some embodiments, the present disclosure provides a transgenic non-human animal, or a cell therefrom, to evaluate the function and/or activity of teriparatide and/or its effect on hPTH1R. Teriparatide (PTH 1-34) is a recombinant form of PTH consisting of amino acids 1-34. It retains all of the biologic activity of the intact PTH (1-84). Teriparatide has been for the treatment of postmenopausal women with osteoporosis at high risk for fracture and, subsequently, for the treatment of osteoporosis in men similarly at high risk for fracture.

In some embodiments, the present disclosure provides a transgenic non-human animal, or a cell therefrom, to evaluate the function and/or activity of abaloparatide and/or its effect on hPTH1R. Abaloparatide (PTHrP 1-34) is a synthetic analog of PTHrP with 76 percent homology. Abaloparatide is known to bind more selectively than teriparatide to the RG conformation of the PTH1R. See Hattersley et al., Binding Selectivity of Abaloparatide for PTH-Type-1-Receptor Conformations and Effects on Downstream Signaling. Endocrinology. 2016 January; 157(1):141-9. In some embodiments, selective binding to the RG conformation of PTH1R confers a more transient response, favoring, e.g., bone formation while minimizing the effects of more prolonged activation (such as hypercalcemia and/or bone resorption).

In some embodiments, the present disclosure provides a transgenic non-human animal, or a cell therefrom, to evaluate the molecular and/or cellular interactions of hPTH1R; one or more ligands of hPTH1R; one or more downstream targets of hPTH1R; and/or combinations thereof. In some embodiments, the molecular and/or cellular interactions of hPTH1R can be referred to as a biomarker of hPTH1R function.

In some embodiments, a biomarker of hPTH1R function can be selected from the set of molecules whose expression profile was found to be indicative of hPTH1R activation, repression, or its otherwise function.

In other embodiments, a biomarker of hPTH1R function can be a polynucleotide or nucleic acid molecule comprising a nucleotide sequence, which codes for a marker protein of the present disclosure, as well as polynucleotides that hybridize with portions of these nucleic acid molecules.

In some embodiments, a biomarker of hPTH1R function may be indicative of the normal baseline state of hPTH1R expression. In some embodiments, e.g., in a disease state, such a biomarker may be different from this baseline state. In some embodiments, said biomarker possesses an expression pattern or profile, which is diagnostic of a disorder such that the expression pattern is found significantly more often in subjects with the disease than in patients without the disease.

In some embodiments, a biomarker of hPTH1R function may be differentially expressed in a subject suffering from a disease state or condition. For example, in some embodiments, a biomarker's abundance level is different in a subject (or a population of subjects) afflicted with a disease or condition relative to the biomarker's level in a healthy or normal subject (or a population of healthy or normal subjects). Differential expression or level of the biomarker includes quantitative, as well as qualitative, differences in the temporal or cellular expression pattern of the biomarker. Methods of measuring different molecules, e.g., gene expression and/or protein level or expression, are well known in the art, and described herein.

In some embodiments, a differentially expressed biomarker of hPTH1R function, alone or in combination with other differentially expressed biomarkers of hPTH1R function, is useful in a variety of different applications in diagnostic, sub-typing, therapeutic, drug development and related areas. The expression patterns and/or levels of one or more differentially expressed biomarkers of hPTH1R function can be described as a fingerprint or a signature of either normal hPTH1R function, or a disease state or condition, disease subtype, and/or stage in the disease state's progression.

In other embodiments, the differential levels of one or more biomarkers of hPTH1R function can be used as a point of reference to compare and characterize unknown samples and samples for which further information is sought.

In some embodiments, the term “decreased level” as used herein, e.g., as it applies to a biomarker of hPTH1R function, refers to a decrease in the abundance level of one or more biomarkers of at least 1%, 2%, 3% 4% 5%, 6% 7%, 8% 9%, 10%, 15%, 20%, 25%, 30%, 40%, 50%, 60%, 70%, 80%, 90%, 100%, or more; or a decrease of greater than 1-fold, 2-fold, 3-fold, 4-fold, 5-fold, 10-fold, 50-fold, 100-fold or more as measured by one or more methods described herein.

In some embodiments, the term “increased level” as used herein, e.g., as it applies to a biomarker of hPTH1R function, refers to an increase in the abundance one or more biomarkers of at least 1%, 2% 3%, 4%, 5%, 6%, 7%, 8% 9%, 10%, 15%, 20%, 25%, 30%, 40%, 50%, 60%, 70%, 80%, 90%, 100%, or more, or an increase of greater than 1-fold, 2-fold, 3-fold, 4-fold, 5-fold, 10-fold, 50-fold, 100-fold or more as measured by one or more methods, such as method described herein.

In some embodiments, a biomarker of hPTH1R function can be determined using an assay selected from the group consisting of co-immunoprecipitation assay; immunofluorescent colocalization assay; photobleaching-based fluorescence resonance energy transfer (FRET); affinity chromatography; PCR; and other well-known methods in the art. See, e.g., Sambrook et al., Molecular Cloning: A Laboratory Manual, 3^{rd} Ed., Cold Spring Harbor Press, Cold Spring Harbor, N.Y. (2001).

In some embodiments, a biomarker of hPTH1R function can be evaluated based on a biological sample taken from a transgenic non-human animal of the present disclosure. For example, in some embodiments, the biological sample can be, for example, cells; tissue (e.g., a tissue sample obtained by biopsy); blood; serum; plasma; urine; sputum; cerebrospinal fluid; lymph tissue or fluid; and/or pancreatic fluid. In other embodiments, the biological sample can be fresh frozen or formalin-fixed paraffin embedded (FFPE) tissue obtained from the non-human animal, such as a tissue sample (e.g., a biopsy). In some embodiments, the biological sample can be obtained from a tissue of interest (e.g., prostate, ovarian, lung, lymph nodes, thymus, spleen, bone marrow, breast, colorectal, pancreatic, cervical, bladder, gastrointestinal, head, and/or neck tissue).

In some embodiments, a biomarker can be a marker of bone and/or mineral metabolism in the blood and/or urine of a transgenic animal. For example, in some embodiments, the biomarker can be, without limitation, one or more of the following: (1) calcium; (2) phosphate; (3) CTX-1 (i.e., C-terminal telopeptides of type I collagen, or the degradation products therefrom); (4) PINP (N-terminal propeptide of type I procollagen); (5) PTH(1-84); (6) 1,25-Dihydroxy Vitamin D; and/or (7) Creatinine.

In some embodiments, the level of a biomarker can be determined by any method known by those having ordinary skill in art.

In some embodiments, RNA from a biological sample may be extracted and analyzed to evaluate a biomarker of hPTH1R function, and/or the level of expression of a heterologous polynucleotide comprising hPTH1R exons 4 to 16. For example, in some embodiments, cell samples, a single cell, and/or tissue samples may be snap frozen in liquid nitrogen until processing. RNA may be extracted using, e.g., Trizol Reagent (available from ThermofisherScientific®; Catalog No. 15596026; 168 Third Avenue, Waltham, MA USA 02451) according to the manufacturer's instructions, and detected directly or converted to cDNA for detection.

In some embodiments, RNA may be amplified using, e.g., MessageAmp II kit (Catalog No. AM1751) available from ThermofisherScientific®, following manufacturer's instructions.

In some embodiments, amplified RNA may be quantified using, e.g., HG-U133A or HG-U133_Plus2 GeneChip® from Affymetrix Inc. (428 Oakmead Pkwy, Sunnyvale, CA USA 94085) or a compatible apparatus, e.g., the GCS3000Dx GeneChip® System from Affymetrix Inc., pursuant to the manufacturer's instructions.

In some embodiments, the resulting biomarker level measurements may be further analyzed and evaluated using statistics programs and/or as described herein. For example, in some embodiments, analysis can be performed using, e.g., R software available from R-Project (http://www.r-project.org) and supplemented with packages available from Bioconductor (http://www.bioconductor.org).

In some embodiments, the level of a heterologous polynucleotide comprising human Parathyroid Hormone 1 Receptor (hPTH1R) exons 4 to 16, wherein said heterologous polynucleotide is operable to encode a human PTH1R protein, and/or one or more of the biomarkers of hPTH1R protein function, may be measured in a biological sample, e.g., a biopsy from a non-human animal such as tissue from the brain, eye, endocrine tissue, ling, proximal digestive tract, gastrointestinal tract, liver, gallbladder, pancreas, kidney, bladder, etc.) obtained from the transgenic non-human animal comprising a heterologous polynucleotide comprising hPTH1R exons 4 to 16, operable to encode a human PTH1R protein, using one or more of the following, without limitation: polymerase chain reaction (PCR); reverse transcriptase PCR (RT-PCR); quantitative real-time PCR (qRT-PCR or q-PCR); an array (e.g., a microarray); a genechip; nanopore sequencing; pyrosequencing; sequencing by synthesis; sequencing by expansion; single molecule real time technology; sequencing by ligation; microfluidics; infrared fluorescence; next generation sequencing (e.g., RNA-Seq techniques); Northern blots; Western blots; Southern blots; NanoString nCounter technologies (e.g., those described in U.S. Patent Application Nos. US 2011/0201515, US 2011/0229888, and US 2013/0017971, each of which is incorporated by reference in its entirety); proteomic techniques (e.g., mass spectrometry or protein arrays); and/or combinations thereof. Additional methods for measuring biomarkers are described in detail below.

Exemplary Assays to Evaluate hPTH1R Function

In some embodiments, the present disclosure provides an assay to identify a candidate agent that modulates the activity or function of a human PTH1R protein (hPTH1R), comprising: (a) obtaining an experimental animal or a cell therefrom; wherein said experimental animal is a transgenic non-human animal having a heterologous polynucleotide comprising human PTH1R exons 4 to 16 that is operable to encode a hPTH1R; and wherein said experimental animal or a cell therefrom is operable to express the hPTH1R; (b) admixing the candidate agent with the hPTH1R present in the experimental animal or cell therefrom; (c) measuring whether said candidate agent modulates the activity or function of said hPTH1R, wherein a modulation in the activity or function of said hPTH1R in the presence of said candidate agent, as compared to the activity or function of said hPTH1R that is not exposed said candidate agent, is indicative that said candidate agent modulates the activity or function of said hPTH1R.

In some embodiments, the present disclosure provides an assay to identify a candidate agent that modulates the activity or function of a hPTH1R, wherein the modulation in the activity or function of the hPTH1R is determined based on a change in the level of one or more of the following: (i) transcription of one or more of the following genes, or promoters thereof: cyclin D1; cyclin A; CREB; E2F transcription factors; or E2F-dependent genes; (ii) phosphorylation of CREB; (iii) one or more proliferating cells; (iv) binding of a parathyroid hormone (PTH), a parathyroid hormone-related peptide (PTHrP); or a fragment thereof; (v) cyclic AMP (cAMP) accumulation; (vi) intracellular free calcium; and/or (vii) inositol phosphate metabolism.

In some embodiments, the present disclosure provides an assay to identify a candidate agent that modulates the activity or function of a hPTH1R, wherein the control animal and the experimental animal are the same type of an animal, wherein said animal is a mammal.

In some embodiments, the present disclosure provides an assay to identify a candidate agent that modulates the activity or function of a hPTH1R, wherein the mammal is selected from the group consisting of: a mouse; a rat; a guinea pig; a hamster; and a gerbil.

In some embodiments, the present disclosure provides an assay to identify a candidate agent that modulates the activity or function of a hPTH1R, wherein the mammal is a mouse.

In some embodiments, the present disclosure provides an assay to identify a candidate agent that modulates the activity or function of a hPTH1R, wherein the mouse is: a 129 mouse; an A mouse; a BALB/c mouse; a C3H mouse; a C57BL mouse; a C57BR mouse; a C57L mouse; a CB17 mouse; a CD-1 mouse; a DBA mouse; an FVB mouse; an SJL mouse; an SWR mouse; any substrain thereof; any hybrid strain thereof; any congenic strain thereof; or any mutant strain thereof.

In some embodiments, the present disclosure provides an assay to identify a candidate agent that modulates the activity or function of a hPTH1R, wherein the mouse is: a C57BL/6 mouse, or a C57BL/10 mouse.

In some embodiments, the present disclosure provides an assay to identify a candidate agent that modulates the activity or function of a hPTH1R, wherein the mouse is a C57BL/6 mouse.

In some embodiments, the present disclosure provides an assay to identify a candidate agent that modulates the activity or function of a hPTH1R, wherein the heterologous polynucleotide comprising human PTH1R exons 4 to 16 is operable to encode a polypeptide having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.1% identical, at least 99.2% identical, at least 99.3% identical, at least 99.4% identical, at least 99.5% identical, at least 99.6% identical, at least 99.7% identical, at least 99.8% identical, at least 99.9% identical, or 100% identical to an amino acid sequence set forth in SEQ ID NOs: 1 or 29.

In some embodiments, the present disclosure provides an assay to identify a candidate agent that modulates the activity or function of a hPTH1R, wherein the heterologous polynucleotide comprising human PTH1R exons 4 to 16 is operable to encode a polypeptide having an amino acid sequence with at least 95% identity to an amino acid sequence as set forth in SEQ ID NO: 1.

In some embodiments, the present disclosure provides an assay to identify a candidate agent that modulates the activity or function of a hPTH1R, wherein the heterologous polynucleotide comprising human PTH1R exons 4 to 16 is operable to encode a polypeptide having an amino acid sequence with at least 95% identity to an amino acid sequence as set forth in SEQ ID NO: 29.

In some embodiments, the present disclosure provides an assay to identify a candidate agent that modulates the activity or function of a hPTH1R, wherein the heterologous polynucleotide comprising human PTH1R exons 4 to 16 is stably integrated in the genome of the transgenic non-human animal, or a cell therefrom.

In some embodiments, the present disclosure provides an assay to identify a candidate agent that modulates the activity or function of a hPTH1R, wherein the heterologous polynucleotide comprising human PTH1R exons 4 to 16 is stably integrated in an endogenous non-human animal PTH1R gene locus.

In some embodiments, the present disclosure provides an assay to identify a candidate agent that modulates the activity or function of a hPTH1R, wherein the heterologous polynucleotide comprising human PTH1R exons 4 to 16 is stably integrated in an endogenous non-human animal PTH1R gene locus that causes a replacement of a genomic DNA segment comprising a non-human animal PTH1R exon 4, with the heterologous polynucleotide comprising human PTH1R exons 4 to 16.

In some embodiments, the present disclosure provides an assay to identify a candidate agent that modulates the activity or function of a hPTH1R, wherein the replacement results in a heterozygous transgenic non-human animal, or a homozygous transgenic non-human animal.

In some embodiments, the present disclosure provides an assay to identify a candidate agent that modulates the activity or function of a hPTH1R, further comprising a control animal or cell therefrom.

In some embodiments, the present disclosure provides an assay to identify a candidate agent that modulates the activity or function of a hPTH1R, wherein a control agent is administered to the control animal or cell therefrom.

In some embodiments, the present disclosure provides an assay to identify a candidate agent that modulates the activity or function of a hPTH1R, wherein the modulation in the activity or function of said hPTH1R in the experimental animal or cell therefrom in the presence of said candidate agent, as compared to the activity or function of said hPTH1R in the control animal or cell therefrom in the presence of the control agent, is indicative that said candidate agent modulates the activity or function of said hPTH1R.

In some embodiments, the present disclosure provides an assay to evaluate human calcemic response to PTH1R agonists. For example, in some embodiments, any of the foregoing non-human transgenic animals can be used to evaluate the response of hPTH1R to one or more PTH1R agonists.

In some embodiments, the present disclosure can be used to determine the effect of one or more PTH1R agonists.

In some embodiments, the present disclosure can be used to determine whether a drug (e.g., a PTH1R agonist) is likely to have a lesser or greater calcemic effect in humans. In some embodiments, information regarding the calcemic effect of a PTH1R agonist can allow for higher or lower dosing (depending on the condition to be treated) of a candidate agent.

In some embodiments, the present disclosure can be used to determine the effect of one or more PTH1R agonists, wherein the information gleaned from the assays provided herein can be used to prognose and/or provide treatment options concerning a variety of disorders that may or may not be caused by inadequate or excessive PTH1R activity.

### Candidate Agents

Candidate agents can be any one or more chemical substances, molecules, nucleotides, polynucleotides, RNA, DNA, peptides, polypeptides, proteins, lipids, glycolipids, enzymes, pharmaceuticals, drugs, prokaryote organisms or eukaryote organisms (and the agents produced from said prokaryote or eukaryote organisms), and/or combinations thereof, that can be screened using an assay and/or other method described herein includes.

In some embodiments, the candidate agent can be a nucleic acid molecule. Nucleic acid molecules used in an assay or a method of screening as described herein can be, for example, an inhibitory nucleic acid molecule. Inhibitory nucleic acid molecules include, for example, a triplex forming oligonucleotide, an aptamer, a ribozyme, a short interfering RNA (siRNA), a micro-RNA (miRNA), or antisense nucleic acid. These types of inhibitory nucleic acid molecules are well known in the art and methods of designing them and making them also are well known in the art. See e.g., International Patent application WO 2004073587; and Haramoto et al., 2007 Oral Dis. 13(1):23-31.

In some embodiments, candidate agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to polypeptide libraries, while the other four approaches are applicable to polypeptide, non-peptide oligomer or small molecule libraries of compounds. See Anticancer Drug Des., 12:145, 1997, the disclosures of which are incorporated herein by reference in its entirety. Such libraries may either be prepared by one of skill in the art, or purchased from commercially available sources See U.S. Pat. Nos. 4,528,266 and 4,359,535; Patent Cooperation Treaty Publication Nos. WO 92/15679, WO 92/15677, WO 90/07862, and WO 90/02809; the disclosures of which are incorporated herein by reference in their entireties.

In some embodiments, candidate agents can be organic molecules. For example, in some embodiments, the candidate agent can be one or more organic molecules selected from either a chemical library, wherein chemicals are assayed individually, or from combinatorial chemical libraries where multiple compounds are assayed at once, then deconvoluted to determine and isolate the most active compounds.

Numerous chemical libraries exist in the art, e.g., as proprietary libraries of pharmaceutical companies, and compounds in such libraries are suitable candidate agents. Representative examples of such combinatorial chemical libraries include those described by U.S. Pat. No. 5,463,564; WO 95/02566; WO 95/24186; WO 95/30642; WO 95/16918; WO 95/16712; U.S. Pat. No. 5,288,514; WO 95/16209; WO 93/20242; WO 95/04277; U.S. Pat. No. 5,506,337; WO 96/00148; Phillips, G. B. and G. P. Wei, “Solid-phase Synthesis of Benzimidazoles,” Tet. Letters 37:4887 90, 1996; Ruhland, B. et al., “Solid-supported Combinatorial Synthesis of Structurally Diverse .beta.-Lactams,” J. Amer. Chem. Soc. 111:253 4, 1996; and Look, G. C. et al., “The Identification of Cyclooxygenase-1 Inhibitors from 4-Thiazolidinone Combinatorial Libraries,” Bioorg and Med Chem. Letters 6:707 12, 1996; the disclosures of which are incorporated herein by reference in their entireties.

In some embodiments, a candidate agent can be a polypeptide, an antibody (e.g., polyclonal or monoclonal; human, or humanized) a small molecule, a nucleic acid molecule, a peptidomimetic, or any combination thereof.

In some embodiments, candidate agents can include, without limitation, fusion proteins, polypeptides, peptidomimetics, prodrugs, receptors, binding agents, ribozymes, small molecules, peptides, antibodies, or other drugs which are screened for the ability to modulate hPTH1R function.

The transgenic non-human animals of the invention can be used in the identification and characterization of candidate agents and the influence of the same on hPTH1R function. In these methods, for example, a candidate agent can be administered to a transgenic non-human animal and the impact of the agent on the function of hPTH1R in the animal can be monitored.

For example, in some embodiments, transgenic non-human animal models of the present disclosure can be used to monitor the effect of a candidate agent in order to determine whether said candidate agent modulates the function of hPTH1R.

In another example, gene- and cell-based therapies for an hPTH1R-associated disease or disorder can be administered in a transgenic non-human animal of the present disclosure, and the animal may be monitored for the effects on the development or progression of the disease and/or disorder, and further can be used to assess the effect and the impact on progression (or reversal) of the same.

With the transgenic non-human animal of the invention, it is possible to test hypotheses that lead to new treatments, diagnostics, protocols, imaging technologies, and medical devices, and to evaluate the function of hPTH1R or variants thereof. Likely activities involving the present disclosure may include evaluating current and future therapeutics for toxicity, pharmacokinetics and efficacy within the same transgenic non-human animal. Medical devices makers may study the efficacy of products in a relevant, diseased setting. And in the context of medical instruments, noninvasive ultrasound imaging may be evaluated to diagnose and chart the development and progression of disease.

### Measuring Gene and Protein Levels

Any of the following techniques, without limitation, can be used to analyze the expression of one or more genes (e.g., a polynucleotide operable to encode hPTH1R, or an hPTH1R protein): reverse transcriptase PCR (RT-PCR); quantitative real-time PCR (qRT-PCR or q-PCR); an array (e.g., a microarray); a genechip; nanopore sequencing; pyrosequencing; sequencing by synthesis; sequencing by expansion; single molecule real time technology; sequencing by ligation; microfluidics; infrared fluorescence; next generation sequencing (e.g., RNA-Seq techniques); Northern blots; Western blots; Southern blots; NanoString nCounter technologies (e.g., those described in U.S. Patent Application Nos. US 2011/0201515, US 2011/0229888, and US 2013/0017971, each of which is incorporated by reference in its entirety); proteomic techniques (e.g., mass spectrometry or protein arrays); and/or combinations thereof.

### Polymerase Chain Reaction (PCR)

Polymerase chain reaction (PCR) and its related techniques are well known to those having ordinary skill in the art. In some embodiments, PCR can be used in several aspects of the present disclosure, e.g., cloning vectors; confirming the presence of a transgene; detecting the level of a biomarker; detecting the level of one or more polynucleotides transcribed in response to a candidate agent; and other methods described herein and known to those having ordinary skill in the art.

In some embodiments, RT-PCR can be used to detect mRNA in a biological sample, and/or compare mRNA levels in different biological samples. In other embodiments, RT-PCR can be used to compare mRNA levels in a first sample and a second sample, with or without treatment of a candidate agent, to characterize patterns of gene expression, to discriminate between closely related mRNAs, to analyze RNA structure, and/or evaluate the effect of said candidate agent on hPTH1R function.

Methods for quantifying mRNA are well known in the art. In some embodiments, the method utilizes RT-PCR. Generally, the first step in gene expression profiling by RT-PCR is the reverse transcription of the RNA template into cDNA, followed by its exponential amplification in a PCR reaction. Two commonly used reverse transcriptases are avian myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase. TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers can be used to generate an amplicon typical of a PCR reaction. A third oligonucleotide, or probe, can be designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

To minimize errors and the effect of sample-to-sample variation, RT-PCR can be performed using an internal standard. The ideal internal standard is expressed at a constant level among different tissues, and is unaffected by the experimental treatment. RNAs commonly used to normalize patterns of gene expression are mRNAs for the housekeeping genes glyceraldehyde-3-phosphate-dehydrogenase (GAPDH), beta-actin, and 18S ribosomal RNA.

A variation of RT-PCR is real time quantitative RT-PCR (qRT-PCR or “real time PCR”), which measures PCR product accumulation through a dual-labeled fluorogenic probe (e.g., TAQMAN® probe). Real time PCR is compatible both with quantitative competitive PCR, where internal competitor for each target sequence is used for normalization, and with quantitative comparative PCR using a normalization gene contained within the sample, or a housekeeping gene for RT-PCR (see Held et al., Genome Research 6:986 994, 1996).

Exemplary methods of qRT-PCR are provided in U.S. Pat. No. 5,538,848, the disclosure of which is incorporated herein by reference in its entirety. Related probes and quantitative amplification procedures are provided in U.S. Pat. Nos. 5,716,784 and 5,723,591, the disclosures of which are incorporated herein by reference in their entireties. Exemplary instruments for carrying out qRT-PCR (e.g., on microtiter plates) are available from PE Applied Biosystems, 850 Lincoln Centre Drive, Foster City, Calif. 94404 under the trademark ABI PRISM® 7700.

In some embodiments, the primers used for the amplification are selected so as to amplify a unique segment of the gene of interest, e.g., a heterologous polynucleotide comprising human Parathyroid Hormone 1 Receptor (hPTH1R) exons 4 to 16; wherein said heterologous polynucleotide is operable to encode a human PTH1R protein.

Primers are commercially available, and can be designed using any of the readily available primer design tools known to those in the art, e.g., OligoPerfect Primer Designer (ThermoFisherScientific®), or Primer-BLAST, a tool available from NCBI that can be used to find specific primers (https://www.ncbi.nlm.nih.gov/tools/primer-blast/).

An alternative quantitative nucleic acid amplification procedure is provided in U.S. Pat. No. 5,219,727, wherein the amount of a target sequence in a sample is determined by simultaneously amplifying the target sequence and an internal standard nucleic acid segment. Thus, the amount of amplified DNA from each segment is determined and compared to a standard curve, to determine the amount of the target nucleic acid segment that was present in the sample prior to amplification.

In some embodiments, the expression of a “housekeeping” gene or “internal control” can also be evaluated. These terms include any constitutively or globally expressed gene whose presence enables an assessment of a heterologous polynucleotide comprising human Parathyroid Hormone 1 Receptor (hPTH1R) exons 4 to 16; wherein said heterologous polynucleotide is operable to encode a human PTH1R protein, and the mRNA levels thereof. Such an assessment includes a determination of the overall constitutive level of gene transcription and a control for variations in RNA recovery. Exemplary housekeeping genes are known to those having ordinary skill in the art, and can be specifically tailored to one's need without undue experimentation.

Exemplary methods of PCR techniques are provided in U.S. Pat. Nos. 4,683,195; 4,683,202; 4,889,818; 5,863,736; 5,538,848; 9,404,150; WIPO Publication No. WO1991002090A1; Gibson et al., A novel method for real time quantitative RT-PCR, Genome Research. 6: 995-1001, 1996; Holland et al., Detection of specific polymerase chain reaction product by utilizing the 5′-3′ exonuclease activity of Thermus aquaticus DNA polymerase, PNAS. 88: 7276-7280, 1991; Livak et al., Oligonucleotides with fluorescent dyes at opposite ends provide a quenched probe system useful for detecting PCR product and nucleic acid hybridization, PCR Methods and Applications 357-362, 1995; and Hahn, Statistical Intervals; a guide for practitioners, p. p 311: John Wiley & Sons. New York, N.Y., 1991; the disclosures of which are incorporated herein by reference in their entireties.

### Illustrative Embodiments

In some embodiments, the present disclosure provides a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein.

In some embodiments, the present disclosure provides a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein, wherein the non-human animal is a mammal.

In some embodiments, the present disclosure provides a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein, wherein the mammal is selected from the group consisting of: a mouse; a rat; a guinea pig; a hamster; and a gerbil.

In some embodiments, the present disclosure provides a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein, wherein the mammal is a mouse.

In some embodiments, the present disclosure provides a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein, wherein the transgenic animal is: a 129 mouse; an A mouse; a BALB/c mouse; a C3H mouse; a C57BL mouse; a C57BR mouse; a C57L mouse; a CB17 mouse; a CD-1 mouse; a DBA mouse; an FVB mouse; an SJL mouse; an SWR mouse; any substrain thereof; any hybrid strain thereof; any congenic strain thereof; or any mutant strain thereof.

In some embodiments, the present disclosure provides a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein, wherein the transgenic animal is a C57BL/6 mouse, or a C57BL/10 mouse.

In some embodiments, the present disclosure provides a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein, wherein the transgenic animal is a C57BL/6 mouse.

In some embodiments, the present disclosure provides a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein, wherein the heterologous polynucleotide comprising human PTH1R exons 4 to 16 is operable to encode a polypeptide having an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical an amino acid sequence as set forth in SEQ ID NO: 1.

In some embodiments, the present disclosure provides a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein, wherein the heterologous polynucleotide comprising human PTH1R exons 4 to 16 is operable to encode a polypeptide having an amino acid sequence that is at least 80% identical, at least 85% identical, at least 90% identical, at least 95% identical, at least 96% identical, at least 97% identical, at least 98% identical, at least 99% identical, or 100% identical to an amino acid sequence as set forth in SEQ ID NO: 1.

In some embodiments, the present disclosure provides a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein, wherein the heterologous polynucleotide comprising human PTH1R exons 4 to 16 is operable to encode a polypeptide having an amino acid sequence that is at least 95% identical to an amino acid sequence as set forth in SEQ ID NO: 1.

In some embodiments, the present disclosure provides a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein, wherein the heterologous polynucleotide comprising human PTH1R exons 4 to 16 is operable to encode a polypeptide having an amino acid sequence that is at least 80% identical, at least 85% identical, at least 90% identical, at least 95% identical, at least 96% identical, at least 97% identical, at least 98% identical, at least 99% identical, or 100% identical to an amino acid sequence as set forth in SEQ ID NO: 29.

In some embodiments, the present disclosure provides a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein, wherein the heterologous polynucleotide comprising human PTH1R exons 4 to 16 is operable to encode a polypeptide having an amino acid sequence that is at least 95% identical to an amino acid sequence as set forth in SEQ ID NO: 29.

In some embodiments, the present disclosure provides a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein, wherein the heterologous polynucleotide comprising human PTH1R exons 4 to 16 is stably integrated in the genome of the non-human animal.

In some embodiments, the present disclosure provides a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein, wherein the heterologous polynucleotide comprising human PTH1R exons 4 to 16 is stably integrated in an endogenous non-human animal PTH1R gene locus.

In some embodiments, the present disclosure provides a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein, wherein the heterologous polynucleotide comprising human PTH1R exons 4 to 16 is stably integrated in an endogenous non-human animal PTH1R gene locus that causes a replacement of a genomic DNA segment comprising non-human animal PTH1R exon 4, with the heterologous polynucleotide comprising human PTH1R exons 4 to 16.

In some embodiments, the replacement results in a heterozygous transgenic non-human animal, or a homozygous transgenic non-human animal.

In some embodiments, the present disclosure provides a non-human recombinant cell comprising: a heterologous polynucleotide comprising human Parathyroid Hormone 1 Receptor (hPTH1R) exons 4 to 16, wherein said heterologous polynucleotide is operable to encode a human PTH1R protein.

In some embodiments, the present disclosure provides a non-human recombinant cell comprising: a heterologous polynucleotide comprising human Parathyroid Hormone 1 Receptor (hPTH1R) exons 4 to 16, wherein said heterologous polynucleotide is operable to encode a human PTH1R protein, wherein the non-human recombinant cell is a mammalian recombinant cell.

In some embodiments, the present disclosure provides a non-human recombinant cell comprising: a heterologous polynucleotide comprising human Parathyroid Hormone 1 Receptor (hPTH1R) exons 4 to 16, wherein said heterologous polynucleotide is operable to encode a human PTH1R protein, wherein the non-human recombinant cell is selected from the group consisting of: a mouse; a rat; a guinea pig; a hamster; and a gerbil.

In some embodiments, the present disclosure provides a non-human recombinant cell comprising: a heterologous polynucleotide comprising human Parathyroid Hormone 1 Receptor (hPTH1R) exons 4 to 16, wherein said heterologous polynucleotide is operable to encode a human PTH1R protein, wherein the non-human recombinant cell is a mouse recombinant cell.

In some embodiments, the present disclosure provides a non-human recombinant cell comprising: a heterologous polynucleotide comprising human Parathyroid Hormone 1 Receptor (hPTH1R) exons 4 to 16, wherein said heterologous polynucleotide is operable to encode a human PTH1R protein, wherein the non-human recombinant cell is: a 129 recombinant cell; an A recombinant cell; a BALB/c recombinant cell; a C3H recombinant cell; a C57BL recombinant cell; a C57BR recombinant cell; a C57L recombinant cell; a CB17 recombinant cell; a CD-1 recombinant cell; a DBA recombinant cell; an FVB recombinant cell; an SJL recombinant cell; an SWR recombinant cell; a cell from any substrain thereof; a cell from any hybrid strain thereof; a cell from any congenic strain thereof; or a cell from any mutant strain thereof.

In some embodiments, the present disclosure provides a non-human recombinant cell comprising: a heterologous polynucleotide comprising human Parathyroid Hormone 1 Receptor (hPTH1R) exons 4 to 16, wherein said heterologous polynucleotide is operable to encode a human PTH1R protein, wherein the non-human recombinant cell is a C57BL/6 mouse recombinant cell, or a C57BL/10 mouse recombinant cell.

In some embodiments, the present disclosure provides a non-human recombinant cell comprising: a heterologous polynucleotide comprising human Parathyroid Hormone 1 Receptor (hPTH1R) exons 4 to 16, wherein said heterologous polynucleotide is operable to encode a human PTH1R protein, wherein the non-human recombinant cell is a C57BL/6 mouse recombinant cell.

In some embodiments, the present disclosure provides a non-human recombinant cell comprising: a heterologous polynucleotide comprising human Parathyroid Hormone 1 Receptor (hPTH1R) exons 4 to 16, wherein said heterologous polynucleotide is operable to encode a human PTH1R protein, wherein the heterologous polynucleotide comprising human PTH1R exons 4 to 16 is operable to encode a polypeptide having an amino acid sequence that is at least 80% identical, at least 85% identical, at least 90% identical, at least 95% identical, at least 96% identical, at least 97% identical, at least 98% identical, at least 99% identical, or 100% identical to an amino acid sequence as set forth in SEQ ID NO: 1.

In some embodiments, the present disclosure provides a non-human recombinant cell comprising: a heterologous polynucleotide comprising human Parathyroid Hormone 1 Receptor (hPTH1R) exons 4 to 16, wherein said heterologous polynucleotide is operable to encode a human PTH1R protein, wherein the heterologous polynucleotide comprising human PTH1R exons 4 to 16 is operable to encode a polypeptide having an amino acid sequence that is at least 95% identical to an amino acid sequence as set forth in SEQ ID NO: 1.

In some embodiments, the present disclosure provides a non-human recombinant cell comprising: a heterologous polynucleotide comprising human Parathyroid Hormone 1 Receptor (hPTH1R) exons 4 to 16, wherein said heterologous polynucleotide is operable to encode a human PTH1R protein, wherein the heterologous polynucleotide comprising human PTH1R exons 4 to 16 is operable to encode a polypeptide having an amino acid sequence that is at least 80% identical, at least 85% identical, at least 90% identical, at least 95% identical, at least 96% identical, at least 97% identical, at least 98% identical, at least 99% identical, or 100% identical to an amino acid sequence as set forth in SEQ ID NO: 29.

In some embodiments, the present disclosure provides a non-human recombinant cell comprising: a heterologous polynucleotide comprising human Parathyroid Hormone 1 Receptor (hPTH1R) exons 4 to 16, wherein said heterologous polynucleotide is operable to encode a human PTH1R protein, wherein the heterologous polynucleotide comprising human PTH1R exons 4 to 16 is operable to encode a polypeptide having an amino acid sequence that at least 95% identical to an amino acid sequence as set forth in SEQ ID NO: 29.

In some embodiments, the present disclosure provides a non-human recombinant cell comprising: a heterologous polynucleotide comprising human Parathyroid Hormone 1 Receptor (hPTH1R) exons 4 to 16, wherein said heterologous polynucleotide is operable to encode a human PTH1R protein, wherein the heterologous polynucleotide comprising human PTH1R exons 4 to 16 is stably integrated in the genome of the non-human recombinant cell.

In some embodiments, the present disclosure provides a non-human recombinant cell comprising: a heterologous polynucleotide comprising human Parathyroid Hormone 1 Receptor (hPTH1R) exons 4 to 16, wherein said heterologous polynucleotide is operable to encode a human PTH1R protein, wherein the heterologous polynucleotide comprising human PTH1R exons 4 to 16 is stably integrated in an endogenous non-human animal PTH1R gene locus.

In some embodiments, the present disclosure provides a non-human recombinant cell comprising: a heterologous polynucleotide comprising human Parathyroid Hormone 1 Receptor (hPTH1R) exons 4 to 16, wherein said heterologous polynucleotide is operable to encode a human PTH1R protein, wherein the heterologous polynucleotide comprising human PTH1R exons 4 to 16 is stably integrated in an endogenous non-human animal PTH1R gene locus that causes a replacement of a genomic DNA segment comprising non-human animal PTH1R exon 4, with the heterologous polynucleotide comprising human PTH1R exons 4 to 16. In some embodiments, the replacement results in a heterozygous recombinant cell, or a homozygous recombinant cell.

In some embodiments, the present disclosure provides a vector comprising: (i) a heterologous polynucleotide comprising a first nucleotide sequence comprising a coding sequence for human Parathyroid Hormone 1 Receptor (hPTH1R) exons 4 to 16 and second nucleotide sequence comprising a polyadenylation signal; (ii) a 5′-homology arm, and a 3′-homology arm, wherein said 5′-homology arm and said 3′-homology arm are located upstream and downstream of the heterologous polynucleotide, respectively; (iii) a third nucleotide sequence operable to encode a diphtheria toxin A protein, or fragment thereof; and a fourth nucleotide sequence operable to encode an neomycin phosphotransferase II (Neo); (iv) an upstream self-deletion anchor (SDA) nucleotide sequence, and a downstream SDA nucleotide sequence; wherein said upstream SDA nucleotide sequence and downstream SDA nucleotide sequences flank the fourth nucleotide sequence; wherein said vector is operable to allow a homologous recombination-mediated integration of the heterologous polynucleotide into an endogenous non-human animal PTH1R gene locus; and wherein said homologous recombination-mediated integration results in a replacement of an endogenous non-human animal genomic DNA segment with the heterologous polynucleotide.

In some embodiments, the present disclosure provides a method of making a transgenic non-human animal comprising: (i) introducing a heterologous polynucleotide comprising human PTH1R exons 4 to 16 into a non-human animal embryonic stem (ES) cell, such that the heterologous polynucleotide integrates into an endogenous non-human animal PTH1R locus; (ii) obtaining a non-human animal ES cell comprising a modified genome, wherein the heterologous polynucleotide has integrated into an endogenous non-human animal PTH1R locus; and (iii) generating a non-human animal using the non-human animal ES cell comprising the modified genome.

In some embodiments of the method of making a transgenic non-human animal of the present disclosure, the non-human animal is a mammal.

In some embodiments of the method of making a transgenic non-human animal of the present disclosure, the non-human animal is selected from the group consisting of: a mouse; a rat; a guinea pig; a hamster; and a gerbil.

In some embodiments of the method of making a transgenic non-human animal of the present disclosure, the non-human animal is a mouse.

In some embodiments of the method of making a transgenic non-human animal of the present disclosure, the non-human animal is: a 129 mouse; an A mouse; a BALB/c mouse; a C3H mouse; a C57BL mouse; a C57BR mouse; a C57L mouse; a CB17 mouse; a CD-1 mouse; a DBA mouse; an FVB mouse; an SJL mouse; an SWR mouse; any substrain thereof; any hybrid strain thereof; any congenic strain thereof; or any mutant strain thereof.

In some embodiments of the method of making a transgenic non-human animal of the present disclosure, the non-human animal is: a C57BL/6 mouse, or a C57BL/10 mouse.

In some embodiments of the method of making a transgenic non-human animal of the present disclosure, the non-human animal is a C57BL/6 mouse.

In some embodiments, the present disclosure provides a method of making a transgenic non-human animal comprising: (i) introducing a heterologous polynucleotide comprising human PTH1R exons 4 to 16 into a non-human animal embryonic stem (ES) cell, such that the heterologous polynucleotide integrates into an endogenous non-human animal PTH1R locus; (ii) obtaining a non-human animal ES cell comprising a modified genome, wherein the heterologous polynucleotide has integrated into an endogenous non-human animal PTH1R locus; and (iii) generating a non-human animal using the non-human animal ES cell comprising the modified genome; wherein the heterologous polynucleotide comprising human PTH1R exons 4 to 16 is operable to encode a human PTH1R protein having an amino acid sequence that is at least 80% identical, at least 85% identical, at least 90% identical, at least 95% identical, at least 96% identical, at least 97% identical, at least 98% identical, at least 99% identical, or 100% identical to an amino acid sequence as set forth in SEQ ID NO: 1.

In some embodiments, the present disclosure provides a method of making a transgenic non-human animal comprising: (i) introducing a heterologous polynucleotide comprising human PTH1R exons 4 to 16 into a non-human animal embryonic stem (ES) cell, such that the heterologous polynucleotide integrates into an endogenous non-human animal PTH1R locus; (ii) obtaining a non-human animal ES cell comprising a modified genome, wherein the heterologous polynucleotide has integrated into an endogenous non-human animal PTH1R locus; and (iii) generating a non-human animal using the non-human animal ES cell comprising the modified genome; wherein the heterologous polynucleotide comprising human PTH1R exons 4 to 16 is operable to encode a human PTH1R protein having an amino acid sequence that is at least 95% identical to an amino acid sequence as set forth in SEQ ID NO: 1.

In some embodiments, the present disclosure provides a method of making a transgenic non-human animal comprising: (i) introducing a heterologous polynucleotide comprising human PTH1R exons 4 to 16 into a non-human animal embryonic stem (ES) cell, such that the heterologous polynucleotide integrates into an endogenous non-human animal PTH1R locus; (ii) obtaining a non-human animal ES cell comprising a modified genome, wherein the heterologous polynucleotide has integrated into an endogenous non-human animal PTH1R locus; and (iii) generating a non-human animal using the non-human animal ES cell comprising the modified genome; wherein the heterologous polynucleotide comprising human PTH1R exons 4 to 16 is operable to encode a human PTH1R protein having an amino acid sequence that is at least 80% identical, at least 85% identical, at least 90% identical, at least 95% identical, at least 96% identical, at least 97% identical, at least 98% identical, at least 99% identical, or 100% identical to an amino acid sequence as set forth in SEQ ID NO: 29.

In some embodiments, the present disclosure provides a method of making a transgenic non-human animal comprising: (i) introducing a heterologous polynucleotide comprising human PTH1R exons 4 to 16 into a non-human animal embryonic stem (ES) cell, such that the heterologous polynucleotide integrates into an endogenous non-human animal PTH1R locus; (ii) obtaining a non-human animal ES cell comprising a modified genome, wherein the heterologous polynucleotide has integrated into an endogenous non-human animal PTH1R locus; and (iii) generating a non-human animal using the non-human animal ES cell comprising the modified genome; wherein the heterologous polynucleotide comprising human PTH1R exons 4 to 16 is operable to encode a human PTH1R protein having an amino acid sequence that is at least 95% identical to an amino acid sequence as set forth in SEQ ID NO: 29.

In some embodiments, the present disclosure provides a method of making a transgenic non-human animal comprising: (i) introducing a heterologous polynucleotide comprising human PTH1R exons 4 to 16 into a non-human animal embryonic stem (ES) cell, such that the heterologous polynucleotide integrates into an endogenous non-human animal PTH1R locus; (ii) obtaining a non-human animal ES cell comprising a modified genome, wherein the heterologous polynucleotide has integrated into an endogenous non-human animal PTH1R locus; and (iii) generating a non-human animal using the non-human animal ES cell comprising the modified genome; wherein the heterologous polynucleotide comprising human PTH1R exons 4 to 16 is stably integrated in the genome of the transgenic non-human animal.

In some embodiments, the present disclosure provides a method of making a transgenic non-human animal comprising: (i) introducing a heterologous polynucleotide comprising human PTH1R exons 4 to 16 into a non-human animal embryonic stem (ES) cell, such that the heterologous polynucleotide integrates into an endogenous non-human animal PTH1R locus; (ii) obtaining a non-human animal ES cell comprising a modified genome, wherein the heterologous polynucleotide has integrated into an endogenous non-human animal PTH1R locus; and (iii) generating a non-human animal using the non-human animal ES cell comprising the modified genome; wherein the heterologous polynucleotide comprising human PTH1R exons 4 to 16 is stably integrated in an endogenous non-human animal PTH1R gene locus.

In some embodiments, the present disclosure provides a method of making a transgenic non-human animal comprising: (i) introducing a heterologous polynucleotide comprising human PTH1R exons 4 to 16 into a non-human animal embryonic stem (ES) cell, such that the heterologous polynucleotide integrates into an endogenous non-human animal PTH1R locus; (ii) obtaining a non-human animal ES cell comprising a modified genome, wherein the heterologous polynucleotide has integrated into an endogenous non-human animal PTH1R locus; and (iii) generating a non-human animal using the non-human animal ES cell comprising the modified genome; wherein the heterologous polynucleotide comprising human PTH1R exons 4 to 16 is stably integrated in an endogenous non-human animal PTH1R gene locus that causes a replacement of a genomic DNA segment comprising non-human animal PTH1R exon 4, with the heterologous polynucleotide comprising human PTH1R exons 4 to 16. In some embodiments, the replacement results in a heterozygous transgenic non-human animal, or a homozygous transgenic non-human animal.

In some embodiments, the present disclosure provides an assay to identify a candidate agent that modulates the activity or function of a human PTH1R protein (hPTH1R), comprising: (a) obtaining an experimental animal or a cell therefrom; wherein said experimental animal is a transgenic non-human animal having a heterologous polynucleotide comprising human PTH1R exons 4 to 16 that is operable to encode a hPTH1R; and wherein said experimental animal or a cell therefrom is operable to express the hPTH1R; (b) admixing the candidate agent with the hPTH1R present in the experimental animal or cell therefrom; (c) measuring whether said candidate agent modulates the activity or function of said hPTH1R, wherein a modulation in the activity or function of said hPTH1R in the presence of said candidate agent, as compared to the activity or function of said hPTH1R that is not exposed said candidate agent, is indicative that said candidate agent modulates the activity or function of said hPTH1R.

In some embodiments of the assay of the present disclosure, the modulation in the activity or function of the hPTH1R is determined based on a change in the level of one or more of the following: (i) transcription of one or more of the following genes, or promoters thereof: cyclin D1; cyclin A; CREB; E2F transcription factors; or E2F-dependent genes; (ii) phosphorylation of CREB; (iii) one or more proliferating cells; (iv) binding of a parathyroid hormone (PTH), a parathyroid hormone-related peptide (PTHrP); or a fragment thereof; (v) cyclic AMP (cAMP) accumulation; (vi) intracellular free calcium; or (vii) inositol phosphate metabolism.

In some embodiments of the assay of the present disclosure, the control animal and the experimental animal are the same type of an animal, wherein said animal is a mammal.

In some embodiments of the assay of the present disclosure, the mammal is selected from the group consisting of: a mouse; a rat; a guinea pig; a hamster; and a gerbil.

In some embodiments of the assay of the present disclosure, the mammal is a mouse.

In some embodiments of the assay of the present disclosure, the mouse is: a 129 mouse; an A mouse; a BALB/c mouse; a C3H mouse; a C57BL mouse; a C57BR mouse; a C57L mouse; a CB17 mouse; a CD-1 mouse; a DBA mouse; an FVB mouse; an SJL mouse; an SWR mouse; any substrain thereof; any hybrid strain thereof; any congenic strain thereof; or any mutant strain thereof.

In some embodiments of the assay of the present disclosure, the mouse is: a C57BL/6 mouse, or a C57BL/10 mouse.

In some embodiments of the assay of the present disclosure, the mouse is a C57BL/6 mouse.

In some embodiments of the assay of the present disclosure, the heterologous polynucleotide comprising human PTH1R exons 4 to 16 is operable to encode a polypeptide having an amino acid sequence that is at least 80% identical, at least 85% identical, at least 90% identical, at least 95% identical, at least 96% identical, at least 97% identical, at least 98% identical, at least 99% identical, or 100% identical to an amino acid sequence as set forth in SEQ ID NO: 1.

In some embodiments of the assay of the present disclosure, the heterologous polynucleotide comprising human PTH1R exons 4 to 16 is operable to encode a polypeptide having an amino acid sequence that is at least 95% identical to an amino acid sequence as set forth in SEQ ID NO: 1.

In some embodiments of the assay of the present disclosure, the heterologous polynucleotide comprising human PTH1R exons 4 to 16 is operable to encode a polypeptide having an amino acid sequence that is at least 80% identical, at least 85% identical, at least 90% identical, at least 95% identical, at least 96% identical, at least 97% identical, at least 98% identical, at least 99% identical, or 100% identical to an amino acid sequence as set forth in SEQ ID NO: 29.

In some embodiments of the assay of the present disclosure, the heterologous polynucleotide comprising human PTH1R exons 4 to 16 is operable to encode a polypeptide having an amino acid sequence that is at least 95% identical to an amino acid sequence as set forth in SEQ ID NO: 29.

In some embodiments of the assay of the present disclosure, the heterologous polynucleotide comprising human PTH1R exons 4 to 16 is stably integrated in the genome of the transgenic non-human animal, or a cell therefrom.

In some embodiments of the assay of the present disclosure, the heterologous polynucleotide comprising human PTH1R exons 4 to 16 is stably integrated in an endogenous non-human animal PTH1R gene locus.

In some embodiments of the assay of the present disclosure, the heterologous polynucleotide comprising human PTH1R exons 4 to 16 is stably integrated in an endogenous non-human animal PTH1R gene locus that causes a replacement of a genomic DNA segment comprising a non-human animal PTH1R exon 4, with the heterologous polynucleotide comprising human PTH1R exons 4 to 16.

In some embodiments of the assay of the present disclosure, the replacement results in a heterozygous transgenic non-human animal, or a homozygous transgenic non-human animal.

In some embodiments of the assay of the present disclosure, the assay further comprising a control animal or cell therefrom.

In some embodiments of the assay of the present disclosure, a control agent is administered to the control animal or cell therefrom.

In some embodiments of the assay of the present disclosure, the modulation in the activity or function of said hPTH1R in the experimental animal or cell therefrom in the presence of said candidate agent, as compared to the activity or function of said hPTH1R in the control animal or cell therefrom in the presence of the control agent, is indicative that said candidate agent modulates the activity or function of said hPTH1R.

In some embodiments, the present disclosure provides a transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein; wherein the human PTH1R protein further comprises a human influenza hemagglutinin (HA) epitope tag.

In some embodiments, the present disclosure provides a non-human recombinant cell comprising a heterologous polynucleotide comprising human Parathyroid Hormone 1 Receptor (hPTH1R) exons 4 to 16; wherein said heterologous polynucleotide is operable to encode a human PTH1R protein; wherein the human PTH1R protein further comprises a human influenza hemagglutinin (HA) epitope tag.

In some embodiments, the present disclosure provides a method of making a transgenic non-human animal comprising: (i) introducing a heterologous polynucleotide comprising human PTH1R exons 4 to 16 into a non-human animal embryonic stem (ES) cell, such that the heterologous polynucleotide integrates into an endogenous non-human animal PTH1R locus; (ii) obtaining a non-human animal ES cell comprising a modified genome, wherein the heterologous polynucleotide has integrated into an endogenous non-human animal PTH1R locus; and (iii) generating a non-human animal using the non-human animal ES cell comprising the modified genome; wherein the hPTH1R protein further comprises a human influenza hemagglutinin (HA) epitope tag.

In some embodiments, the present disclosure provides an assay to identify a candidate agent that modulates the activity or function of a human PTH1R protein (hPTH1R), comprising: (a) obtaining an experimental animal or a cell therefrom; wherein said experimental animal is a transgenic non-human animal having a heterologous polynucleotide comprising human PTH1R exons 4 to 16 that is operable to encode a hPTH1R; and wherein said experimental animal or a cell therefrom is operable to express the hPTH1R; (b) admixing the candidate agent with the hPTH1R present in the experimental animal or cell therefrom; (c) measuring whether said candidate agent modulates the activity or function of said hPTH1R, wherein a modulation in the activity or function of said hPTH1R in the presence of said candidate agent, as compared to the activity or function of said hPTH1R that is not exposed said candidate agent, is indicative that said candidate agent modulates the activity or function of said hPTH1R; and wherein the hPTH1R further comprises a human influenza hemagglutinin (HA) epitope tag.

In some embodiments, the present disclosure provides a transgenic mouse comprising a heterologous polynucleotide comprising human Parathyroid Hormone 1 Receptor (hPTH1R) exons 4 to 16; wherein said heterologous polynucleotide is operable to encode a human PTH1R protein having an amino acid sequence as set forth in SEQ ID NO: 1.

In some embodiments, the present disclosure provides a transgenic mouse comprising a heterologous polynucleotide comprising human Parathyroid Hormone 1 Receptor (hPTH1R) exons 4 to 16; wherein said heterologous polynucleotide is operable to encode a human PTH1R protein having an amino acid sequence as set forth in SEQ ID NO: 29.

In some embodiments, the present disclosure provides a non-human recombinant cell comprising: a heterologous polynucleotide comprising human Parathyroid Hormone 1 Receptor (hPTH1R) exons 4 to 16, wherein said heterologous polynucleotide is operable to encode a human PTH1R protein having an amino acid sequence as set forth in SEQ ID NO: 1.

In some embodiments, the present disclosure provides a non-human recombinant cell comprising: a heterologous polynucleotide comprising human Parathyroid Hormone 1 Receptor (hPTH1R) exons 4 to 16, wherein said heterologous polynucleotide is operable to encode a human PTH1R protein having an amino acid sequence as set forth in SEQ ID NO: 29.

### EXAMPLES

The Examples in this specification are not intended to, and should not be used to, limit the invention; they are provided only to illustrate the invention.

The Examples in this specification are not intended to, and should not be used to, limit the invention; they are provided only to illustrate the invention.

### Example 1. Targeting Strategy

A heterologous polynucleotide comprising human Parathyroid Hormone 1 Receptor (hPTH1R) exons 4 to 16, wherein said heterologous polynucleotide is operable to encode a human PTH1R protein (hereinafter referred to as the “transgene”), was stably inserted in the genome of a non-human animal. The non-human animal selected was a mouse, i.e., a C57BL/6 mouse.

C57BL/6 mice are well-known in the art, and commercially available (e.g., a large catalog of C57BL/6 mice are available from CYAGEN®, 2255 Martin Avenue, Suite E Santa Clara, CA 95050-2709, USA).

A knock-in model was devised, wherein the heterologous polynucleotide comprising hPTH1R exons 4 to 16 would be knocked-in at mouse exon 4 and part of intron 4, thus replacing those mouse endogenous segments with a cassette comprising a coding sequence encoding human PTH1R exons 4-16 (SEQ ID NO: 4), and a poly-A tail.

The foregoing coding sequence comprises the sequence “TACCCT TACGAT GTTCCG GACTAC GCG” (SEQ ID NO: 7) (nucleotide positions 187-213), which encodes a human influenza hemagglutinin (HA) epitope tag, and results in the replacement of mouse amino acid residues 88-96 “YPESEEDKE” (SEQ ID NO: 3) with the human amino acid residues “YPYDVPDYA” (SEQ ID NO: 2).

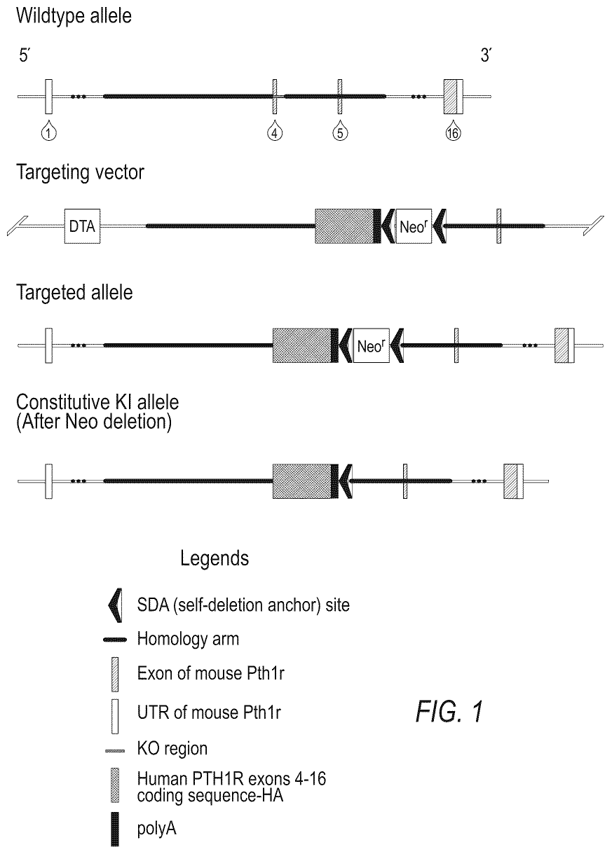

The cells selected for targeting were C57BL/6 embryonic stem cells (ESCs). To engineer the targeting vector, homology arms were generated by PCR using BAC clone RP24-68N11 or RP23-278G23 from the C57BL/6 library as a template. A diagram showing the targeting strategy is provided in FIG. 1.

In the targeting vector, a neomycin phosphotransferase II (Neo) cassette was flanked by self-deletion anchor (SDA) sites and used for a positive selection marker. Diphtheria toxin A (DTA) was used as a negative selection marker.

### Example 2. Generation of Transgenic Animals

Transgenic animals were generated as follows: First, a polynucleotide comprising a first nucleotide sequence comprising a human Parathyroid Hormone 1 Receptor (hPTH1R) exons 4 to 16, wherein said heterologous polynucleotide is operable to encode a human PTH1R protein; and a second nucleotide sequence comprising a polyadenylation signal; and a 5′-homology arm (flanking the N-terminus) and 3′-homology arm (flanking the C-terminus) of the abovementioned polynucleotide, and targeting the mouse loci described above, were cloned into a targeting vector, and confirmed via restriction digest and sequencing.

Next, embryonic stem cells (ESC) were transformed with the vector; here, C57BL/6 ESCs were used for gene targeting with the vector as described above. The vector was electroporated into ESCs, followed by appropriate drug selection and isolation of drug-resistant clones. Successful transformation was confirmed via Southern Blotting. ESCs can be isolated from mouse embryos, or ordered from commercial sources. An exemplary ESC line, the C57BL/6 Mouse Embryonic Stem Cells (Catalog No. MUBES-01001), is available from CYAGEN® (2255 Martin Avenue, Suite E Santa Clara, CA 95050-2709, USA), and is isolated from the inner cell mass of a C57BL/6 blastocyst (obtained at 3.5 days post coitus).

The transformed ESCs were screened by PCR to identify clones containing the human PTH1R coding sequence, which were then further confirmed by Southern blot. Two targeted ES cell clones were identified and confirmed: 1A6 and 1F11, which were then subsequently selected for blastocyst microinjection in order to produce the founder generation (F0).

A cell obtained from the 1A6 or 1F11 clone population were then injected into the blastocysts of C57BL/6 albino embryos, which were subsequently reimplanted into CD-1 pseudo-pregnant females.

Founder animals (F0) were identified by their coat color, and their germline transmission was confirmed by breeding with C57BL/6 females; thus, the heterozygote knock-in positive (KI/+) mice were confirmed as germline-transmitted via crossbreeding F0 founder mice with wild-type. The homozygotes (KI/KI) were acquired by mating the heterozygotes (KI/+) with each other.

### Example 3. Assessment of Heterozygous Transgenic Animals

### Knock-In (KI) Product

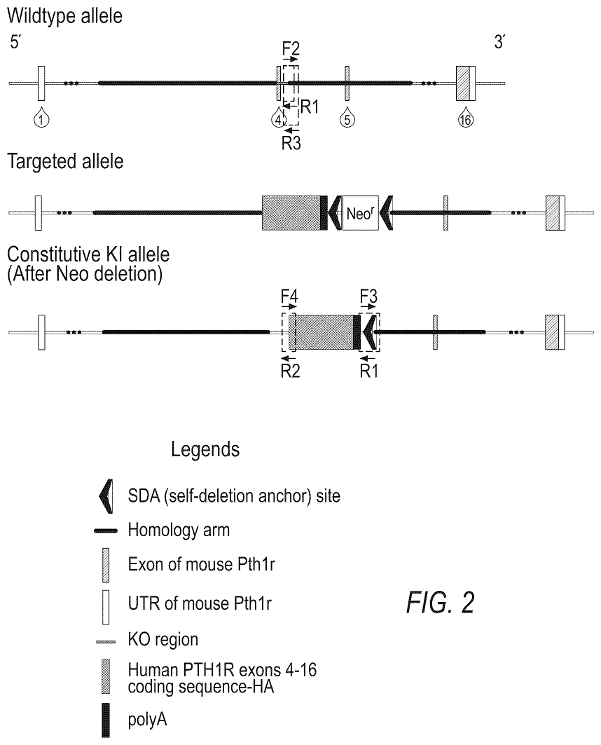

The genotyping strategy used to assess heterozygous transgenic animals is presented in FIG. 2.

To confirm the successful knock-in (KI) of the transgene (i.e., the presence of the targeted allele), PCR was performed using the following primers:

(SEQ ID NO: 8)
F4: 5′-GACTCCCCACATTCTCTCTGAAG-3′
(SEQ ID NO: 9)
R2: 5′-GCGTAGTCCGGAACATCGTAA-3′

Polymerase chain reaction (PCR) conditions were as follows. The reaction mix consisted of: mouse genomic DNA (1.5 μL); forward primer (10 μM) (1.0 μL); reverse primer (10 μM) (1.0 μL); Premix Taq Polymerase (12.5 μL); and ddH₂O (9.0 μL); for a total of 25.0 μL. Cycling conditions included an initial denaturation step of 94° C. for 3 min, followed by 33 or 35 cycles of a denaturation step of 94° C. for 30 seconds; an annealing step of 62° C. for 35 seconds; and an extension step of 72° C. for 35 seconds; followed by an additional extension step of 72° C. for 5 minutes. The expected PCR product using the abovementioned primers is 340 bp for the presences of the targeted allele, and, with no product for the WT allele.

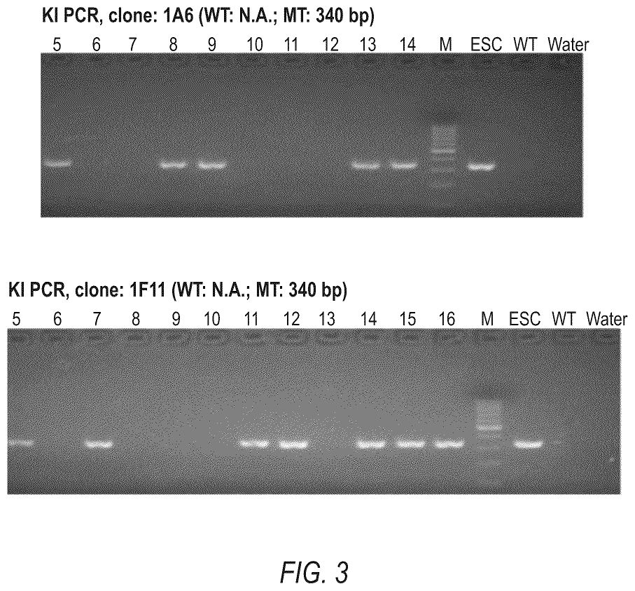

The results of the KI PCR assessment for clones 1A6 and 1F11 are shown in FIG. 3. Here, bands corresponding to about a 340 bp PCR product are shown for pups 5 #, 8 #, 9 #, 13 # and 14 #(top gel) derived from clone 1A6, thus confirming successful knock-in. Likewise, the bottom gel shows successful knock-in of the transgene in pups 5 #, 7 #, 11 # and 14 #, derived from clone 1F11.

### Wildtype Allele

A PCR was run to determine the presence of the WT allele. Here the primers used were as follows:

(SEQ ID NO: 10)
F2: 5′-GATCCTTACCTTCCGGGACTC-3′
(SEQ ID NO: 11)
R3: 5′-AGTTCTAGGGATGCTGGTTCTATG-3′

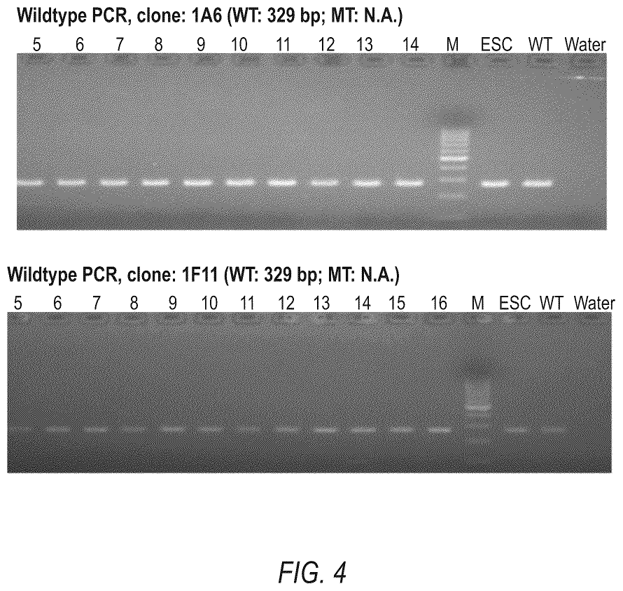

PCR reaction mix components (except for primers) and cycling conditions were the same as described above. The expected PCR product was 329 bp, with no product expected for the targeted allele. As shown in FIG. 4, all of the pups derived from the 1A6 clone and the 1F11 clone have a 329 bp PCR product present.

### Neo Deletion

Because the Neo cassette is flanked by SDA sites, it is self-deleted in germ cells; accordingly, the offspring are Neo cassette-free. To confirm that the offspring are Neo cassette free, a Neo deletion PCR was run using the following primers directed to the targets flanking the Neo cassette with an expected product of 407 bp:

(SEQ ID NO: 12)
F3: 5′-CATAGAAAAGCCTTGACTTGAGGTT-3′
(SEQ ID NO: 13)
R1: 5′-TCTCTTTAAGGAAGTTGGCCCAG-3′

PCR reaction mix components (except for primers) and cycling conditions were the same as described above. The expected PCR product is 407 bp.

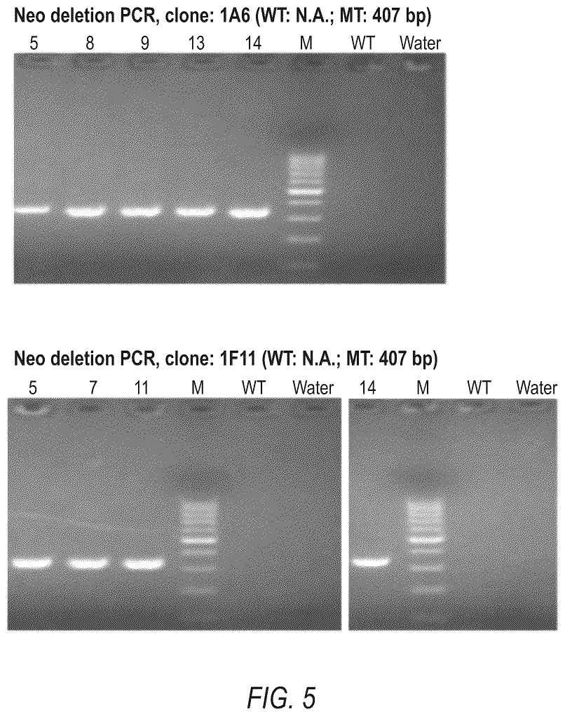

The results of the Neo deletion PCR are shown in FIG. 5. Here, the gels show the successful deletion of the Neo cassette in pups 5 #, 8 #, 9 #, 13 # and 14 #(top gel) from clone 1A6, and pups 5 #, 7 #, 11 # and 14 #, derived from clone 1F11. FIG. 5

### Summary and Suggested Breeding and Genotyping Assay

A total of five pups (5 #, 8 #, 9 #, 13 # and 14 #) from clone 1A6 and four pups (5 #, 7 #, 11 # and 14 #) from clone 1F11 were identified positive by PCR screening for KI, wildtype and Neo deletion, the positive pups were reconfirmed by PCR screening for Neo deletion.

To generate homozygous transgenic mice, heterozygous mice may be intercrossed and subsequently genotyped using the following primer strategy:

(SEQ ID NO: 12)
F3: 5′-CATAGAAAAGCCTTGACTTGAGGTT-3′;
(SEQ ID NO: 13)
R1: 5′-TCTCTTTAAGGAAGTTGGCCCAG-3′
(SEQ ID NO: 10)
F2: 5′-GATCCTTACCTTCCGGGACTC-3′

The foregoing primers are expected to yield the following PCR products in the offspring: a wildtype PCR product of 265 bp; a homozygote PCR product of 407 bp; and heterozygote PCR products of 407 bp/265 bp.

### Example 4. Assessment of Homozygous Transgenic Animals

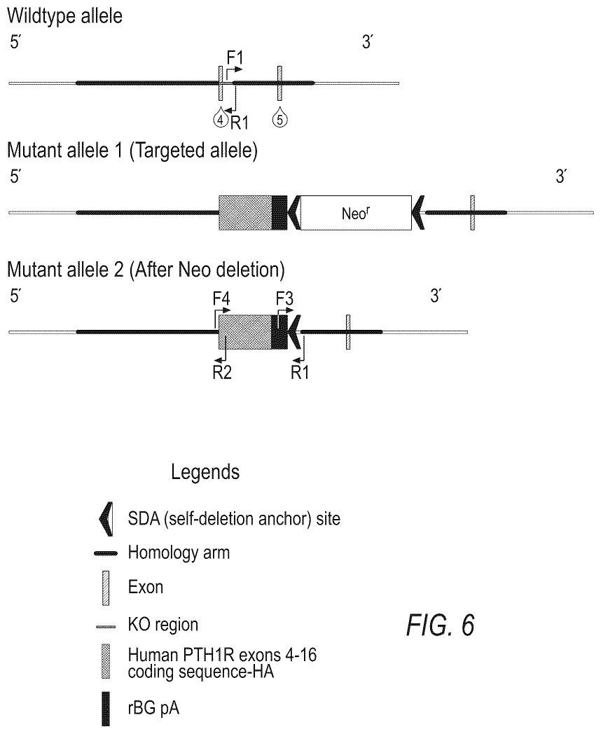

After confirming correctly targeted ES clones via Southern Blotting, clones were selected for blastocyst microinjection to produce the founder generation. The heterozygotes (KI/+) were confirmed as germline-transmitted via crossbreeding F0 founder mice with wild-type. The homozygotes (KI/KI) were acquired by mating the heterozygotes (KI/+) with each other. In the end, 4 male and 1 female homozygotes (KI/KI) were confirmed. The genotyping strategy used to assess heterozygous transgenic animals is presented in FIG. 6.

### Knock-In (KI) Product

To confirm the successful knock-in (KI) of the transgene (i.e., the presence of the targeted allele), PCR was performed using the following primers:

(SEQ ID NO: 8)
F4: 5′-GACTCCCCACATTCTCTCTGAAG-3′
(SEQ ID NO: 9)
R2: 5′-GCGTAGTCCGGAACATCGTAA-3′

PCR conditions were as follows. The reaction mix consisted of: Mouse genomic DNA (1.5 μL); Forward primer (10 μM) (1.0 μL); Reverse primer (10 μM) (1.0 μL); Premix Taq Polymerase (12.5 μL); and ddH₂O (9.0 μL); for a total of 25.0 μL. Cycling Conditions included an initial denaturation step of 94° C. for 3 min, followed by 33 or 35 cycles of a denaturation step of 94° C. for 30 seconds; an annealing step of 62° C. for 35 seconds; and an extension step of 72° C. for 35 seconds; followed by an additional extension step of 72° C. for 5 minutes. The expected PCR product using the abovementioned primers is 340 bp for the presences of the targeted allele, and, with no product for the WT allele.

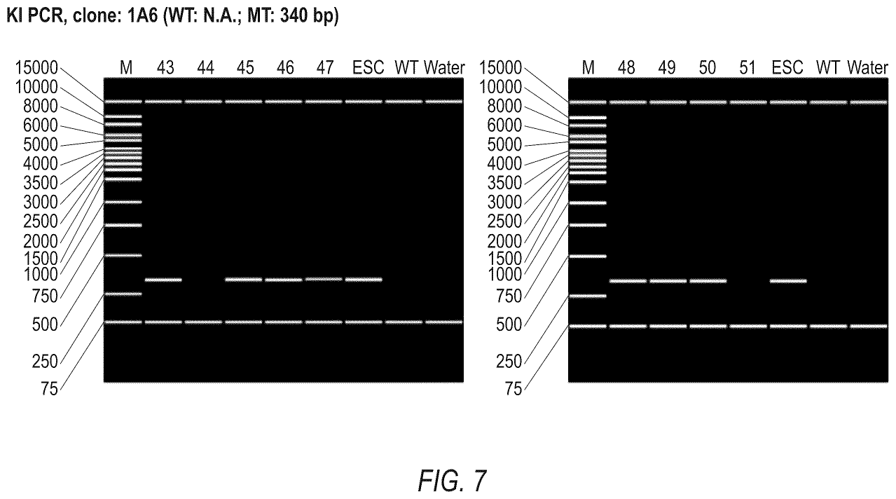

The results of the KI PCR assessment for clone 1A6 is shown in FIG. 7. Here, bands corresponding to about a 340 bp PCR product is shown for pups (43 #, 45 #, 46 #, 48 # and 50 #) from clone 1A6, thus confirming successful knock-in. FIG. 7.

### Wildtype Allele

A PCR was run to determine the presence of the WT allele. Here the primers used were as follows:

(SEQ ID NO: 16)
F1: 5′-CAGGCGATCCTTACCTTCCG-3′
(SEQ ID NO: 13)
R1: 5′-TCTCTTTAAGGAAGTTGGCCCAG-3′

PCR reaction mix components (except for primers) and cycling conditions were the same as described above. The expected PCR product for the WT allele was 270 bp, with no product expected for the targeted allele.

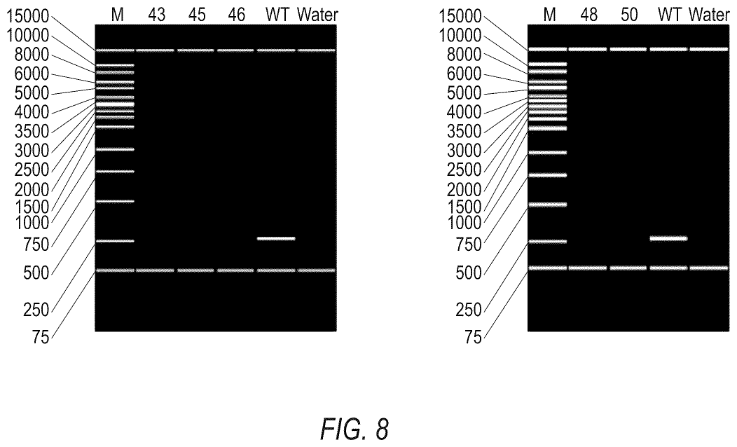

The results of the WT PCR are shown in FIG. 8. Five pups (43 #, 45 #, 46 #, 48 # and 50 #) from clone 1A6 were identified positive by PCR screening for the WT allele, as indicated by a lack of presence of a 270 bp PCR product. FIG. 8.

### Neo Deletion

Because the Neo cassette is flanked by SDA sites, it is self-deleted in germ cells; accordingly, the offspring are Neo cassette-free. To confirm that the offspring are Neo cassette free, a Neo deletion PCR was run using the following primers directed to targets flanking Neo cassette, having an expected product size of 407 bp:

(SEQ ID NO: 12)
F3: 5′-CATAGAAAAGCCTTGACTTGAGGTT-3′
(SEQ ID NO: 13)
R1: 5′-TCTCTTTAAGGAAGTTGGCCCAG-3′

PCR reaction mix components (except for primers) and cycling conditions were the same as described above. The expected PCR product is 407 bp.

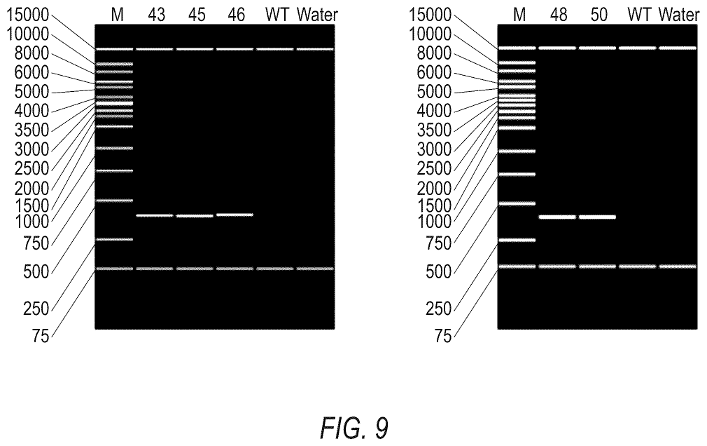

The results of the Neo deletion PCR are shown in FIG. 9. Pups 43 #, 45 #, 46 #, 48 # and 50 # from clone 1A6 show the expected 407 bp PCR product, indicating successful Neo cassette deletion.

### Summary and Suggested Breeding and Genotyping Assay

A total of five pups (43 #, 45 #, 46 #, 48 # and 50 #) from clone 1A6 were identified positive by PCR screening for Neo cassette deletion, lack of WT gene, and successful KI.

A suggested breeding strategy to generate homozygous targeted mice is to intercross heterozygous mice, and use the following primers:

(SEQ ID NO: 16)
F1: 5′-CAGGCGATCCTTACCTTCCG-3′
(SEQ ID NO: 12)
F3: 5′-CATAGAAAAGCCTTGACTTGAGGTT-3′
(SEQ ID NO: 13)
R1: 5′-TCTCTTTAAGGAAGTTGGCCCAG-3′

As shown in FIG. 6, using the foregoing primers is expected to yield a WT PCR fragment of 270 bp, and a targeted allele fragment of 407 bp; individual pups can be distinguished by performing PCR and observing the presence and/or absence of one of these products. For example, because the wild-type allele does not have the F2 (which comes from human insert and is the heterozygous allele), there is no product expected.

### Example 5. DNA Sequence of the 3′ Junction Region

Pursuant to the breeding and genotyping assay described in the examples above, an intercross was performed to generate homozygous mice. The homozygous mice were then screened with the following primers:

(SEQ ID NO: 12)
F3: 5′-CATAGAAAAGCCTTGACTTGAGGTT-3′
(SEQ ID NO: 13)
R1: 5′-TCTCTTTAAGGAAGTTGGCCCAG-3′

As shown in FIG. 6, the abovementioned primers is expected to yield a targeted allele fragment of 407 bp. Homozygous mice were analyzed via PCR to determine the presence of a 407 bp PCR product using the F3 and R1 primers.

Genomic DNA was extracted from tissue isolated from the tails of three homozygous hPTH1R knock-in mice (mouse #1: C57BL-KI-hP1R-1-15; mouse #2: C57BL-KI-hP1R-2-16; and mouse #3: CD1-KI-hP1R-XL130). The genomic DNA from each mouse was then PCR-amplified using primers F2 (SEQ ID NO: 18) and R1 (SEQ ID NO: 17).

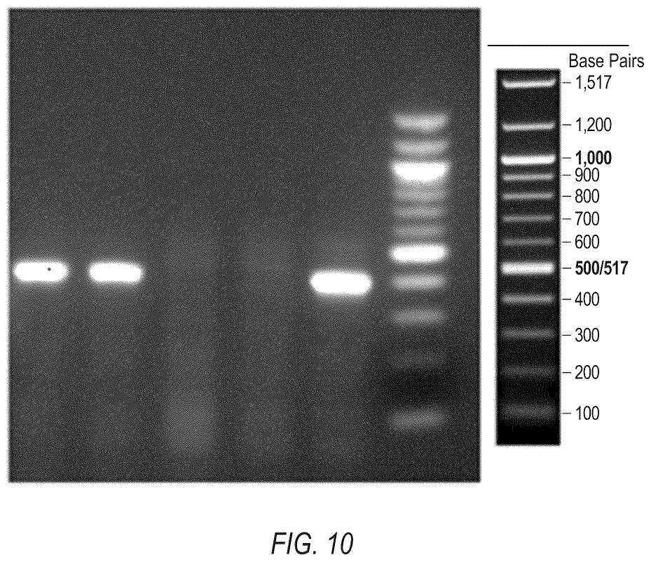

A gel showing the PCR results is provided in FIG. 10. The results of the PCR are summarized in the table below.

TABLE 1
PCR results for 3′ junction region analysis. Here, the
results of the PCR analysis performed on 3 homozygous knock-in
mice reveal are shown. The expected PCR product size (in base
pairs, “bp”) corresponds with the results shown in the gel.

| Lane | | | Expected band | |
|---|---|---|---|---|
| No. | Sample | Primers | size (bp) | Result (bp) |
| 1 | C57BL-KI-hP1R-1-15 | F3 and R1 | 407 | ~407 |
| 2 | C57BL-KI-hP1R-2-16 | F3 and R1 | 407 | ~407 |
| 3 | C57BL-WT-1 | F3 and R1 | None | None |
| 4 | C57BL-WT-1 | F3 and R1 | None | None |
| 5 | CD1-KI-hP1R-XL130 | F3 and R1 | 407 | ~407 |
| 6 | Ladder | NA | NA | NA |

As shown in FIG. 10 and the table above, the PCR product corresponds to the expected PCR product size in the homozygous mice (i.e., 407 bp), thus confirming successful integration of the transgene in the mouse genome.

Next, the three PCR products were analyzed by DNA sequencing in six reactions that used the F2 or RI primers. DNA sequencing was performed using the Sanger sequencing method: i.e., a cycle sequencing reaction using the Applied Biosystems BigDye v3.1 Cycle Sequencing Kit, which employs a fluorescently-labeled dideoxy-nucleotide chain termination method to generate extension products from DNA templates. Extension products were purified using SPRI technology. Subsequently, fragment separation and sequence detection was carried out by capillary electrophoresis on the 96-well capillary matrix of an ABI3730XL DNA Analyzer, followed by post-detection processing. In the final analysis step, a combination of software base calling and manual inspection of the individual trace files is employed to warrant the highest possible quality of the generated data.

The six resulting DNA sequences obtained were aligned and further analyzed to derive a consensus sequence using the Clustal-O and EMBOSS.Cons software tools (EMBL-EBI, Wellcome Genome Campus, Hinxton, Cambridgeshire, CB10 1SD, UK) EMBOSS CONS tool (www.ebi.ac.uk/Tools/msa/emboss_cons/). The Consensus sequence was further optimized manually with the criteria that at each nucleotide position, the nucleotide used in the consensus sequence was present in at least two of the six independent sequence reads.

The consensus sequence for the 3′ junction region and the alignment with the six sequences obtained are shown below. The 3′ junction alignment includes sequence from the intron-3/Exon-4 region of the mouse genome (mouse chromosome ID=ENSMUSG00000032492; GRCm39:9:110560172:110560900:1; obtained at the following website: https://useast.ensembl.org/Mus_musculus/Transcript/Exons?db=core; g=ENSMUSG0000003 2492). The F2-R1 region contains the engineered rabbit β-globin polyadenylation signal (rBG-pA) used for termination and polyadenylation of the hPTH1R exons 4 to 16 mRNA transcript. The rBG-pA is joined to a portion of the 5′ end of intron 4 of the mouse PTH1R gene. FIG. 6. The consensus DNA sequence derived from the six DNA sequences (entitled “Consensus-F2.rBG_R1.Int4_3′Junction”) is provided below, and set forth in SEQ ID NO: 23:

(SEQ ID NO: 23)
TGAGGTTAGATTTTTTTTATATTTTGTTTTGTGTTATTTTTTTCTTTAA
CATCCCTAAAATTTTCCTTACATGTTTTACTAGCCAGATTTTTCCTCCT
CTCCTGACTACTCCCAGTCATAGCTGTCCCTCTTCTCTTATGGAGATCC
TGGAGGGACCTAATAACTTCGTATAGCATACATTATACGAAGTTATATT
AAGGGTTATTGAATATGATCGGAATTGGGCTGCAGGAATTCGATAGCTT
GGCTGCAGGTCGACGTACGTAGCAAGCTTGATGGGCCCTGGTACCCCGG
GGTCCCAGTGGATTTAGATGGGGTTGGGCAAGCCAGGGACTTTGCTGAG
GGC

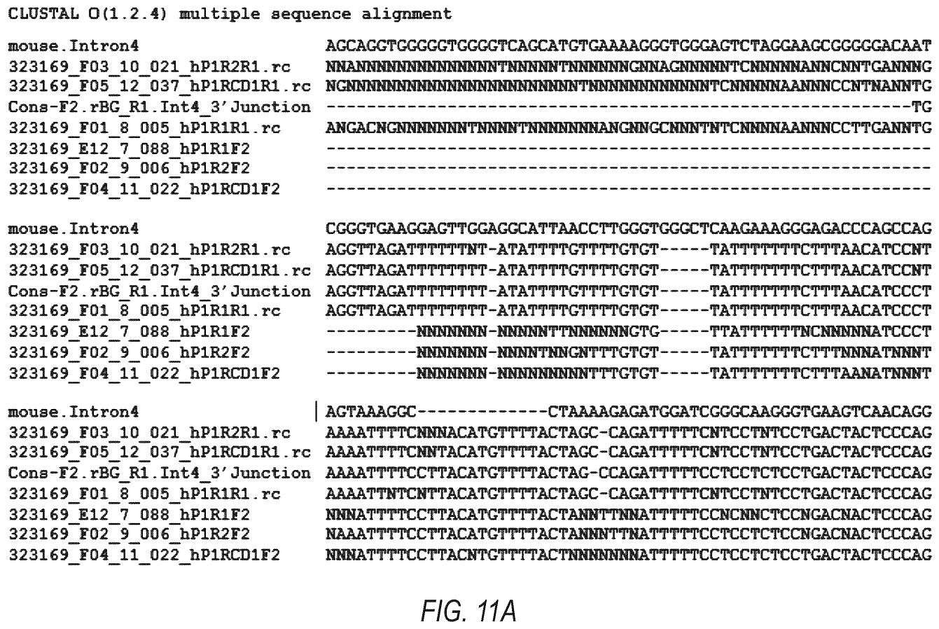

The six obtained DNA sequences were aligned with the derived consensus sequence using a CLUSTAL alignment, which revealed regions and sites of overlap (identity) for the six sequences and confirmed the expected sequence in those regions and sites. FIG. 11. The CLUSTAL program is well described by Higgins et al. Gene 73:237 244 (1988); Higgins et al. CABIOS 5:151-153 (1989); Corpet et al. Nucleic Acids Res. 16:10881-90 (1988); Huang et al. CABIOS 8:155-65 (1992); and Pearson et al. Meth. Mol. Biol. 24:307-331 (1994), the disclosures of which are incorporated herein by reference in their entireties.

### Example 6. DNA Sequence of the HA-Tag hPTH1R Region

Using the genomic DNA extracted from tissue isolated from the tails of the three homozygous mice described above (mouse #1: C57BL-KI-hP1R-1-15; mouse #2: C57BL-KI-hP1R-2-16; and mouse #3: CD1-KI-hP1R-XL130), as described above, the region of the HA-tag was evaluated.

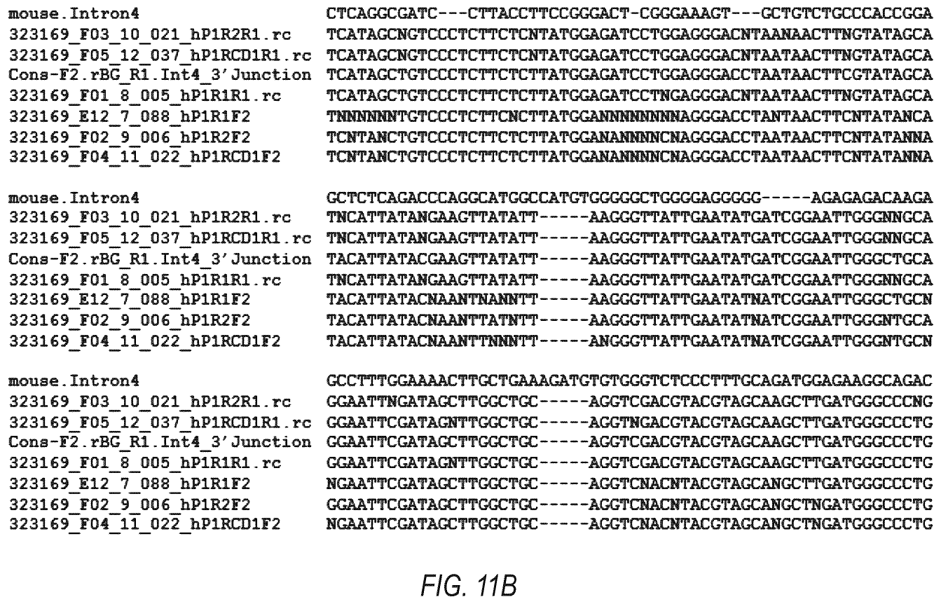

The genomic DNA was PCR-amplified using primers F4 (Intron-3 forward) (SEQ ID NO: 14) and R-291 (hPTH1R residue 291 Reverse) (SEQ ID NO: 24). A schematic of the hPTH1R knock-in genome showing the location of the F4 and R-291 primer sites is shown in FIG. 12.

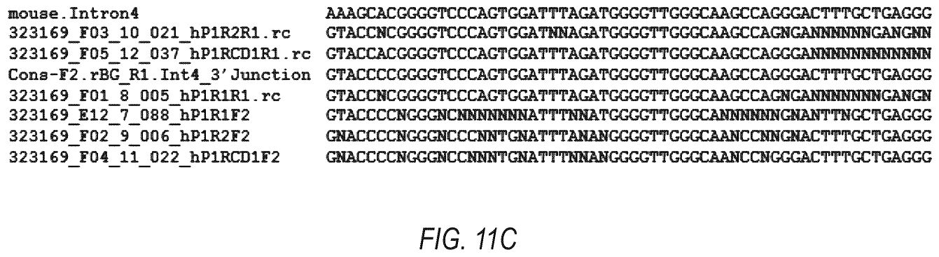

A gel showing the PCR results is provided in FIG. 13. The results of the PCR are summarized in the table below.

TABLE 2
PCR results for HA-tag hPTH1R region analysis. Here, the results
showed two PCR products that were ~900 bp and ~400 bp.

| Lane | | | Expected band | |
|---|---|---|---|---|
| No. | Sample | Primers | size (bp) | Result (bp) |
| 1 | Ladder | NA | NA | NA |
| 2 | C57BL-KI-hP1R-1-15 | F4, R-291 | 928 | ~900 (~400) |
| 3 | C57BL-KI-hP1R-2-16 | F4, R-291 | 928 | ~900 (~400) |
| 4 | CD1-KI-hP1R-XL130 | F4, R-291 | 928 | ~900 (~400) |
| 5 | Ladder | NA | NA | NA |

As shown in FIG. 13 and the table above, the PCR yielded two products: one product around 900 bp, and the other product around 400 bp.

The PCR products were analyzed by DNA sequencing as described above in Example 5, using the F4 primer. The resulting DNA sequences were aligned using the CLUSTAL O tool, and a consensus sequence was derived using the EMBOSS CONS tool, as described above. The consensus sequence (entitled “Consensus.F3_R-291 Sequence.vers 3”) was further revised via visual inspection, then translated into protein sequence and aligned with the hPTH1R-HA and mouse Pth1r protein sequences. The consensus sequence, Consensus.F3_R-291 Sequence.vers 3, is provided below and in SEQ ID NO: 25:

(SEQ ID NO: 25)
GCCGCTGGGGGCACCAGGTGAGGTGGTGGCTGTGCCCTGTCCGGACTAC
ATTTATGACTTCAATCACAAAGGCCATGCCTACCGACGCTGTGACCGCA
ATGGCAGCTGGGAgCTGGTGCCTGGGCACAACAGGACGTGGGCCAACTA
CAGCGAGTGTGTCaaATTTCTCACCAATGAGACTCGTGAACGGGAgGTG
TTTGACCGCCTGGGCATGATTTACACCGTGGGCTACTCCGTGTCCCTGG
CGTCCCTCACCGTAgCTGTGCTCATCCTGGCCTACTTTAGCGGCTGCAC
TGCACGCGCAaCTACaTCCACATGCACCTGTTcCTGTCCtTCATGCTGC
G

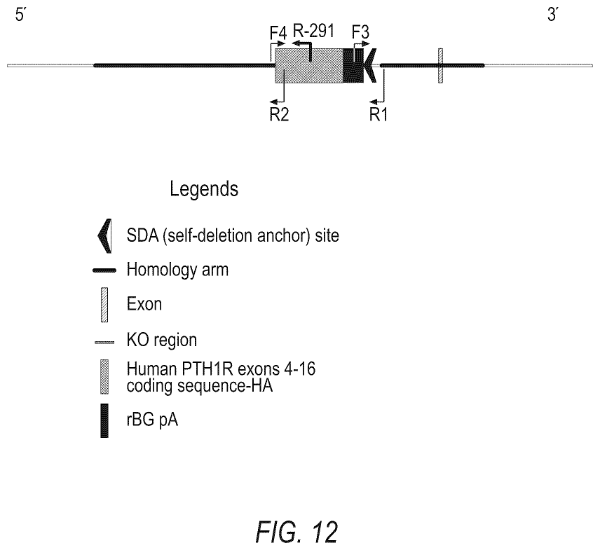

The consensus sequence above was translated into an amino acid sequence, and compared with the amino acid sequences of the hPTH1R-HA and mouse PTH1R proteins. FIG. 14. In the CLUSTAL O protein alignment, the HA tag is highlighted in red, and residues unique to the WT mouse PTH1R protein are highlighted in blue. None of the residues unique to the WT mouse PTH1R protein were found in the translated consensus sequence. FIG. 14.

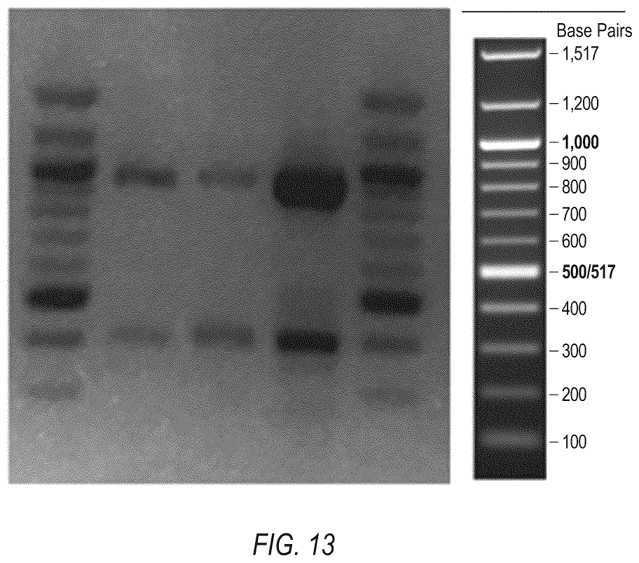

A CLUSTAL-O alignment of the consensus F4-R-291 DNA sequence and the sequences obtained from the three knock-in mice for the F4-R-291 PCR products, is provided in FIG. 15. These sequences confirm the expected nucleotide sequence structure and position of the hP1R-KI replacement cassette.

The sequence analysis performed here confirms the presence of the following protein sequence region highlighted in bold:

(SEQ ID NO: 30)
*
MGTARIAPGLALLLCCPVLSSAYALVDADDVMTKEEQIFLLHRAQAQCE
KRLKEVLQRPASIMESDKGWTSASTSGKPRKDKASGKLYPYDVPDYAAP
TGSRYRGRPCLPEWDHILCWPLGAPGEVVAVPCPDYIYDENHKGHAYRR
CDRNGSWELVPGHNRTWANYSECVKELTNETREREVEDRLGMIYTVGYS
VSLASLTVAVLILAYFRRLHCTRNYIHMHLFLSFMLRAVSIFVKDAVLY
SGATLDEAERLTEEELRAIAQAPPPPATAAAGYAGCRVAVTFFLYFLAT
NYYWILVEGLYLHSLIFMAFFSEKKYLWGFTVFGWGLPAVEVAVWVSVR
ATLANTGCWDLSSGNKKWIIQVPILASIVLNFILFINIVRVLATKLRET
NAGRCDTRQQYRKLLKSTLVLMPLFGVHYIVFMATPYTEVSGTLWQVQM
HYEMLFNSFQGFFVAIIYCFCNGEVQAEIKKSWSRWTLALDFKRKARSG
SSSYSYGPMVSHTSVTNVGPRVGLGLPLSPRLLPTATTNGHPQLPGHAK
PGTPALETLETTPPAMAAPKDDGFLNGSCSGLDEEASGPERPPALLQEE
WETVM*

In the foregoing sequence, position V26 is indicated with an asterisk. The regions underlined and in italics were confirmed via direct DNA sequence analysis using the R-291 primer for residues Q57-E155. And, regions highlighted in bold were confirmed using the F3 primer for residues P119-F212. The asterisk indicates a stop codon.

In the foregoing sequence, residues M1-A22, i.e., “MGTARIAPGLALLLCCPVLSSAYA” (SEQ ID NO: 26) correspond to a signal sequence. The short segment following the signal sequence, i.e., Y23-A24-L25, corresponds to a portion of the mature mouse PTH1R protein that is encoded by exon 3, and thus is not part of the hPTH1R-KI sequence, which starts at codon 4. Accordingly, the hPTH1R-KI protein construct thus has the mouse signal peptide and the mouse Y23-L25 segment joined to V26 of hPTH1R. The signal is cleaved off and the Y23-L25 sequences are the same in mouse and humans, thus having no effect on in terms of receptor function/specificity; see, e.g., a comparison of the mouse and human PTH1R residues in positions 1-25:

Mouse PTH1R residues 1-25:
(SEQ ID NO: 31)
MGTARIAPSLALLLCCPVLSSAYAL;
Human PTHIR residues 1-25:
(SEQ ID NO: 32)
MGTARIAPGLALLLCCPVLSSAYAL.

Consequently, the heterologous transgene knocked into the mouse encodes a protein starting at Met-1 of the mouse PTH1R, and including mouse residues from Met-1 to L25, which is joined to residue V26 of the human PTH1R (also comprising an HA sequence), and ending at Met593 (followed by a stop codon). The mouse signal peptide, M1-A22 is removed, and the Y23-L25 sequence is the same in mouse and humans, so the resulting mature PTH1R construct contains exactly the same PTH1R sequence as present in humans.

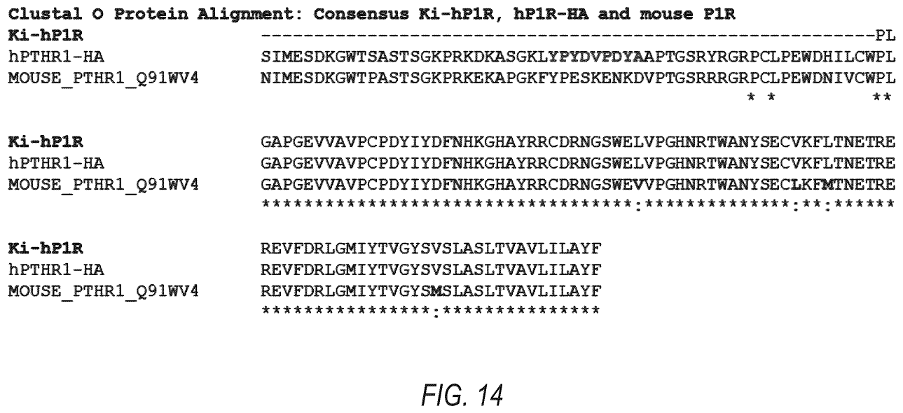

A summary of the foregoing construct is provided in FIG. 16.

### Example 7. DNA Sequence Analysis of the hPTH1R Knock-In Allele

The sequence of the heterologous polynucleotide comprising human Parathyroid Hormone 1 Receptor (hPTH1R) exons 4 to 16, wherein said heterologous polynucleotide is operable to encode a human PTH1R protein, was then re-evaluated using Sanger sequencing analysis according to the methods described above. Briefly, genomic DNA was obtained from the tail tissue of homozygous hPTH1R-KI mice and PCR was performed using the list of primers in the table below.

TABLE 3
Primers used in sequence Analysis of the hPTHIR knock-in allele.

| SEQ ID | | | | | |
|---|---|---|---|---|---|
| NO. | Name | Sequence 5′-3′ | 5′ nt | 3′ nt | Length |
| 33 | F3-Int3 (Fwd) | GACTCCCCACATTCTCTCTGAAG | 1 | 23 | 22 |
| 34 | F-HA86 (Fwd) | ggaagctcTACCCTTACGATGTT | 307 | 329 | 22 |
| 35 | R2-HA95 (Rev) | GCGTAGTCCGGAACATCGTAA | 340 | 320 | 20 |
| 36 | F-1255 (Fwd) | CATCTTCGTCAAGGACGCTGTG | 761 | 782 | 21 |
| 37 | R-291 (Rev) | AGGAAGTAAAGGAAGAAGGTCACAG | 928 | 904 | 24 |
| 38 | F-mid.ins (Fwd) | GGCGTCCACTACATTGTCTTCATG | 1305 | 1328 | 23 |
| 39 | R-mid.ins (Rev) | CATGAAGACAATGTAGTGGACGCC | 1328 | 1305 | 23 |
| 40 | F-M611 (Fwd) | GAAGAGTGGGAGACAGTCATG | 1812 | 1832 | 20 |
| 41 | R-M611 (Rev) | CATGACTGTCTCCCACTCTTC | 1832 | 1812 | 20 |
| 42 | F2-rbg (Fwd) | CATAGAAAAGCCTTGACTTGAGGTT | 2194 | 2218 | 24 |
| 43 | R-rbg (Rev) | AACCTCAAGTCAAGGCTTTTCTATG | 2218 | 2194 | 24 |
| 44 | R1.int4 (Rev) | TCTCTTTAAGGAAGTTGGCCCAG | 2600 | 2578 | 22 |

5′ nt and 3′ nt refers to 5′ nucleotide and 3′ nucleotide, respectively, and indicates the nucleotide positions in the nucleotide sequence of the hPTHIR KI region shown in Table 4 below.

Next, PCR products were then analyzed via Sanger sequencing as described in Example 5 (Applied Biosystems BigDye v3.1 Cycle Sequencing Sanger sequencing analyses). The sequence of the entire insert with flanking regions of mouse Intron 3 and mouse intron 4 is displayed in the table below.

TABLE 4
Nucleotide sequence of hPTH1R KI region (SEQ ID NO: 45). The sequence was obtained by Sanger sequence
analysis of PCR products generated using the primers shown in Table 3.

| SEQ ID NO: 45 (>Seq122821h4) | Nt | Note | Primer |
|---|---|---|---|
| gactccccacattctctctgaagggatcacttttcgaaagg | 41 | Intron 3 | (F3-Int3) |
| ggggaaatccctggggaggttgcatgagtttggaaccagctgcctcacctggaagtgctg | 101 | Intron 3 | |
| cctacagtctgacctttggtttggcaggtggatgcagatgacgtcatgactaaagaggaa | 161 | hP1R | |
| cagatcttcctgctgcaccgtgctcaggcccagtgcgaaaaacggctcaaggaggtcctg | 221 | hP1R | |
| cagaggccagccagcataatggaatcagacaagggatggacatctgcgtccacatcaggg | 281 | hP1R | |
| aagcccaggaaagataaggcatctgggaagctctacccttacgatgttccggactacgcg | 341 | hP1R | (HA-F.86) |
| gcacccactggcagcaggtaccgagggcgcccctgtctgccggaatgggaccacatcctg | 401 | hP1R | |
| tgctggccgctgggggcaccaggtgaggtggtggctgtgccctgtccggactacatttat | 461 | hP1R | |
| gacttcaatcacaaaggccatgcctaccgacgctgtgaccgcaatggcagctgggagctg | 521 | hP1R | |
| gtgcctgggcacaacaggacgtgggccaactacagcgagtgtgtcaaatttctcaccaat | 581 | hP1R | |
| gagactcgtgaacgggaggtgtttgaccgcctgggcatgatttacaccgtgggctactcc | 641 | hP1R | |
| gtgtccctggcgtccctcaccgtagctgtgctcatcctggcctactttaggcggctgcac | 701 | hP1R | |
| tgcacgcgcaactacatccacatgcacctgttcctgtccttcatgctgcgcgccgtgagc | 761 | hP1R | |
| atcttcgtcaaggacgctgtgctctactctggcgccacgcttgatgaggctgagcgcctc | 821 | hP1R | (F-I255.A263) |
| accgaggaggagctgcgcgccatcgcccaggcgcccccgccgcctgccaccgccgctgcc | 881 | hP1R | |
| ggctacgcgggctgcagggtggctgtgaccttcttcctttacttcctggccaccaactac | 941 | hP1R | |
| tactggattctggtggaggggctgtacctgcacagcctcatcttcatggccttcttctca | 1001 | hP1R | |
| gagaagaagtacctgtggggcttcacagtcttcggctggggtctgcccgctgtcttcgtg | 1061 | hP1R | |
| gctgtgtgggtcagtgtcagagctaccctggccaacaccgggtgctgggacttgagctcc | 1121 | hP1R | |
| gggaacaaaaagtggatcatccaggtgcccatcctggcctccattgtgctcaacttcatc | 1181 | hP1R | |
| ctcttcatcaatatcgtccgggtgctcgccaccaagctgcgggagaccaacgccggccgg | 1241 | hP1R | |
| tgtgacacacggcagcagtaccggaagctgctcaaatccacgctggtgctcatgcccctc | 1301 | hP1R | |
| tttggcgtccactacattgtcttcatggccacaccatacaccgaggtctcagggacgctc | 1361 | hP1R | (mid.ins-G418-F) |
| tggcaagtccagatgcactatgagatgctcttcaactccttccagggattttttgtcgca | 1421 | hP1R | |
| atcatatactgtttctgcaatggcgaggtacaagctgagatcaagaaatcttggagccgc | 1481 | hP1R | |
| tggacactggcactggacttcaagcgaaaggcacgcagcgggagcagcagctatagctac | 1541 | hP1R | |
| ggccccatggtgtcccacacaagtgtgaccaatgtcggcccccgtgtgggactcggcctg | 1601 | hP1R | |
| cccctcagcccccgcctactgcccactgccaccaccaacggccaccctcagctgcctggc | 1661 | hP1R | |
| catgccaagccagggaccccagccctggagaccctcgagaccacaccacctgccatggct | 1721 | hP1R | |
| gctcccaaggacgatgggttcctcaacggctcctgctcaggcctggacgaggaggcctct | 1781 | hP1R | |
| gggcctgagcggccacctgccctgctacaggaagagtgggagacagtcatgtgaTCCTCA | 1841 | hP1R | (FC-M611) |
| GGTGCAGGCTGCCTATCAGAAGGTGGTGGCTGGTGTGGCCAATGCCCTGGCTCACAAATA | 1901 | rbGPA | |
| CCACTGAGATCTTTTTCCCTCTGCCAAAAATTATGGGGACATCATGAAGCCCCTTGAGCA | 1961 | rbGPA | |
| TCTGACTTCTGGCTAATAAAGGAAATTTATTTTCATTGCAATAGTGTGTTGGAATTTTTT | 2021 | rbGPA | |
| GTGTCTCTCACTCGGAAGGACATATGGGAGGGCAAATCATTTAAAACATCAGAATGAGTA | 2081 | rbGPA | |
| TTTGGTTTAGAGTTTGGCAACATATGCCCATATGCTGGCTGCCATGAACAAAGGTTGGCT | 2141 | rbGPA | |
| ATAAAGAGGTCATCAGTATATGAAACAGCCCCCTGCTGTCCATTCCTTATTCCATAGAAA | 2201 | rbGPA | |
| AGCCTTGACTTGAGGTTAGATTTTTTTTATATTTTGTTTTGTGTTATTTTTTTCTTTAAC | 2261 | rbGPA | (F2-rbg) |
| ATCCCTAAAATTTTCCTTACATGTTTTACTAGCCAGATTTTTCCTCCTCTCCTGACTACT | 2321 | rbGPA | |
| 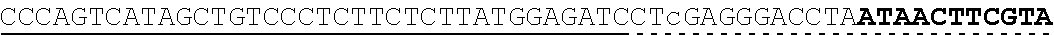 | 2381 | SDA/LOXP | |
| 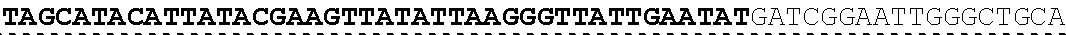 | 2441 | SDA/LOXP | |
| 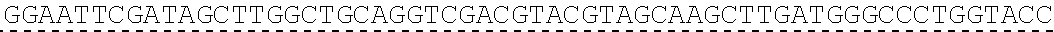 | 2501 | Intron 4 | |
| aCGGGGTCCCAGTGGATTTAGATGGGGTTGGGCAAGCCAGGGACTTTGCTGAGGGCgCTG | 2561 | Intron 4 | |
| GtCCaaacagggtgggctgGGCCAACTTCCTTAAAGAGA | 2600 | Intron 4 | (R1.int4) |

Nucleotide position number (Nt) and sequence corresponding to mouse Intron3 (underlined), hPTH1R cDNA
(regular text), rabbit beta globin poly A (underlined and italic), a vector-derived self-deleting anchor
containing a LOX-P site (dotted underline) and mouse intron 4 (italic) are indicated in the right-hand
columns. Primers sequences are indicated in bold. The hPTH1R cDNA (regular text) encodes hPTH1R V26-M593
(SEQ ID NO: 46)

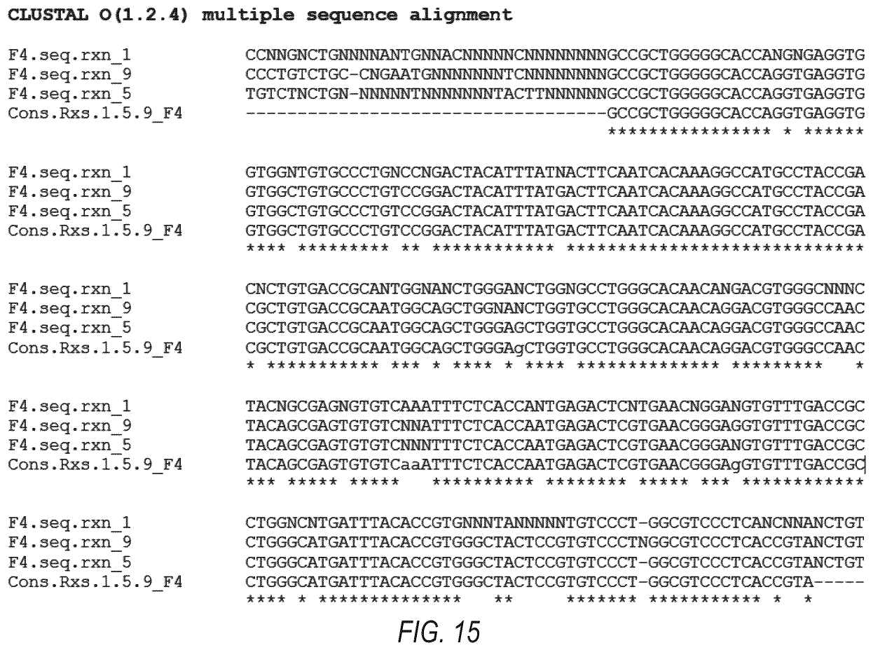

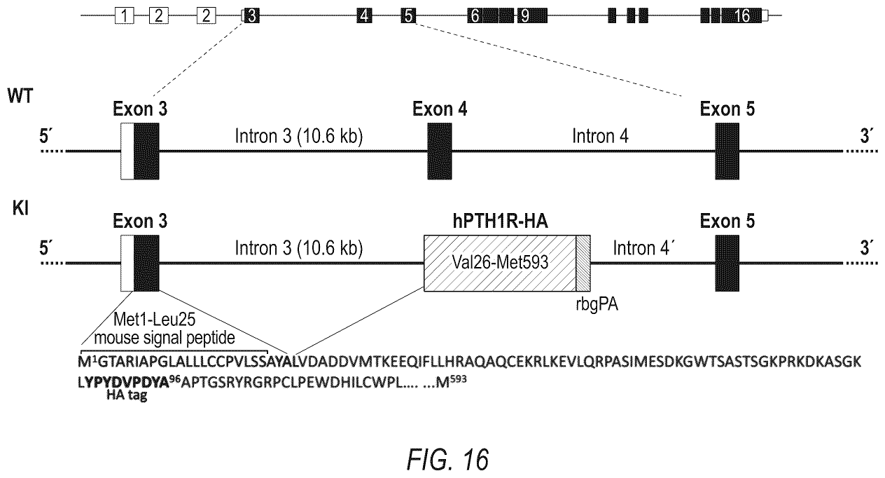

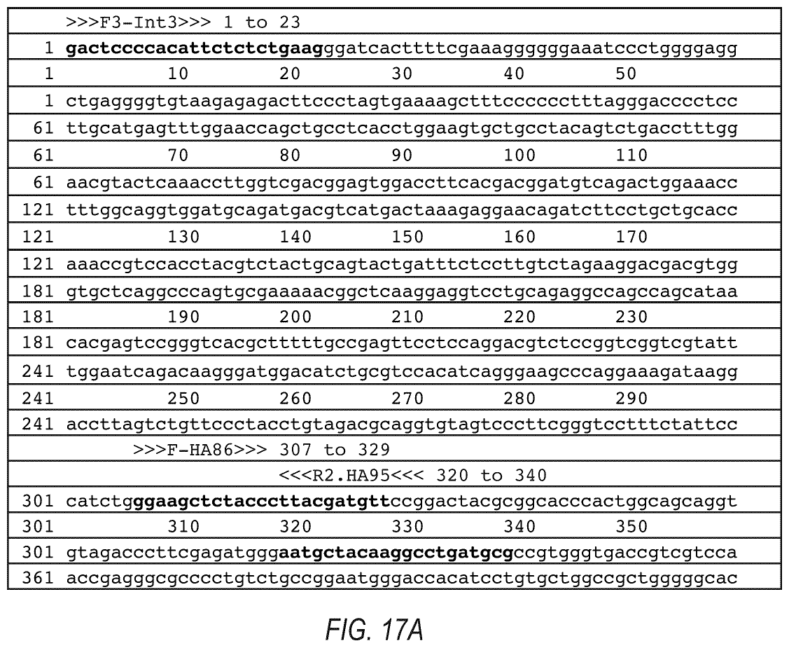

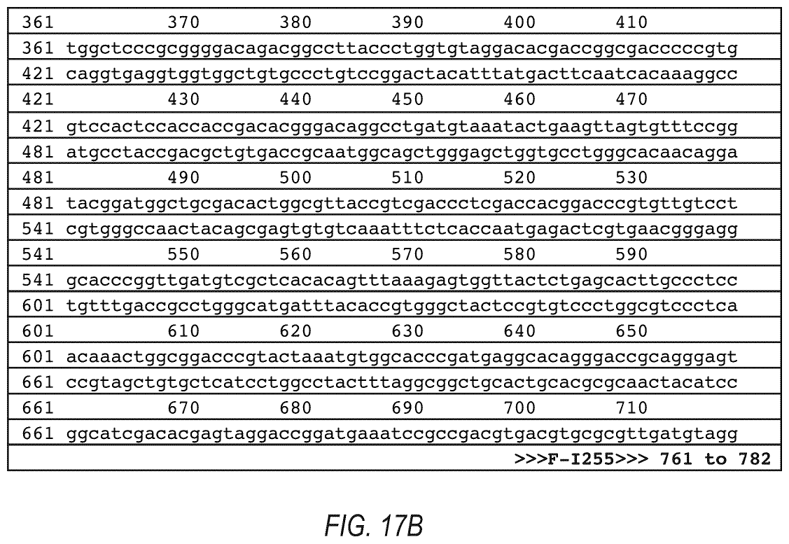

The locations in the DNA sequence of primers used for PCR and DNA sequence analysis are shown in FIGS. 17-19. An alignment of the HA-PTH1R protein sequences encoded by the knock in allele and the mouse PTH1R is displayed in FIG. 20.

### Example 8. Western Blot Analysis of hPTH1R in Kidneys of hTPH1R-KI Mice

The PTH1R is expressed in cells of the distal and proximal renal tubules where it acts to regulate Ca and Pi transport as well as the expression of enzymes involved in the synthesis and metabolism of 1.25(OH)₂ Vitamin D (See Hannan et al., The calcium-sensing receptor in physiology and in calcitropic and noncalcitropic diseases. Nature reviews Endocrinology. 2018; 15(1):33-51). Accordingly, whether the hPTH1R protein was expressed in the kidneys of adult KI mice was evaluated.

Whole kidney lysates prepared from two wild-type mice (WT-1, WT-2) and two homozygous mice comprising a heterologous polynucleotide comprising human Parathyroid Hormone 1 Receptor (hPTH1R) exons 4 to 16 (hereinafter “hPTH1R-KI” mice), were evaluated.

Kidneys were isolated from two wild-type mice (WT-1, WT-2) and two hPTH1R-KI mice (KI-1. KI-2) at ˜20 weeks of age, dissected on ice to remove the capsule, and placed in homogenization buffer (10 mM Tris-HCl, pH 7.8, supplemented with 1 mM EDTA, 1×-protease inhibitor cocktail (Bimake Inc. 100×, Cat. No. B14001), 1 mM DTT, 1 mM NaF, 0.2 mM Vanadate (Sodium Orthovanadate, i.e. “Vanadate,” available from New England Biolabs®, Catalog No. P0758S; 240 County Road, Ipswich, MA 01938-2723 USA), 1% dodecylmaltoside (Sigma Aldrich; Catalog No. 862312) and 1 μM LA-PTH.

The tissue was homogenized using a Kimble Pellet Pestle Motor at 4° C. for 4 minutes. The homogenates were centrifuged at 1000×g for 10 min at 4° C. and the supernatants were collected and centrifuged at 14,000×g for 30 min at 4° C. The supernatants were removed, the pellets were resuspended in 600 μL homogenization buffer, and the protein concentrations were determined by Bradford assay. The samples were then mixed with 2× Laemmeli buffer, incubated at room temperature for 30 min, and after a brief storage at −80° C., a sample volume containing 40 μg of protein was loaded onto an 8% acrylamide-SDS gel; after electrophoresis, the gels were processed for western blotting using HRP-conjugated anti-HA mouse monoclonal antibody (Biolegend, Catalog No. 901520) diluted 1:500 and HRP chemiluminescent substrate reagent (ThermoFisher; Catalog No. 34095; 168 Third Avenue, Waltham, MA USA 02451); and the processed blots were imaged using an Azure biosystems model C600 analyzer. Duplicate portions of the same samples were run on a separate gel and processed for Western blotting using an anti-Glyceraldehyde-3-phosphate dehydrogenase (GAPDH) antibody (Cell Signaling Technology®, Catalog No. 14C10; 3 Trask Lane, Danvers, MA 01923 USA) and a horseradish peroxidase (HRP)-conjugated goat anti-rabbit-IgG secondary antibody (Cell Signaling Technology®, Catalog No. 7074S).

The blots were imaged by HRP-activated chemiluminescence using an Azure Biosystems model C600 analyzer according to the manufacturer's instructions.

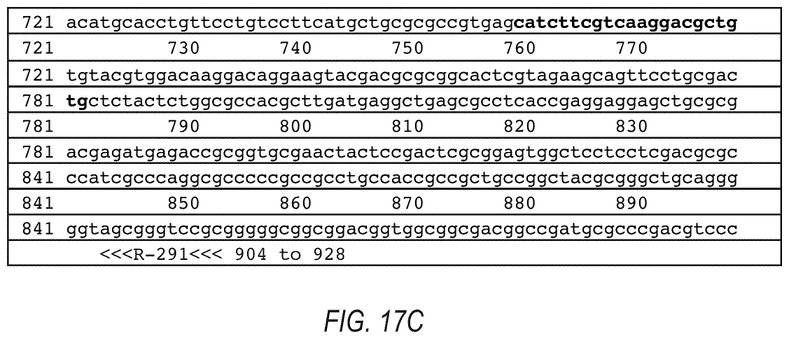

As shown in FIG. 21, the western blot analysis using anti-HA antibody revealed a ˜64KD band that corresponds to the approximate predicted size of the HA-tagged hPTH1R protein in the lanes containing kidney homogenates prepared from hPTH1R-KI mice only, and a slightly higher molecular weight band of ˜66 kD that corresponds to the predicted size of the endoglycosylated HA-tagged hPTH1R protein, in the lanes containing kidney homogenates prepared from hPTH1R-KI mice only, and not in lanes containing kidney homogenates prepared from WT mice. The western blot analysis of HA-tagged hPTH1R in kidneys of hPTH1R-KI, using anti-HA antibody, reveals a ˜70 kD band which corresponds to the approximate predicted size of the HA-tagged hPTH1R, and only in the lanes containing kidney homogenates prepared from hPTH1R-KI mice. FIG. 21.

### Example 9. Body Weight Analysis

The homozygous hPTH1R-KI mice were fertile and grew normally on standard rodent diet (1.1% Ca, 0.8% Pi) with body weights comparable to age-matched wild-type controls out to at least one year of age.

Briefly, transgenic hPTH1R-KI mice were generated as described in the Examples above, and body weight was observed in WT and hPTH1R-KI mice at 8, 16, 24, and 56 weeks of age.

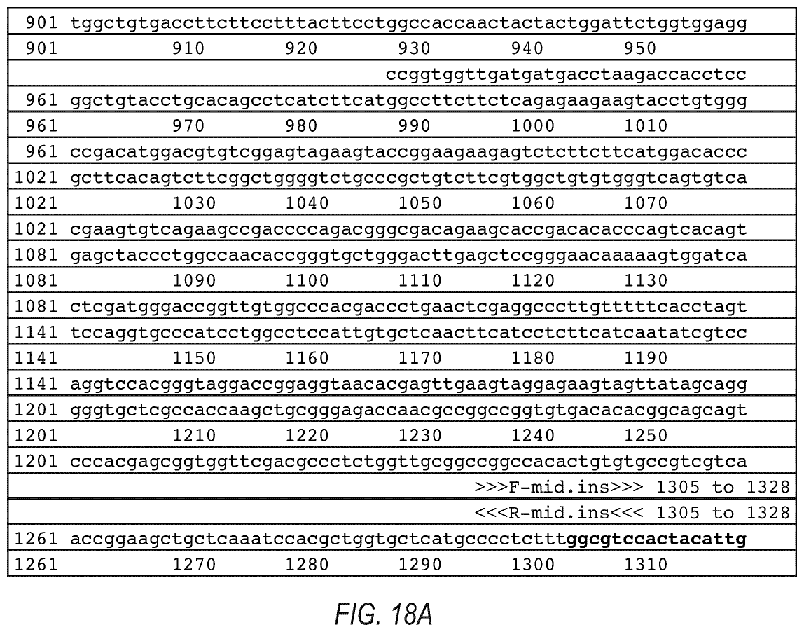

As shown in FIG. 22, body weights hPTH1R-KI mice did not substantially differ from WT controls, supporting the notion that the heterologous polynucleotide comprising human Parathyroid Hormone 1 Receptor (hPTH1R) exons 4 to 16 functions appropriately when knocked-in to a transgenic mouse. FIG. 22.

### Example 10. Microcomputed Tomography (uCT) Analysis of Bone Quality in hPTH1R-KI Mice

A bone analysis was performed on 6-month-old mice, and 13-month-old mice. Microcomputed tomography was performed on dissected femurs isolated from WT and hPTH1R-KI mice at age 26 weeks (˜6 months) and at 13 months of age. A micro-tomographic imaging system (μCT 40, Scanco Medical AG, Bruttisellen, Switzerland) was used to analyze the bone quality. Samples were scanned with a 10-pm isotropic voxel size, 70 kV peak potential (kVp), 114 pA X-ray tube intensity, and 300 ms integration time. Intramedullary bone and total volume were assessed in the distal femoral metaphysis, in a region beginning at the peak of the growth plate and extending proximally for 1.5 mm (150 transverse slices); at the mid-shaft, analysis was performed on a 0.5 mm long region (50 transverse slices) to measure total area (TtAr) and cortical bone area (Ct.Ar). The bone area was normalized to the total area at each slice, and the mean value reported as the cortical bone area fraction (Ct.Ar/TtAr, %).

TABLE 5
Microcomputed tomography (μCT) results in 6-month-old mice. Trabecular bone volume
relative to tissue volume (BV/TV, %) at the metaphyses was measured in a 1.5 mm-thick
region (150 adjacent cross-sectional planes, 0.01 mm/plane) interior to the cortices
and extending from the edge of the growth plate towards the mid-shaft. Cortical bone
area relative to tissue area (BA/TA, %) at the mid-shaft was measured as the mean of
the areas of 50 adjacent cross-sectional planes (0.01 mm/plane) spanning a 0.5 mm-thick region.

| | Females | | | | Males | | | |
|---|---|---|---|---|---|---|---|---|
| | WT | | KI | | WT | | KI | |
| | | n | | n | | n | | n |
| Length (mm) | 15.8 ± 0.2 | 3 | 15.7 ± 0.2 | 5 | 14.7 ± 0.7 | 7 | 15.3 ± 0.4 | 5 |
| Distal metaphysis | 3.8 ± 1.4 | 3 | 3.8 ± 0.6 | 5 | 10.6 ± 3.9 | 7 | 11.5 ± 4.2 | 5 |
| Trabecular bone volume | | | | | | | | |
| (BV/TV, %) | | | | | | | | |
| Mid-shaft | 50.4 ± 0.5 | 3 | 50.9 ± 1.0 | 5 | 43.0 ± 1.9 | 7 | 44.7 ± 1.9 | 5 |
| Cortical bone area (BA/TA, | | | | | | | | |
| %) | | | | | | | | |
| Mid-shaft | 0.21 ± 0.01 | 3 | 0.20 ± 0.01 | 5 | 0.16 ± 0.01 | 7 | 0.19 ± 0.01 | 5 |
| Cortical thickness (mm) | | | | | | | | |

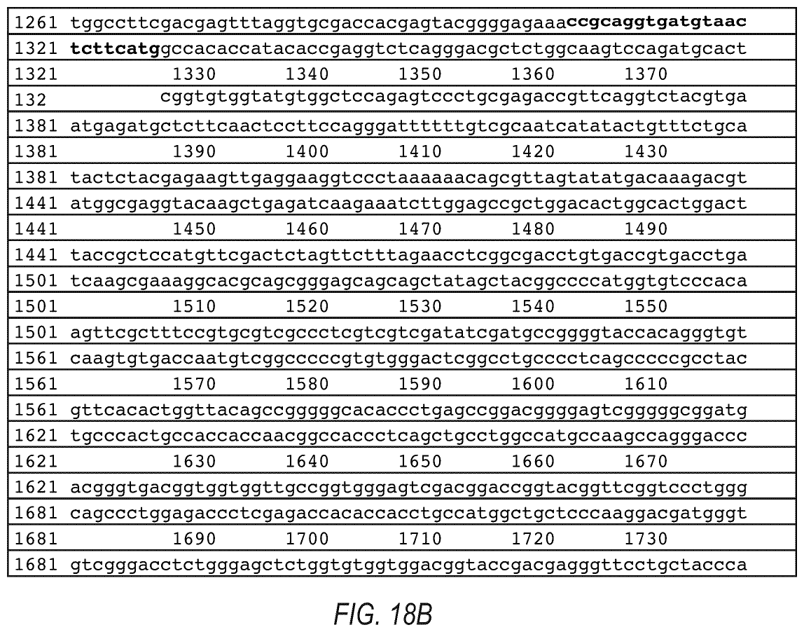

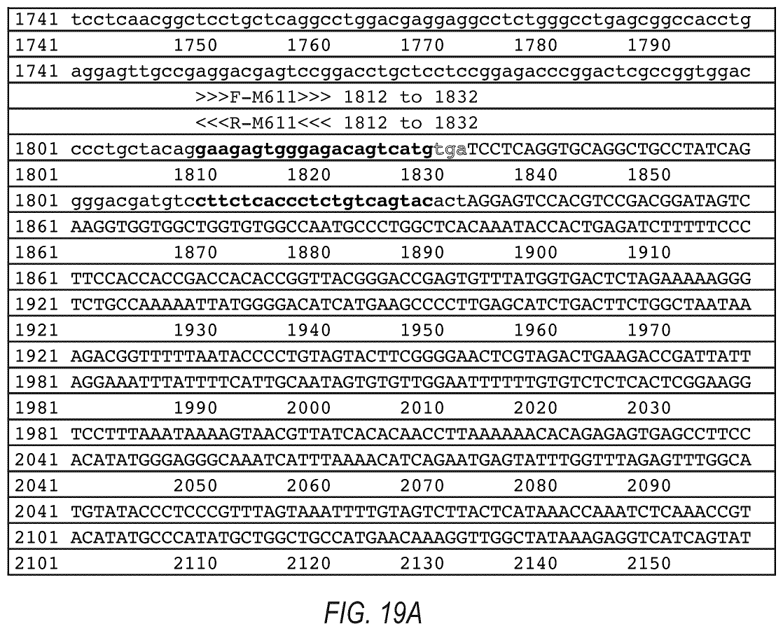

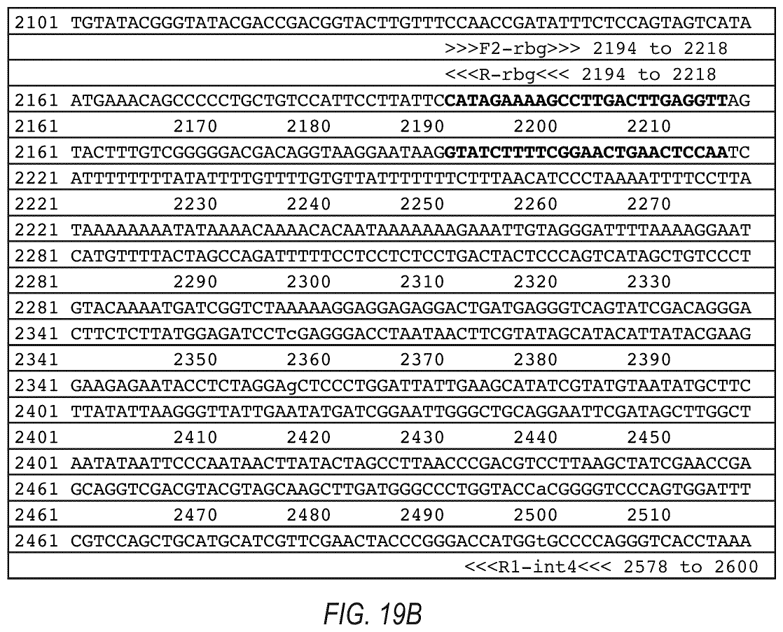

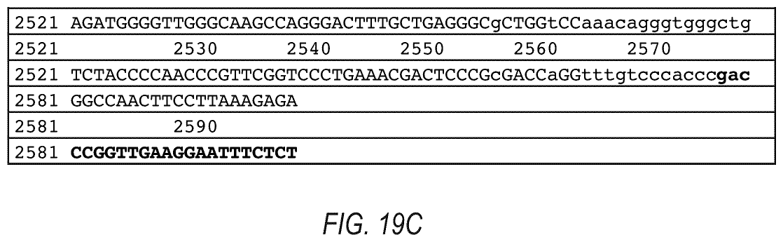

FIG. 23 shows the representative sagittal views of the distal femur in 6-month-old wild-type (WT) and hPTH1R-KI (KI) mice. FIGS. 24-27 shows the quantification of the results gleaned from the microcomputed tomography (μCT) analysis of bone parameters in 6-month-old mice.

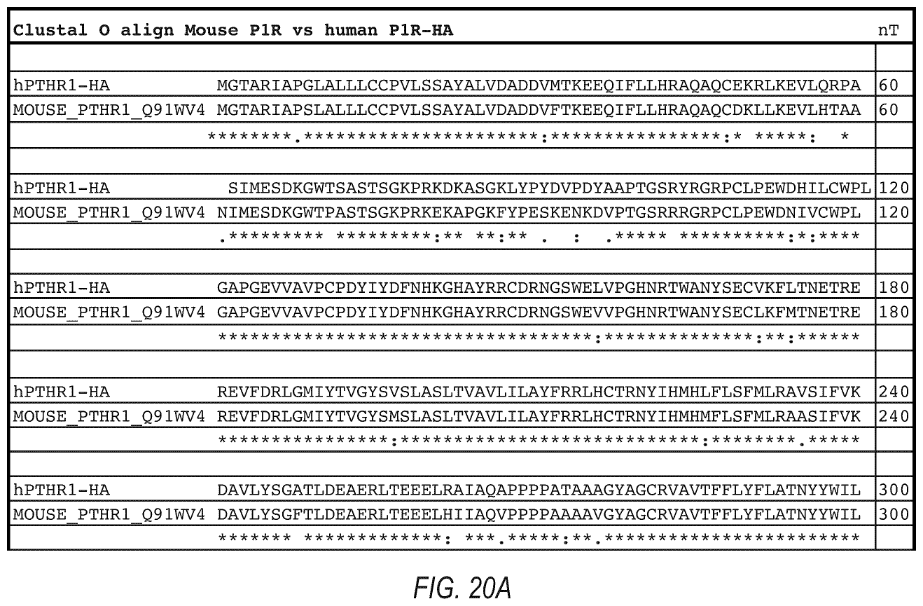

Quantitative analysis of bone parameters revealed no significant difference in the length of the femurs, the trabecular bone volume at the femoral distal metaphysis, the cortical bone area at the femoral mid-shaft, or other trabecular or cortical bone parameters analyzed for the KI and wild-type mice, except for cortical bone thickness which was slightly greater in the KI mice. FIG. 27.

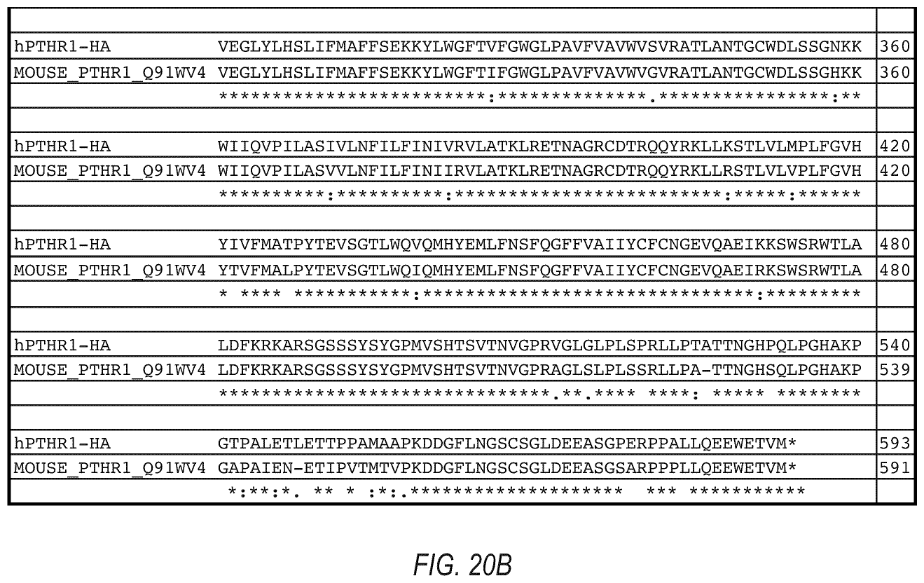

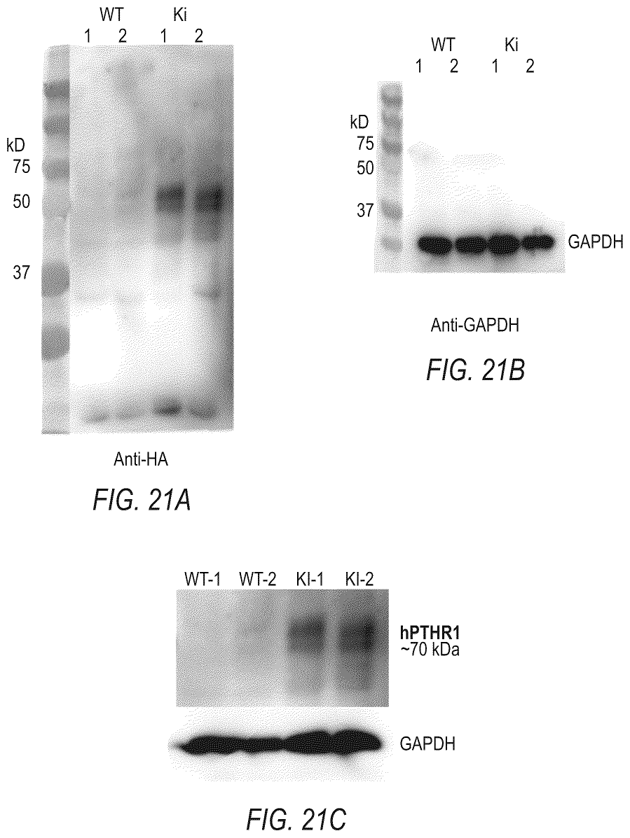

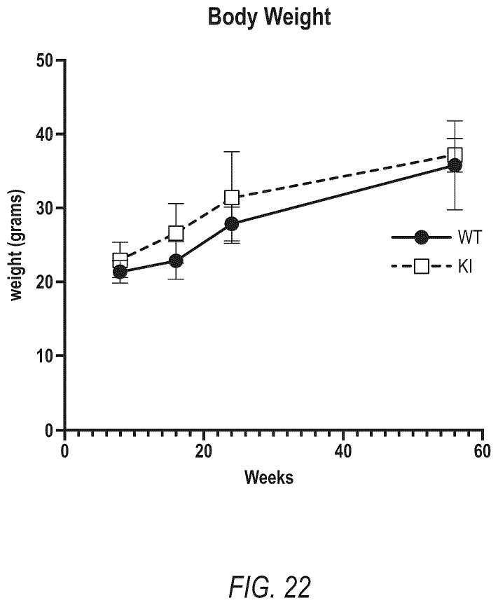

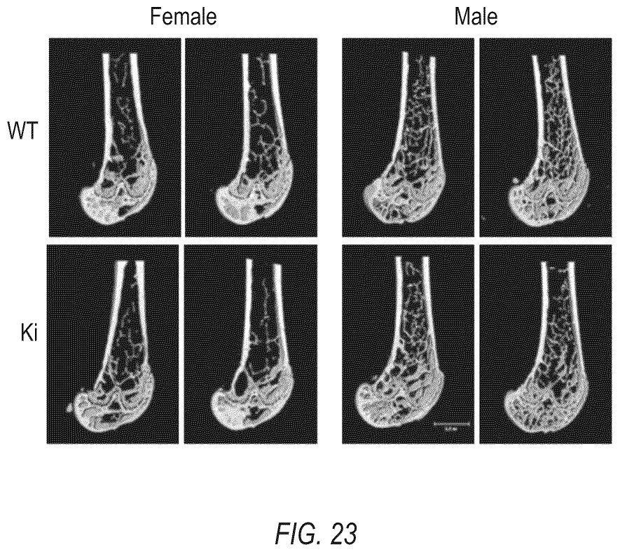

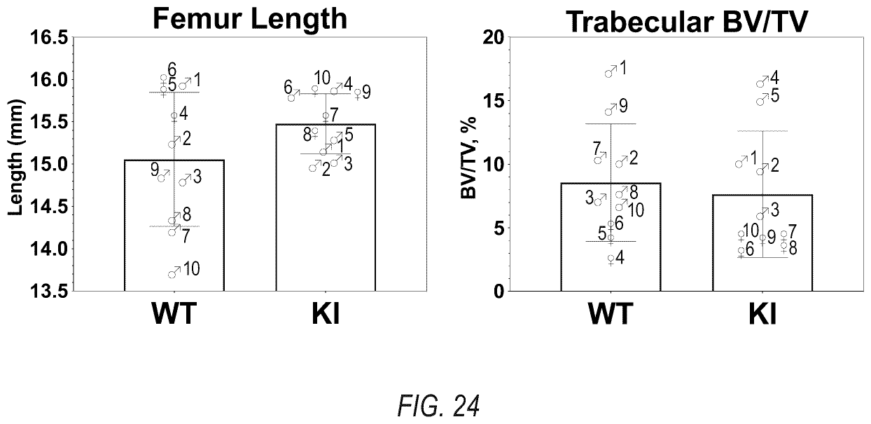

PTH and PTHrP-based analogs are anabolic in bone when administered by daily injections and are therefore used to treat bone loss associated with age-related osteoporosis. To assess the potential usefulness of the hPTH1R-KI mice of the present disclosure as a model for conducting studies on age-related effects of PTH analogs in vivo, the baseline properties of bones in a subset of the mice at age 13 months were analyzed. FIG. 28 shows the representative sagittal views of the distal femur in 13-month-old wild-type (WT) and hPTH1R-KI (KI) mice FIGS. 29-32 shows the quantification of the results gleaned from the microcomputed tomography (μCT) analysis of bone parameters in 13-month-old mice.

As bone mass in C57Bl/6 mice does not increase past the age of about 12 months to, an age of about 13 months represents the approximate age at which age-related bone loss will start to occur. Consistent with the findings in 6-month-old mice, μCT analysis of the femurs revealed no significant difference between the 13-month-old hPTH1R-KI and WT mice in any calculated trabecular or cortical parameter. FIGS. 29-32. Taken together, the foregoing data establishes that hPTH1R-KI mice maintain normal bone homeostasis for at least 13-months after birth; thus, the data presented here supports the use of the mouse model for evaluating age-related effects of candidate bone modulators on skeletal function and structure.

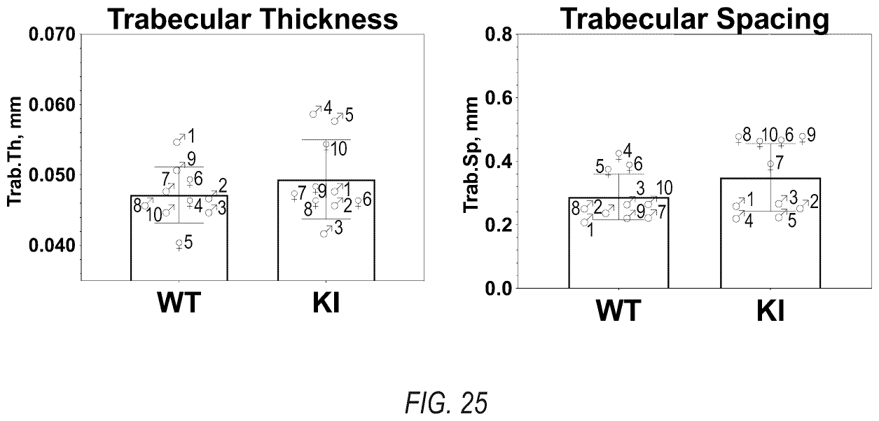

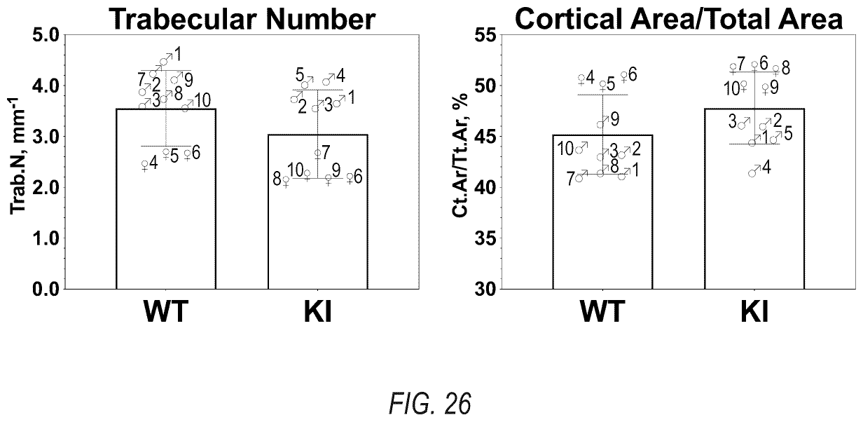

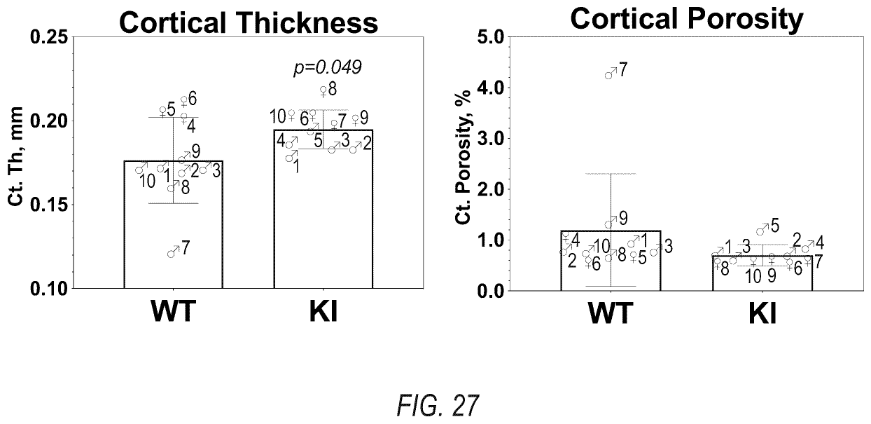

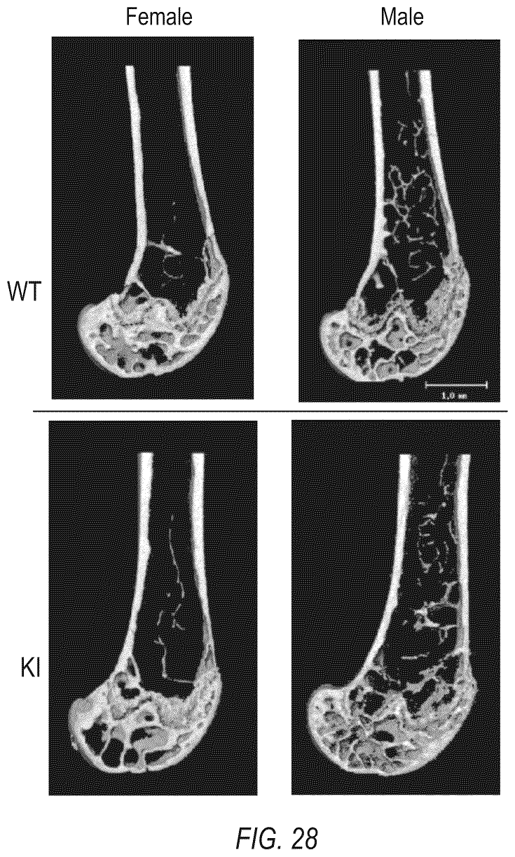

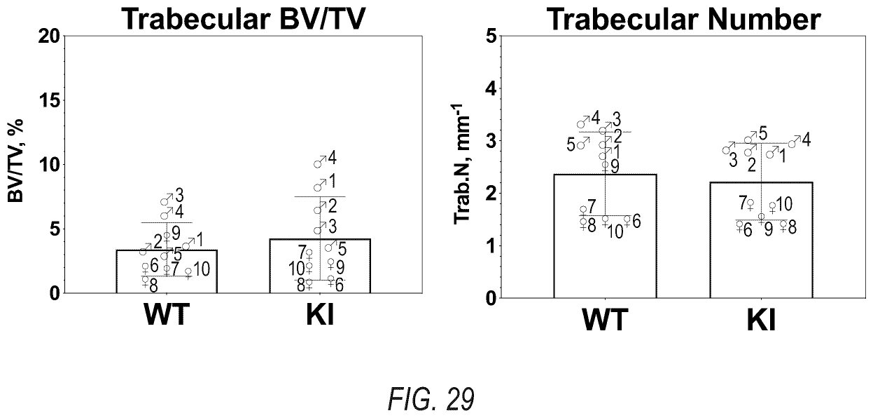

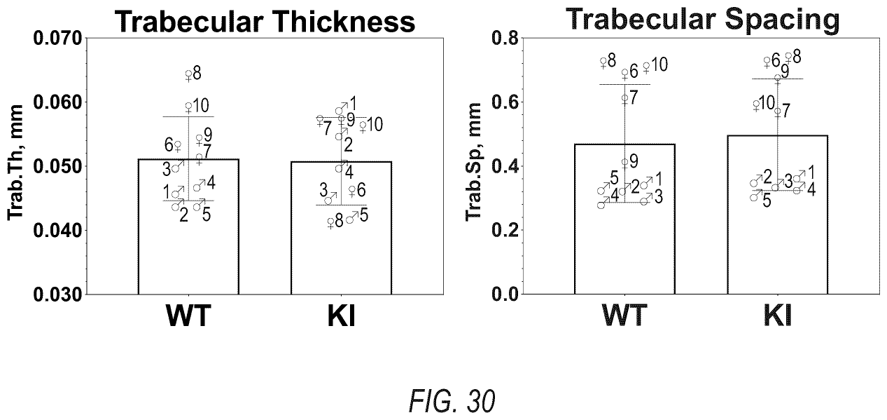

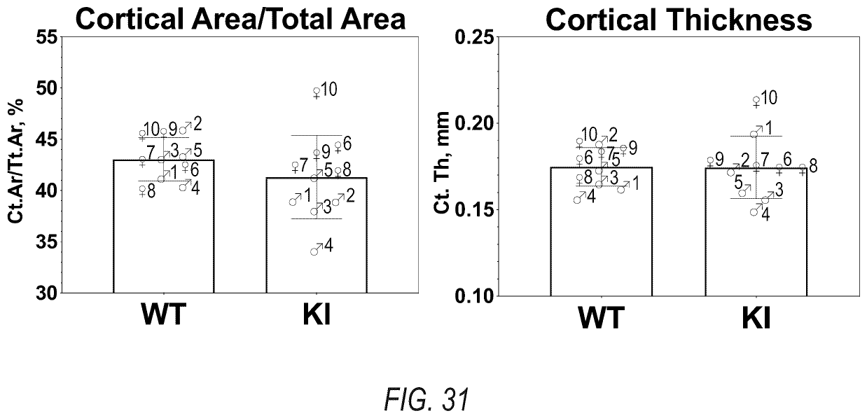

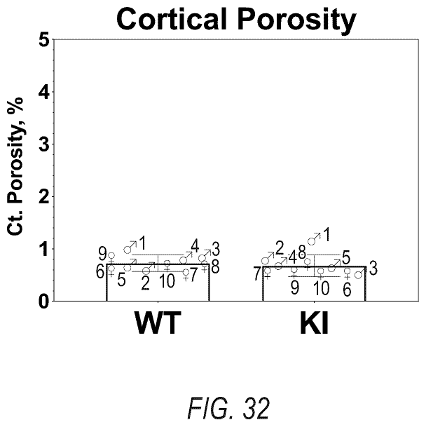

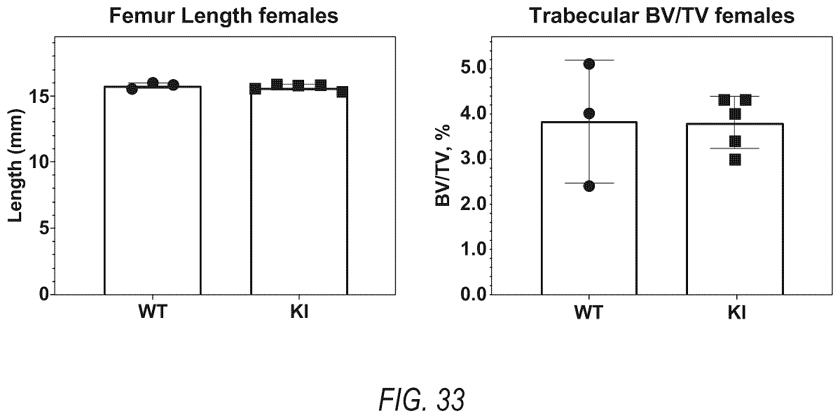

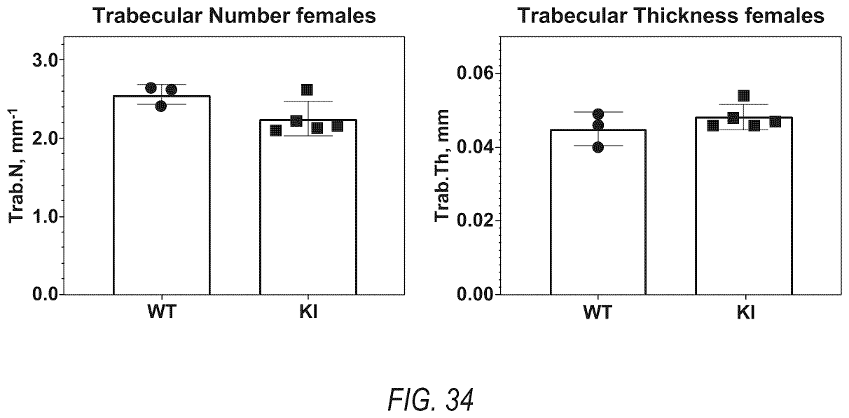

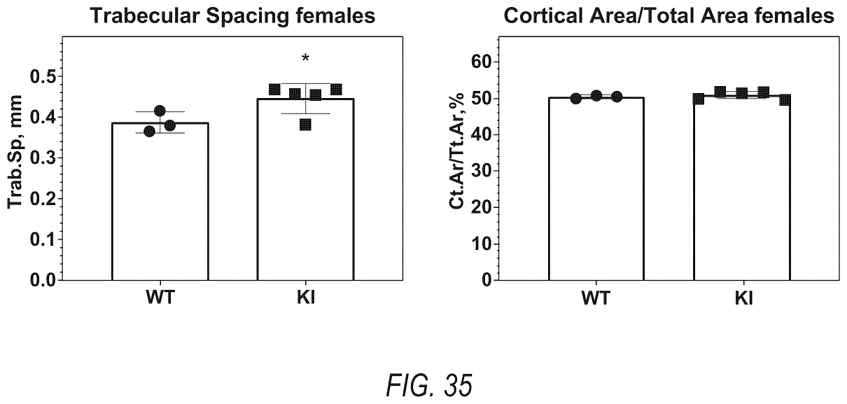

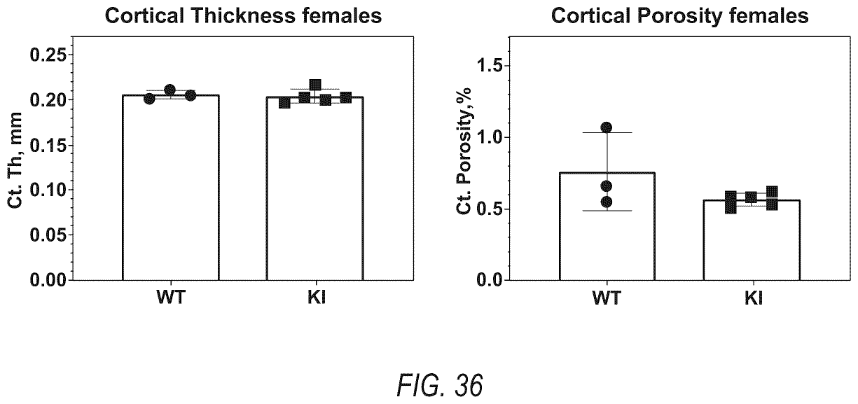

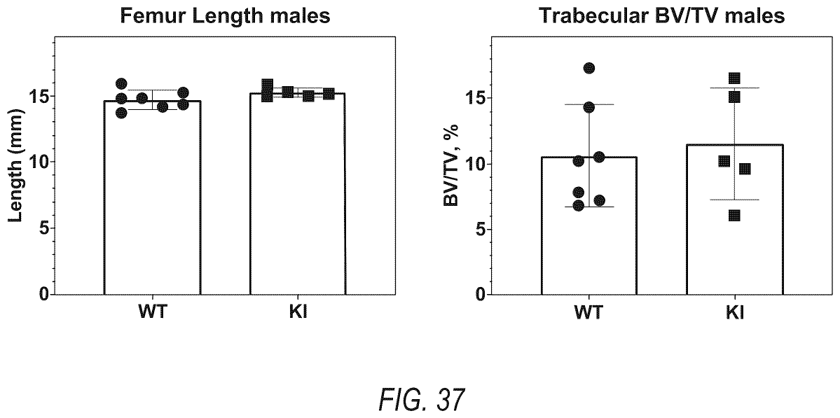

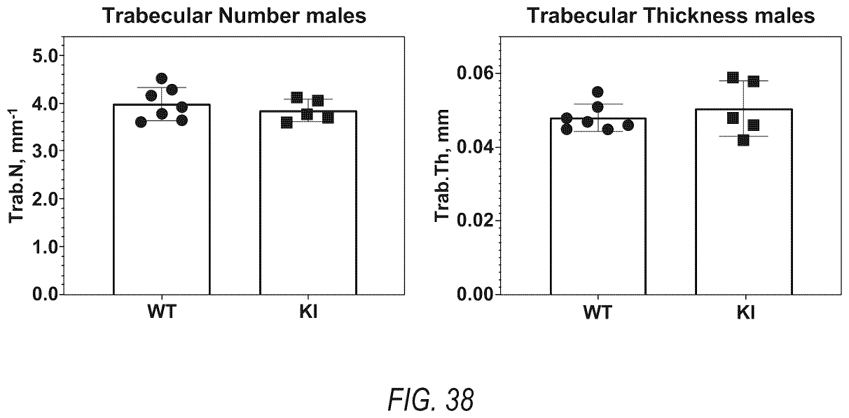

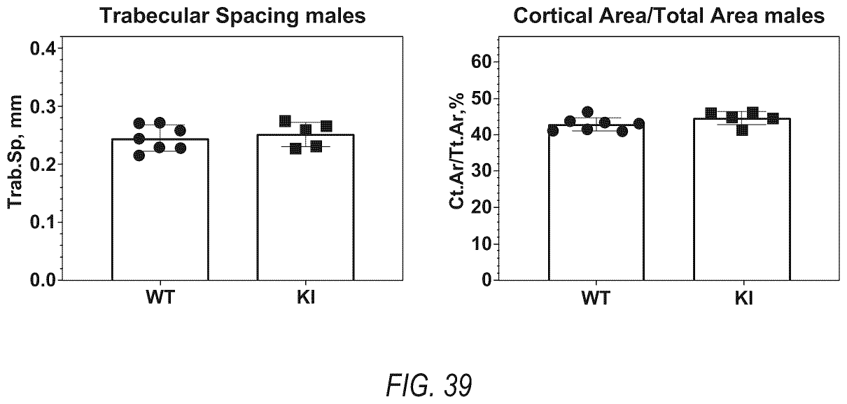

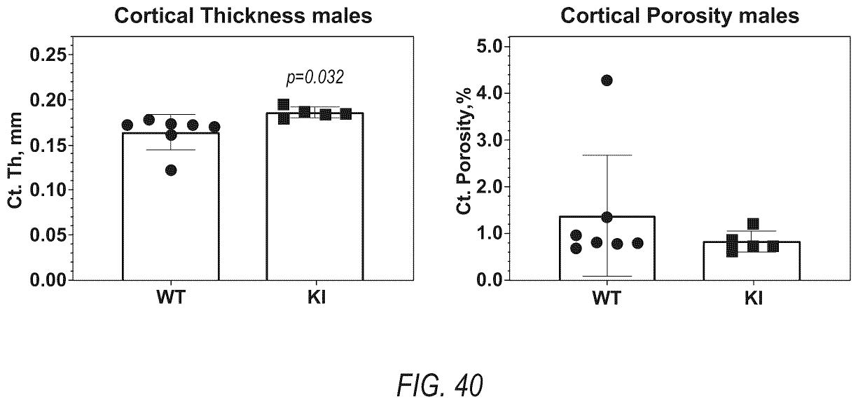

Several trabecular and cortical bone parameters tended to vary between genders, but this occurred in both wild-type and KI mice. Separating the data according to gender did not result in any significant difference between wild-type and KI groups, except for a slight increase in cortical area over total area in the male hPTH1R-KI mice vs. male WT mice. FIGS. 33-40 show the quantification of bone parameters in 6-month-old hPTH1R-KI and WT mice, and FIGS. 41-48 show the quantification of bone parameters in 13-month-old hPTH1R-KI and WT mice.

### Example 11. uCT Analysis of Skulls in hPTH1R-KI Mice

Microcomputed tomography was performed on skulls isolated from WT and hPTH1R-KI mice at 6 months of age using a micro-tomographic imaging system (μCT 40, Scanco Medical AG, Bruttisellen, Switzerland), as described in Example 10.

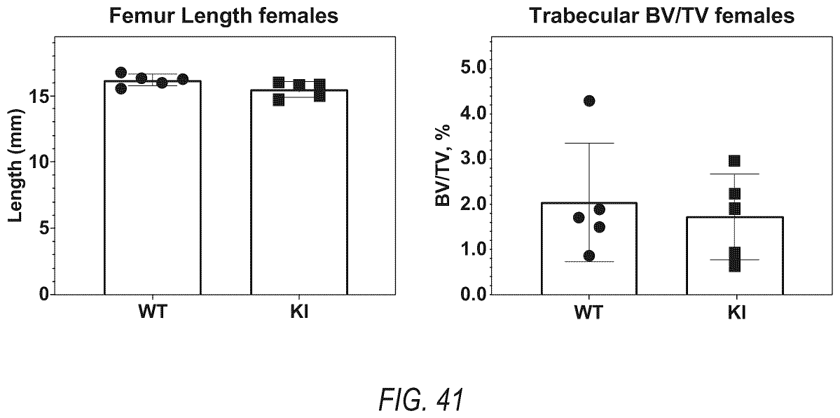

The results of the μCT skull analysis is shown in FIG. 49, which depicts a μCT 3D reconstruction of the side and superior views of skulls from WT and hPTH1R-KI mice at age 6 months. Here, three representative mice from the WT and hPTH1R-KI groups are shown (n=3). The top row shows CT images of skulls obtained from WT mice. The images of the WT skulls were obtained from two males: 1 WTM1 and 2 WTM2; and one female: 4 WTF1. The bottom row (hPTH1R-KI) shows the transgenic mice comprising a heterologous polynucleotide comprising human Parathyroid Hormone 1 Receptor (hPTH1R) exons 4 to 16, wherein said heterologous polynucleotide is operable to encode a human PTH1R protein. The transgenic mice skull images on the bottom row were obtained from two males: 1 hP1RM1 and 2 hP1RM1; and one female: 6 hP1RF1.

As shown in FIG. 49, the μCT of the skulls reveals no difference between WT and hPTH1R-KI mice (n=3), thus further demonstrating the utility of the present disclosure in evaluating hPTH1R function in a transgenic non-human animal.

In summary, the Examples above demonstrate that hPTH1R-KI mice maintain normal bone and mineral ion homeostasis. Calcemic responses differ between WT and hPTH1R-KI mice, with the hPTH1R-KI mice profile agreeing with predictions from studies performed on the human PTH1R (Hattersley et al. 2016), while responses in WT mice expressing the endogenous rodent PTH receptor do not. These results support the use of the transgenic mice of the present disclosure over traditional mouse models to assess PTH/PTHrP analogs as treatments for PTH1R-mediated diseases in humans.

### Example 12. Biomarker Analysis in Blood and Urine of WT and hPTH1R-KI Mice

PTH1R-mediated signaling is responsible for maintaining normal levels of calcium (Ca) and inorganic phosphorus (Pi) in the blood through actions in bone and kidney. Here, the baseline serum and urine levels of both Ca and Pi, as well as serum levels of the bone turnover markers CTX-I and PINP, in both 5-month- and 13-month-old WT control and hPTH1R-KI mice, were evaluated.

Wild-type C57BL/6n and C57BL/6n-hPTH1R-KI mice were euthanized at 5-months or 13-months of age, and cardiac blood was collected from the aorta using a 0.3 cc micro-insulin syringe with a 31 gauge needle. The blood was placed into a plastic tube and centrifuged at 8.000×g for 15 minutes at 4° C. and the supernatant (serum) was collected and placed into a new plastic tube and frozen at −80° C.

The samples were thawed and an appropriate volume removed for assay of the following biomarkers: (1) calcium; (2) phosphate; (3) CTX-1 (i.e., C-terminal telopeptides of type I collagen, or the degradation products therefrom); (4) PINP (N-terminal propeptide of type I procollagen); (5) PTH(1-84); (6) 1,25-Dihydroxy Vitamin D; and (7) Creatinine.

Assays of the foregoing biomarkers were performed using the following assay kits: LiquiColor colorimetric calcium Kit (Stanbio Laboratory, Catalog No. 0150); colorimetric phosphate assay kit (AbCam, Catalog No. ab65622; 1 Kendall Square, Suite B2304, Cambridge, MA 02139-1517 USA); RatLaps, CTX-1 (C-terminal telopeptides of type I collagen) Enzyme-immunoassay (EIA) kit (ImmunoDiagnosticSystems Limited, Tyne & Wear, UK, Catalog No. AC-06F1); PINP (N-terminal propeptide of type I procollagen) EIA kit (ImmunoDiagnosticSystems; Catalog No. AC-33F1); PTH(1-84) ELISA Kit (Quidel Inc.; Catalog No. 60-2305; 9975 Summers Ridge Road, San Diego, CA 92121 USA); 1,25-Dihydroxy Vitamin D EIA kit (ImmunoDiagnosticSystems Limited; Catalog No. AC-62F1); and Stanbio Creatinine LiquiColor Test (Stanbio Laboratory, Catalog No. 0430-500); all according to the manufacturer's instructions.

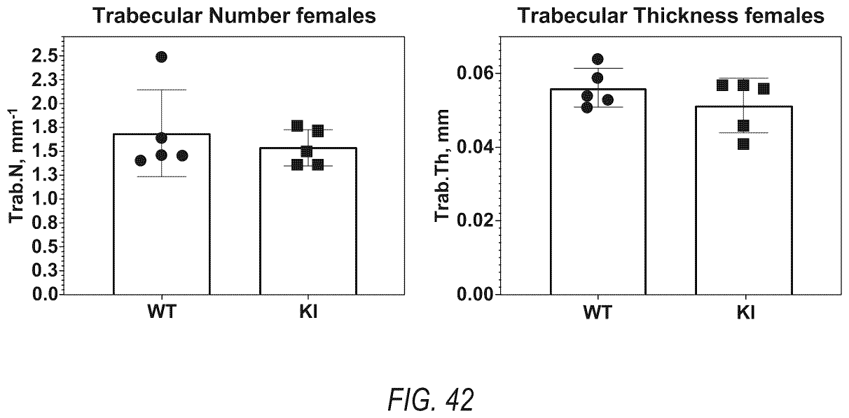

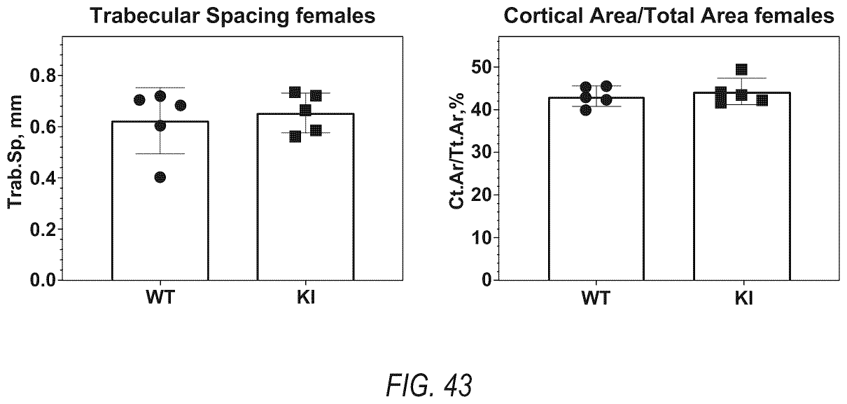

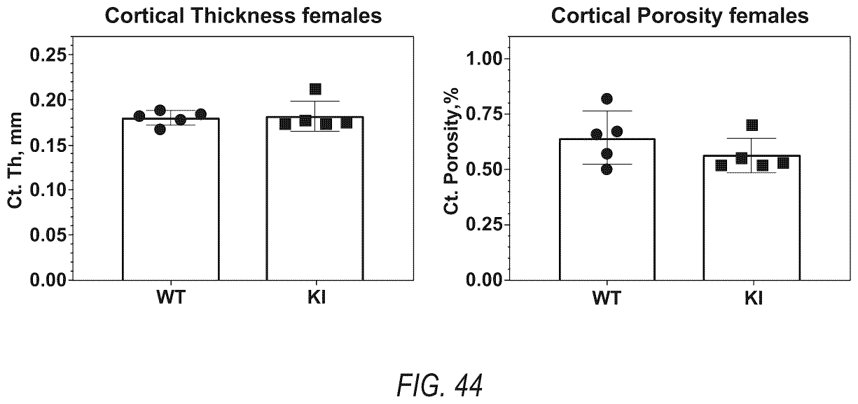

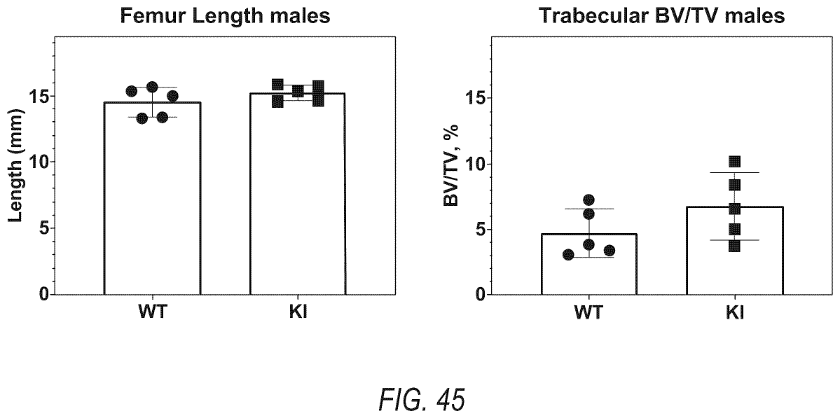

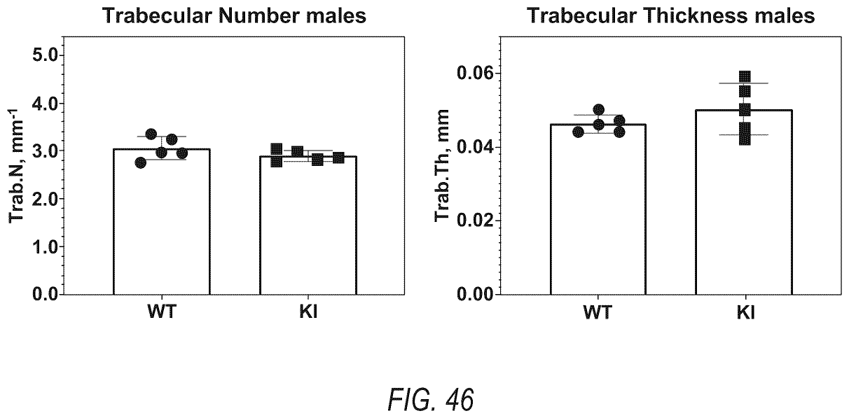

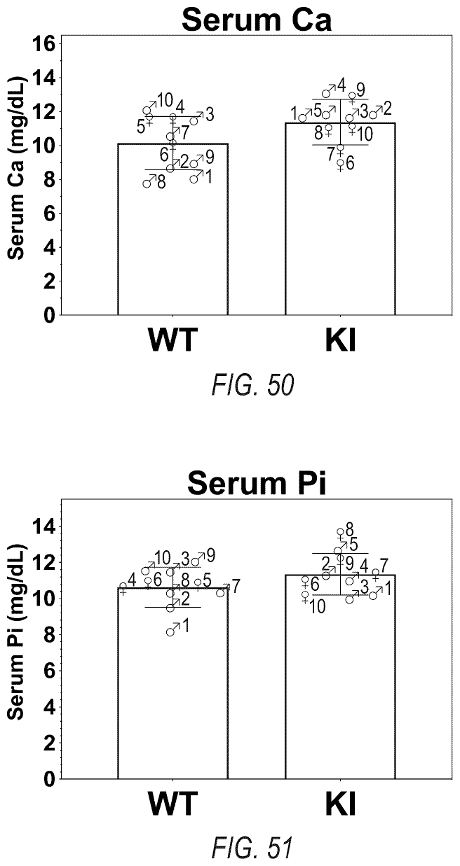

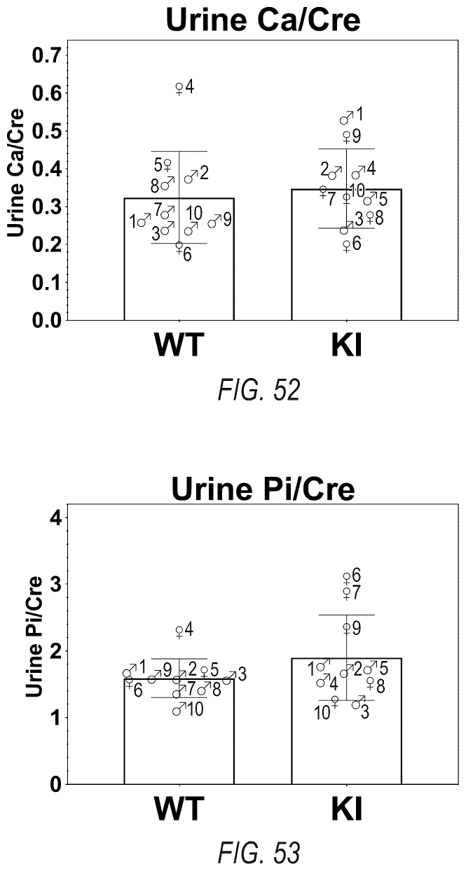

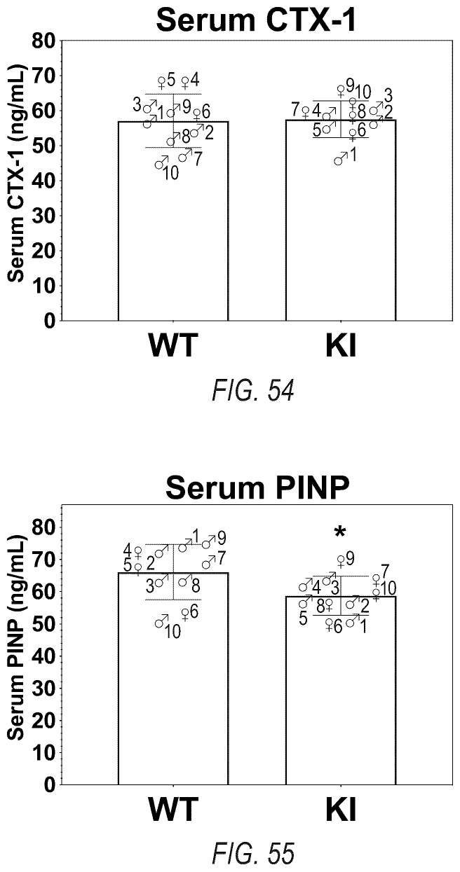

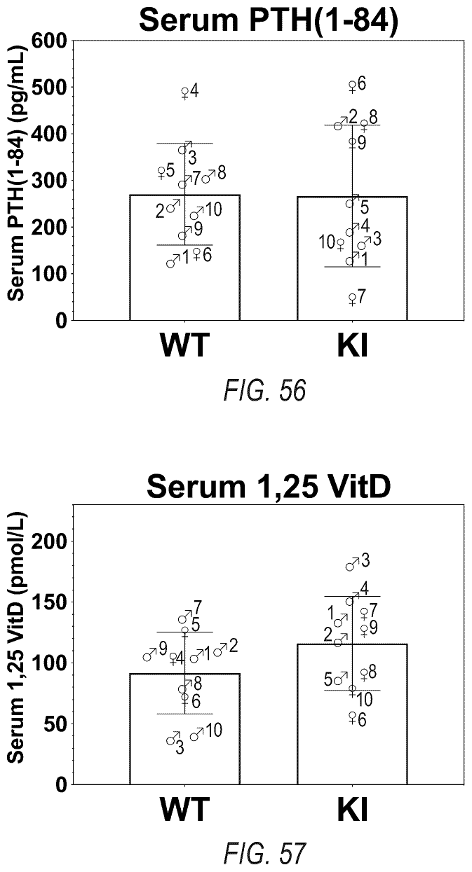

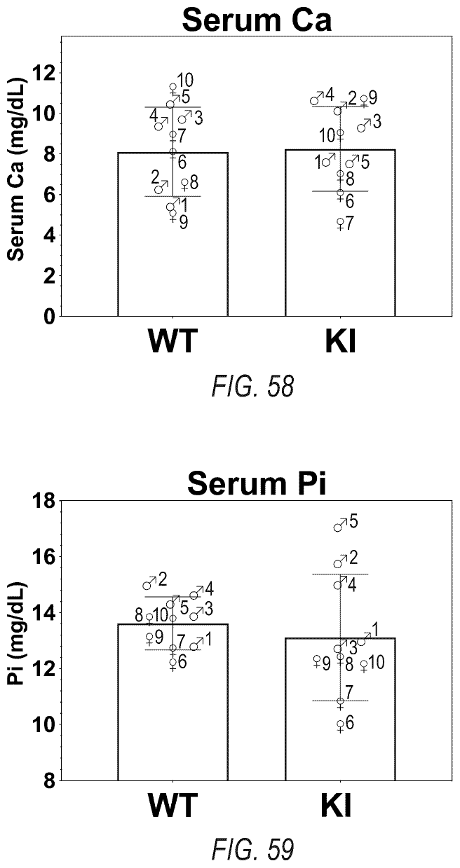

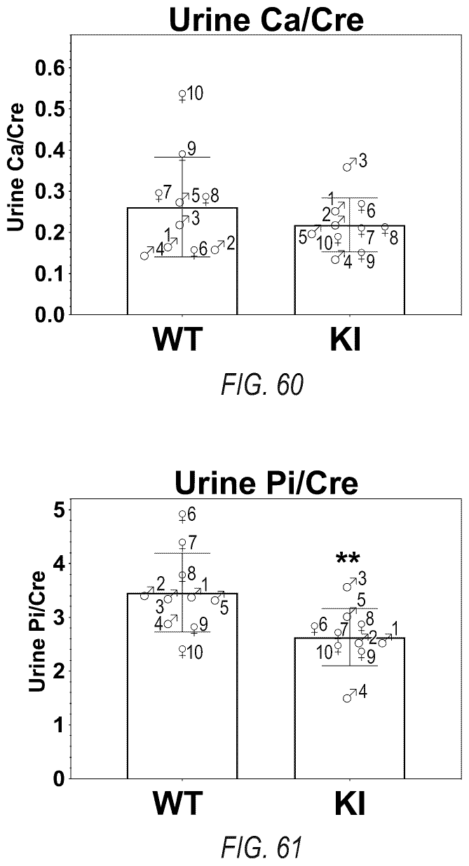

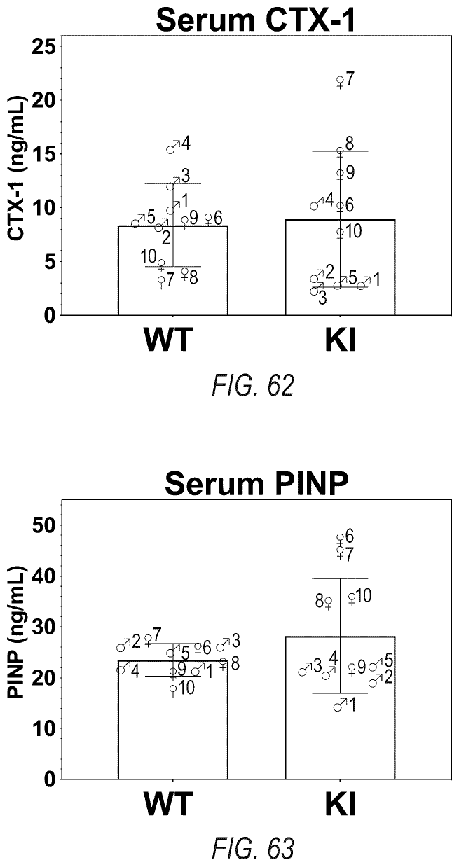

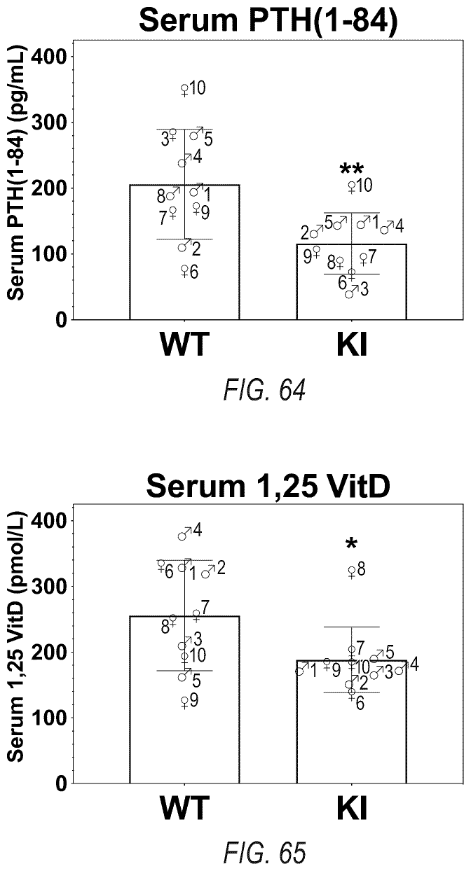

The results of the biomarker assays for 5-month-old mice are shown in FIGS. 50-57. The results of the biomarker assays for 13-month-old mice are shown in FIGS. 58-65.

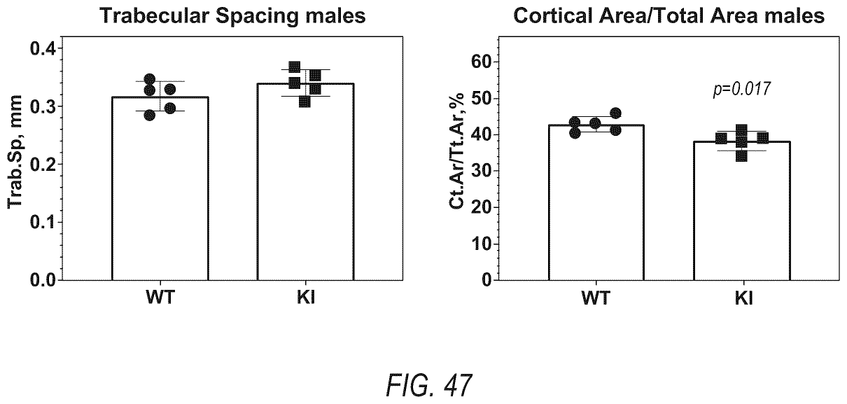

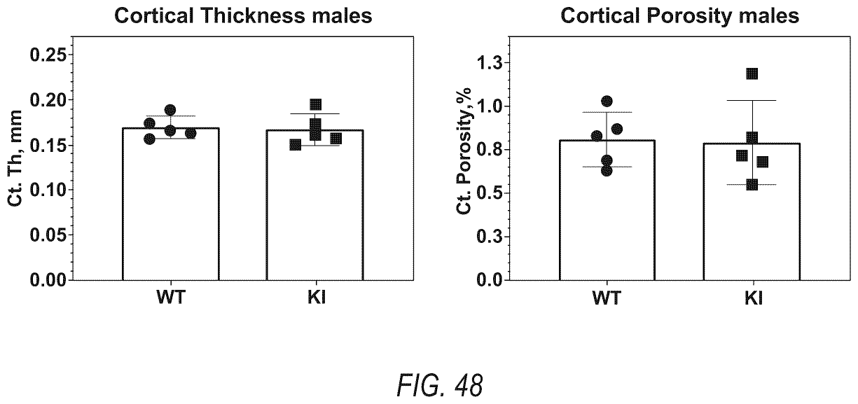

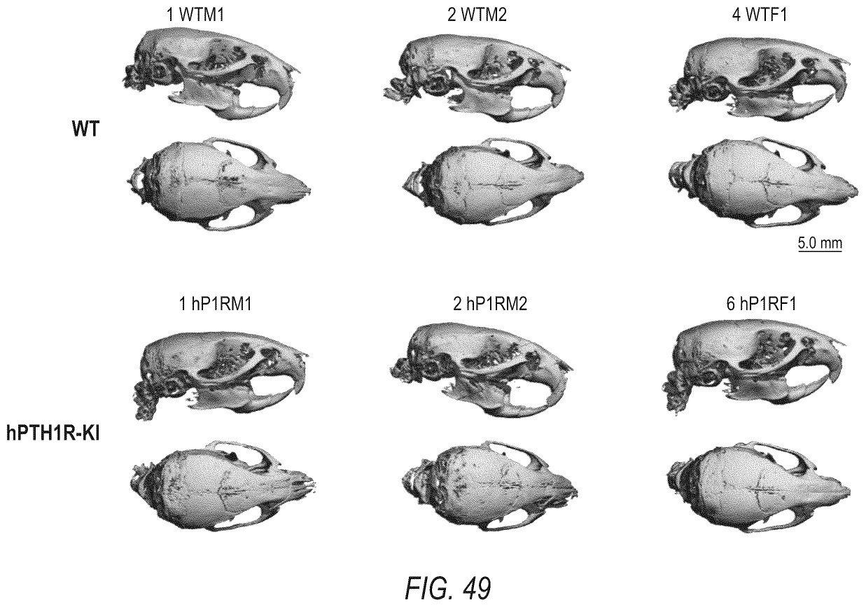

In agreement with the μCT results shown in Examples 11 and 12, the 6-month-old WT and hPTH1R-KI mice display similar levels of Ca and Pi in both serum and urine; these findings further support the notion that the heterologous polynucleotide comprising human Parathyroid Hormone 1 Receptor (hPTH1R) exons 4 to 16 of the present disclosure, functions appropriately in bone and kidney. There was also no significant difference between the levels of the bone resorption marker CTX-I in 6-month-old hPTH1R-KI and WT mice, while a marginal decrease in the levels of the bone formation marker PINP was observed in the hPTH1R-KI vs. WT mice (p=0.046). FIG. 55. Importantly, 6-month-old hPTH1R-KI and WT mice exhibited similar baseline serum levels of endogenous PTH(1-84) and 1.25(OH)₂ VitaminD, indicating normal hormonal regulation of Ca and Pi levels in the hPTH1R-KI mice. FIGS. 56-57.

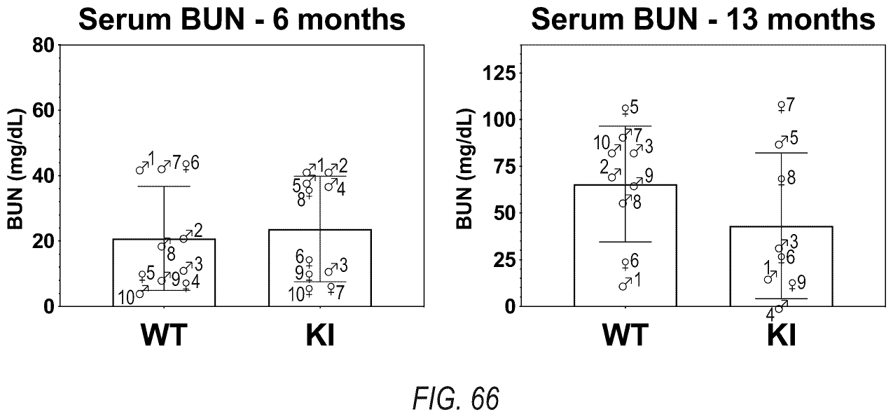

At age 13-months, the serum levels of Ca, Pi, CTX-1 and PINP, and urine Ca levels in hPTH1R-KI mice were comparable to those in WT mice; however, the hPTH1R-KI mice had lower serum levels of PTH(1-84) and 1.25(OH)₂ VitaminD as well as lower urine phosphate levels as compared to the WT control mice. FIGS. 58-65. Lower levels of 1.25(OH)₂ VitaminD and a decrease in urinary phosphate excretion could indicate kidney dysfunction in the older KI mice; however, the levels of blood urea nitrogen (BUN), which increase as glomerular filtration declines and thus provide a read-out of kidney function, were not different between 13-month-old hPTH1R-KI and WT mice. FIG. 66. While the reason for these changes is unclear, it is conceivable that a lower circulating level of endogenous PTH(1-84) in the 13-month-old hPTH1R-KI mice lead to reduced levels of PTH1R signaling in kidney proximal tubule cells and hence to decreased rates of urinary phosphate excretion as well as synthesis of 1.25(OH)₂ VitaminD.

### Example 13. Serum calcium (Ca⁺⁺) response to PTH ligand analog injection in hPTH1R-KI and Wild-type (WT) mice

The utility of the hPTH1R-KI mice of the present disclosure as a model for predicting the behavior of different PTH and PTHrP analog peptides was evaluated. Ligand-induced PTH1R signaling in bone and kidney results in a rapid increase in blood levels of free ionized calcium (Ca⁺⁺) and a decrease in blood levels of inorganic phosphorus (Pi); changes that occur through the promotion of the release of Ca and Pi from bone mineral stores, the reabsorption of Ca from the renal filtrate, and a suppression of Pi reabsorption in the kidney. Thus, blood Ca⁺⁺ and Pi levels were measured in WT and hPTH1R-KI mice before and at times after injection of PTH ligands.

The PTH ligands evaluated were human Parathyroid Hormone Fragment 1-34, or “PTH (1-34)”; Parathyroid hormone-related protein 1-36, or “PTHrP (1-36)”; and the PTHrP(1-34)-based analog, Abaloparatide.

Parathyroid Hormone (PTH) (1-34) (Human) is a highly purified peptide that can be chemically synthesized or expressed recombinantly. Parathyroid hormone is the most important endocrine regulator of calcium and phosphorus concentration in extracellular fluid. PTH is secreted from cells of the parathyroid glands, and finds its major target cells in bone and kidney. PTH is believed to be involved in at least three processes: enhancing absorption of calcium from the small intestine, mobilization of calcium from bone, and suppression of calcium loss in urine. PTH (1-34) is a peptide fragment (34 amino acids) of the naturally occurring human parathyroid hormone that is an important regulator of calcium and phosphorus metabolism. See Bieglmayer C, Prager G, and Niederle B, “Kinetic analyses of parathyroid hormone clearance as measured by three rapid immunoassays during parathyroidectomy.” Clin Chem. 2002 October; 48(10):1731-8; Poole K, and Reeve J, Parathyroid hormone—a bone anabolic and catabolic agent. Curt Opin Pharmacol. 2005 December; 5(6):612-7; Coetzee M, and Kruger M C, Osteoprotegerin-receptor activator of nuclear factor-kappaB ligand ratio: a new approach to osteoporosis treatment? South Med J. 2004 May; 97(5):506-11.

An exemplary full length PTH human peptide is provided herein, having an amino acid sequence of:

(SEQ ID NO: 19)
“MIPAKDMAKVMIVMLAICFLTKSDGKSVKKRSVSEIQLMHNLGKHLNS
MERVEWLRKKLQDVHNFVALGAPLAPRDAGSQRPRKKEDNVLVESHEKS
LGEADKADVNVLTKAKSQ”.
(UniProt No. P01270)

An exemplary PTH (1-34) peptide is provided, having the amino acid sequence of: “SVSEIQLMHNLGKHLNSMERVEWLRKKLQDVHNF” (SEQ ID NO: 20).

PTHrP is another ligand that can bind to PTH1R. Parathyroid hormone-related protein shares some homology with PTH at their N-terminal ends, and both proteins bind to the same G-protein coupled receptor, PTH1R. Despite a common receptor, PTH primarily acts as an endocrine regulator of calcium homeostasis whereas PTHrP plays a fundamental paracrine role in the mediation of endochondral bone development. See Kronenberg, “PTHrP and skeletal development,” Ann N Y Acad Sci 1068:1-13 (2006).

The differential effects of these proteins may be related not only to differential tissue expression, but also to distinct receptor binding properties. See Pioszak et al., “Structural basis for parathyroid hormone-related protein binding to the parathyroid hormone receptor and design of conformation-selective peptides,” J Biol Chem 284:28382-28391 (2009); Okazaki et al., “Prolonged signaling at the parathyroid hormone receptor by peptide ligands targeted to a specific receptor conformation,” Proc Natl Acad Sci USA 105:16525-16530 (2008); and Dean et al., “Altered selectivity of parathyroid hormone (PTH) and PTH-related protein (PTHrP) for distinct conformations of the PTH/PTHrP receptor,” Mol Endocrinol 22:156-166 (2008).

Over the past several years, PTHrP and its secretory forms (PTHrP(1-36), PTHrP(38-94), and osteostatin), as well as analogues thereof, have been investigated as potential treatments for osteoporosis. Subcutaneous injection of PTHrP and its derivatives and analogues has been reported to be effective for treating osteoporosis and/or improving bone healing. See Horwitz et al., “Parathyroid hormone-related protein for the treatment of postmenopausal osteoporosis: defining the maximal tolerable dose,” J Clin Endocrinol Metab 95:1279-1287 (2010); Horwitz et al., “Safety and tolerability of subcutaneous PTHrP(1-36) in healthy human volunteers: a dose escalation study,” Osteoporos Int 17:225-230 (2006); Bostrom et al., “Parathyroid hormone-related protein analog RS-66271 is an effective therapy for impaired bone healing in rabbits on corticosteroid therapy,” Bone 26:437-442 (2000); and Augustine et al., “Parathyroid hormone and parathyroid hormone-related protein analogs as therapies for osteoporosis,” Curt Osteoporos Rep 11:400-406 (2013).

There are three principal secretory forms of PTHrP: PTHrP (1-36), PTHrP (38-94), and osteostatin (PTHrP[107-139]), which arise from the endoproteolytic cleavage of the initial translation product. Each of these secretory forms is believed to have one or more of its own receptors that mediates the normal paracrine, autocrine and endocrine actions. PTHrP (1-36) is composed of residues 37-72 of the PTHrP protein.

An exemplary human PTHrP peptide is provided, having the amino acid sequence of: “AVSEHQLLHDKGKSIQDLRRRFFLHHLIAEIHTAEI” (SEQ ID NO: 21) (UniProt No. P12272).

Abaloparatide is a synthetic PTHrP analogue that is a 34-amino acid peptide with 76% homology with parathyroid hormone-related protein (PTHrP) (1-34) and 41% homology to PTH (1-34). Abaloparatide is a potent and selective activator of the PTH1R signaling pathway. Abaloparatide is differentiated from PTH and PTHrP ligands based on its affinity and greater selectivity for the G protein-dependent (RG) (versus the G-independent (R0)) receptor conformation of PTH1R; this selectivity may produce a more transient stimulation of osteoblast c-AMP compared to PTH, resulting in less of an effect on bone resorption and less hypercalcemia.

Abaloparatide has shown potent anabolic activity with decreased bone resorption, less calcium-mobilizing potential, and improved room temperature stability. See Obaidi et al., “Pharmacokinetics and Pharmacokinetics and pharmacodynamic of subcutaneously (SC) administered doses of BA058, a bone mass density restoring agent in healthy postmenopausal women,” AAPS Abstract W5385 (2010). Studies performed in animals have demonstrated marked bone anabolic activity following administration of abaloparatide, with complete reversal of bone loss in ovariectomy-induced osteopenic rats and monkeys. See Doyle et al., “BA058, a novel human PTHrP analog: reverses ovariectomy-induced bone loss and strength at the lumbar spine in aged cynomolgus monkeys,” J Bone Miner Res 28 (Suppl 1) (2013a); and Doyle et al., “Long term effect of BA058, a hovel human PTHrP analog, restores bone mass in the aged osteopenic ovariectomized cynomolgus monkey,” J Bone Miner Res 28 (Suppl 1) (2013a).

Abaloparatide has been developed as a promising anabolic agent for the treatment of osteopenia (e.g., glucocorticoid-induced osteopenia), osteoporosis (e.g. glucocorticoid-induced osteoporosis), and/or osteoarthritis.

An exemplary Abaloparatide sequence is provided, having an amino acid sequence of: “AVSEHQLLHDKGKSIQDLRRRELLEKLLXKLHTA”; wherein X is 2-Aminoisobutyric acid, or α-aminoisobutyric acid (Aib) (SEQ ID NO: 22) (PubChem CID No. 76943386).

Blood ionized calcium (Ca⁺⁺) levels were measured in homozygous mice comprising a heterologous polynucleotide comprising human Parathyroid Hormone 1 Receptor (hPTH1R) exons 4 to 16 (hereinafter “hPTH1R-KI” mice), and WT C57BL/6(n) mice, at age 10 weeks. Mice were treated in accordance with the ethical guidelines adopted by the Massachusetts General Hospital (MGH). Mice were injected subcutaneously in the interscapular region with vehicle (5 mM citrate, 150 mM NaCl, 0.05% Tween80, pH 5.0), or vehicle containing either PTH(1-34), PTHrP(1-36) or abaloparatide, each peptide at a dose of 40 nmol/kg of body weight, with five animals per group.

Blood was collected from a ˜2 mm tail vein incision and placed into heparinized capillary tube (Multi-cap-S, Siemens Healthcare Diagnostics Inc, Catalog No. 05656514) just prior to injection (t=0), and at 1, 2, 4 and 8 hours after injection, then immediately analyzed for pH-adjusted ionized calcium using a Siemens RapidLab 348 Ca²⁺/pH analyzer.

Data was processed using Microsoft Excel 2016 and GraphPad Prism 8.0. Data is represented as means+/−standard error, and statistical significance was assessed using a two-tailed Students t-test. Two replicate experiments were performed on separated days for each set of 20 hPTH1R-KI and 20 WT control mice n hPTH1R-KI mice. The replicate experiments gave essentially the same result for the two groups, and the data (n=10) were combined as means+SEM.

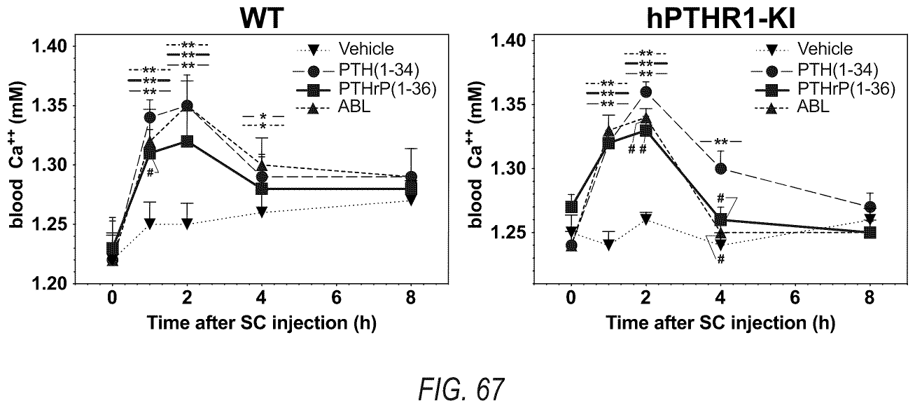

Injection into 10-week-old WT mice resulted in similar increases in blood Ca⁺⁺ levels in response to all three peptides with peak increases occurring 1-2 hours post-injection and Ca⁺⁺ levels returning to baseline by 4 hours FIG. 67. Injection of the three peptides into 10-week-old hPTH1R-KI mice resulted in similar increases in blood Ca* at the one hour time point; however, at 2- and 4-hours post-injection, the blood Ca⁺⁺ levels in KI mice injected with PTHrP(1-36) or abaloparatide were significantly lower than those in KI mice injected with PTH(1-34). FIG. 67. Moreover, where the levels remained elevated at 4 hours in hPTH1R-KI mice injected with PTH(1-34), they had returned to baseline in mice injected with PTHrP(1-36) or abaloparatide. FIG. 67. Accordingly, in hPTH1R-KI mice, PTH(1-34) induced a more prolonged calcemic response than either of the other two ligands tested. Such a difference in response duration observed for PTH(1-34) as compared to the other two ligands was not seen in the WT mice. The data thus supports the notion that the hPTH1R-KI mice can be used to help functionally differentiate between structurally distinct PTH and PTHrP ligand analogs in vivo. In particular, they support a more a transient functional response induced by PTHrP(1-36) or abaloparatide, versus a more prolonged response induced by PTH(1-34), that is predicted by the altered binding and signaling actions of these peptides observed in cells expressing the human, but not the rodent PTH1R.

### Example 14. Serum Phosphorus (Pi) Response to PTH Ligand Analog Injection in hPTH1R-KI and Wild-Type (WT) Mice

To assess changes to blood Pi in response to PTH ligand injection, 10-week-old hPTH1R-KI and wild-type mice were injected with PTH(1-34), PTHrP(1-36), or Abaloparatide (see Example 13) each at a dose of 40 nmol/kg (n=5 mice per group). Tail vein blood was collected into a 1 mL microcentrifuge tubes containing 3 μL 0.5 mM EDTA on ice at t=0, 1, 2, 4, and 8 hours post-injection. The blood samples were centrifuged at 8,000 rpm at 4° C. for 15 minutes, and plasma supernatant was collected and frozen at −80° C. Plasma phosphate was analyzed using a colorimetric phosphate assay kit (AbCam. UK, Catalog No. ab65622).

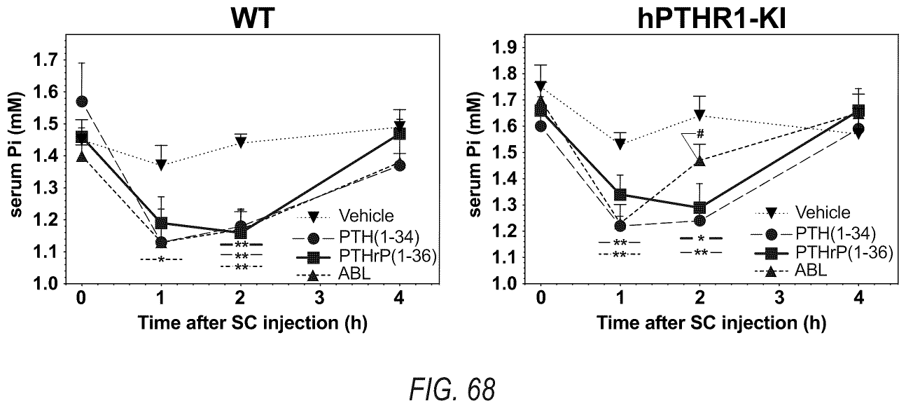

Ligand-induced phosphaturic responses were assessed in the hPTH1R-KI and WT mice by measuring time-dependent changes in blood inorganic phosphorus (Pi) after ligand injection. As with the calcemic responses, the phosphaturic responses induced by all three peptides in WT mice were highly comparable, as each resulted in a significant decrease in serum Pi at 1-2 hours post-injection and a recovery to baseline levels by 4 hours. FIG. 68. Injection of the peptides into hPTH1R-KI mice, however, again resulted in a discernable difference in the duration of the responses induced by the test ligands. FIG. 68. All three peptides reduced serum Pi to similar levels at one-hour post-injection in the hPTH1R-KI mice, but while the Pi levels in the mice injected with PTH(1-34) remained low at 2-hours post-injection, they had returned to near-vehicle control levels by 2 hours in the KI mice injected with abaloparatide. FIG. 68.

The phosphaturic response induced by PTHrP(1-36) was similar to that induced by PTH(1-34) in this assay. The phosphaturic response profiles observed for PTH(1-34) and abaloparatide in the KI mice mirror the calcemic response profiles obtained for these two peptides in the hPTH1R-KI mice, as they again demonstrate a more prolonged activity in vivo for PTH(1-34) as compared to abaloparatide, which was not apparent in the WT mice. The differences in the durations of the responses induced by the two ligands in the KI mice are not likely due to variations in pharmacokinetic properties of the two ligands, as previous analyses have shown comparable rates of clearance from the blood for PTH(1-34) and abaloparatide, and indeed these rates are not expected to differ between the KI and WT mice used here.

### Example 15. Antagonist Responses in hPTH1R-KI and WT Mice

PTH and PTHrP agonist peptides having a deletion of their first six amino acid residues results in the peptides function switching to one that can act as a competitive antagonist and/or an inverse agonist; thus, these N-terminus-truncated peptides have potential therapeutic utility towards diseases caused by PTH1R hyperactivation. Assessing the efficacy of such a PTH antagonist peptides in vivo, however, can be difficult: due at least in part to a relatively low binding affinity of N-terminus- truncated PTH peptides, relative to the intact peptide, along with a more rapid rate of clearance of the truncated peptide from circulation.

In view of the foregoing examples demonstrating the capacity of hPTH1R-KI mice to better distinguish between PTH and PTHrP agonist analogs via the duration of the ligand-induced calcemic response (relative to WT mice), the hPTH1R-KI mice of the present disclosure were evaluated to determine whether they could likewise be used to assess responses to PTH1R antagonist peptides in vivo

Two test antagonist peptides were evaluated: (1) LA-PTH(7-36), and (2) and [Leu¹¹,dTrp¹²,Trp²³,Tyr³⁶]-PTHrP(7-36).

LA-PTH(7-36) is an N-terminus-truncated variant of the long-acting PTH(1-14)/PTHrP(15-36) hybrid peptide (called LA-PTH), which forms highly stable complexes with PTH1R in vitro, thereby inducing prolonged calcemic responses in vivo (see Zhao et al., Structure and dynamics of the active human parathyroid hormone receptor-1. Science. 2019; 364(6436):148-153; Maeda et al., Critical role of parathyroid hormone (PTH) receptor-1 phosphorylation in regulating acute responses to PTH. PNAS. 2013; 110(15):5864-5869; Shimizu et al., Pharmacodynamic Actions of a Long-Acting PTH Analog (LA-PTH) in Thyroparathyroidectomized (TPTX) Rats and Normal Monkeys. J Bone Miner Res. 2016; 7:1405-1412).

The antagonist “[Leu” ¹¹,dTrp¹²,Trp²³,Tyr³⁶]-PTHrP(7-36)” is a PTHrP(7-36)-derived antagonist that also functions as an inverse agonist on constitutively active mutant PTH receptors.

Briefly, to assess the responses to antagonist PTH analogs, 3-month-old WT or hPTH1R-KI mice were injected subcutaneously with either (1) vehicle, (2) vehicle containing PTH(1-34) alone at 40 nmol/kg, or (3) PTH(1-34) at 40 nmol/kg together with an antagonist peptide, LA-PTH(7-36) or [Leu¹¹,dTrp¹²,Trp²³,Tyr³⁶]-PTHrP(7-36), each antagonist at a dose of 500 nmol/kg (n=5 mice per group). Blood was collected from the tail vein and calcium was measured as described above in Example 13.

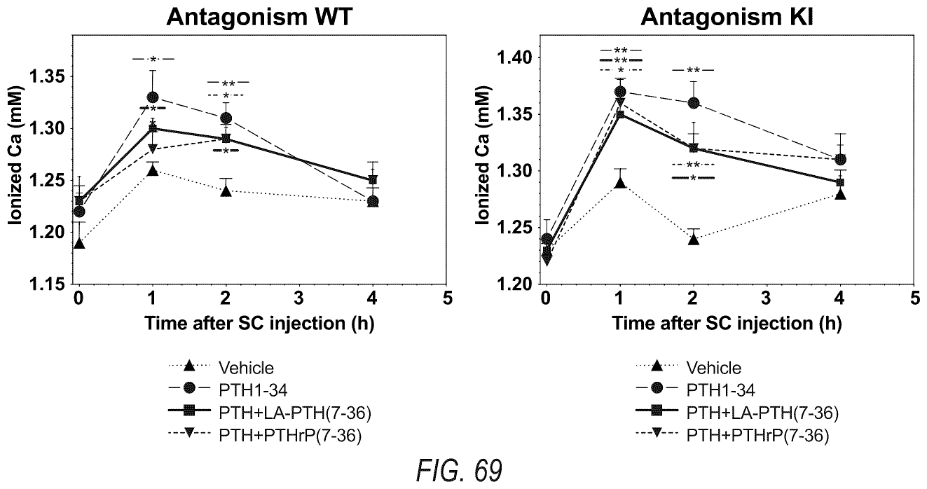

In both WT and hPTH1R-KI mice, injection of PTH(1-34) alone induced the expected rise in blood calcium levels, and these responses were blunted but only marginally by co-injection with either antagonist peptide. FIG. 69. Although differences were not significant, at two hours post injection, the blood calcium levels in the hPTH1R-KI mice co-injected with either antagonist tended to be lower than those in the hPTH1R-KI mice injected with the agonist alone. In the WT mice, the blood calcium levels at two hours appeared similar for the agonist-injected and antagonist-co-injected groups. While this data does not reveal a marked improvement in the capacity of the hPTH1R-KI mice to report efficacy of such N-terminus-truncated antagonist peptides, it does suggest an alternative and/or complementary path for assessing such competitive antagonist ligands for potential efficacy on the human PTH1R in vivo.

1. A transgenic non-human animal comprising a heterologous polynucleotide comprising human Parathyroid Hormone 1 Receptor (hPTH1R) exons 4 to 16; wherein said heterologous polynucleotide is operable to encode a human PTH1R protein.

2. The transgenic non-human animal of claim 1, wherein the non-human animal is a mammal.

3. The transgenic non-human animal of claim 2, wherein the mammal is selected from the group consisting of: a mouse; a rat; a guinea pig; a hamster; and a gerbil.

4. The transgenic non-human animal of claim 3, wherein the mammal is a mouse.

5. The transgenic non-human animal of claim 4, wherein the mouse is: a 129 mouse; an A mouse; a BALB/c mouse; a C3H mouse; a C57BL mouse; a C57BR mouse; a C57L mouse; a CB17 mouse; a CD-1 mouse; a DBA mouse; an FVB mouse; an SJL mouse; an SWR mouse; any substrain thereof; any hybrid strain thereof; any congenic strain thereof; or any mutant strain thereof.

6. The transgenic non-human animal of claim 4, wherein the mouse is: a C57BL/6 mouse, or a C57BL/10 mouse.

7. The transgenic non-human animal of claim 6, wherein the mouse is a C57BL/6 mouse.

8. The transgenic non-human animal of claim 1, wherein the heterologous polynucleotide comprising human PTH1R exons 4 to 16 is operable to encode a polypeptide having an amino acid sequence that is at least 80% identical, at least 85% identical, at least 90% identical, at least 95% identical, at least 96% identical, at least 97% identical, at least 98% identical, at least 99% identical, or 100% identical to an amino acid sequence as set forth in SEQ ID NO: 1.

9. The transgenic non-human animal of claim 1, wherein the heterologous polynucleotide comprising human PTH1R exons 4 to 16 is operable to encode a polypeptide having an amino acid sequence that is at least 95% identical to an amino acid sequence as set forth in SEQ ID NO: 1.

10. The transgenic non-human animal of claim 1, wherein the heterologous polynucleotide comprising human PTH1R exons 4 to 16 is operable to encode a polypeptide having an amino acid sequence that is at least 80% identical, at least 85% identical, at least 90% identical, at least 95% identical, at least 96% identical, at least 97% identical, at least 98% identical, at least 99% identical, or 100% identical to an amino acid sequence as set forth in SEQ ID NO: 29.

11. The transgenic non-human animal of claim 1, wherein the heterologous polynucleotide comprising human PTH1R exons 4 to 16 is operable to encode a polypeptide having an amino acid sequence that is at least 95% identical to an amino acid sequence as set forth in SEQ ID NO: 29.

12. The transgenic non-human animal of claim 1, wherein the heterologous polynucleotide comprising human PTH1R exons 4 to 16 is stably integrated in the genome of the non-human animal.

13. The transgenic non-human animal of claim 12, wherein the heterologous polynucleotide comprising human PTH1R exons 4 to 16 is stably integrated in an endogenous non-human animal PTH1R gene locus.

14. The transgenic non-human animal of claim 13, wherein the heterologous polynucleotide comprising human PTH1R exons 4 to 16 is stably integrated in an endogenous non-human animal PTH1R gene locus that causes a replacement of a genomic DNA segment comprising non-human animal PTH1R exon 4, with the heterologous polynucleotide comprising human PTH1R exons 4 to 16.

15. The transgenic non-human animal of claim 14, wherein the replacement results in a heterozygous transgenic non-human animal, or a homozygous transgenic non-human animal.

16. A non-human recombinant cell comprising: a heterologous polynucleotide comprising human Parathyroid Hormone 1 Receptor (hPTH1R) exons 4 to 16, wherein said heterologous polynucleotide is operable to encode a human PTH1R protein.

17. The non-human recombinant cell of claim 16, wherein the non-human recombinant cell is a mammalian recombinant cell.

18. The non-human recombinant cell of claim 17, wherein the mammalian recombinant cell is selected from the group consisting of: a mouse; a rat; a guinea pig; a hamster; and a gerbil.

19. The non-human recombinant cell of claim 18, wherein the mammalian recombinant cell is a mouse recombinant cell.

20. The non-human recombinant cell of claim 19, wherein the mouse recombinant cell is: a 129 recombinant cell; an A recombinant cell; a BALB/c recombinant cell; a C3H recombinant cell; a C57BL recombinant cell; a C57BR recombinant cell; a C57L recombinant cell; a CB17 recombinant cell; a CD-1 recombinant cell; a DBA recombinant cell; an FVB recombinant cell; an SJL recombinant cell; an SWR recombinant cell; a cell from any substrain thereof; a cell from any hybrid strain thereof; a cell from any congenic strain thereof; or a cell from any mutant strain thereof.

21. The non-human recombinant cell of claim 20, wherein the mouse recombinant cell is a C57BL/6 mouse recombinant cell, or a C57BL/10 mouse recombinant cell.

22. The non-human recombinant cell of claim 21, wherein the mouse recombinant cell is a C57BL/6 mouse recombinant cell.

23. The non-human recombinant cell of claim 16, wherein the heterologous polynucleotide comprising human PTH1R exons 4 to 16 is operable to encode a polypeptide having an amino acid sequence that is at least 80% identical, at least 85% identical, at least 90% identical, at least 95% identical, at least 96% identical, at least 97% identical, at least 98% identical, at least 99% identical, or 100% identical to an amino acid sequence as set forth in SEQ ID NO: 1.

24. The non-human recombinant cell of claim 16, wherein the heterologous polynucleotide comprising human PTH1R exons 4 to 16 is operable to encode a polypeptide having an amino acid sequence that is at least 95% identical to an amino acid sequence as set forth in SEQ ID NO: 1.

25. The non-human recombinant cell of claim 16, wherein the heterologous polynucleotide comprising human PTH1R exons 4 to 16 is operable to encode a polypeptide having an amino acid sequence that is at least 80% identical, at least 85% identical, at least 90% identical, at least 95% identical, at least 96% identical, at least 97% identical, at least 98% identical, at least 99% identical, or 100% identical to an amino acid sequence as set forth in SEQ ID NO: 29.

26. The non-human recombinant cell of claim 16, wherein the heterologous polynucleotide comprising human PTH1R exons 4 to 16 is operable to encode a polypeptide having an amino acid sequence that at least 95% identical to an amino acid sequence as set forth in SEQ ID NO: 29.

27. The non-human recombinant cell claim 16, wherein the heterologous polynucleotide comprising human PTH1R exons 4 to 16 is stably integrated in the genome of the non-human recombinant cell.

28. The non-human recombinant cell of claim 27, wherein the heterologous polynucleotide comprising human PTH1R exons 4 to 16 is stably integrated in an endogenous non-human animal PTH1R gene locus.

29. The non-human recombinant cell of claim 28, wherein the heterologous polynucleotide comprising human PTH1R exons 4 to 16 is stably integrated in an endogenous non-human animal PTH1R gene locus that causes a replacement of a genomic DNA segment comprising non-human animal PTH1R exon 4, with the heterologous polynucleotide comprising human PTH1R exons 4 to 16.

30. The non-human recombinant cell of claim 29, wherein the replacement results in a heterozygous recombinant cell, or a homozygous recombinant cell.

31. A vector comprising:
(i) a heterologous polynucleotide comprising a first nucleotide sequence comprising a coding sequence for human Parathyroid Hormone 1 Receptor (hPTH1R) exons 4 to 16 and second nucleotide sequence comprising a polyadenylation signal;
(ii) a 5′-homology arm, and a 3′-homology arm, wherein said 5′-homology arm and said 3′-homology arm are located upstream and downstream of the heterologous polynucleotide, respectively;
(iii) a third nucleotide sequence operable to encode a diphtheria toxin A protein, or fragment thereof; and a fourth nucleotide sequence operable to encode an neomycin phosphotransferase II (Neo);
(iv) an upstream self-deletion anchor (SDA) nucleotide sequence, and a downstream SDA nucleotide sequence; wherein said upstream SDA nucleotide sequence and downstream SDA nucleotide sequences flank the fourth nucleotide sequence;
wherein said vector is operable to allow a homologous recombination-mediated integration of the heterologous polynucleotide into an endogenous non-human animal PTH1R gene locus;
wherein said homologous recombination-mediated integration results in a replacement of an endogenous non-human animal genomic DNA segment with the heterologous polynucleotide.

32. A method of making a transgenic non-human animal comprising:
(i) introducing a heterologous polynucleotide comprising human PTH1R exons 4 to 16 into a non-human animal embryonic stem (ES) cell, such that the heterologous polynucleotide integrates into an endogenous non-human animal PTH1R locus;
(ii) obtaining a non-human animal ES cell comprising a modified genome, wherein the heterologous polynucleotide has integrated into an endogenous non-human animal PTH1R locus; and
(iii) generating a non-human animal using the non-human animal ES cell comprising the modified genome.

33. The method of claim 32, wherein the non-human animal is a mammal.

34. The method of claim 33, wherein the mammal is selected from the group consisting of: a mouse; a rat; a guinea pig; a hamster; and a gerbil.

35. The method of claim 34, wherein the mammal is a mouse.

36. The method of claim 35, wherein the mouse is: a 129 mouse; an A mouse; a BALB/c mouse; a C3H mouse; a C57BL mouse; a C57BR mouse; a C57L mouse; a CB17 mouse; a CD-1 mouse; a DBA mouse; an FVB mouse; an SJL mouse; an SWR mouse; any substrain thereof; any hybrid strain thereof; any congenic strain thereof; or any mutant strain thereof.

37. The method of claim 36, wherein the mouse is: a C57BL/6 mouse, or a C57BL/10 mouse.

38. The method of claim 37, wherein the mouse is a C57BL/6 mouse.

39. The method of claim 32, wherein the heterologous polynucleotide comprising human PTH1R exons 4 to 16 is operable to encode a human PTH1R protein having an amino acid sequence that is at least 80% identical, at least 85% identical, at least 90% identical, at least 95% identical, at least 96% identical, at least 97% identical, at least 98% identical, at least 99% identical, or 100% identical to an amino acid sequence as set forth in SEQ ID NO: 1.

40. The method of claim 32, wherein the heterologous polynucleotide comprising human PTH1R exons 4 to 16 is operable to encode a human PTH1R protein having an amino acid sequence that is at least 95% identical to an amino acid sequence as set forth in SEQ ID NO: 1.

41. The method of claim 32, wherein the heterologous polynucleotide comprising human PTH1R exons 4 to 16 is operable to encode a human PTH1R protein having an amino acid sequence that is at least 80% identical, at least 85% identical, at least 90% identical, at least 95% identical, at least 96% identical, at least 97% identical, at least 98% identical, at least 99% identical, or 100% identical to an amino acid sequence as set forth in SEQ ID NO: 29.

42. The method of claim 32, wherein the heterologous polynucleotide comprising human PTH1R exons 4 to 16 is operable to encode a human PTH1R protein having an amino acid sequence that is at least 95% identical to an amino acid sequence as set forth in SEQ ID NO: 29.

43. The method of claim 32, wherein the heterologous polynucleotide comprising human PTH1R exons 4 to 16 is stably integrated in the genome of the transgenic non-human animal.

44. The method of claim 43, wherein the heterologous polynucleotide comprising human PTH1R exons 4 to 16 is stably integrated in an endogenous non-human animal PTH1R gene locus.

45. The method of claim 44, wherein the heterologous polynucleotide comprising human PTH1R exons 4 to 16 is stably integrated in an endogenous non-human animal PTH1R gene locus that causes a replacement of a genomic DNA segment comprising non-human animal PTH1R exon 4, with the heterologous polynucleotide comprising human PTH1R exons 4 to 16.

46. The method of claim 45, wherein the replacement results in a heterozygous transgenic non-human animal, or a homozygous transgenic non-human animal.

47. An assay to identify a candidate agent that modulates the activity or function of a human PTH1R protein (hPTH1R), comprising:
(a) obtaining an experimental animal or a cell therefrom;
wherein said experimental animal is a transgenic non-human animal having a heterologous polynucleotide comprising human PTH1R exons 4 to 16 that is operable to encode a hPTH1R; and
wherein said experimental animal or a cell therefrom is operable to express the hPTH1R;
(b) admixing the candidate agent with the hPTH1R present in the experimental animal or cell therefrom;
(c) measuring whether said candidate agent modulates the activity or function of said hPTH1R,
wherein a modulation in the activity or function of said hPTH1R in the presence of said candidate agent, as compared to the activity or function of said hPTH1R that is not exposed said candidate agent, is indicative that said candidate agent modulates the activity or function of said hPTH1R.

48. The assay of claim 47, wherein the modulation in the activity or function of the hPTH1R is determined based on a change in the level of one or more of the following:
(i) transcription of one or more of the following genes, or promoters thereof: cyclin D1; cyclin A; CREB; E2F transcription factors; or E2F-dependent genes;
(ii) phosphorylation of CREB;
(iii) one or more proliferating cells;
(iv) binding of a parathyroid hormone (PTH), a parathyroid hormone-related peptide (PTHrP); or a fragment thereof;
(v) cyclic AMP (cAMP) accumulation;
(vi) intracellular free calcium; or
(vii) inositol phosphate metabolism.

49. The assay of claim 47, wherein the control animal and the experimental animal are the same type of an animal, wherein said animal is a mammal.

50. The assay of claim 49, wherein the mammal is selected from the group consisting of: a mouse; a rat; a guinea pig; a hamster; and a gerbil.

51. The assay of claim 50, wherein the mammal is a mouse.

52. The assay of claim 51, wherein the mouse is: a 129 mouse; an A mouse; a BALB/c mouse; a C3H mouse; a C57BL mouse; a C57BR mouse; a C57L mouse; a CB17 mouse; a CD-1 mouse; a DBA mouse; an FVB mouse; an SJL mouse; an SWR mouse; any substrain thereof; any hybrid strain thereof; any congenic strain thereof; or any mutant strain thereof.

53. The assay of claim 52, wherein the mouse is: a C57BL/6 mouse, or a C57BL/10 mouse.

54. The assay of claim 53, wherein the mouse is a C57BL/6 mouse.

55. The assay of claim 47, wherein the heterologous polynucleotide comprising human PTH1R exons 4 to 16 is operable to encode a polypeptide having an amino acid sequence that is at least 80% identical, at least 85% identical, at least 90% identical, at least 95% identical, at least 96% identical, at least 97% identical, at least 98% identical, at least 99% identical, or 100% identical to an amino acid sequence as set forth in SEQ ID NO: 1.

56. The assay of claim 47, wherein the heterologous polynucleotide comprising human PTH1R exons 4 to 16 is operable to encode a polypeptide having an amino acid sequence that is at least 95% identical to an amino acid sequence as set forth in SEQ ID NO: 1.

57. The assay of claim 47, wherein the heterologous polynucleotide comprising human PTH1R exons 4 to 16 is operable to encode a polypeptide having an amino acid sequence that is at least 80% identical, at least 85% identical, at least 90% identical, at least 95% identical, at least 96% identical, at least 97% identical, at least 98% identical, at least 99% identical, or 100% identical to an amino acid sequence as set forth in SEQ ID NO: 29.

58. The assay of claim 47, wherein the heterologous polynucleotide comprising human PTH1R exons 4 to 16 is operable to encode a polypeptide having an amino acid sequence that is at least 95% identical to an amino acid sequence as set forth in SEQ ID NO: 29.

59. The assay of claim 47, wherein the heterologous polynucleotide comprising human PTH1R exons 4 to 16 is stably integrated in the genome of the transgenic non-human animal, or a cell therefrom.

60. The assay of claim 59, wherein the heterologous polynucleotide comprising human PTH1R exons 4 to 16 is stably integrated in an endogenous non-human animal PTH1R gene locus.

61. The assay of claim 60, wherein the heterologous polynucleotide comprising human PTH1R exons 4 to 16 is stably integrated in an endogenous non-human animal PTH1R gene locus that causes a replacement of a genomic DNA segment comprising a non-human animal PTH1R exon 4, with the heterologous polynucleotide comprising human PTH1R exons 4 to 16.

62. The assay of claim 61, wherein the replacement results in a heterozygous transgenic non-human animal, or a homozygous transgenic non-human animal.

63. The assay of claim 47, further comprising a control animal or cell therefrom.

64. The assay of claim 63, wherein a control agent is administered to the control animal or cell therefrom.

65. The assay of claim 64, wherein the modulation in the activity or function of said hPTH1R in the experimental animal or cell therefrom in the presence of said candidate agent, as compared to the activity or function of said hPTH1R in the control animal or cell therefrom in the presence of the control agent, is indicative that said candidate agent modulates the activity or function of said hPTH1R.

66. The transgenic non-human animal of any one of claims 1-15, wherein the human PTH1R protein further comprises a human influenza hemagglutinin (HA) epitope tag.

67. The non-human recombinant cell of any one of claims 16-30, wherein the human PTH1R protein further comprises a human influenza hemagglutinin (HA) epitope tag.

68. The method of any one of claims 32-46, wherein the hPTH1R protein further comprises a human influenza hemagglutinin (HA) epitope tag.

69. The assay of any one of claims 47-65, wherein the hPTH1R further comprises a human influenza hemagglutinin (HA) epitope tag.

70. A transgenic mouse comprising a heterologous polynucleotide comprising human Parathyroid Hormone 1 Receptor (hPTH1R) exons 4 to 16; wherein said heterologous polynucleotide is operable to encode a human PTH1R protein having an amino acid sequence as set forth in SEQ ID NO: 1.

71. A transgenic mouse comprising a heterologous polynucleotide comprising human Parathyroid Hormone 1 Receptor (hPTH1R) exons 4 to 16; wherein said heterologous polynucleotide is operable to encode a human PTH1R protein having an amino acid sequence as set forth in SEQ ID NO: 29.

72. A non-human recombinant cell comprising: a heterologous polynucleotide comprising human Parathyroid Hormone 1 Receptor (hPTH1R) exons 4 to 16, wherein said heterologous polynucleotide is operable to encode a human PTH1R protein having an amino acid sequence as set forth in SEQ ID NO: 1.

73. A non-human recombinant cell comprising: a heterologous polynucleotide comprising human Parathyroid Hormone 1 Receptor (hPTH1R) exons 4 to 16, wherein said heterologous polynucleotide is operable to encode a human PTH1R protein having an amino acid sequence as set forth in SEQ ID NO: 29.


SEQUENCE LISTING (46 sequences)

SEQ ID NO: 1 - protein, 568 aa, Artificial sequence
  human PTH1R protein (exons 4-16)
VDADDVMTKEEQIFLLHRAQAQCEKRLKEVLQRPASIMESDKGWTSASTSGKPRKDKASGKLYPESEEDKEAPTGSRYRGRPCLPEWDHILCWPLGAPGEVVAVPCPDYIYDFNHKGHAYRRCDRNGSWELVPGHNRTWANYSECVKFLTNETREREVFDRLGMIYTVGYSVSLASLTVAVLILAYFRRLHCTRNYIHMHLFLSFMLRAVSIFVKDAVLYSGATLDEAERLTEEELRAIAQAPPPPATAAAGYAGCRVAVTFFLYFLATNYYWILVEGLYLHSLIFMAFFSEKKYLWGFTVFGWGLPAVFVAVWVSVRATLANTGCWDLSSGNKKWIIQVPILASIVLNFILFINIVRVLATKLRETNAGRCDTRQQYRKLLKSTLVLMPLFGVHYIVFMATPYTEVSGTLWQVQMHYEMLFNSFQGFFVAIIYCFCNGEVQAEIKKSWSRWTLALDFKRKARSGSSSYSYGPMVSHTSVTNVGPRVGLGLPLSPRLLPTATTNGHPQLPGHAKPGTPALETLETTPPAMAAPKDDGFLNGSCSGLDEEASGPERPPALLQEEWETVM

SEQ ID NO: 2 - protein, 9 aa, Artificial sequence
  human influenza hemagglutinin(HA)epitope tag
YPYDVPDYA

SEQ ID NO: 3 - protein, 9 aa, Artificial sequence
  human residues 88-96
YPESEEDKE

SEQ ID NO: 4 - DNA, 1707 bp, Artificial sequence
  Human PTH1R exons 4˜16 coding sequence-HA
gtggatgcagatgacgtcatgactaaagaggaacagatcttcctgctgcaccgtgctcaggcccagtgcgaaaaacggctcaaggaggtcctgcagaggccagccagcataatggaatcagacaagggatggacatctgcgtccacatcagggaagcccaggaaagataaggcatctgggaagctctacccttacgatgttccggactacgcggcacccactggcagcaggtaccgagggcgcccctgtctgccggaatgggaccacatcctgtgctggccgctgggggcaccaggtgaggtggtggctgtgccctgtccggactacatttatgacttcaatcacaaaggccatgcctaccgacgctgtgaccgcaatggcagctgggagctggtgcctgggcacaacaggacgtgggccaactacagcgagtgtgtcaaatttctcaccaatgagactcgtgaacgggaggtgtttgaccgcctgggcatgatttacaccgtgggctactccgtgtccctggcgtccctcaccgtagctgtgctcatcctggcctactttaggcggctgcactgcacgcgcaactacatccacatgcacctgttcctgtccttcatgctgcgcgccgtgagcatcttcgtcaaggacgctgtgctctactctggcgccacgcttgatgaggctgagcgcctcaccgaggaggagctgcgcgccatcgcccaggcgcccccgccgcctgccaccgccgctgccggctacgcgggctgcagggtggctgtgaccttcttcctttacttcctggccaccaactactactggattctggtggaggggctgtacctgcacagcctcatcttcatggccttcttctcagagaagaagtacctgtggggcttcacagtcttcggctggggtctgcccgctgtcttcgtggctgtgtgggtcagtgtcagagctaccctggccaacaccgggtgctgggacttgagctccgggaacaaaaagtggatcatccaggtgcccatcctggcctccattgtgctcaacttcatcctcttcatcaatatcgtccgggtgctcgccaccaagctgcgggagaccaacgccggccggtgtgacacacggcagcagtaccggaagctgctcaaatccacgctggtgctcatgcccctctttggcgtccactacattgtcttcatggccacaccatacaccgaggtctcagggacgctctggcaagtccagatgcactatgagatgctcttcaactccttccagggattttttgtcgcaatcatatactgtttctgcaatggcgaggtacaagctgagatcaagaaatcttggagccgctggacactggcactggacttcaagcgaaaggcacgcagcgggagcagcagctatagctacggccccatggtgtcccacacaagtgtgaccaatgtcggcccccgtgtgggactcggcctgcccctcagcccccgcctactgcccactgccaccaccaacggccaccctcagctgcctggccatgccaagccagggaccccagccctggagaccctcgagaccacaccacctgccatggctgctcccaaggacgatgggttcctcaacggctcctgctcaggcctggacgaggaggcctctgggcctgagcggccacctgccctgctacaggaagagtgggagacagtcatgtga

SEQ ID NO: 5 - DNA, 2238 bp, Mus musculus
attaatttttcttttcaagcccgagctgggacttaggccgtttcctgtcccccaccagctgtgtccttgctcaggcctgaattggagacacctctctgaagttccatggcctccccatggccaacctgagtcagctcctcaacccgaggccccgtttgaaaggcctgtggattccagatgagaagactgagaccaggataccgggagagtcctagtgggcaggacacacaattgggccggcggcggctgccccgagggacgcggccctaggcggtggcgatggggaccgcccggatcgcacccagcctggcgctccttctctgctgcccagtgctcagctccgcatatgcgctggtggacgcagacgatgtctttaccaaagaggaacagattttcctgctgcaccgtgcccaggcgcaatgtgacaagctgctcaaggaagttctgcacacagcagccaacataatggagtcagacaaagggtggacgccagcatctacgtcagggaagcccaggaaagagaaggcaccgggaaagttctaccccgagtctaaagagaacaaggatgtgcccaccggcagcaggcgccgagggcgtccctgtctgccagagtgggacaacatcgtttgctggccattgggggcaccaggtgaagtggtggcagtaccttgtcccgattacatttatgacttcaaccacaaaggccatgcctacagacgctgcgaccgcaatggcagctgggaggtggttccagggcacaaccggacgtgggccaactacagcgagtgcctcaagttcatgaccaatgagactcgggaacgggaggtatttgaccgcctgggcatgatctacaccgtgggatattccatgtctcttgcctccctcaccgtggctgtgctcatcctagcctattttaggcggctgcactgcacgcgcaactacatccacatgcacatgttcctgtcgtttatgctgcgcgccgcgagcatcttcgtgaaggacgctgtgctctactctggcttcacgctggatgaggccgagcgcctcacggaggaagagttgcatatcatcgcgcaggtgccgcctccgcccgccgctgccgccgttggctacgctggctgccgtgtggccgtgaccttcttcctctacttcctggctaccaactactactggattctggtggagggactgtacttacacagcctcatcttcatggcctttttctcagagaagaagtatctgtggggcttcaccatctttggctggggtctgccggctgtcttcgtggctgtgtgggtcggtgtcagagcaaccttggccaacactgggtgctgggacctgagctctgggcacaagaagtggatcatccaggtgcccatcctggcatctgttgtgctcaacttcatcctctttatcaacatcatccgggtgcttgccactaagcttcgggagaccaatgcgggccggtgtgacaccaggcagcagtaccggaagctgctcaggtccacgttggtgcttgtgccactcttcggtgtccactacaccgtcttcatggccttgccgtacaccgaggtctcagggacactgtggcagatccagatgcactatgagatgctcttcaactccttccagggattttttgttgccatcatatactgtttctgcaatggtgaggtgcaggcagagattaggaagtcttggagccgctggacactggcattggacttcaagcgtaaagcacgaagtgggagtagcagctacagctatggcccaatggtgtcacacacgagtgtgaccaatgtgggcccccgtgcaggactcagccttccccttagcccccgcctgcttcctgccaccaccaatggccactcccagctgcctggccacgccaagccgggcgctccagccattgagaacgaaaccataccagttactatgacagttcccaaggacgacggcttccttaatggctcctgctcgggtctggatgaggaggcctctgggtctgcgcggccacctccattgttgcaggaagaatgggaaacagtcatgtgactgggcatcaaggggctggactgctggcatgggcagatggacagatggaccaagaagccagagtttggctggctgtctattcaggactggaccaggaagacaaaaggaaaagagaaaaaaaaaaaaaaaaagaagggagctgtgtgtggcttcctgtgttgtgctcaagaaaaaaaaaaaaaa

SEQ ID NO: 6 - DNA, 2153 bp, Homo sapien
agcactatccttgcttaggcctgagccacagaagctgctcagggactatccatggcctccccgtggccaacttgagtctgctctgcagctttaggcccgacttggaaggcccatgggctgcagatgaggaaactgaggtccagacagccgaagagtggtagtgtccaggacacacaactgggccggcggcggcggctgccccgagggacgcggccctaggcggtggcgatggggaccgcccggatcgcacccggcctggcgctcctgctctgctgccccgtgctcagctccgcgtacgcgctggtggatgcagatgacgtcatgactaaagaggaacagatcttcctgctgcaccgtgctcaggcccagtgcgaaaaacggctcaaggaggtcctgcagaggccagccagcataatggaatcagacaagggatggacatctgcgtccacatcagggaagcccaggaaagataaggcatctgggaagctctaccctgagtctgaggaggacaaggaggcacccactggcagcaggtaccgagggcgcccctgtctgccggaatgggaccacatcctgtgctggccgctgggggcaccaggtgaggtggtggctgtgccctgtccggactacatttatgacttcaatcacaaaggccatgcctaccgacgctgtgaccgcaatggcagctgggagctggtgcctgggcacaacaggacgtgggccaactacagcgagtgtgtcaaatttctcaccaatgagactcgtgaacgggaggtgtttgaccgcctgggcatgatttacaccgtgggctactccgtgtccctggcgtccctcaccgtagctgtgctcatcctggcctactttaggcggctgcactgcacgcgcaactacatccacatgcacctgttcctgtccttcatgctgcgcgccgtgagcatcttcgtcaaggacgctgtgctctactctggcgccacgcttgatgaggctgagcgcctcaccgaggaggagctgcgcgccatcgcccaggcgcccccgccgcctgccaccgccgctgccggctacgcgggctgcagggtggctgtgaccttcttcctttacttcctggccaccaactactactggattctggtggaggggctgtacctgcacagcctcatcttcatggccttcttctcagagaagaagtacctgtggggcttcacagtcttcggctggggtctgcccgctgtcttcgtggctgtgtgggtcagtgtcagagctaccctggccaacaccgggtgctgggacttgagctccgggaacaaaaagtggatcatccaggtgcccatcctggcctccattgtgctcaacttcatcctcttcatcaatatcgtccgggtgctcgccaccaagctgcgggagaccaacgccggccggtgtgacacacggcagcagtaccggaagctgctcaaatccacgctggtgctcatgcccctctttggcgtccactacattgtcttcatggccacaccatacaccgaggtctcagggacgctctggcaagtccagatgcactatgagatgctcttcaactccttccagggattttttgtcgcaatcatatactgtttctgcaatggcgaggtacaagctgagatcaagaaatcttggagccgctggacactggcactggacttcaagcgaaaggcacgcagcgggagcagcagctatagctacggccccatggtgtcccacacaagtgtgaccaatgtcggcccccgtgtgggactcggcctgcccctcagcccccgcctactgcccactgccaccaccaacggccaccctcagctgcctggccatgccaagccagggaccccagccctggagaccctcgagaccacaccacctgccatggctgctcccaaggacgatgggttcctcaacggctcctgctcaggcctggacgaggaggcctctgggcctgagcggccacctgccctgctacaggaagagtgggagacagtcatgtgaccaggcgctgggggctggacctgctgacatagtggatggacagatggaccaaaagatgggtggttgaatgatttcccactcagggctggggccaagaggaaaaacagggaaaaaaagaaaaaaaaaagaaaaaggaaaaggaa

SEQ ID NO: 7 - DNA, 27 bp, Homo sapien
tacccttacgatgttccggactacgcg

SEQ ID NO: 8 - DNA, 23 bp, Artificial sequence
  F4
gactccccacattctctctgaag

SEQ ID NO: 9 - DNA, 21 bp, Artificial sequence
  R2
gcgtagtccggaacatcgtaa

SEQ ID NO: 10 - DNA, 21 bp, Artificial sequence
  F2
gatccttaccttccgggactc

SEQ ID NO: 11 - DNA, 24 bp, Artificial sequence
  R3
agttctagggatgctggttctatg

SEQ ID NO: 12 - DNA, 25 bp, Artificial sequence
  F3
catagaaaagccttgacttgaggtt

SEQ ID NO: 13 - DNA, 23 bp, Artificial sequence
  R1
tctctttaaggaagttggcccag

SEQ ID NO: 14 - DNA, 23 bp, Artificial sequence
  F4
gactccccacattctctctgaag

SEQ ID NO: 15 - DNA, 21 bp, Artificial sequence
  R2
gcgtagtccggaacatcgtaa

SEQ ID NO: 16 - DNA, 20 bp, Artificial sequence
  F1
caggcgatccttaccttccg

SEQ ID NO: 17 - DNA, 23 bp, Artificial sequence
  R1
tctctttaaggaagttggcccag

SEQ ID NO: 18 - DNA, 25 bp, Artificial sequence
  F3
catagaaaagccttgacttgaggtt

SEQ ID NO: 19 - protein, 115 aa, Homo sapien
MIPAKDMAKVMIVMLAICFLTKSDGKSVKKRSVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 20 - protein, 34 aa, Homo sapien
SVSEIQLMHNLGKHLNSMERVEWLRKKLQDVHNF

SEQ ID NO: 21 - protein, 36 aa, Homo sapien
AVSEHQLLHDKGKSIQDLRRRFFLHHLIAEIHTAEI

SEQ ID NO: 22 - protein, 34 aa, Artificial sequence
  Abaloparatide
  MISC_FEATURE (29)..(29): Xaa can be any naturally occurring amino acid
AVSEHQLLHDKGKSIQDLRRRELLEKLLXKLHTA

SEQ ID NO: 23 - DNA, 346 bp, Artificial sequence
  3 Junction consensus sequence
tgaggttagattttttttatattttgttttgtgttatttttttctttaacatccctaaaattttccttacatgttttactagccagatttttcctcctctcctgactactcccagtcatagctgtccctcttctcttatggagatcctggagggacctaataacttcgtatagcatacattatacgaagttatattaagggttattgaatatgatcggaattgggctgcaggaattcgatagcttggctgcaggtcgacgtacgtagcaagcttgatgggccctggtaccccggggtcccagtggatttagatggggttgggcaagccagggactttgctgagggc

SEQ ID NO: 24 - DNA, 25 bp, Artificial sequence
  R-291 reverse primer
aggaagtaaaggaagaaggtcacag

SEQ ID NO: 25 - DNA, 344 bp, Artificial sequence
  Consensus.F3_R-291 Sequence.vers 3
gccgctgggggcaccaggtgaggtggtggctgtgccctgtccggactacatttatgacttcaatcacaaaggccatgcctaccgacgctgtgaccgcaatggcagctgggagctggtgcctgggcacaacaggacgtgggccaactacagcgagtgtgtcaaatttctcaccaatgagactcgtgaacgggaggtgtttgaccgcctgggcatgatttacaccgtgggctactccgtgtccctggcgtccctcaccgtagctgtgctcatcctggcctactttagcggctgcactgcacgcgcaactacatccacatgcacctgttcctgtccttcatgctgcg

SEQ ID NO: 26 - protein, 22 aa, Homo sapien
MGTARIAPGLALLLCCPVLSSA

SEQ ID NO: 27 - DNA, 2153 bp, Homo sapien; identical to SEQ ID NO: 6

SEQ ID NO: 28 - protein, 593 aa, Homo sapien
MGTARIAPGLALLLCCPVLSSAYALVDADDVMTKEEQIFLLHRAQAQCEKRLKEVLQRPASIMESDKGWTSASTSGKPRKDKASGKLYPYDVPDYAAPTGSRYRGRPCLPEWDHILCWPLGAPGEVVAVPCPDYIYDFNHKGHAYRRCDRNGSWELVPGHNRTWANYSECVKFLTNETREREVFDRLGMIYTVGYSVSLASLTVAVLILAYFRRLHCTRNYIHMHLFLSFMLRAVSIFVKDAVLYSGATLDEAERLTEEELRAIAQAPPPPATAAAGYAGCRVAVTFFLYFLATNYYWILVEGLYLHSLIFMAFFSEKKYLWGFTVFGWGLPAVFVAVWVSVRATLANTGCWDLSSGNKKWIIQVPILASIVLNFILFINIVRVLATKLRETNAGRCDTRQQYRKLLKSTLVLMPLFGVHYIVFMATPYTEVSGTLWQVQMHYEMLFNSFQGFFVAIIYCFCNGEVQAEIKKSWSRWTLALDFKRKARSGSSSYSYGPMVSHTSVTNVGPRVGLGLPLSPRLLPTATTNGHPQLPGHAKPGTPALETLETTPPAMAAPKDDGFLNGSCSGLDEEASGPERPPALLQEEWETVM

SEQ ID NO: 29 - protein, 568 aa, Artificial sequence
  human PTH1R protein (exons 4-16)-HA
VDADDVMTKEEQIFLLHRAQAQCEKRLKEVLQRPASIMESDKGWTSASTSGKPRKDKASGKLYPYDVPDYAAPTGSRYRGRPCLPEWDHILCWPLGAPGEVVAVPCPDYIYDFNHKGHAYRRCDRNGSWELVPGHNRTWANYSECVKFLTNETREREVFDRLGMIYTVGYSVSLASLTVAVLILAYFRRLHCTRNYIHMHLFLSFMLRAVSIFVKDAVLYSGATLDEAERLTEEELRAIAQAPPPPATAAAGYAGCRVAVTFFLYFLATNYYWILVEGLYLHSLIFMAFFSEKKYLWGFTVFGWGLPAVFVAVWVSVRATLANTGCWDLSSGNKKWIIQVPILASIVLNFILFINIVRVLATKLRETNAGRCDTRQQYRKLLKSTLVLMPLFGVHYIVFMATPYTEVSGTLWQVQMHYEMLFNSFQGFFVAIIYCFCNGEVQAEIKKSWSRWTLALDFKRKARSGSSSYSYGPMVSHTSVTNVGPRVGLGLPLSPRLLPTATTNGHPQLPGHAKPGTPALETLETTPPAMAAPKDDGFLNGSCSGLDEEASGPERPPALLQEEWETVM

SEQ ID NO: 30 - protein, 593 aa, Artificial sequence; identical to SEQ ID NO: 28
  HA-Tag hPTH1R

SEQ ID NO: 31 - protein, 25 aa, Artificial sequence
  Mouse PTH1R residues 1-25
MGTARIAPSLALLLCCPVLSSAYAL

SEQ ID NO: 32 - protein, 25 aa, Artificial sequence
  Human PTH1R residues 1-25
MGTARIAPGLALLLCCPVLSSAYAL

SEQ ID NO: 33 - DNA, 23 bp, Artificial sequence
  F3-Int3 (Fwd)
gactccccacattctctctgaag

SEQ ID NO: 34 - DNA, 23 bp, Artificial sequence
  F-HA86 (Fwd)
ggaagctctacccttacgatgtt

SEQ ID NO: 35 - DNA, 21 bp, Artificial sequence
  R2-HA95 (Rev)
gcgtagtccggaacatcgtaa

SEQ ID NO: 36 - DNA, 22 bp, Artificial sequence
  F-I255 (Fwd)
catcttcgtcaaggacgctgtg

SEQ ID NO: 37 - DNA, 25 bp, Artificial sequence
  R-291 (Rev)
aggaagtaaaggaagaaggtcacag

SEQ ID NO: 38 - DNA, 24 bp, Artificial sequence
  F-mid.ins (Fwd)
ggcgtccactacattgtcttcatg

SEQ ID NO: 39 - DNA, 24 bp, Artificial sequence
  R-mid.ins (Rev)
catgaagacaatgtagtggacgcc

SEQ ID NO: 40 - DNA, 21 bp, Artificial sequence
  F-M611 (Fwd)
gaagagtgggagacagtcatg

SEQ ID NO: 41 - DNA, 21 bp, Artificial sequence
  R-M611 (Rev)
catgactgtctcccactcttc

SEQ ID NO: 42 - DNA, 25 bp, Artificial sequence
  F2-rbg (Fwd)
catagaaaagccttgacttgaggtt

SEQ ID NO: 43 - DNA, 25 bp, Artificial sequence
  R-rbg (Rev)
aacctcaagtcaaggcttttctatg

SEQ ID NO: 44 - DNA, 23 bp, Artificial sequence
  R1.int4 (Rev)
tctctttaaggaagttggcccag

SEQ ID NO: 45 - DNA, 2600 bp, Artificial sequence
  Nucleotide Sequence of hP1R KI region
gactccccacattctctctgaagggatcacttttcgaaaggggggaaatccctggggaggttgcatgagtttggaaccagctgcctcacctggaagtgctgcctacagtctgacctttggtttggcaggtggatgcagatgacgtcatgactaaagaggaacagatcttcctgctgcaccgtgctcaggcccagtgcgaaaaacggctcaaggaggtcctgcagaggccagccagcataatggaatcagacaagggatggacatctgcgtccacatcagggaagcccaggaaagataaggcatctgggaagctctacccttacgatgttccggactacgcggcacccactggcagcaggtaccgagggcgcccctgtctgccggaatgggaccacatcctgtgctggccgctgggggcaccaggtgaggtggtggctgtgccctgtccggactacatttatgacttcaatcacaaaggccatgcctaccgacgctgtgaccgcaatggcagctgggagctggtgcctgggcacaacaggacgtgggccaactacagcgagtgtgtcaaatttctcaccaatgagactcgtgaacgggaggtgtttgaccgcctgggcatgatttacaccgtgggctactccgtgtccctggcgtccctcaccgtagctgtgctcatcctggcctactttaggcggctgcactgcacgcgcaactacatccacatgcacctgttcctgtccttcatgctgcgcgccgtgagcatcttcgtcaaggacgctgtgctctactctggcgccacgcttgatgaggctgagcgcctcaccgaggaggagctgcgcgccatcgcccaggcgcccccgccgcctgccaccgccgctgccggctacgcgggctgcagggtggctgtgaccttcttcctttacttcctggccaccaactactactggattctggtggaggggctgtacctgcacagcctcatcttcatggccttcttctcagagaagaagtacctgtggggcttcacagtcttcggctggggtctgcccgctgtcttcgtggctgtgtgggtcagtgtcagagctaccctggccaacaccgggtgctgggacttgagctccgggaacaaaaagtggatcatccaggtgcccatcctggcctccattgtgctcaacttcatcctcttcatcaatatcgtccgggtgctcgccaccaagctgcgggagaccaacgccggccggtgtgacacacggcagcagtaccggaagctgctcaaatccacgctggtgctcatgcccctctttggcgtccactacattgtcttcatggccacaccatacaccgaggtctcagggacgctctggcaagtccagatgcactatgagatgctcttcaactccttccagggattttttgtcgcaatcatatactgtttctgcaatggcgaggtacaagctgagatcaagaaatcttggagccgctggacactggcactggacttcaagcgaaaggcacgcagcgggagcagcagctatagctacggccccatggtgtcccacacaagtgtgaccaatgtcggcccccgtgtgggactcggcctgcccctcagcccccgcctactgcccactgccaccaccaacggccaccctcagctgcctggccatgccaagccagggaccccagccctggagaccctcgagaccacaccacctgccatggctgctcccaaggacgatgggttcctcaacggctcctgctcaggcctggacgaggaggcctctgggcctgagcggccacctgccctgctacaggaagagtgggagacagtcatgtgatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcctcgagggacctaataacttcgtatagcatacattatacgaagttatattaagggttattgaatatgatcggaattgggctgcaggaattcgatagcttggctgcaggtcgacgtacgtagcaagcttgatgggccctggtaccacggggtcccagtggatttagatggggttgggcaagccagggactttgctgagggcgctggtccaaacagggtgggctgggccaacttccttaaagaga

SEQ ID NO: 46 - DNA, 1707 bp, Artificial sequence; identical to SEQ ID NO: 4
  hPTHR1 cDNA encoding hPTH1R V26-M593